(19) United States
(12) Patent Application Publication
(10) Pub. No.: US 2026/0109708 A1
(43) Pub. Date: Apr. 23, 2026

(54) COMPOUNDS AND COMPOSITIONS USEFUL AS DEGRADERS OF MK2 KINASE

(71) Applicant: Celgene Corporation, Princeton, NJ (US)

(72) Inventors: Farid VAN DER MEI, Princeton, NJ (US); Guobin MIAO, Princeton, NJ (US); Lixin QIAO, Andover, MA (US)

(21) Appl. No.: 19/154,293

(22) PCT Filed: Feb. 6, 2024
(86) PCT No.: PCT/US2024/014652

§ 371 Date: Aug. 6, 2025

Related U.S. Application Data
(60) Provisional application No. 63/483,569, filed on Feb. 7, 2023.

(51) Int. Cl. C07D 495/14; A61K 31/551; A61K 31/553; C07D 498/14; C07D 519/00

(52) U.S. Cl. CPC C07D 495/14; A61K 31/551; A61K 31/553; C07D 498/14; C07D 519/00

(57) ABSTRACT

The present disclosure provides compounds and compositions useful for degrading MK2 kinase.

### CROSS-REFERENCE

This application claims the benefit of and priority to U.S. Provisional Patent Application No. 63/483,569 filed on Feb. 7, 2023, the entire contents of which are hereby incorporated by reference herein.

### INCORPORATION BY REFERENCE OF SEQUENCE LISTING

The present application is being filed with a Sequence Listing in electronic format. The Sequence Listing is provided as a file entitled 055920-574001WO_SeqList_ST26.xml, created on Jan. 30, 2024, and is 3 kilobytes in size. The information in electronic format of the Sequence Listing is incorporated herein by reference in its entirety.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

Protein degradation is a highly regulated and essential process that maintains cellular homeostasis. The selective identification and removal of damaged, misfolded, or excess proteins is achieved via the ubiquitin-proteasome pathway (UPP). The UPP is central to the regulation of almost all cellular processes, including antigen processing, apoptosis, biogenesis of organelles, cell cycling, DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection. Covalent attachment of multiple ubiquitin molecules by an E3 ubiquitin ligase to a terminal lysine residue marks the protein for proteasome degradation, where the protein is digested into small peptides and eventually into its constituent amino acids that serve as building blocks for new proteins. Defective proteasomal degradation has been linked to a variety of disorders including cancer and others.

Cereblon forms part of an E3 ubiquitin ligase complex which interacts with damaged DNA binding protein 1, forming an E3 ubiquitin ligase complex with Cullin 4 and the E2-binding protein ROC1 (known as RBX1) where it functions as a substrate receptor to select proteins for ubiquitination. The binding of lenalidomide to cereblon facilitates subsequent binding of cereblon to Ikaros and Aiolos, leading to their ubiquitination and degradation by the proteasome (see Lu, G. et al. “The myeloma drug lenalidomide promotes the cereblon-dependent destruction of Ikaros proteins” Science, 2014, 343:305-309; Krönke, J. et al. “Lenalidomide causes selective degradation of IKZF1 and IKZF3 in multiple myeloma cells” Science, 2014, 343:301-305).

Mitogen-activated protein kinase-activated protein kinase 2 (MAPKAP K2 or MK2) mediates multiple p38 MAPK-dependent cellular responses. MK2 (SEQ ID NO. 1) is an important intracellular regulator of the production of cytokines, such as tumor necrosis factor alpha (TNF-α), interleukin 6 (IL-6) and interferon gamma (IFNγ), that are involved in many acute and chronic inflammatory diseases, e.g., rheumatoid arthritis and inflammatory bowel disease. MK2 resides in the nucleus of non-stimulated cells and upon stimulation, it translocates to the cytoplasm and phosphorylates and activates tuberin and HSP27. MK2 is also implicated in heart failure, brain ischemic injury, the regulation of stress resistance and the production of TNF-α. (see Deak et al., EMBO. 17:4426-4441 (1998); Shi et al., Biol. Chem. 383:1519-1536 (2002); Staklatvala., Curr. Opin. Pharmacol. 4:372-377 (2004), and Shiroto et al., J. Mol. Cardiol. 38:93-97 (2005)).

SEQ ID NO. 1:
MLSNSQGQSPPVPFPAPAPPPQPPTPALPHPPAQPPPPPPQQFPQFHVKS
GLQIKKNAIIDDYKVTSQVLGLGINGKVLQIFNKRTQEKFALKMLQDCPK
ARREVELHWRASQCPHIVRIVDVYENLYAGRKCLLIVMECLDGGELFSRI
QDRGDQAFTEREASEIMKSIGEAIQYLHSINIAHRDVKPENLLYTSKRPN
AILKLTDFGFAKETTSHNSLTTPCYTPYYVAPEVLGPEKYDKSCDMWSLG
VIMYILLCGYPPFYSNHGLAISPGMKTRIRMGQYEFPNPEWSEVSEEVKM
LIRNLLKTEPTQRMTITEFMNHPWIMQSTKVPQTPLHTSRVLKEDKERWE
DVKEEMTSALATMRVDYEQIKIKKIEDASNPLLLKRRKKARALEAAALA
H.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Given the importance of p38a and MK2 in many cellular processes, the activity of both kinases should be controlled. Accordingly, there remains a need to find protein kinase degraders useful as therapeutic agents in the degradation of MK2 and p38a.

### SUMMARY OF THE INVENTION

In certain embodiments, the present disclosure provides a compound of Formula I:

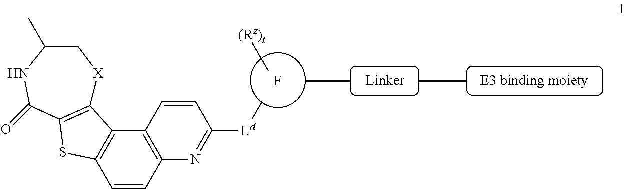

or a pharmaceutically acceptable salt thereof, wherein X, R^{z}, L^{d}, Ring F, t, the Linker and E3 binding moiety are as defined infra.

In some embodiments, the present disclosure provides a pharmaceutical composition comprising a compound as described herein, or a pharmaceutically acceptable salt thereof, and a pharmaceutically acceptable carrier, excipient, or vehicle. In some embodiments, a provided pharmaceutical composition is suitable for oral, parenteral, mucosal, transdermal or topical administration.

In some embodiments, the present disclosure provides a method of and degrading MK2 kinase, or a mutant thereof, the method comprising contacting a biological sample with a compound of formula I, or a pharmaceutically acceptable salt thereof.

In some embodiments, the present disclosure provides a method of treating a MK2-mediated disorder, the method comprising administering to a patient in need thereof a compound of formula I, or a pharmaceutically acceptable salt thereof. Such disorders or conditions include, among others, ankylosing spondylitis, rheumatoid arthritis, psoriatic arthritis and psoriasis.

### DETAILED DESCRIPTION

### 1. General Description of Compounds of the Invention

In certain embodiments, the present disclosure provides irreversible degraders of MK2. In some embodiments, such compounds include those of the formulae described herein, or a pharmaceutically acceptable salt thereof, wherein each variable is as defined and described herein.

In certain embodiments, the present disclosure provides a compound of Formula I:

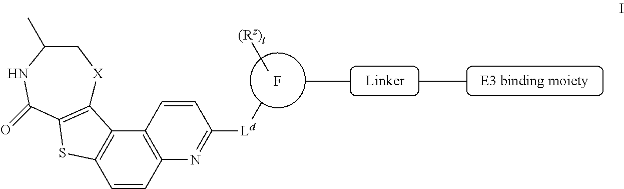

or a pharmaceutically acceptable salt thereof, wherein:
the Linker is a bivalent group;
the E3 binding moiety is a moiety that binds to an E3 ubiquitin ligase protein; Ring F is phenylene, a 5- to 6-membered heteroarylene ring having 1-4 heteroatoms independently selected from nitrogen, oxygen, and sulfur, or a 3- to 7-membered saturated or partially unsaturated heterocyclylene having 1-3 heteroatoms independently selected from nitrogen, oxygen, and sulfur;
L^{d} is selected from a covalent bond, —O—, —S—, —N(R)—, and C1-6 aliphatic;
X is selected from —O—, —S—, and —N(R)—;
R^{z} is selected from halogen, —OR, —SR, —CN, —NO2, —SO2NR, —SO2R, —SOR, —C(O)R, —CO2R, —C(O)N(R)2, —NRC(O)R, —NRC(O)OR, —NRC(O)N(R)2, —NRSO2R, —N(R)2, or an optionally substituted group selected from the group consisting of C1-6 aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur;
each R is independently hydrogen or optionally substituted C1-6 aliphatic; and
t is 0, 1, 2, or 3.

### 2. Compounds and Definitions

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “carbocyclic” (or “cycloaliphatic” or “carbocycle” or “cycloalkyl”) refers to a monocyclic C₃-C₈ hydrocarbon that is completely saturated or that contains one or more units of unsaturation, but which is not aromatic. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

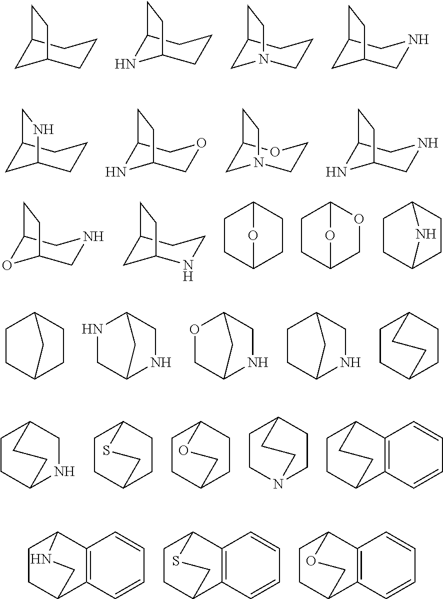

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, and include integers from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members, The term “aryl” may be used interchangeably with the term “aryl ring.” In certain embodiments of the present disclosure, “aryl” refers to an aromatic ring system and exemplary groups include phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, including 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Exemplary heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Exemplary groups include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, including one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds provided herein may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

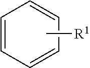

refers to at least

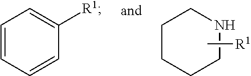

refers to at least

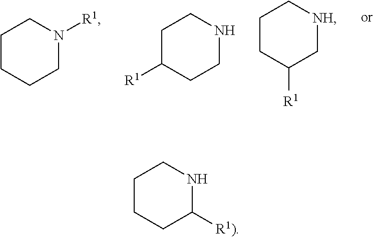

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure include those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂, —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each RI is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of RI, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of RI are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of this disclosure. Unless otherwise stated, all tautomeric forms of the compounds provided herein are within the scope of this disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

As used herein, the term “inhibitor” is defined as a compound that binds to and/or inhibits the target protein kinase, MK2, with measurable affinity. In certain embodiments, an inhibitor has an IC₅₀ and/or binding constant of less than about 50 μM, less than about 1 μM, less than about 500 nM, less than about 100 nM, or less than about 10 nM.

As used herein the term “degradation”, “degrading”, “MK2 degradation” refers to process by which MK2 proteins are destroyed in a cell in order to maintain protein homeostasis, or an equilibrium of proteins in the human body.

The terms “measurable affinity” and “measurably degrade,” as used herein, means a measurable change in MK2 activity between a sample comprising a compound of the present disclosure, or composition thereof, and MK2, and an equivalent sample comprising MK2, in the absence of said compound, or composition thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Inhibition of activity of a protein kinase, for example, MK2 or a mutant thereof, in a biological sample is useful for a variety of purposes that are known to one of skill in the art. Examples of such purposes include, but are not limited to, blood transfusion, organ transplantation, biological specimen storage, and biological assays.

As used herein, a “disease or disorder associated with MK2” or, alternatively, “an MK2-mediated disease or disorder” means any disease or other deleterious condition in which MK2, or a mutant thereof, is known or suspected to play a role.

The term “subject”, as used herein, means a mammal and includes human and animal subjects, such as domestic animals (e.g., horses, dogs, cats, etc.). The terms “subject” and “patient” are used interchangeably. In some embodiments, the “patient” or “subject” means an animal, including a mammal, and a human.

As use herein, the phrase “compound of the disclosure”, “degraders of the disclosure”, “degraders”, refers to those compounds which are disclosed herein, both generically and specifically.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The amount of compounds of the present disclosure that may be combined with the carrier materials to produce a composition in a single dosage form will vary depending upon the host treated, the particular mode of administration, etc. Provided compositions may further be formulated so that a dosage of between 0.01 to about 100 mg/kg, or about 0.1 mg/kg to about 50 mg/kg, and from about 1 mg/kg to about 25 mg/kg, of subject body weight/day of the degrader can be administered to a patient receiving these compositions to obtain the desired therapeutic effect. The amount of a compound of the present disclosure in the composition will also depend upon the particular compound in the composition.

The expression “unit dosage form” as used herein refers to a physically discrete unit of a provided compound and/or compositions thereof appropriate for the subject to be treated. It will be understood, however, that the total daily usage of the active agent (i.e., compounds and compositions of the present disclosure) will be decided by the attending physician within the scope of sound medical judgment. The specific effective dose level for any particular subject (i.e., patient) or organism will depend upon a variety of factors including the disorder being treated and the severity of the disorder; activity of specific active agent employed; specific composition employed; age, body weight, general health, sex and diet of the subject; time of administration, route of administration, and rate of excretion of the specific active agent employed; duration of the treatment; and like factors well known in the medical arts.

The term “parenteral” as used herein includes subcutaneous, intravenous, intramuscular, intra-articular, intra-synovial, intrasternal, intrathecal, intrahepatic, intralesional and intracranial injection or infusion techniques.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of an MK2-mediated diseases or disorder.

### 3. Description of Exemplary Embodiments

In some embodiments, the present disclosure provides a compound of formula I:

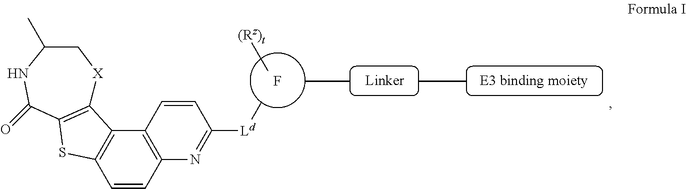

or a pharmaceutically acceptable salt thereof, wherein:
the Linker is a bivalent group;
the E3 binding moiety is a moiety that binds to an E3 ubiquitin ligase protein; Ring F is phenylene, a 5- to 6-membered heteroarylene ring having 1-4 heteroatoms independently selected from nitrogen, oxygen, and sulfur, or a 3- to 7-membered saturated or partially unsaturated heterocyclylene having 1-3 heteroatoms independently selected from nitrogen, oxygen, and sulfur;
L^{d} is selected from a covalent bond, —O—, —S—, —N(R)—, and C₁₋₆ aliphatic; X is selected from —O—, —S—, and —N(R)—;
R^{z} is selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C1-6 aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur;
each R is independently hydrogen or optionally substituted C1-6 aliphatic; and
t is 0, 1, 2, or 3.

In some embodiments, Ring F is phenylene.

Accordingly, in some embodiments, the present disclosure provides a compound of Formula I-a:

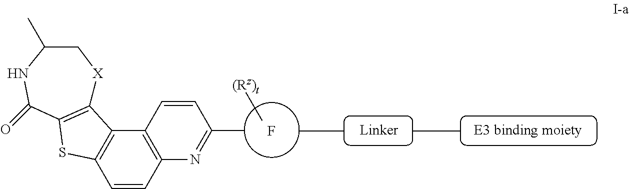

or a pharmaceutically acceptable salt thereof.

Accordingly, in some embodiments, the present disclosure provides a compound of Formula I-b:

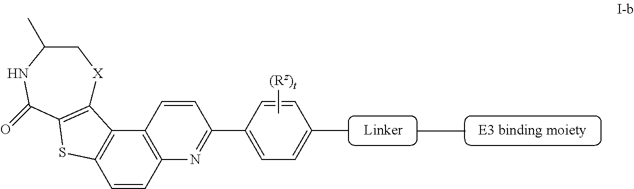

or a pharmaceutically acceptable salt thereof.

In some embodiments, the present disclosure provides a compound of Formula I-c:

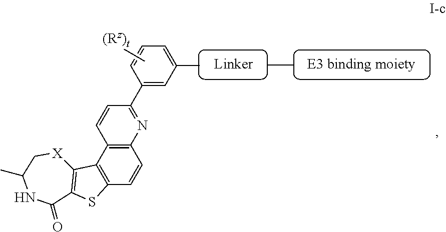

or a pharmaceutically acceptable salt thereof.

In some embodiments, the present disclosure provides a compound of Formula I-d:

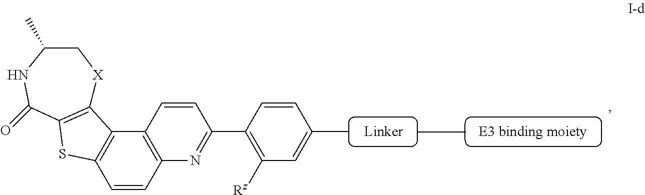

or a pharmaceutically acceptable salt thereof.

As defined generally above, X is selected from —O—, —S—, and —N(R)—.

In some embodiments of Formula I, Formula I-a, Formula I-b, Formula I-c, and Formula I-d, X is —O—. In some embodiments of Formula I, Formula I-a, Formula I-b, Formula I-c, and Formula I-d, X is —S—. In some embodiments of Formula I, Formula I-a, Formula I-b, Formula I-c, and Formula I-d, X is —N(R)—. In some such embodiments, X is —N(H)—.

In some embodiments, the present disclosure provides a compound of Formulae I-a, I-a-i, I-a-ii, I-a-iii, I-a-iv, I-a-v, I-b, I-b-i, I-b-ii, I-b-iii, I-b-iv, I-b-v, I-c, I-c-i, I-c-ii, I-c-iii, I-c-iv, I-c-v, I-d, I-d-i, and I-d-ii:

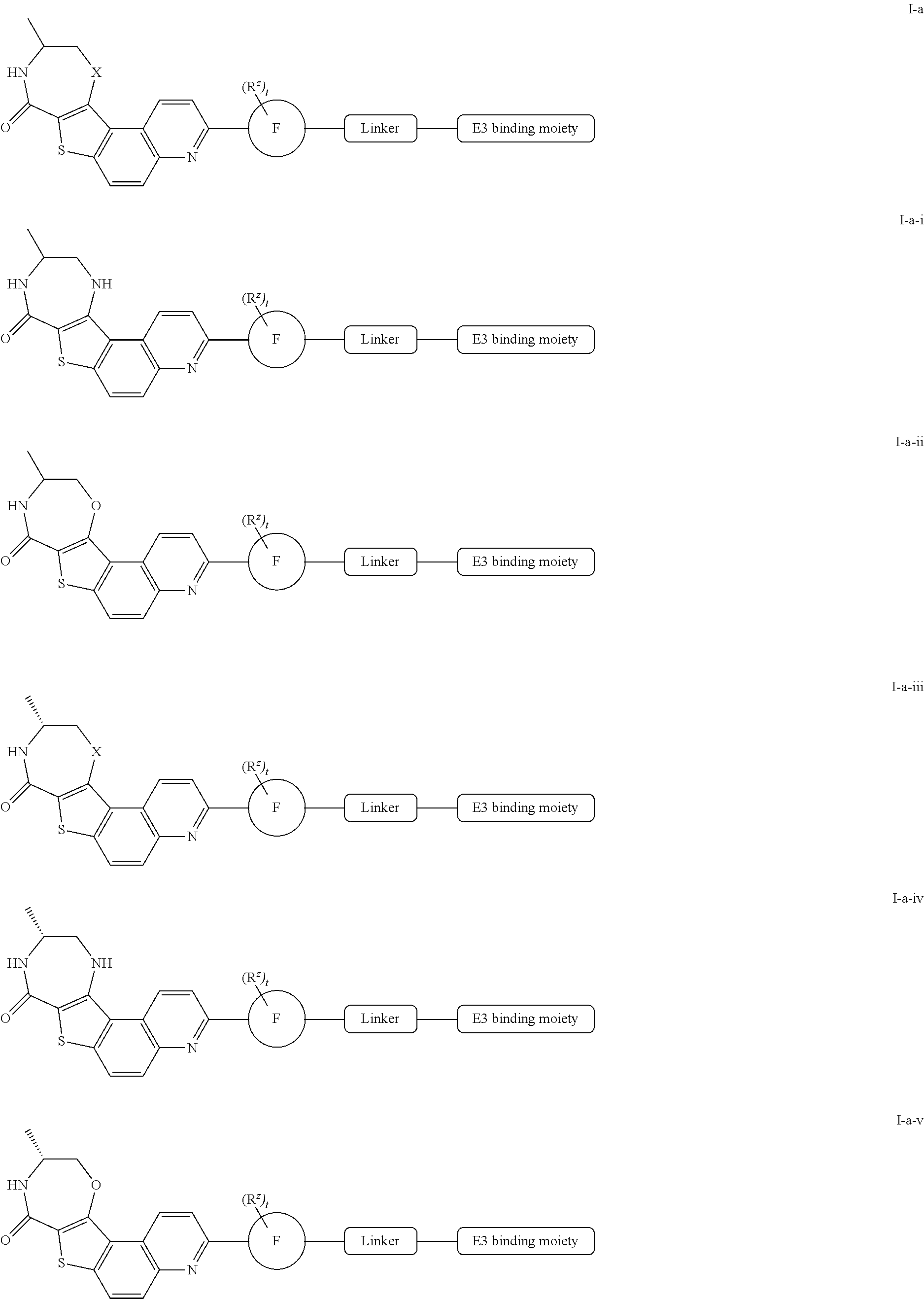

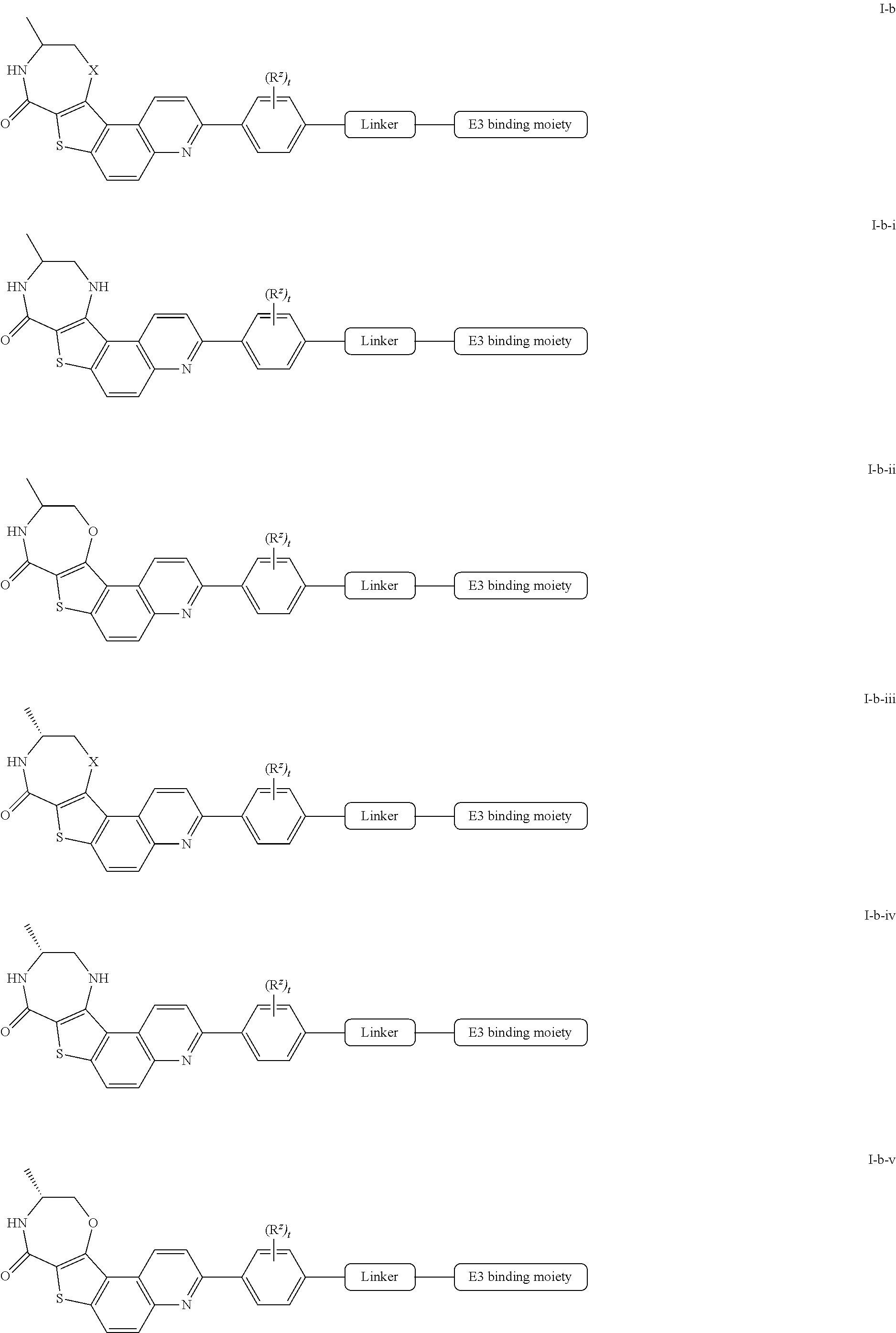

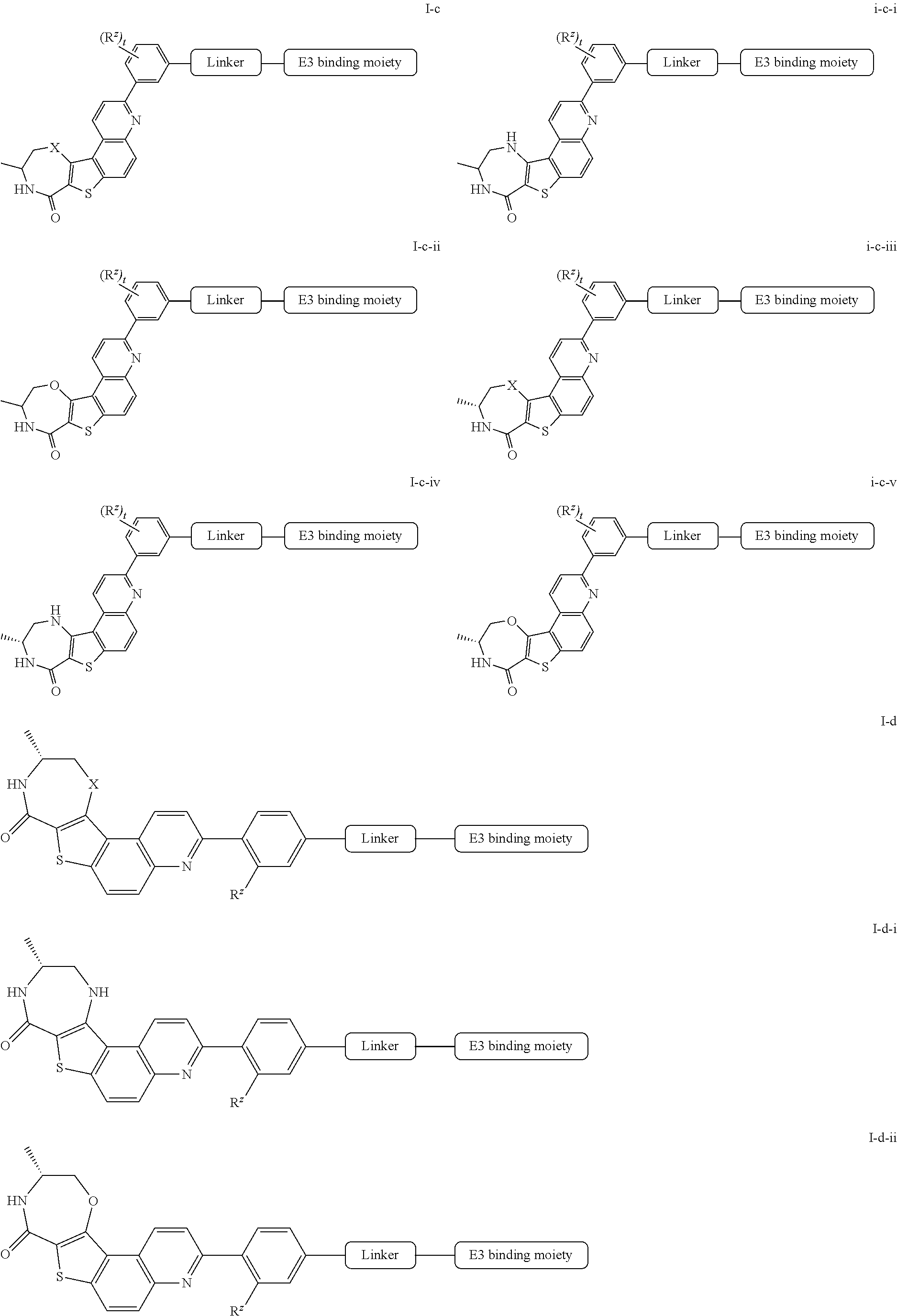

or a pharmaceutically acceptable salt thereof.

As defined generally above, Ring F is a phenylene, a 5- to 6-membered heteroarylene ring having 1-4 heteroatoms independently selected from nitrogen, oxygen, and sulfur, or a 3- to 7-membered saturated or partially unsaturated heterocyclylene having 1-3 heteroatoms independently selected from nitrogen, oxygen, and sulfur.

In some embodiments of any Formulae described herein, Ring F is phenylene. In some embodiments of any Formulae described herein, Ring F is a 5- to 6-membered heteroarylene ring having 1-4 heteroatoms independently selected from nitrogen, oxygen, and sulfur. In some embodiments of any Formulae described herein, Ring F is a 5-membered heteroarylene ring having 1-4 heteroatoms independently selected from nitrogen, oxygen, and sulfur. In some embodiments of any Formulae described herein, Ring F is a 5-membered heteroarylene ring having 1-3 heteroatoms independently selected from nitrogen, oxygen, and sulfur. In some embodiments of any Formulae described herein, Ring F is a 5-membered heteroarylene ring having 1-2 heteroatoms independently selected from nitrogen, oxygen, and sulfur. In some embodiments of any Formulae described herein, Ring F is a 6-membered heteroarylene ring having 1-2 nitrogen atoms.

In some embodiments of any Formulae described herein, Ring F is a 3- to 7-membered saturated or partially unsaturated heterocyclylene having 1-3 heteroatoms independently selected from nitrogen, oxygen, and sulfur. In some embodiments of any Formulae described herein, Ring F is a 3-membered saturated heterocyclylene having 1 heteroatom selected from nitrogen, oxygen, and sulfur. In some embodiments of any Formulae described herein, Ring F is a 4-membered saturated heterocyclylene having 1 heteroatom selected from nitrogen, oxygen, and sulfur. In some embodiments of any Formulae described herein, Ring F is a 5-membered saturated or partially unsaturated heterocyclylene having 1 heteroatom selected from nitrogen, oxygen, and sulfur. In some embodiments of any Formulae described herein, Ring F is a 6-membered saturated or partially unsaturated heterocyclylene having 1-2 heteroatoms independently selected from nitrogen, oxygen, and sulfur.

As defined generally above, L^{d} is selected from a covalent bond, —O—, —S—, —N(R)—, and C₁₋₆ aliphatic. In some embodiments of any Formulae described herein, L^{d} is a covalent bond. In some embodiments of any Formulae described herein, L^{d} is selected from —O—, —S—, —N(R)—, and C₁₋₆ aliphatic. In some embodiments of any Formulae described herein, L^{d} is —O—. In some embodiments of any Formulae described herein, L^{d} is —S—. In some embodiments of any Formulae described herein, L^{d} is —N(R)—. In some embodiments of any Formulae described herein, L^{d} IS C₁₋₆ aliphatic. In some embodiments of any Formulae described herein, La is C₁₋₄ aliphatic. In some embodiments of any Formulae described herein, L^{d} is C₁₋₂ aliphatic. In some such embodiments, L^{d} is —CH₂—, —CH(CH)—, or —CH₂CH₂—.

As defined generally above, R^{z} is selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur. In some embodiments of any Formulae described herein, R^{z} is halogen. In some embodiments of any Formulae described herein, R^{z} is —OR. In some embodiments of any Formulae described herein, R^{z} is —SR. In some embodiments of any Formulae described herein, R^{z} is —CN. In some embodiments of any Formulae described herein, R^{z} is —NO₂. In some embodiments of any Formulae described herein, R^{z} is —SO₂NR. In some embodiments of any Formulae described herein, R^{z} is —SO₂R. In some embodiments of any Formulae described herein, R^{z} is —SOR. In some embodiments of any Formulae described herein, R^{z} is —C(O)R. In some embodiments of any Formulae described herein, R^{z} is —CO₂R. In some embodiments of any Formulae described herein, R^{z} is —C(O)N(R)₂. In some embodiments of any Formulae described herein, R^{z} is —NRC(O)R. In some embodiments of any Formulae described herein, R^{z} is —NRC(O)OR. In some embodiments of any Formulae described herein, R^{z} is —NRC(O)N(R)₂. In some embodiments of any Formulae described herein, R^{z} is —NRSO₂R. In some embodiments of any Formulae described herein, R^{z} is —N(R)₂.

In some embodiments of any Formulae described herein, R^{z} is optionally substituted C₁₋₆ aliphatic. In some embodiments of any Formulae described herein, R^{z} is optionally substituted C₁₋₄ aliphatic. In some embodiments of any Formulae described herein, R^{z} is optionally substituted C₁₋₂ aliphatic. In some embodiments of any Formulae described herein, R^{z} is —CH₃ or CH₂CH₃. In some embodiments of any Formulae described herein, R^{z} is C₁₋₆ aliphatic optionally substituted with halogen. In some such embodiments, R^{z} is —CF₃, —CF₂H, —CFH₂, or —CH₂CF₃.

In some embodiments of any Formulae described herein, R^{z} is optionally substituted phenyl.

In some embodiments of any Formulae described herein, R^{z} is an optionally substituted 3- to 8-membered saturated or partially unsaturated carbocyclic ring. In some embodiments of any Formulae described herein, R^{z} is an optionally substituted 3-membered saturated carbocyclic ring. In some embodiments of any Formulae described herein, R^{z} is an optionally substituted 4-membered saturated carbocyclic ring. In some embodiments of any Formulae described herein, R^{z} is an optionally substituted 5-membered saturated or partially unsaturated carbocyclic ring. In some embodiments of any Formulae described herein, R^{z} is an optionally substituted 6-membered saturated or partially unsaturated carbocyclic ring. In some embodiments of any Formulae described herein, R^{z} is an optionally substituted 7-membered saturated or partially unsaturated carbocyclic ring. In some embodiments of any Formulae described herein, R^{z} is an optionally substituted 8-membered saturated or partially unsaturated carbocyclic ring.

In some embodiments of any Formulae described herein, R^{z} is an optionally substituted 4- to 7-membered heterocyclic ring having 1-2 heteroatoms independently selected from nitrogen, oxygen, or sulfur. In some embodiments of any Formulae described herein, R^{z} is an optionally substituted 4-membered heterocyclic ring having 1 heteroatom selected from nitrogen, oxygen, or sulfur. In some embodiments of any Formulae described herein, R^{z} is an optionally substituted 5-membered heterocyclic ring having 1-2 heteroatoms independently selected from nitrogen, oxygen, or sulfur. In some embodiments of any Formulae described herein, R^{z} is an optionally substituted 6-membered heterocyclic ring having 1-2 heteroatoms independently selected from nitrogen, oxygen, or sulfur. In some embodiments of any Formulae described herein, R^{z} is an optionally substituted 7-membered heterocyclic ring having 1-2 heteroatoms independently selected from nitrogen, oxygen, or sulfur.

In some embodiments of any Formulae described herein, R^{z} is an optionally substituted 5- to 6-membered monocyclic heteroaryl ring having 1-4 heteroatoms independently selected from nitrogen, oxygen, or sulfur. In some embodiments of any Formulae described herein, R^{z} is an optionally substituted 5-membered monocyclic heteroaryl ring having 1-4 heteroatoms independently selected from nitrogen, oxygen, or sulfur. In some embodiments of any Formulae described herein, R^{z} is an optionally substituted 5-membered monocyclic heteroaryl ring having 1-3 heteroatoms independently selected from nitrogen, oxygen, or sulfur. In some embodiments of any Formulae described herein, R^{z} is an optionally substituted 5-membered monocyclic heteroaryl ring having 1-2 heteroatoms independently selected from nitrogen, oxygen, or sulfur. In some embodiments of any Formulae described herein, R^{z} is an optionally substituted 6-membered monocyclic heteroaryl ring having 1-2 nitrogen atoms.

As defined generally above, each R is independently hydrogen or optionally substituted C₁₋₆ aliphatic. In some embodiments of any Formulae described herein, R is hydrogen. In some embodiments of any Formulae described herein, R is optionally substituted C₁₋₆ aliphatic. In some embodiments of any Formulae described herein, R is optionally substituted C₁₋₄ aliphatic. In some embodiments of any Formulae described herein, R is optionally substituted C₁₋₂ aliphatic. In some such embodiments, R is —CH₃, —CH₂CH₃, —CF₃, —CF₂H, —CFH₂, or —CH₂CF₃.

As defined generally above, each of t is 0, 1, 2, or 3. In some embodiments of any Formulae described herein, t is 0. In some embodiments of any Formulae described herein, t is 1. In some embodiments of any Formulae described herein, t is 2. In some embodiments of any Formulae described herein, t is 3.

### Linkers

As defined above, the Linker is a bivalent group that links the E3 binding moiety to the rest of the compound. In some embodiments, the Linker is an optionally substituted bivalent C₂₋₂₀ straight or branched aliphatic chain, wherein one, two, three, four, or five methylene units of the aliphatic chain are optionally and independently replaced by a group selected from —N(R)—, —C(O)—, —C(O)N(R)—, - a bivalent 3- to 6-membered monocyclic saturated ring having 0-2 heteroatoms independently selected from nitrogen, a bivalent 6- to 8-membered saturated or partially unsaturated bridged bicyclic, fused bicyclic or spirofused heterocyclic ring having 1-3 heteroatoms independently selected from nitrogen, each monocyclic ring, bridged bicyclic ring, fused bicyclic ring, spirofused ring, or phenylene is substituted by 0-4 instances of R^{L}, wherein R^{L} is independently selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur.

In some embodiments, the Linker is an optionally substituted bivalent C₂₋₂₀ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}. In some embodiments, the Linker is an optionally substituted bivalent C₂₋₂₀ straight or branched aliphatic chain, wherein two, three, four, or five methylene units of the aliphatic chain are optionally and independently replaced by a group selected from —C(O)N(R)—, —N(R)—, —C(O)—,

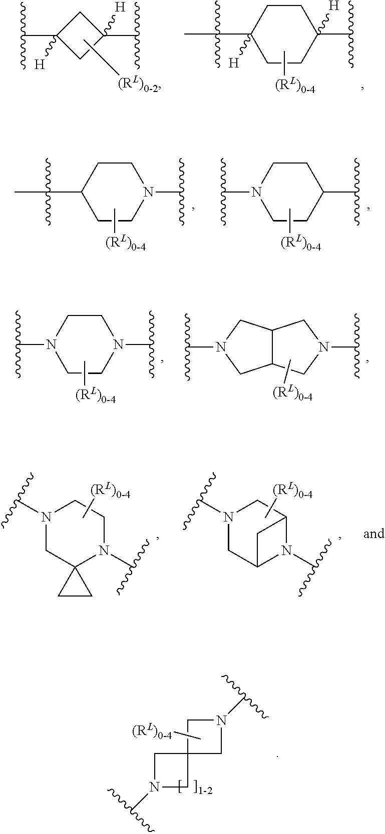

In some embodiments, the Linker is an optionally substituted bivalent C₂₋₂₀ straight or branched aliphatic chain, wherein two, three, four, or five methylene units of the aliphatic chain are optionally and independently replaced by a group selected from —C(O)N(R)—, —N(R)—, —C(O)—,

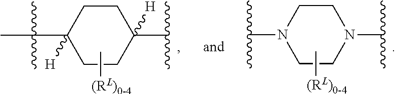

In some embodiments, the Linker is an optionally substituted bivalent C₃₋₁₇ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}. In some embodiments, the Linker is an optionally substituted bivalent C₃₋₁₇ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments, the Linker is an optionally substituted bivalent C₃₋₁₇ straight or branched aliphatic chain, wherein two, three, four, or five methylene units of the aliphatic chain are optionally and independently replaced by a group selected from —C(O)N(R)—, —N(R)—, —C(O)—,

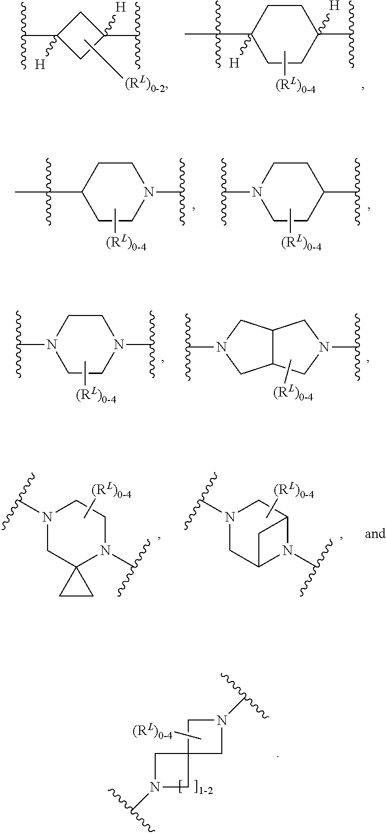

In some embodiments, the Linker is an optionally substituted bivalent C₃₋₁₇ straight or branched aliphatic chain, wherein two, three, four, or five methylene units of the aliphatic chain are optionally and independently replaced by a group selected from —C(O)N(R)—, —N(R)—, —C(O)—,

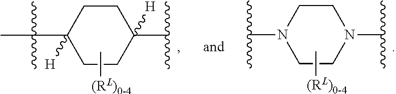

In some embodiments, the Linker is an optionally substituted bivalent C₂₋₁₀ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}. In some embodiments, the Linker is an optionally substituted bivalent C₂₋₁₀ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments, the Linker is an optionally substituted bivalent C₂₋₁₀ straight or branched aliphatic chain, wherein two, three, four, or five methylene units of the aliphatic chain are optionally and independently replaced by a group selected from —C(O)N(R)—, —N(R)—, —C(O)—,

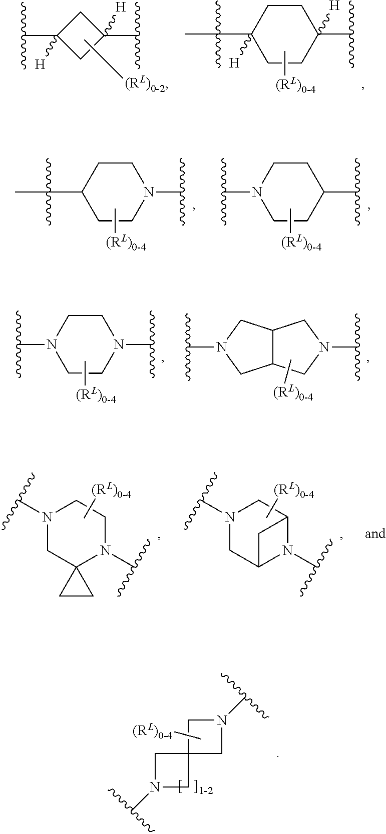

In some embodiments, the Linker is an optionally substituted bivalent C₂₋₁₀ straight or branched aliphatic chain, wherein two, three, four, or five methylene units of the aliphatic chain are optionally and independently replaced by a group selected from —C(O)N(R)—, —N(R)—, —C(O)—,

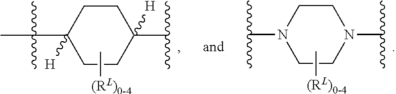

In some embodiments, the Linker is an optionally substituted bivalent C₂₋₆ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}. In some embodiments, the Linker is an optionally substituted bivalent C₂₋₆ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments, the Linker is an optionally substituted bivalent C₂₋₆ straight or branched aliphatic chain, wherein two, three, four, or five methylene units of the aliphatic chain are optionally and independently replaced by a group selected from —C(O)N(R)—, —N(R)—, —C(O)—,

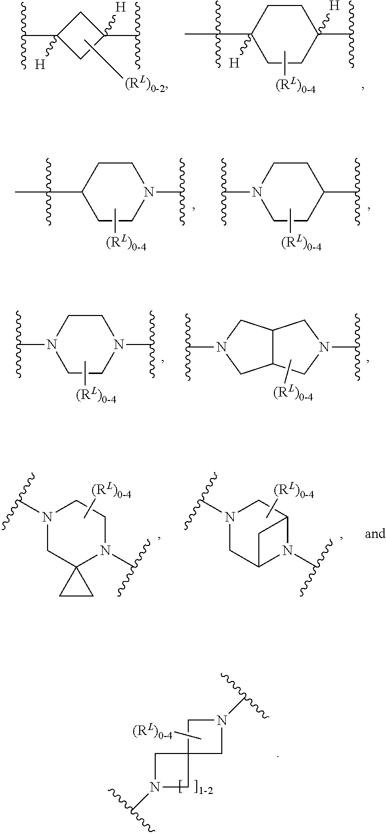

In some embodiments, the Linker is an optionally substituted bivalent C₂₋₆ straight or branched aliphatic chain, wherein two, three, four, or five methylene units of the aliphatic chain are optionally and independently replaced by a group selected from —C(O)N(R)—, —N(R)—, —C(O)—,

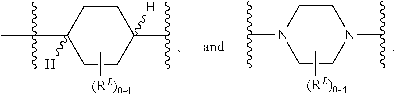

In some embodiments, the Linker is an optionally substituted bivalent C₄₋₆ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}. In some embodiments, the Linker is an optionally substituted bivalent C₄₋₆ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments, the Linker is an optionally substituted bivalent C₄₋₆ straight or branched aliphatic chain, wherein two, three, four, or five methylene units of the aliphatic chain are optionally and independently replaced by a group selected from —C(O)N(R)—, —N(R)—, —C(O)—,

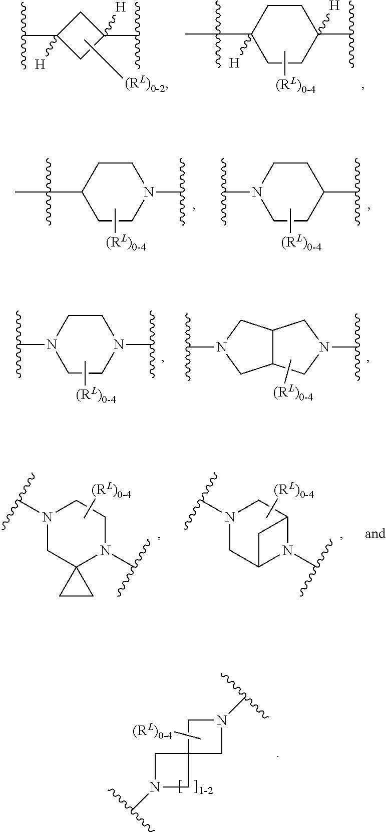

In some embodiments, the Linker is an optionally substituted bivalent C₄₋₆ straight or branched aliphatic chain, wherein two, three, four, or five methylene units of the aliphatic chain are optionally and independently replaced by a group selected from —C(O)N(R)—, —N(R)—, —C(O)—,

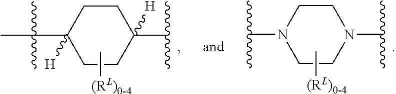

In some embodiments, the Linker is selected from the group consisting of:

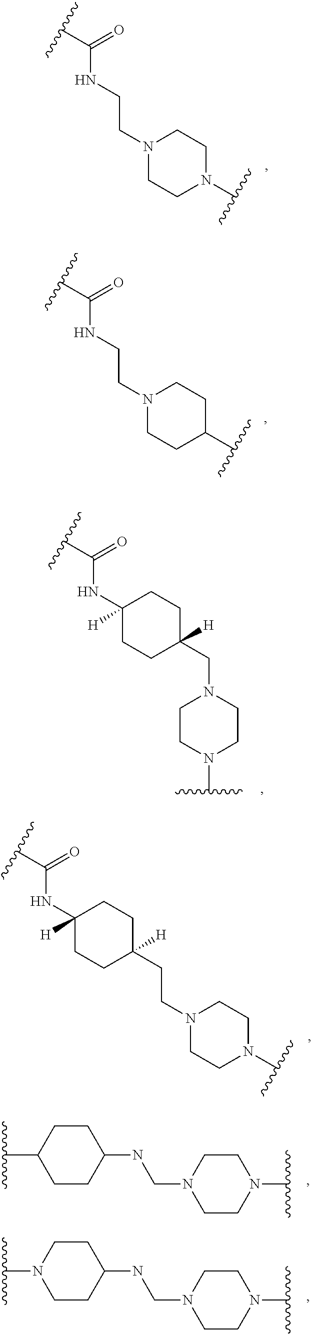 [SMILES: *C(=O)NCCN1CCN(*)CC1 ; *C(=O)NCCN1CCC(*)CC1 ; *C(=O)N[C@H]1CC[C@H](CN2CCN(*)CC2)CC1 ; *C(=O)N[C@H]1CC[C@H](CCN2CCN(*)CC2)CC1 ; *C1CCC(NCN2CCN(*)CC2)CC1 ; *N1CCC(NCN2CCN(*)CC2)CC1]

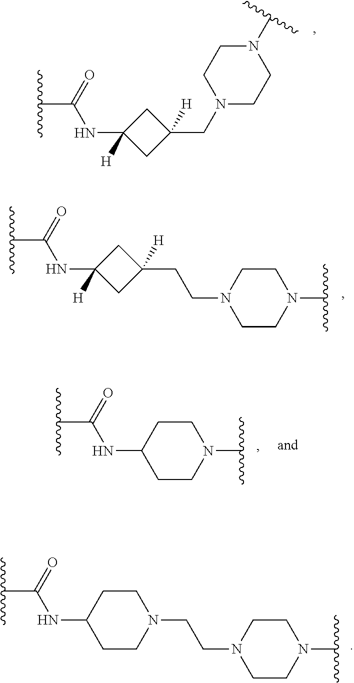 [SMILES: *C(=O)N[C@H]1C[C@H](CN2CCN(*)CC2)C1 ; *C(=O)N[C@H]1C[C@H](CCN2CCN(*)CC2)C1 ; *C(=O)NC1CCN(*)CC1 ; *C(=O)NC1CCN(CCN2CCN(*)CC2)CC1]

In some embodiments, the Linker is selected from the group consisting of:

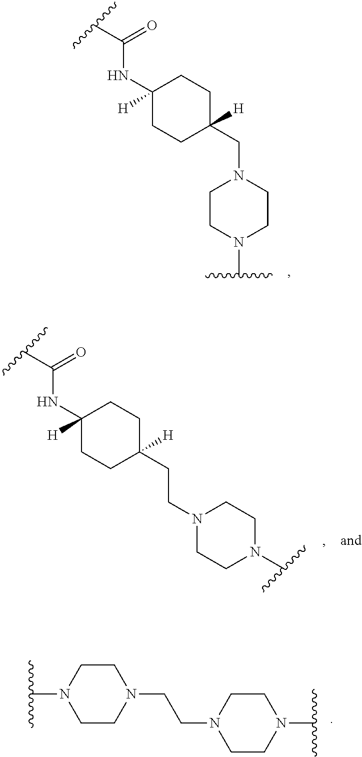 [SMILES: *C(=O)N[C@H]1CC[C@H](CN2CCN(*)CC2)CC1 ; *C(=O)N[C@H]1CC[C@H](CCN2CCN(*)CC2)CC1 ; *N1CCN(CCN2CCN(*)CC2)CC1]

### E3 Binding Moieties

The proteasome is a large protein complex responsible for degradation of intracellular proteins. Polymerization of ubiquitin, a key molecule known to work in concert with the proteasome, serves as a degradation signal for numerous target proteins; the destruction of a protein is initiated by covalent attachment of a chain consisting of several copies of ubiquitin (more than four ubiquitin molecules), through the concerted actions of a network of proteins, including the E1 (ubiquitin-activating), E2 (ubiquitin-conjugating) and E3 (ubiquitin-ligating) enzymes. The polymerized ubiquitin chain acts as a signal that shuttles the target proteins to the proteasome, where the substrate is proteolytically broken down. The set of E3 proteins is highly diverse, because each E3 enzyme selectively recognizes a protein substrate for ubiquitylation. The ubiquitin-proteasome system (UPS) controls almost all basic cellular processes—such as progression through the cell cycle, signal transduction, cell death, immune responses, metabolism, protein quality control and development—by degrading short-lived regulatory or structurally aberrant proteins. Cereblon (CRBN) is a substrate receptor of the CRL4^{CRBN} E3 ubiquitin ligase and induces cell death by targeting key neo-substrates for ubiquitination and subsequent degradation.

In some embodiments, compounds disclosed herein degrade MK2 kinase via the ubiquitin-proteasome system.

As defined generally above, the E3 binding moiety is a moiety that binds to an E3 ubiquitin ligase protein. In some embodiments, the E3 binding moiety is a cereblon protein binding moiety.

In some embodiments, the cereblon protein binding moiety is selected from the group consisting of:

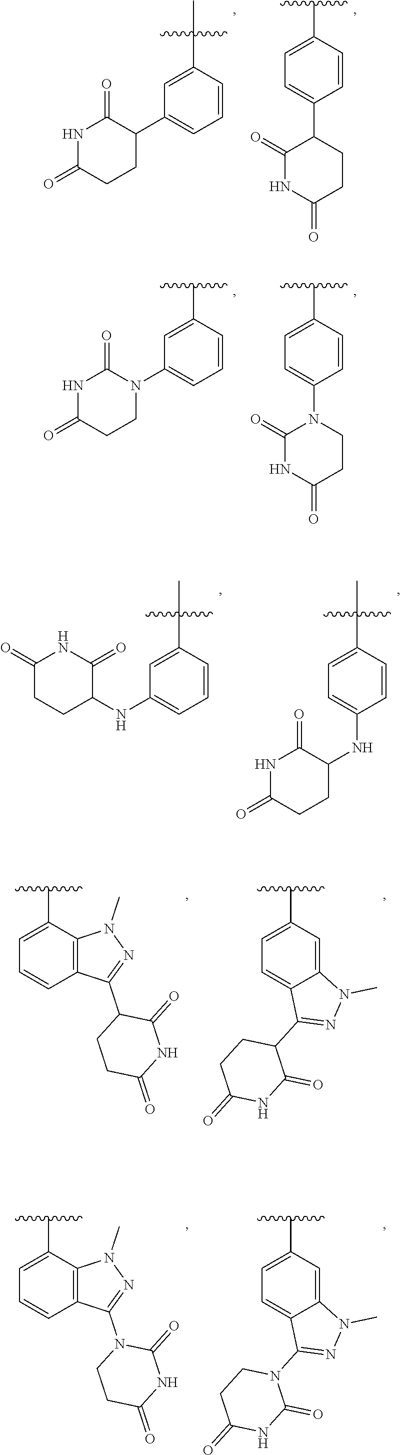

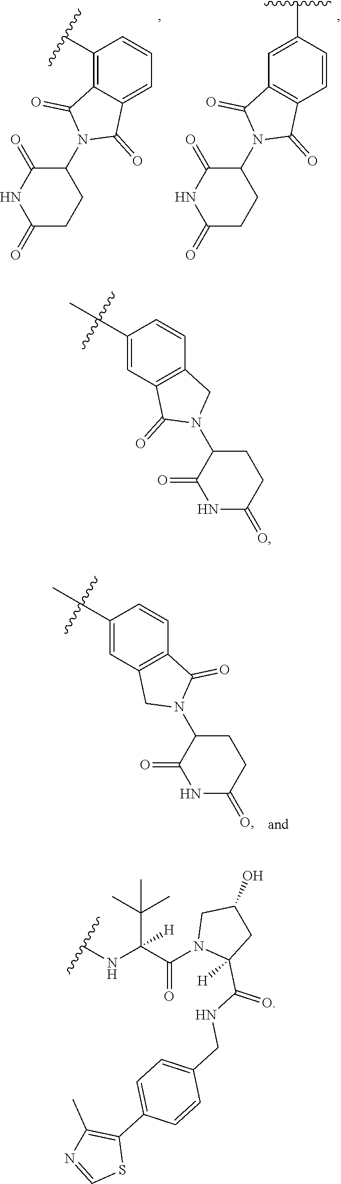 [SMILES: *c1cccc2c1C(=O)N(C1CCC(=O)NC1=O)C2=O ; *c1ccc2c(c1)C(=O)N(C1CCC(=O)NC1=O)C2=O ; *c1ccc2c(c1)C(=O)N(C1CCC(=O)NC1=O)C2 ; *c1ccc2c(c1)CN(C1CCC(=O)NC1=O)C2=O ; *N[C@H](C(=O)N1C[C@H](O)C[C@H]1C(=O)NCc1ccc(-c2scnc2C)cc1)C(C)(C)C]

In some embodiments, the cereblon protein binding moiety is selected from the group consisting of:

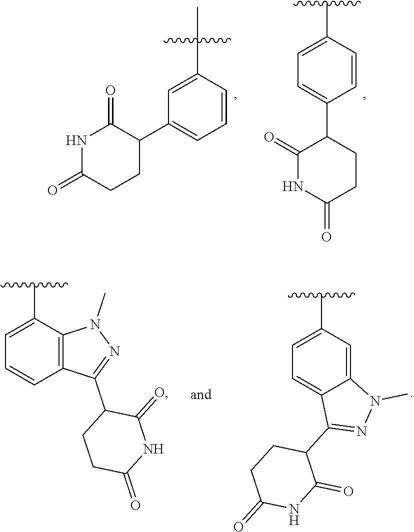

In some embodiments, a compound of Formula I is selected from Table 1.

TABLE 1
Exemplary Compounds

| | |
|---|---|
| 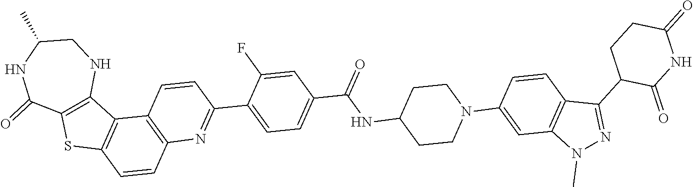 [SMILES: C[C@@H]1CNc2c(sc3ccc4nc(-c5ccc(C(=O)NC6CCN(c7ccc8c(C9CCC(=O)NC9=O)nn(C)c8c7)CC6)cc5F)ccc4c23)C(=O)N1] | Ex 1 |
| 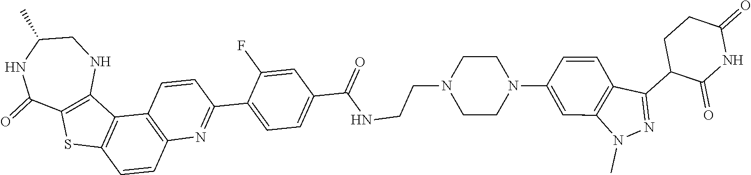 [SMILES: C[C@@H]1CNc2c(sc3ccc4nc(-c5ccc(C(=O)NCCN6CCN(c7ccc8c(C9CCC(=O)NC9=O)nn(C)c8c7)CC6)cc5F)ccc4c23)C(=O)N1] | Ex 2 |
| 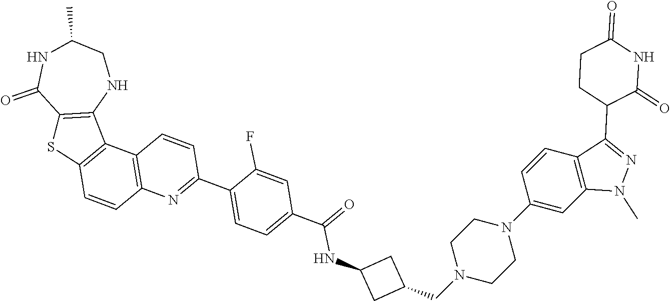 [SMILES: C[C@@H]1CNc2c(sc3ccc4nc(-c5ccc(C(=O)N[C@H]6C[C@H](CN7CCN(c8ccc9c(C%10CCC(=O)NC%10=O)nn(C)c9c8)CC7)C6)cc5F)ccc4c23)C(=O)N1] | Ex 3 |
| 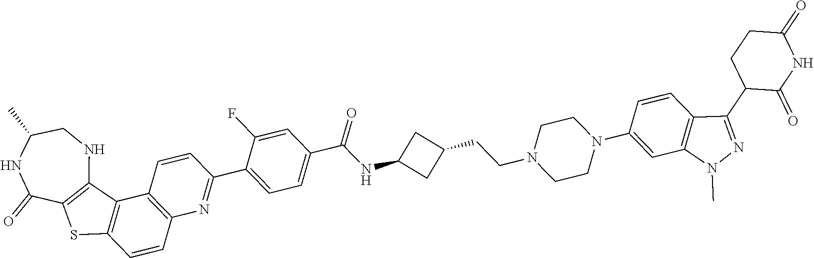 [SMILES: C[C@@H]1CNc2c(sc3ccc4nc(-c5ccc(C(=O)N[C@H]6C[C@H](CCN7CCN(c8ccc9c(C%10CCC(=O)NC%10=O)nn(C)c9c8)CC7)C6)cc5F)ccc4c23)C(=O)N1] | Ex 4 |
| 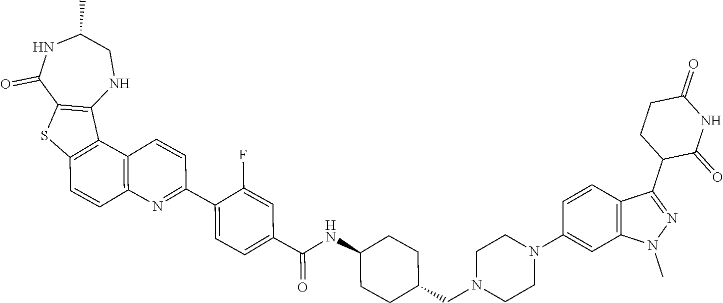 [SMILES: C[C@@H]1CNc2c(sc3ccc4nc(-c5ccc(C(=O)N[C@H]6CC[C@H](CN7CCN(c8ccc9c(C%10CCC(=O)NC%10=O)nn(C)c9c8)CC7)CC6)cc5F)ccc4c23)C(=O)N1] | Ex 5 |
| 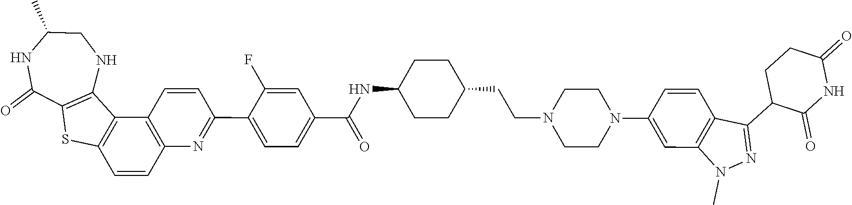 [SMILES: C[C@@H]1CNc2c(sc3ccc4nc(-c5ccc(C(=O)N[C@H]6CC[C@H](CCN7CCN(c8ccc9c(C%10CCC(=O)NC%10=O)nn(C)c9c8)CC7)CC6)cc5F)ccc4c23)C(=O)N1] | Ex 6 |
| 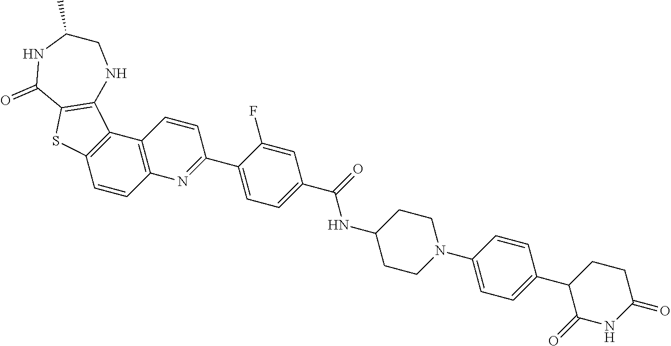 [SMILES: C[C@@H]1CNc2c(sc3ccc4nc(-c5ccc(C(=O)NC6CCN(c7ccc(C8CCC(=O)NC8=O)cc7)CC6)cc5F)ccc4c23)C(=O)N1] | Ex 7 |
| 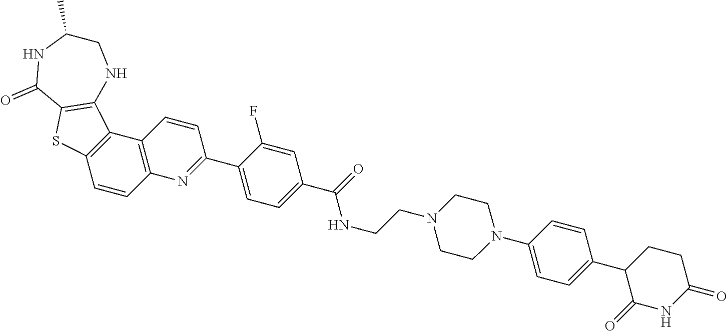 [SMILES: C[C@@H]1CNc2c(sc3ccc4nc(-c5ccc(C(=O)NCCN6CCN(c7ccc(C8CCC(=O)NC8=O)cc7)CC6)cc5F)ccc4c23)C(=O)N1] | Ex 8 |
| 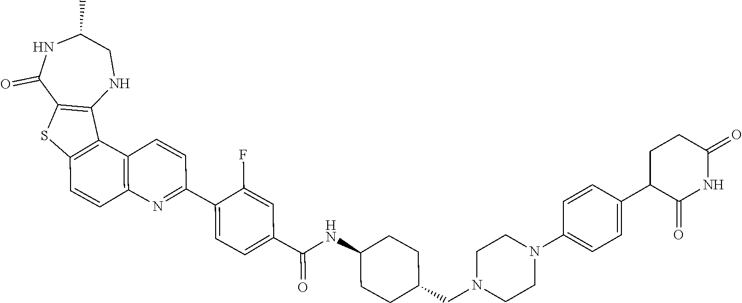 [SMILES: C[C@@H]1CNc2c(sc3ccc4nc(-c5ccc(C(=O)N[C@H]6CC[C@H](CN7CCN(c8ccc(C9CCC(=O)NC9=O)cc8)CC7)CC6)cc5F)ccc4c23)C(=O)N1] | Ex 9 |
| 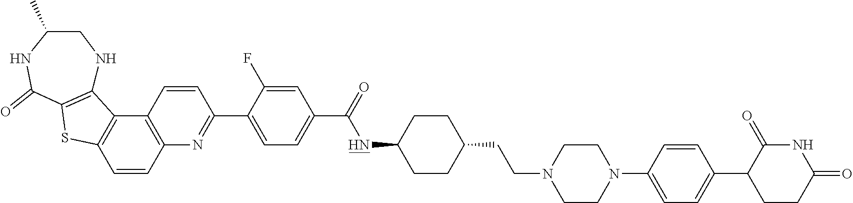 [SMILES: C[C@@H]1CNc2c(sc3ccc4nc(-c5ccc(C(=O)N[C@H]6CC[C@H](CCN7CCN(c8ccc(C9CCC(=O)NC9=O)cc8)CC7)CC6)cc5F)ccc4c23)C(=O)N1] | Ex 10 |
| 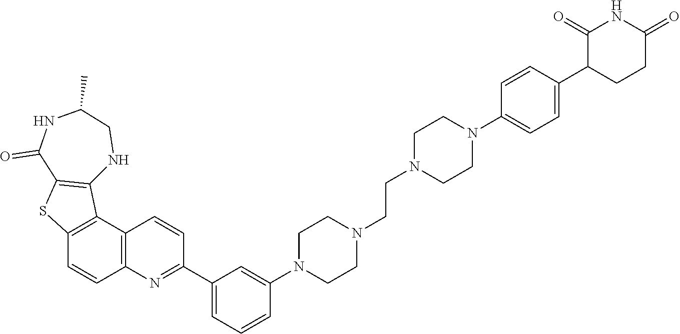 [SMILES: C[C@@H]1CNc2c(sc3ccc4nc(-c5cccc(N6CCN(CCN7CCN(c8ccc(C9CCC(=O)NC9=O)cc8)CC7)CC6)c5)ccc4c23)C(=O)N1] | Ex 11 |
| 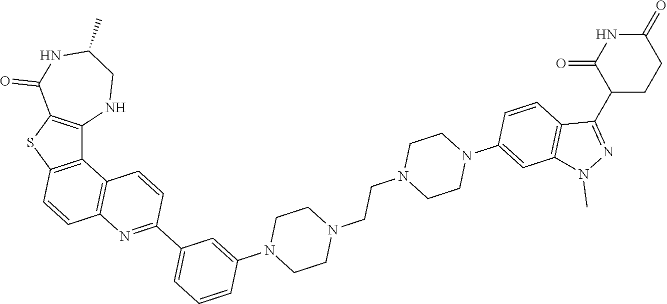 [SMILES: C[C@@H]1CNc2c(sc3ccc4nc(-c5cccc(N6CCN(CCN7CCN(c8ccc9c(C%10CCC(=O)NC%10=O)nn(C)c9c8)CC7)CC6)c5)ccc4c23)C(=O)N1] | Ex 12 |
| 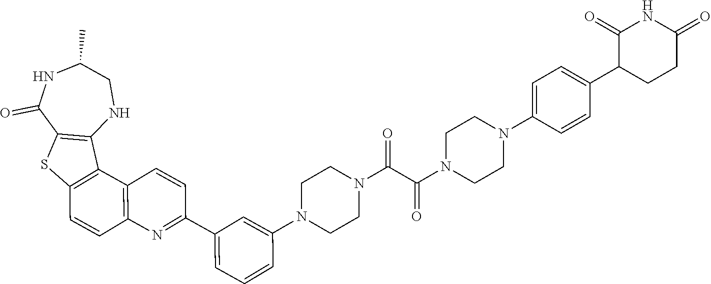 [SMILES: C[C@@H]1CNc2c(sc3ccc4nc(-c5cccc(N6CCN(C(=O)C(=O)N7CCN(c8ccc(C9CCC(=O)NC9=O)cc8)CC7)CC6)c5)ccc4c23)C(=O)N1] | Ex 13 |
| 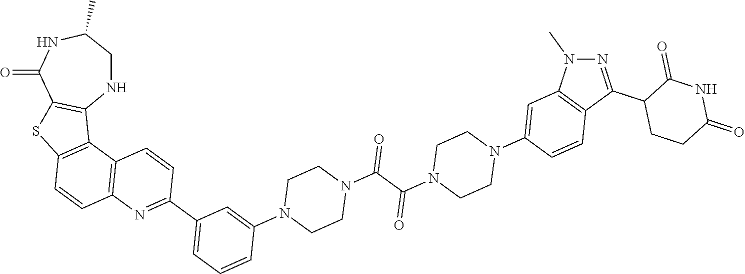 [SMILES: C[C@@H]1CNc2c(sc3ccc4nc(-c5cccc(N6CCN(C(=O)C(=O)N7CCN(c8ccc9c(C%10CCC(=O)NC%10=O)nn(C)c9c8)CC7)CC6)c5)ccc4c23)C(=O)N1] | Ex 14 |
| 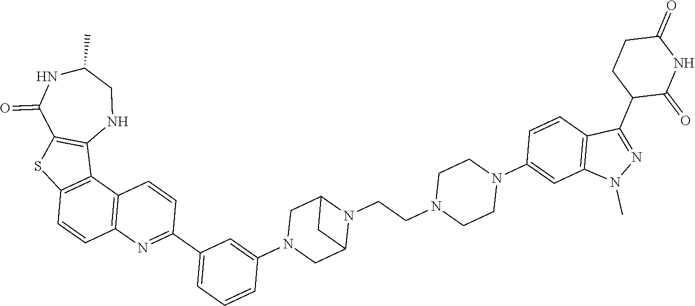 [SMILES: C[C@@H]1CNc2c(sc3ccc4nc(-c5cccc(N6CC7CC(C6)N7CCN6CCN(c7ccc8c(C9CCC(=O)NC9=O)nn(C)c8c7)CC6)c5)ccc4c23)C(=O)N1] | Ex 15 |
| 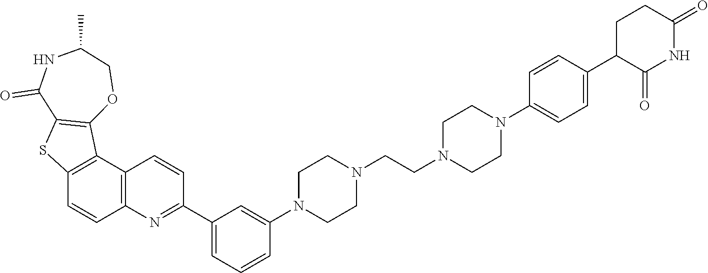 [SMILES: C[C@@H]1COc2c(sc3ccc4nc(-c5cccc(N6CCN(CCN7CCN(c8ccc(C9CCC(=O)NC9=O)cc8)CC7)CC6)c5)ccc4c23)C(=O)N1] | Ex 16 |
| 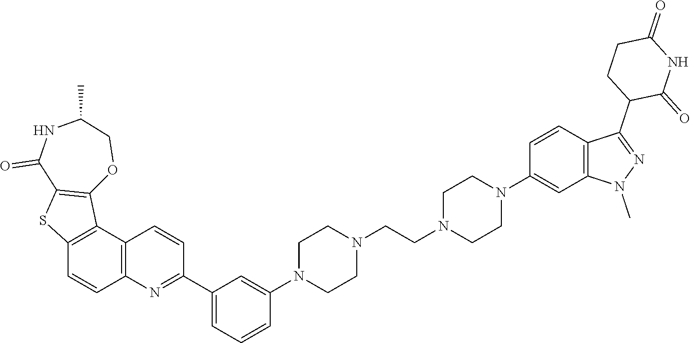 [SMILES: C[C@@H]1COc2c(sc3ccc4nc(-c5cccc(N6CCN(CCN7CCN(c8ccc9c(C%10CCC(=O)NC%10=O)nn(C)c9c8)CC7)CC6)c5)ccc4c23)C(=O)N1] | Ex 17 |
| 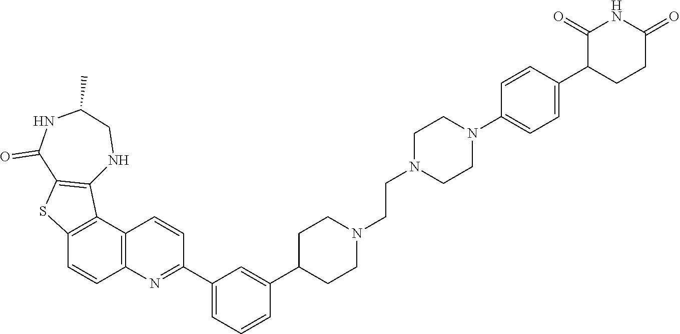 [SMILES: C[C@@H]1CNc2c(sc3ccc4nc(-c5cccc(C6CCN(CCN7CCN(c8ccc(C9CCC(=O)NC9=O)cc8)CC7)CC6)c5)ccc4c23)C(=O)N1] | Ex 18 |
| 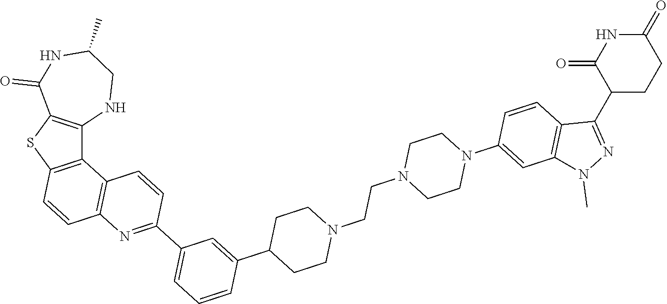 [SMILES: C[C@@H]1CNc2c(sc3ccc4nc(-c5cccc(C6CCN(CCN7CCN(c8ccc9c(C%10CCC(=O)NC%10=O)nn(C)c9c8)CC7)CC6)c5)ccc4c23)C(=O)N1] | Ex 19 |
| 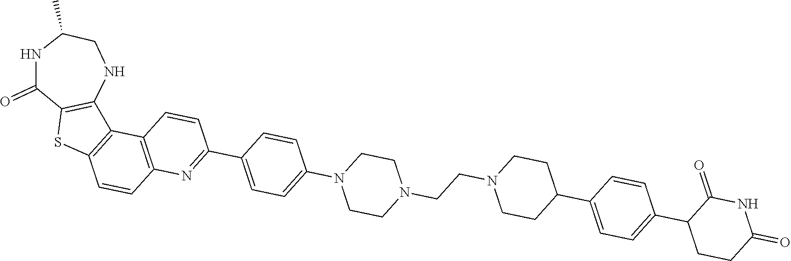 [SMILES: C[C@@H]1CNc2c(sc3ccc4nc(-c5ccc(N6CCN(CCN7CCC(c8ccc(C9CCC(=O)NC9=O)cc8)CC7)CC6)cc5)ccc4c23)C(=O)N1] | Ex 20 |
| 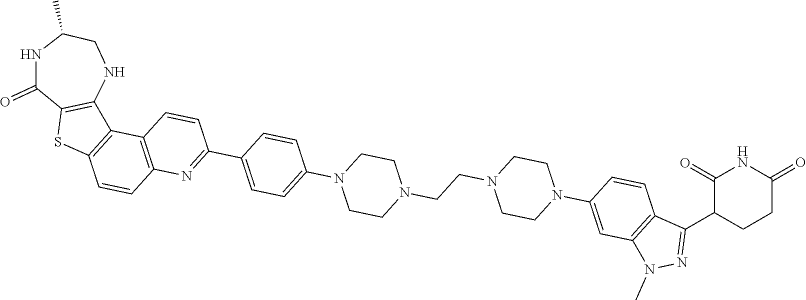 [SMILES: C[C@@H]1CNc2c(sc3ccc4nc(-c5ccc(N6CCN(CCN7CCN(c8ccc9c(C%10CCC(=O)NC%10=O)nn(C)c9c8)CC7)CC6)cc5)ccc4c23)C(=O)N1] | Ex 21 |
| 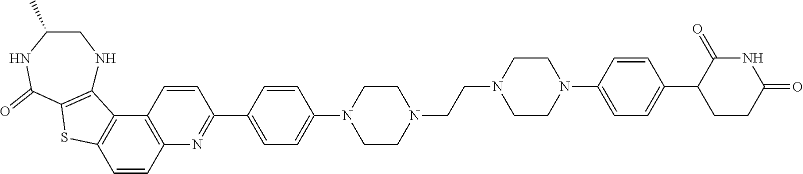 [SMILES: C[C@@H]1CNc2c(sc3ccc4nc(-c5ccc(N6CCN(CCN7CCN(c8ccc(C9CCC(=O)NC9=O)cc8)CC7)CC6)cc5)ccc4c23)C(=O)N1] | Ex 22 |
| 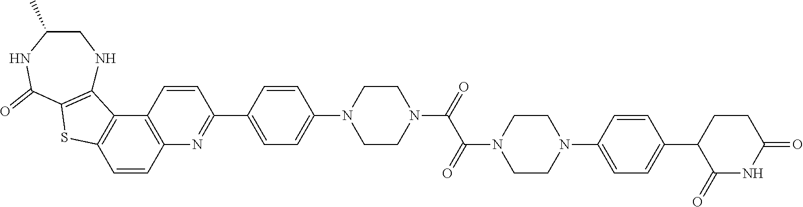 [SMILES: C[C@@H]1CNc2c(sc3ccc4nc(-c5ccc(N6CCN(C(=O)C(=O)N7CCN(c8ccc(C9CCC(=O)NC9=O)cc8)CC7)CC6)cc5)ccc4c23)C(=O)N1] | Ex 23 |
| 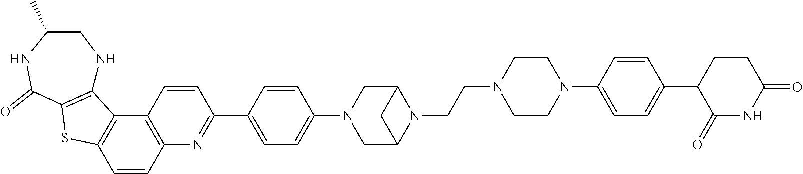 [SMILES: C[C@@H]1CNc2c(sc3ccc4nc(-c5ccc(N6CC7CC(C6)N7CCN6CCN(c7ccc(C8CCC(=O)NC8=O)cc7)CC6)cc5)ccc4c23)C(=O)N1] | Ex 24 |
| 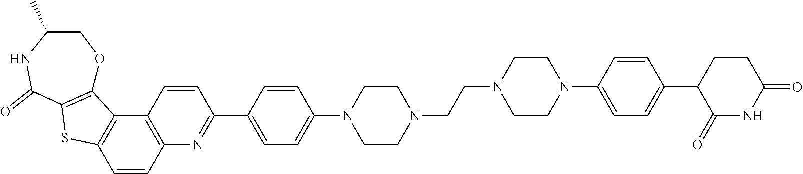 [SMILES: C[C@@H]1COc2c(sc3ccc4nc(-c5ccc(N6CCN(CCN7CCN(c8ccc(C9CCC(=O)NC9=O)cc8)CC7)CC6)cc5)ccc4c23)C(=O)N1] | Ex 25 |
| 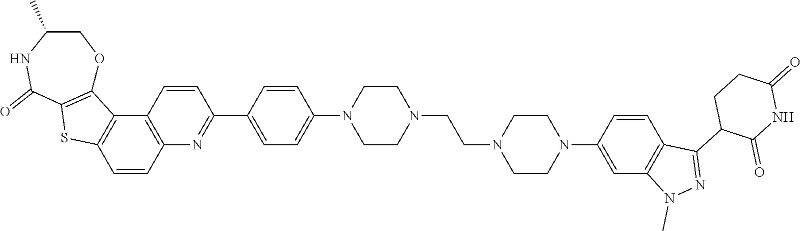 [SMILES: C[C@@H]1COc2c(sc3ccc4nc(-c5ccc(N6CCN(CCN7CCN(c8ccc9c(C%10CCC(=O)NC%10=O)nn(C)c9c8)CC7)CC6)cc5)ccc4c23)C(=O)N1] | Ex 26 |
| 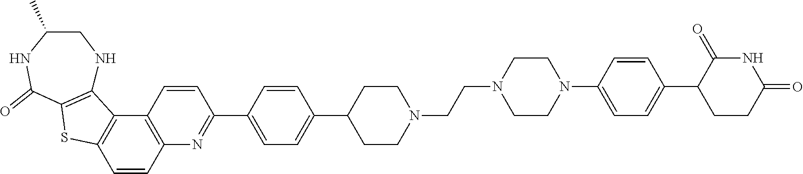 [SMILES: C[C@@H]1CNc2c(sc3ccc4nc(-c5ccc(C6CCN(CCN7CCN(c8ccc(C9CCC(=O)NC9=O)cc8)CC7)CC6)cc5)ccc4c23)C(=O)N1] | Ex 27 |
| 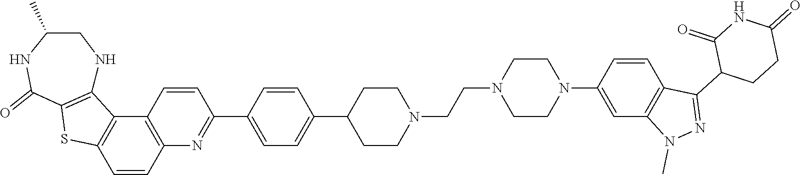 [SMILES: C[C@@H]1CNc2c(sc3ccc4nc(-c5ccc(C6CCN(CCN7CCN(c8ccc9c(C%10CCC(=O)NC%10=O)nn(C)c9c8)CC7)CC6)cc5)ccc4c23)C(=O)N1] | Ex 28 |
| 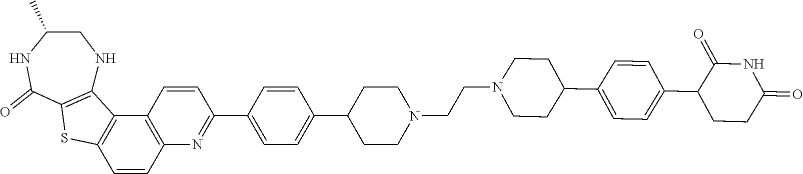 [SMILES: C[C@@H]1CNc2c(sc3ccc4nc(-c5ccc(C6CCN(CCN7CCC(c8ccc(C9CCC(=O)NC9=O)cc8)CC7)CC6)cc5)ccc4c23)C(=O)N1] | Ex 29 |
| 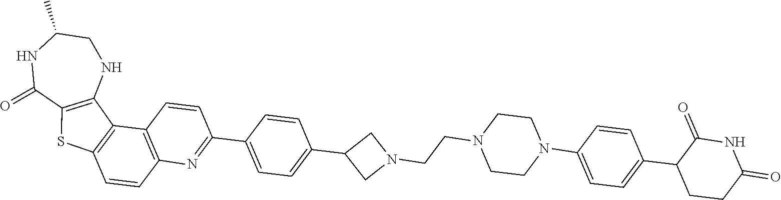 [SMILES: C[C@@H]1CNc2c(sc3ccc4nc(-c5ccc(C6CN(CCN7CCN(c8ccc(C9CCC(=O)NC9=O)cc8)CC7)C6)cc5)ccc4c23)C(=O)N1] | Ex 30 |
| 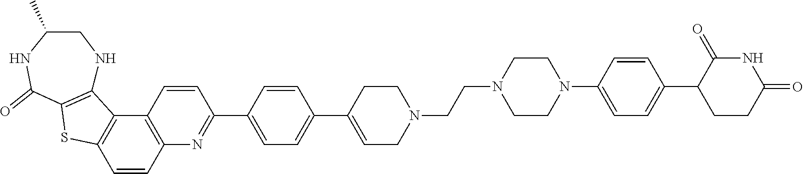 [SMILES: C[C@@H]1CNc2c(sc3ccc4nc(-c5ccc(C6=CCN(CCN7CCN(c8ccc(C9CCC(=O)NC9=O)cc8)CC7)CC6)cc5)ccc4c23)C(=O)N1] | Ex 31 |
| 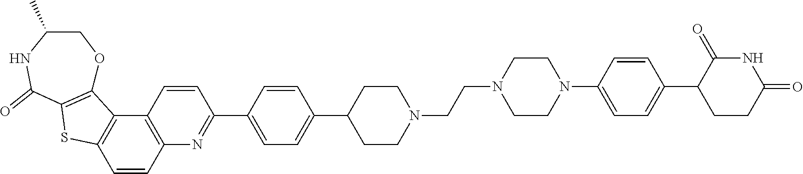 [SMILES: C[C@@H]1COc2c(sc3ccc4nc(-c5ccc(C6CCN(CCN7CCN(c8ccc(C9CCC(=O)NC9=O)cc8)CC7)CC6)cc5)ccc4c23)C(=O)N1] | Ex 32 |
| 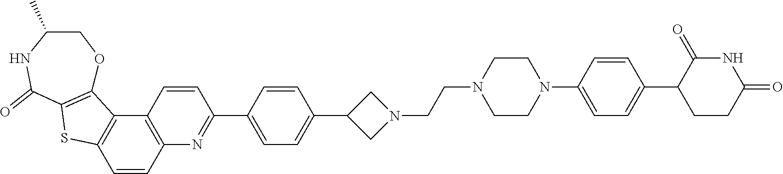 [SMILES: C[C@@H]1COc2c(sc3ccc4nc(-c5ccc(C6CN(CCN7CCN(c8ccc(C9CCC(=O)NC9=O)cc8)CC7)C6)cc5)ccc4c23)C(=O)N1] | Ex 33 |
| 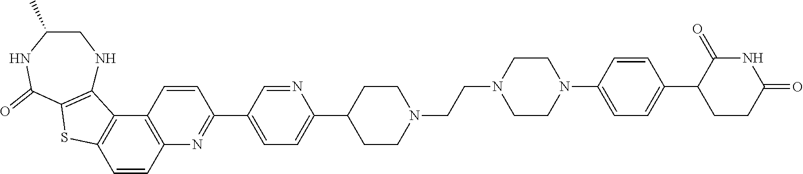 [SMILES: C[C@@H]1CNc2c(sc3ccc4nc(-c5ccc(C6CCN(CCN7CCN(c8ccc(C9CCC(=O)NC9=O)cc8)CC7)CC6)nc5)ccc4c23)C(=O)N1] | Ex 34 |
| 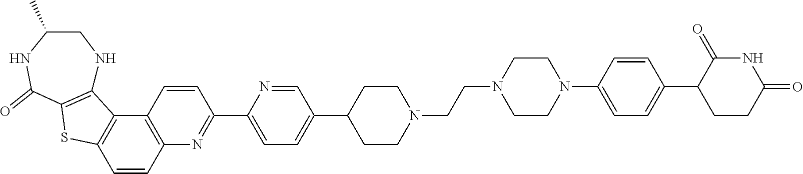 [SMILES: C[C@@H]1CNc2c(sc3ccc4nc(-c5ccc(C6CCN(CCN7CCN(c8ccc(C9CCC(=O)NC9=O)cc8)CC7)CC6)cn5)ccc4c23)C(=O)N1] | Ex 35 |
| 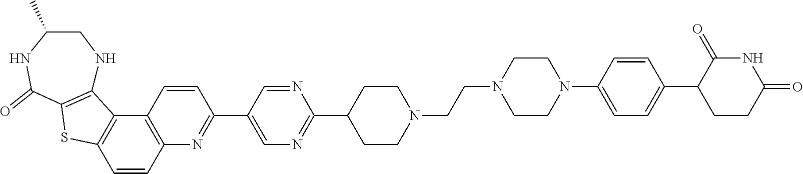 [SMILES: C[C@@H]1CNc2c(sc3ccc4nc(-c5cnc(C6CCN(CCN7CCN(c8ccc(C9CCC(=O)NC9=O)cc8)CC7)CC6)nc5)ccc4c23)C(=O)N1] | Ex 36 |
| 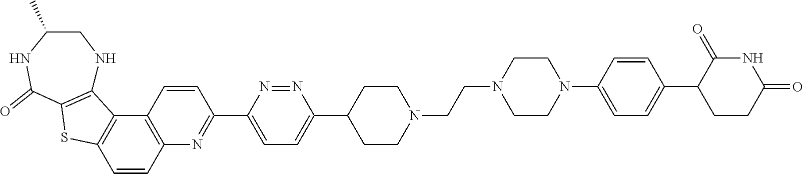 [SMILES: C[C@@H]1CNc2c(sc3ccc4nc(-c5ccc(C6CCN(CCN7CCN(c8ccc(C9CCC(=O)NC9=O)cc8)CC7)CC6)nn5)ccc4c23)C(=O)N1] | Ex 37 |
| 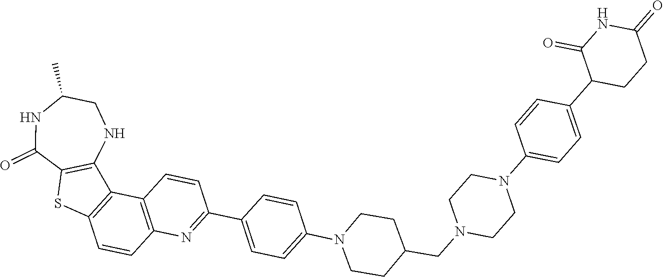 [SMILES: C[C@@H]1CNc2c(sc3ccc4nc(-c5ccc(N6CCC(CN7CCN(c8ccc(C9CCC(=O)NC9=O)cc8)CC7)CC6)cc5)ccc4c23)C(=O)N1] | Ex 38 |
| 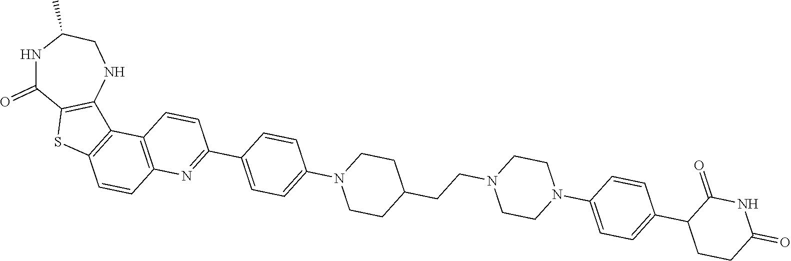 [SMILES: C[C@@H]1CNc2c(sc3ccc4nc(-c5ccc(N6CCC(CCN7CCN(c8ccc(C9CCC(=O)NC9=O)cc8)CC7)CC6)cc5)ccc4c23)C(=O)N1] | Ex 39 |
| 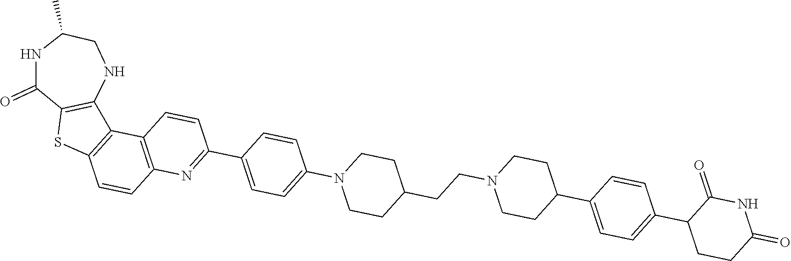 [SMILES: C[C@@H]1CNc2c(sc3ccc4nc(-c5ccc(N6CCC(CCN7CCC(c8ccc(C9CCC(=O)NC9=O)cc8)CC7)CC6)cc5)ccc4c23)C(=O)N1] | Ex 40 |
| 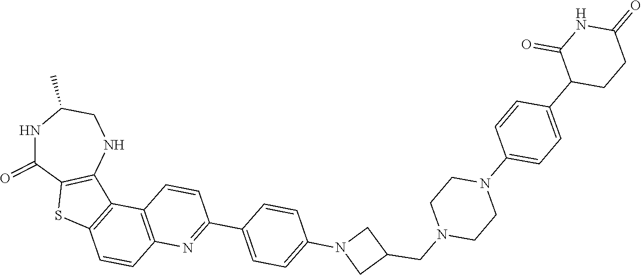 [SMILES: C[C@@H]1CNc2c(sc3ccc4nc(-c5ccc(N6CC(CN7CCN(c8ccc(C9CCC(=O)NC9=O)cc8)CC7)C6)cc5)ccc4c23)C(=O)N1] | Ex 41 |
| 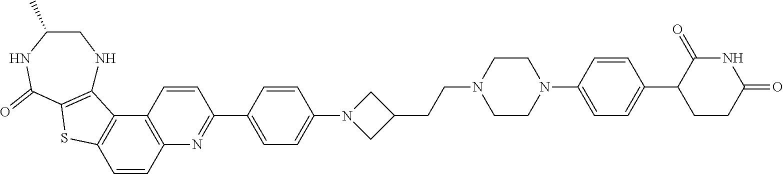 [SMILES: C[C@@H]1CNc2c(sc3ccc4nc(-c5ccc(N6CC(CCN7CCN(c8ccc(C9CCC(=O)NC9=O)cc8)CC7)C6)cc5)ccc4c23)C(=O)N1] | Ex 42 |
| 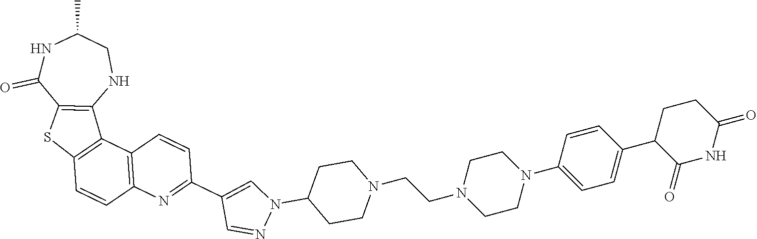 [SMILES: C[C@@H]1CNc2c(sc3ccc4nc(-c5cnn(C6CCN(CCN7CCN(c8ccc(C9CCC(=O)NC9=O)cc8)CC7)CC6)c5)ccc4c23)C(=O)N1] | Ex 43 |
| 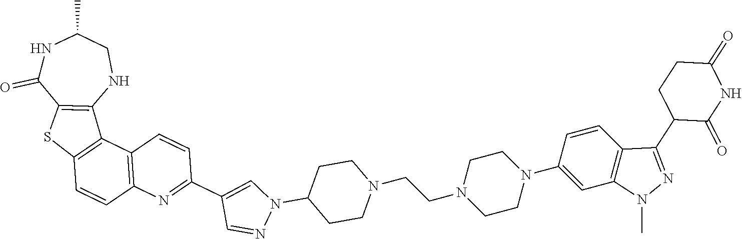 [SMILES: C[C@@H]1CNc2c(sc3ccc4nc(-c5cnn(C6CCN(CCN7CCN(c8ccc9c(C%10CCC(=O)NC%10=O)nn(C)c9c8)CC7)CC6)c5)ccc4c23)C(=O)N1] | Ex 44 |
| 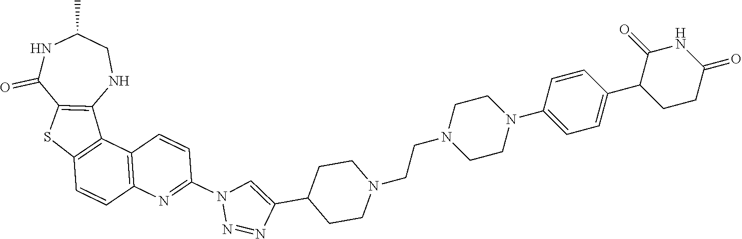 [SMILES: C[C@@H]1CNc2c(sc3ccc4nc(-n5cc(C6CCN(CCN7CCN(c8ccc(C9CCC(=O)NC9=O)cc8)CC7)CC6)nn5)ccc4c23)C(=O)N1] | Ex 45 |
| 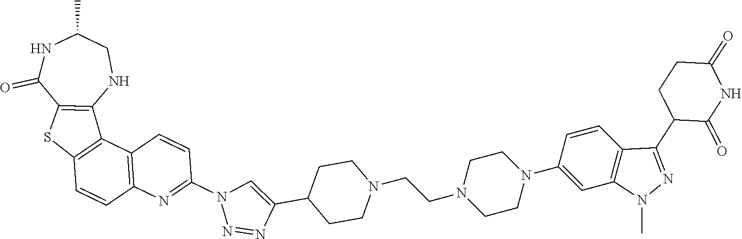 [SMILES: C[C@@H]1CNc2c(sc3ccc4nc(-n5cc(C6CCN(CCN7CCN(c8ccc9c(C%10CCC(=O)NC%10=O)nn(C)c9c8)CC7)CC6)nn5)ccc4c23)C(=O)N1] | Ex 46 |
| 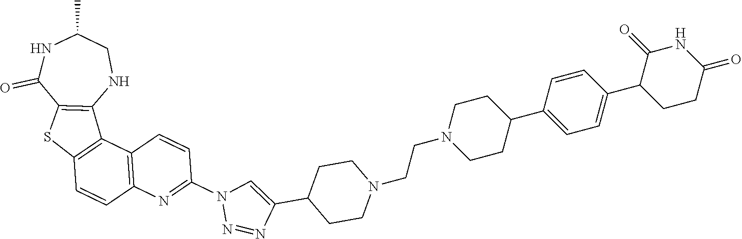 [SMILES: C[C@@H]1CNc2c(sc3ccc4nc(-n5cc(C6CCN(CCN7CCC(c8ccc(C9CCC(=O)NC9=O)cc8)CC7)CC6)nn5)ccc4c23)C(=O)N1] | Ex 47 |
| 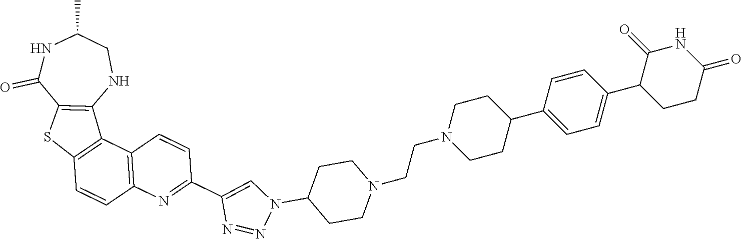 [SMILES: C[C@@H]1CNc2c(sc3ccc4nc(-c5cn(C6CCN(CCN7CCC(c8ccc(C9CCC(=O)NC9=O)cc8)CC7)CC6)nn5)ccc4c23)C(=O)N1] | Ex 48 |
| 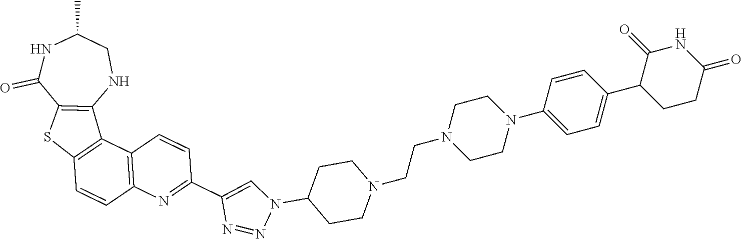 [SMILES: C[C@@H]1CNc2c(sc3ccc4nc(-c5cn(C6CCN(CCN7CCN(c8ccc(C9CCC(=O)NC9=O)cc8)CC7)CC6)nn5)ccc4c23)C(=O)N1] | Ex 49 |
| 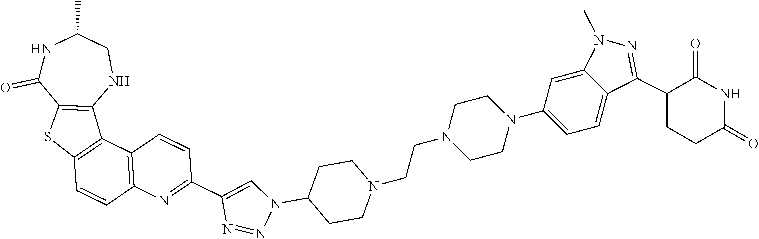 [SMILES: C[C@@H]1CNc2c(sc3ccc4nc(-c5cn(C6CCN(CCN7CCN(c8ccc9c(C%10CCC(=O)NC%10=O)nn(C)c9c8)CC7)CC6)nn5)ccc4c23)C(=O)N1] | Ex 50 |
| 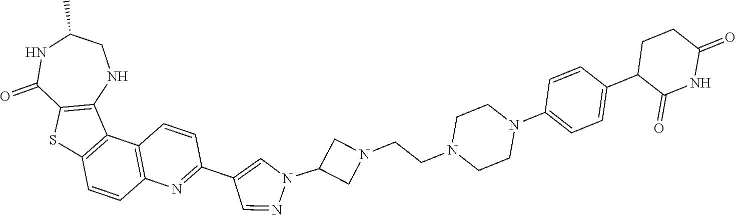 [SMILES: C[C@@H]1CNc2c(sc3ccc4nc(-c5cnn(C6CN(CCN7CCN(c8ccc(C9CCC(=O)NC9=O)cc8)CC7)C6)c5)ccc4c23)C(=O)N1] | Ex 51 |
| 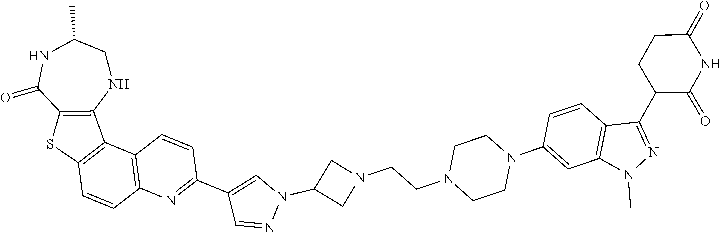 [SMILES: C[C@@H]1CNc2c(sc3ccc4nc(-c5cnn(C6CN(CCN7CCN(c8ccc9c(C%10CCC(=O)NC%10=O)nn(C)c9c8)CC7)C6)c5)ccc4c23)C(=O)N1] | Ex 52 |
| 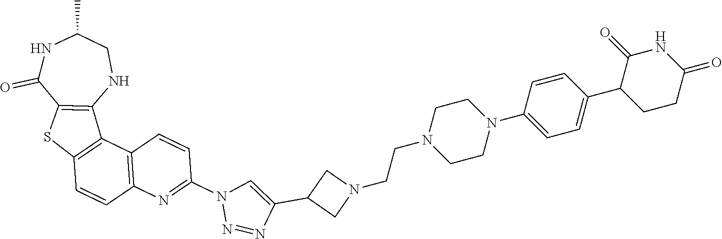 [SMILES: C[C@@H]1CNc2c(sc3ccc4nc(-n5cc(C6CN(CCN7CCN(c8ccc(C9CCC(=O)NC9=O)cc8)CC7)C6)nn5)ccc4c23)C(=O)N1] | Ex 53 |
| 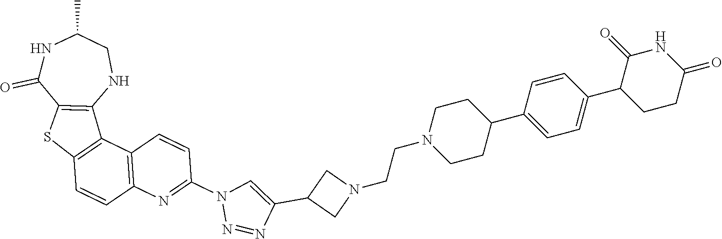 [SMILES: C[C@@H]1CNc2c(sc3ccc4nc(-n5cc(C6CN(CCN7CCC(c8ccc(C9CCC(=O)NC9=O)cc8)CC7)C6)nn5)ccc4c23)C(=O)N1] | Ex 54 |
| 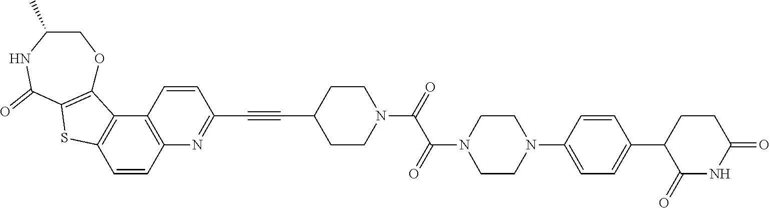 [SMILES: C[C@@H]1COc2c(sc3ccc4nc(C#CC5CCN(C(=O)C(=O)N6CCN(c7ccc(C8CCC(=O)NC8=O)cc7)CC6)CC5)ccc4c23)C(=O)N1] | Ex 55 |

or a pharmaceutically acceptable salt thereof.

In some embodiments, the compounds or pharmaceutically acceptable salts of the present disclosure have the Formula:

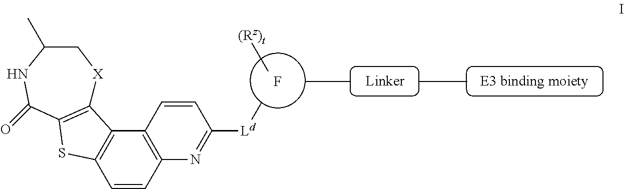

or a pharmaceutically acceptable salt thereof, wherein:
the Linker is a bivalent group;
the E3 binding moiety is a moiety that binds to an E3 ubiquitin ligase protein;
Ring F is phenylene, a 5- to 6-membered heteroarylene ring having 1-4 heteroatoms independently selected from nitrogen, oxygen, and sulfur, or a 3- to 7-membered saturated or partially unsaturated heterocyclylene having 1-3 heteroatoms independently selected from nitrogen, oxygen, and sulfur;
L^{d} is selected from a covalent bond, —O—, —S—, —N(R)—, and C₁₋₆ aliphatic;
X is selected from —O—, —S—, and —N(R)—;
R^{z} is selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur;
each R is independently hydrogen or optionally substituted C₁₋₆ aliphatic; and
t is 0, 1, 2, or 3.

In some embodiments of the compounds of the present disclosure, the Ring F is phenylene.

In some embodiments of the compounds of the present disclosure L^{d} is a covalent bond.

In some embodiments of the compounds of the present disclosure X is —O—.

In some embodiments of the compounds of the present disclosure, X is —N(R)—.

In some embodiments of the compounds of the present disclosure, R is hydrogen.

In some embodiments of the compounds of the present disclosure, R^{z} is selected from halogen, —OR, —SR, —CN, —NO₂, —SO₂NR, —SO₂R, —SOR, —C(O)R, —CO₂R, and —C(O)N(R)₂.

In some embodiments of the compounds of the present disclosure, R^{z} is selected from halogen, —OR, —SR, —CN, and —NO₂.

In some embodiments of the compounds of the present disclosure, t is 0.

In some embodiments of the compounds of the present disclosure, t is 1.

In some embodiments of the compounds of the present disclosure, the compound is a compound of any of Formulae I-a, I-a-i, I-a-ii, I-a-iii, I-a-iv, I-a-v, I-b, I-b-i, I-b-ii, I-b-iii, I-b-iv, I-b-v, I-c, I-c-i, I-c-ii, I-c-iii, I-c-iv, I-c-v, I-d, I-d-i, and I-d-ii:

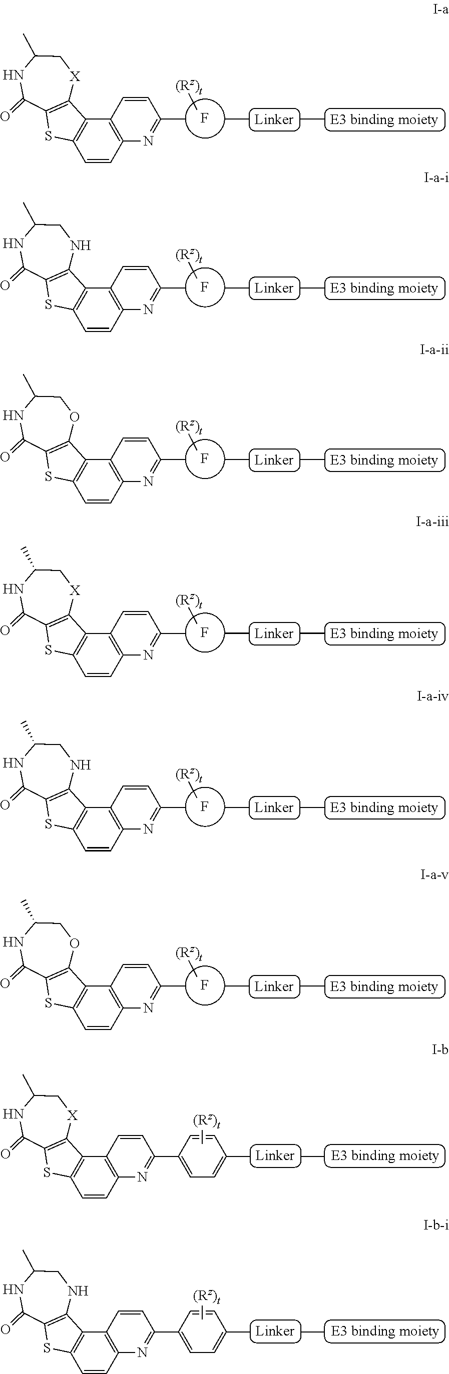

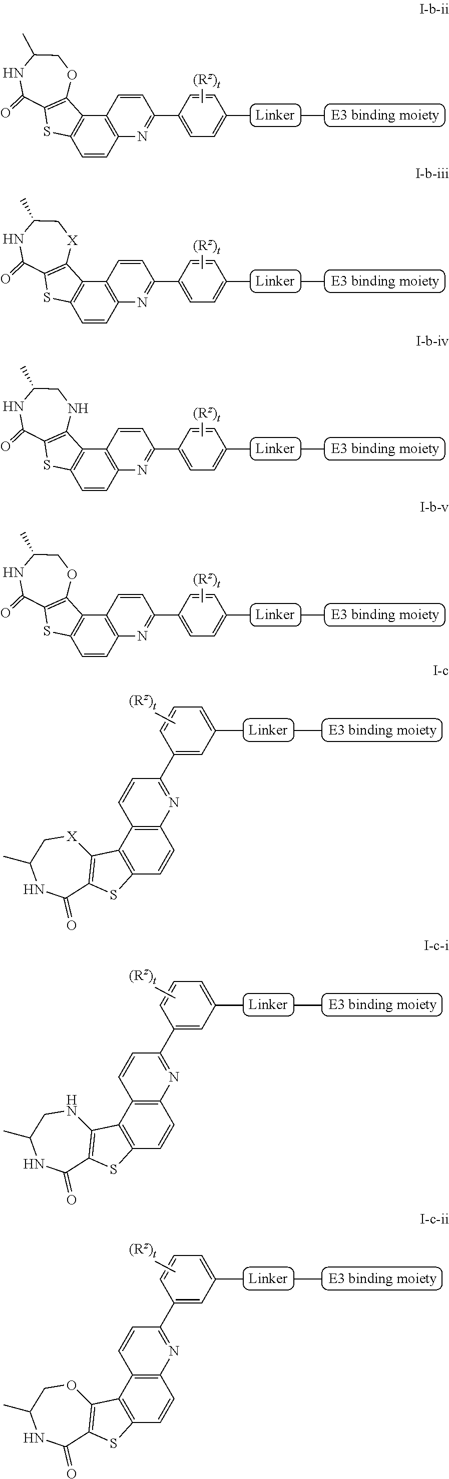

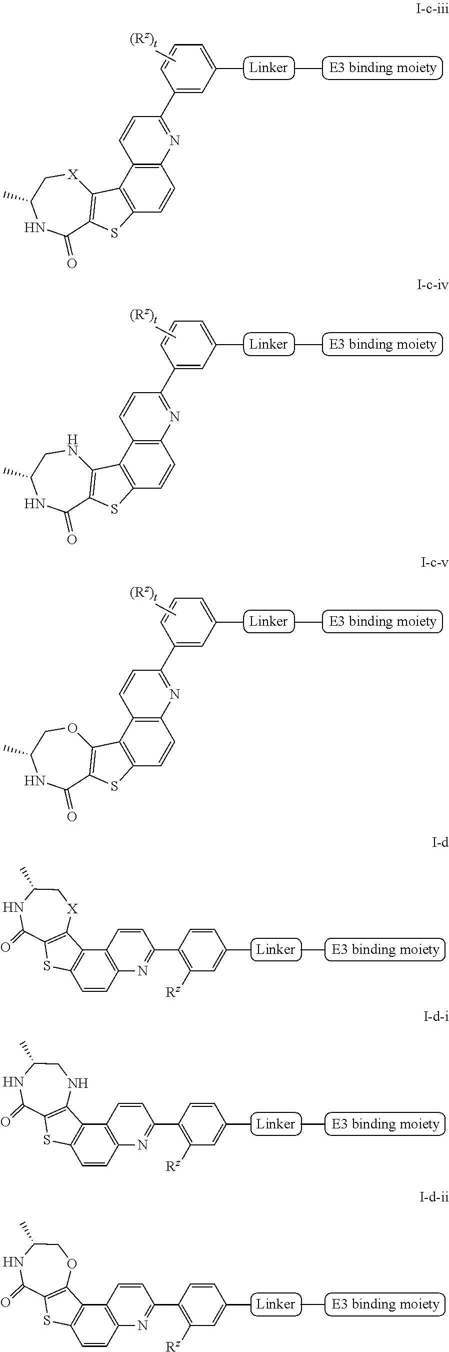

or a pharmaceutically acceptable salt thereof.

In some embodiments of the compounds of the present disclosure, the Linker is an optionally substituted bivalent C₂₋₂₀ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}.

and
R^{L} is independently selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur.

In some embodiments of the compounds of the present disclosure, the Linker is an optionally substituted bivalent C₂₋₂₀ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments of the compounds of the present disclosure, the Linker is an optionally substituted bivalent C₂₋₂₀ straight or branched aliphatic chain, wherein two, three, four, or five methylene units of the aliphatic chain are optionally and independently replaced by a group selected from —C(O)N(R)—, —N(R)—, —C(O)—,

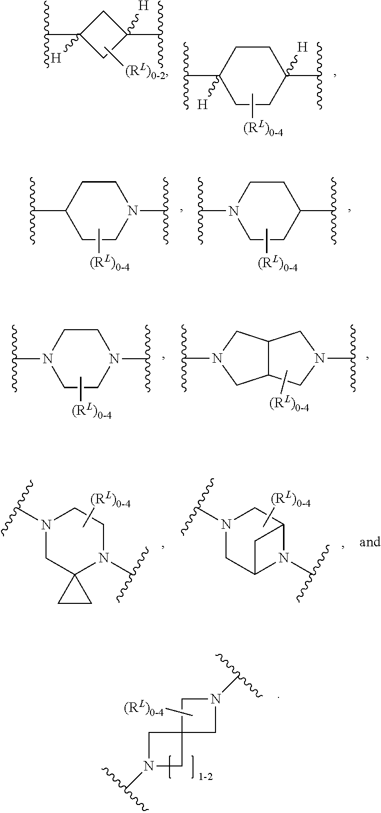

In some embodiments of the compounds of the present disclosure, the Linker is an optionally substituted bivalent C₂₋₂₀ straight or branched aliphatic chain, wherein two, three, four, or five methylene units of the aliphatic chain are optionally and independently replaced by a group selected from —C(O)N(R)—, —N(R)—, —C(O)—,

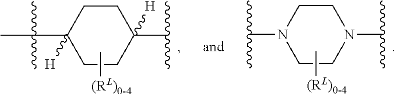

In some embodiments of the compounds of the present disclosure, the Linker is an optionally substituted bivalent C₃₋₁₇ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}.

In some embodiments of the compounds of the present disclosure, the Linker is an optionally substituted bivalent C₃₋₁₇ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments of the compounds of the present disclosure, the Linker is an optionally substituted bivalent C₃₋₁₇ straight or branched aliphatic chain, wherein two, three, four, or five methylene units of the aliphatic chain are optionally and independently replaced by a group selected from —C(O)N(R)—, —N(R)—, —C(O)—,

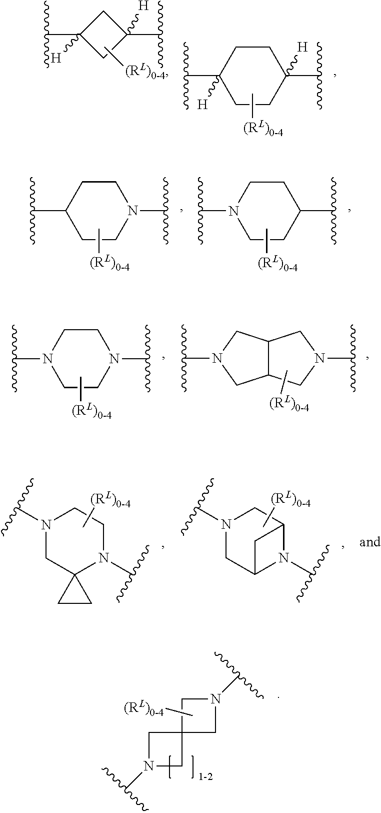

In some embodiments of the compounds of the present disclosure, the Linker is an optionally substituted bivalent C₃₋₁₇ straight or branched aliphatic chain, wherein two, three, four, or five methylene units of the aliphatic chain are optionally and independently replaced by a group selected from —C(O)N(R)—, —N(R)—, —C(O)—,

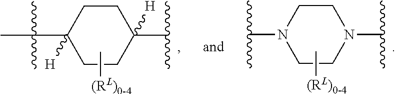

In some embodiments of the compounds of the present disclosure, the Linker is an optionally substituted bivalent C₂₋₁₀ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}.

In some embodiments of the compounds of the present disclosure, the Linker is an optionally substituted bivalent C₂₋₁₀ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments of the compounds of the present disclosure, the Linker is an optionally substituted bivalent C₂₋₁₀ straight or branched aliphatic chain, wherein two, three, four, or five methylene units of the aliphatic chain are optionally and independently replaced by a group selected from —C(O)N(R)—, —N(R)—, —C(O)—,

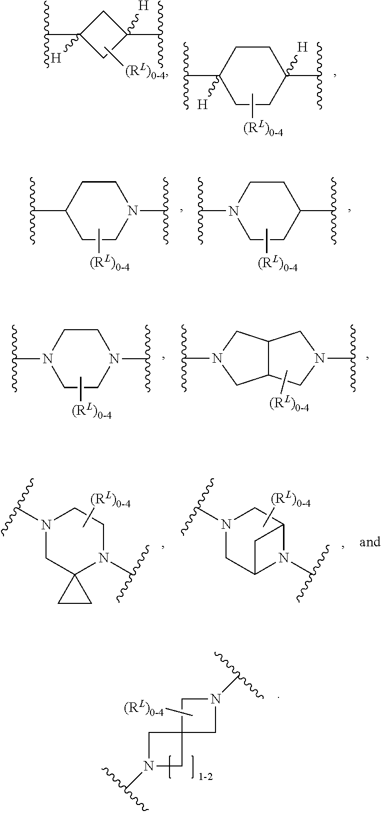

In some embodiments of the compounds of the present disclosure, the Linker is an optionally substituted bivalent C₂₋₁₀ straight or branched aliphatic chain, wherein two, three, four, or five methylene units of the aliphatic chain are optionally and independently replaced by a group selected from —C(O)N(R)—, —N(R)—, —C(O)—,

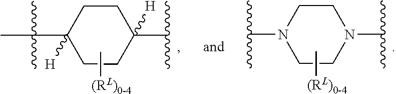

In some embodiments of the compounds of the present disclosure, the Linker is an optionally substituted bivalent C₂₋₆ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}.

In some embodiments of the compounds of the present disclosure, the Linker is an optionally substituted bivalent C₂₋₆ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments of the compounds of the present disclosure, the Linker is an optionally substituted bivalent C₂₋₆ straight or branched aliphatic chain, wherein two, three, four, or five methylene units of the aliphatic chain are optionally and independently replaced by a group selected from —C(O)N(R)—, —N(R)—, —C(O)—,

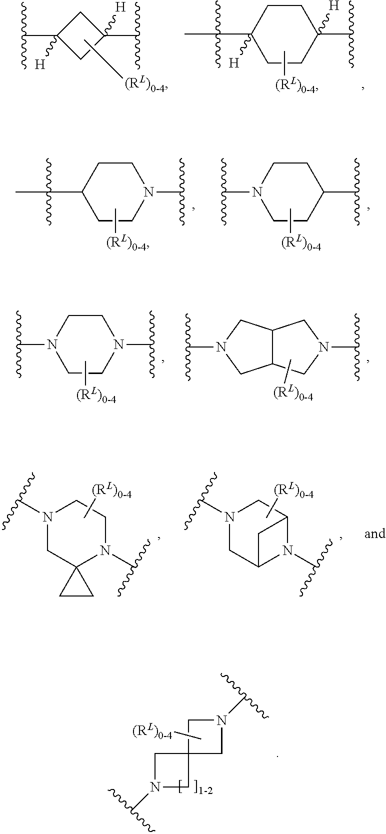

In some embodiments of the compounds of the present disclosure, the Linker is an optionally substituted bivalent C₂₋₆ straight or branched aliphatic chain, wherein two, three, four, or five methylene units of the aliphatic chain are optionally and independently replaced by a group selected from —C(O)N(R)—, —N(R)—, —C(O)—,

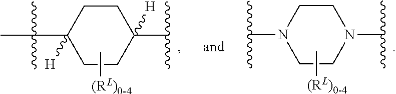

In some embodiments of the compounds of the present disclosure, the Linker is an optionally substituted bivalent C₄₋₆ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}.

In some embodiments of the compounds of the present disclosure, the Linker is an optionally substituted bivalent C₄₋₆ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

In some embodiments of the compounds of the present disclosure, the Linker is an optionally substituted bivalent C₄₋₆ straight or branched aliphatic chain, wherein two, three, four, or five methylene units of the aliphatic chain are optionally and independently replaced by a group selected from —C(O)N(R)—, —N(R)—, —C(O)—,

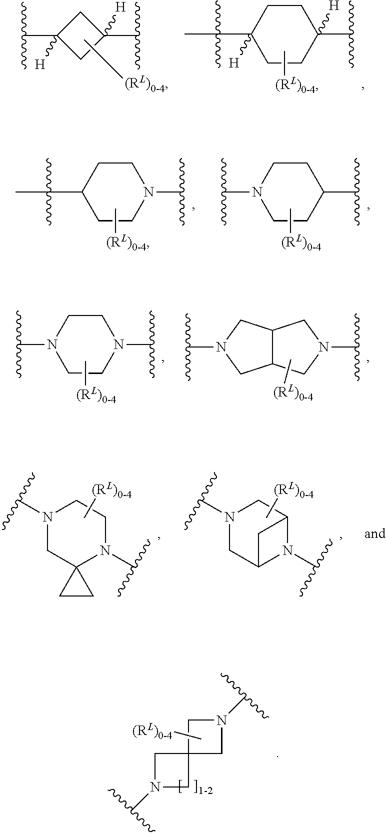

In some embodiments of the compounds of the present disclosure, the Linker is an optionally substituted bivalent C₄₋₆ straight or branched aliphatic chain, wherein two, three, four, or five methylene units of the aliphatic chain are optionally and independently replaced by a group selected from —C(O)N(R)—, —N(R)—, —C(O)—,

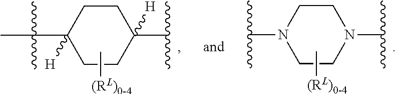

In some embodiments of the compounds of the present disclosure, the Linker is selected from the group consisting of:

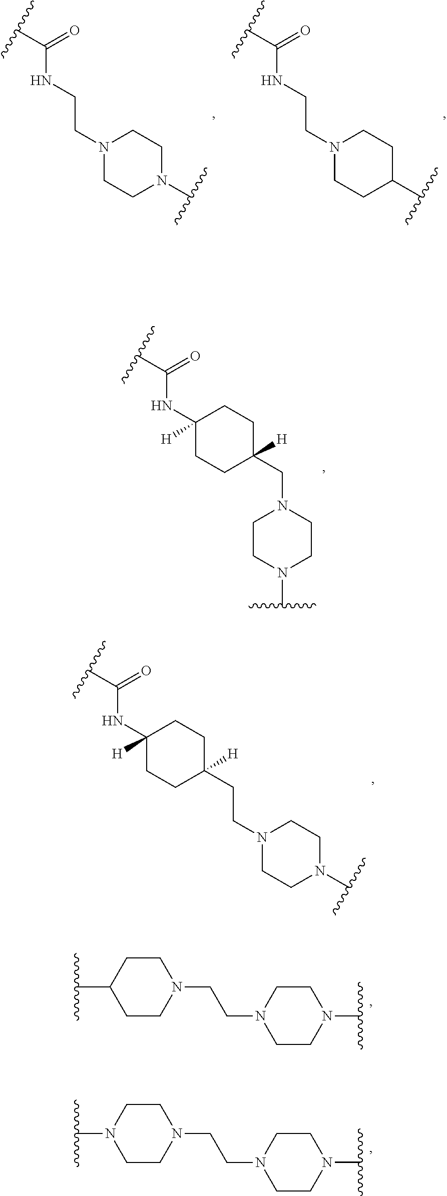 [SMILES: *C(=O)NCCN1CCN(*)CC1 ; *C(=O)NCCN1CCC(*)CC1 ; *C(=O)N[C@H]1CC[C@H](CN2CCN(*)CC2)CC1 ; *C(=O)N[C@H]1CC[C@H](CCN2CCN(*)CC2)CC1 ; *C1CCN(CCN2CCN(*)CC2)CC1 ; *N1CCN(CCN2CCN(*)CC2)CC1]

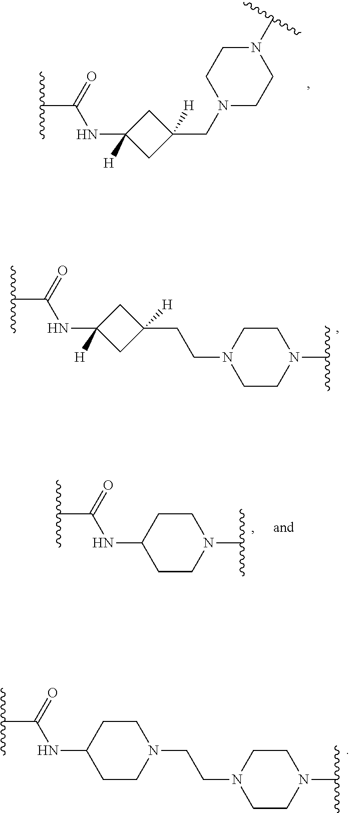 [SMILES: *C(=O)N[C@H]1C[C@H](CN2CCN(*)CC2)C1 ; *C(=O)N[C@H]1C[C@H](CCN2CCN(*)CC2)C1 ; *C(=O)NC1CCN(*)CC1 ; *C(=O)NC1CCN(CCN2CCN(*)CC2)CC1]

In some embodiments of the compounds of the present disclosure, the Linker is selected from the group consisting of:

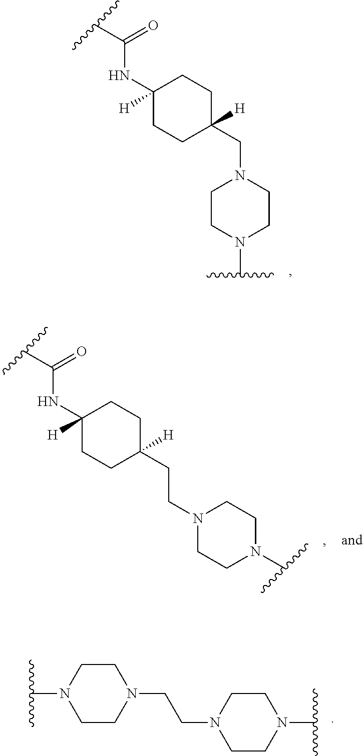 [SMILES: *C(=O)N[C@H]1CC[C@H](CN2CCN(*)CC2)CC1 ; *C(=O)N[C@H]1CC[C@H](CCN2CCN(*)CC2)CC1 ; *N1CCN(CCN2CCN(*)CC2)CC1]

In some embodiments of the compounds of the present disclosure, the E3 binding moiety is a cereblon protein binding moiety.

In some embodiments of the compounds of the present disclosure, the cereblon protein binding moiety is selected from:

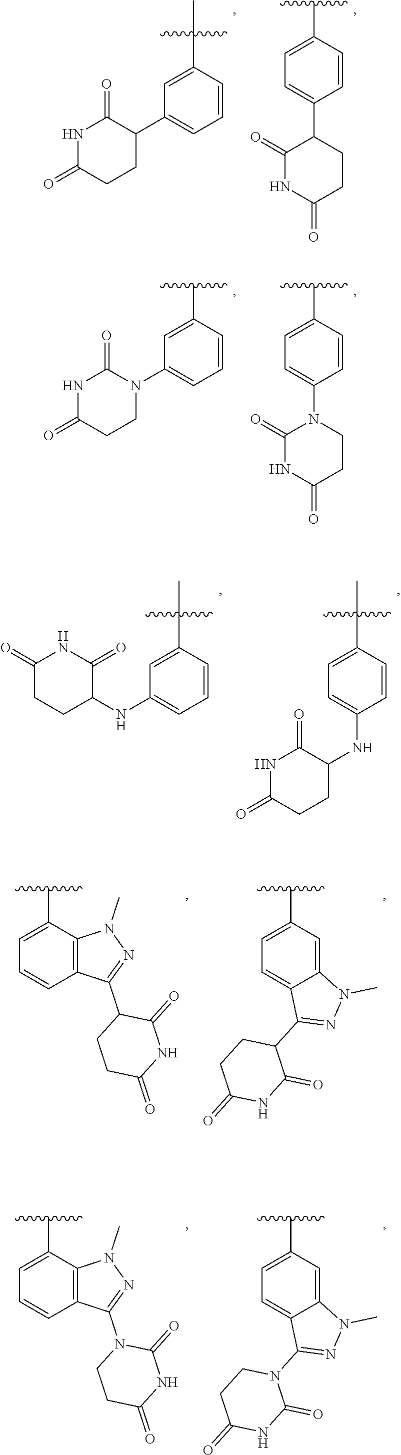

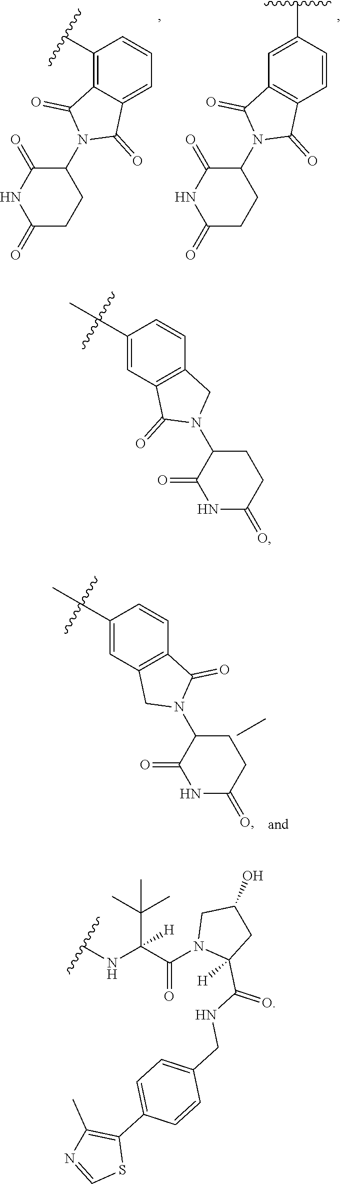

In some embodiments of the compounds of the present disclosure, the cereblon protein binding moiety is selected from:

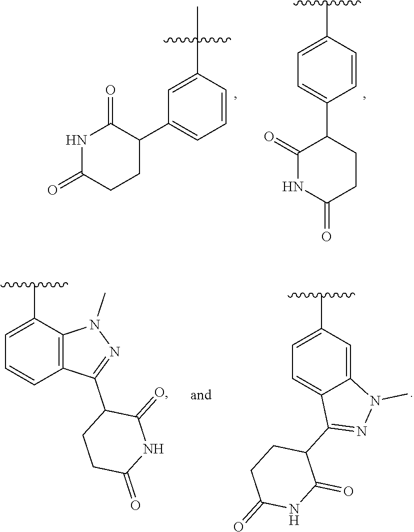

In some embodiments, the compound or pharmaceutically acceptable salt is selected from Table 1:

TABLE 1
Exemplary Compounds

| | |
|---|---|
| 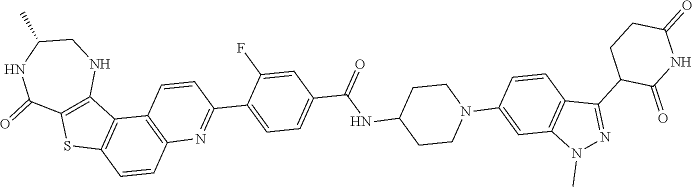 [SMILES: C[C@@H]1CNc2c(sc3ccc4nc(-c5ccc(C(=O)NC6CCN(c7ccc8c(C9CCC(=O)NC9=O)nn(C)c8c7)CC6)cc5F)ccc4c23)C(=O)N1] | Ex 1 |
| 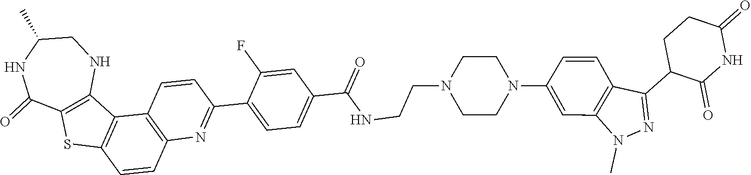 [SMILES: C[C@@H]1CNc2c(sc3ccc4nc(-c5ccc(C(=O)NCCN6CCN(c7ccc8c(C9CCC(=O)NC9=O)nn(C)c8c7)CC6)cc5F)ccc4c23)C(=O)N1] | Ex 2 |
| 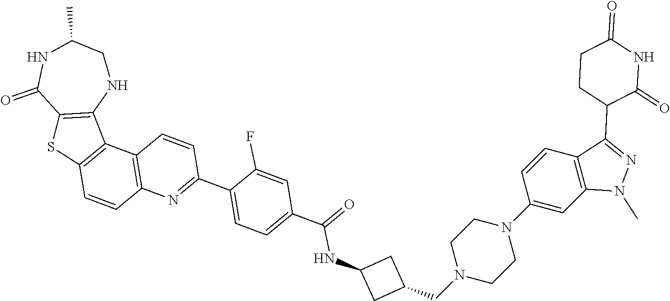 [SMILES: C[C@@H]1CNc2c(sc3ccc4nc(-c5ccc(C(=O)N[C@H]6C[C@H](CN7CCN(c8ccc9c(C%10CCC(=O)NC%10=O)nn(C)c9c8)CC7)C6)cc5F)ccc4c23)C(=O)N1] | Ex 3 |
| 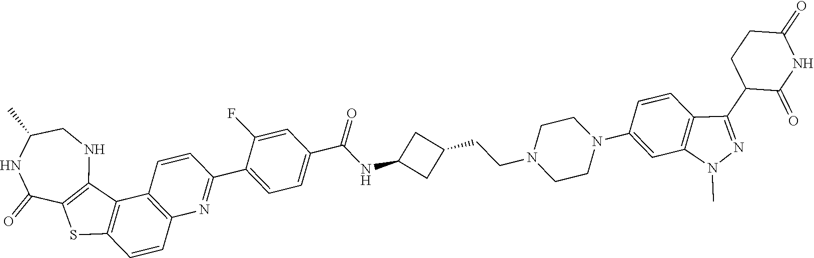 [SMILES: C[C@@H]1CNc2c(sc3ccc4nc(-c5ccc(C(=O)N[C@H]6C[C@H](CCN7CCN(c8ccc9c(C%10CCC(=O)NC%10=O)nn(C)c9c8)CC7)C6)cc5F)ccc4c23)C(=O)N1] | Ex 4 |
| 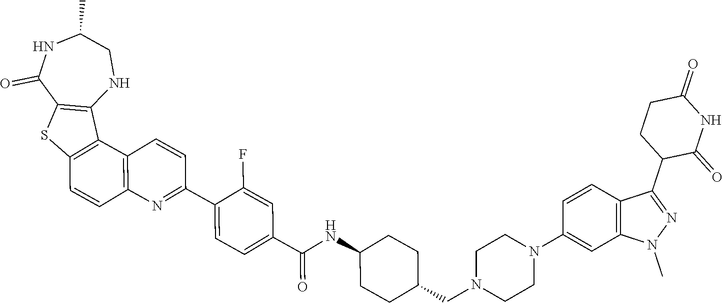 [SMILES: C[C@@H]1CNc2c(sc3ccc4nc(-c5ccc(C(=O)N[C@H]6CC[C@H](CN7CCN(c8ccc9c(C%10CCC(=O)NC%10=O)nn(C)c9c8)CC7)CC6)cc5F)ccc4c23)C(=O)N1] | Ex 5 |
| 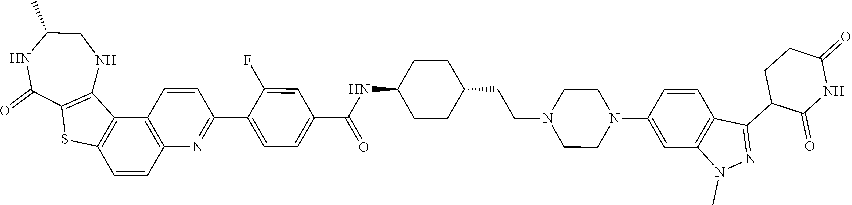 [SMILES: C[C@@H]1CNc2c(sc3ccc4nc(-c5ccc(C(=O)N[C@H]6CC[C@H](CCN7CCN(c8ccc9c(C%10CCC(=O)NC%10=O)nn(C)c9c8)CC7)CC6)cc5F)ccc4c23)C(=O)N1] | Ex 6 |
| 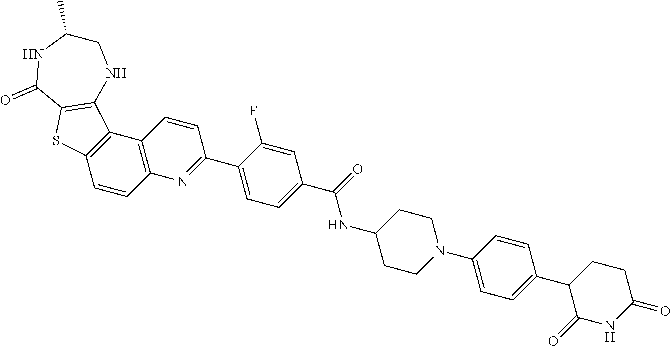 [SMILES: C[C@@H]1CNc2c(sc3ccc4nc(-c5ccc(C(=O)NC6CCN(c7ccc(C8CCC(=O)NC8=O)cc7)CC6)cc5F)ccc4c23)C(=O)N1] | Ex 7 |
| 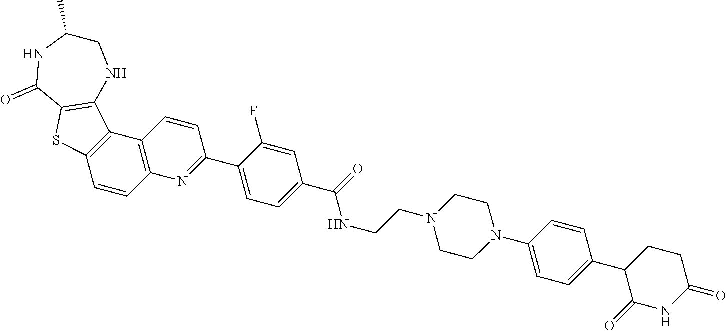 [SMILES: C[C@@H]1CNc2c(sc3ccc4nc(-c5ccc(C(=O)NCCN6CCN(c7ccc(C8CCC(=O)NC8=O)cc7)CC6)cc5F)ccc4c23)C(=O)N1] | Ex 8 |
| 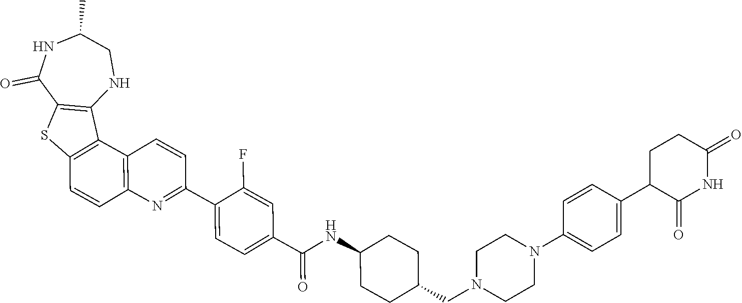 [SMILES: C[C@@H]1CNc2c(sc3ccc4nc(-c5ccc(C(=O)N[C@H]6CC[C@H](CN7CCN(c8ccc(C9CCC(=O)NC9=O)cc8)CC7)CC6)cc5F)ccc4c23)C(=O)N1] | Ex 9 |
| 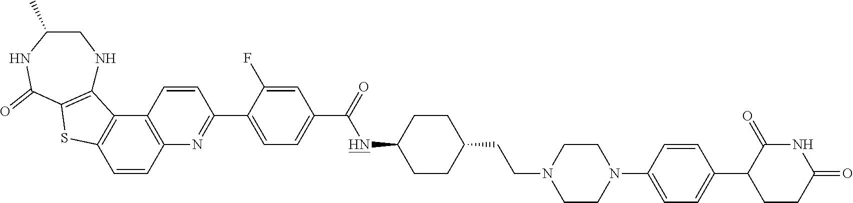 [SMILES: C[C@@H]1CNc2c(sc3ccc4nc(-c5ccc(C(=O)N[C@H]6CC[C@H](CCN7CCN(c8ccc(C9CCC(=O)NC9=O)cc8)CC7)CC6)cc5F)ccc4c23)C(=O)N1] | Ex 10 |
| 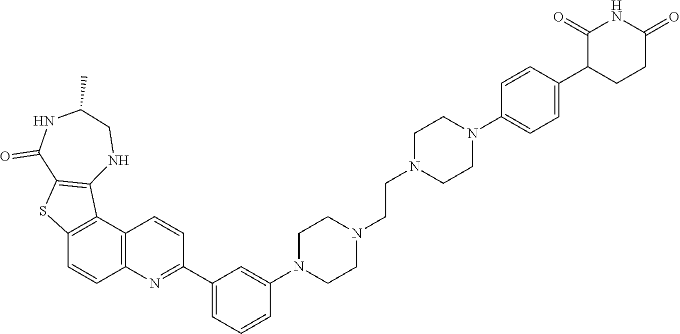 [SMILES: C[C@@H]1CNc2c(sc3ccc4nc(-c5cccc(N6CCN(CCN7CCN(c8ccc(C9CCC(=O)NC9=O)cc8)CC7)CC6)c5)ccc4c23)C(=O)N1] | Ex 11 |
| 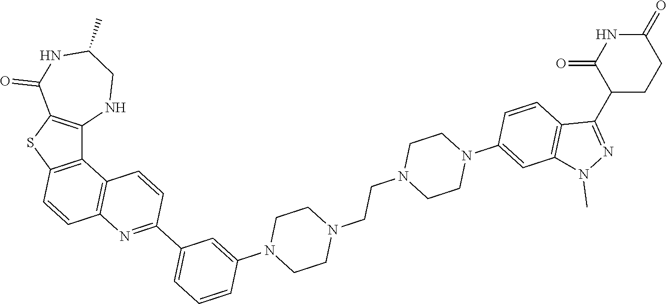 [SMILES: C[C@@H]1CNc2c(sc3ccc4nc(-c5cccc(N6CCN(CCN7CCN(c8ccc9c(C%10CCC(=O)NC%10=O)nn(C)c9c8)CC7)CC6)c5)ccc4c23)C(=O)N1] | Ex 12 |
| 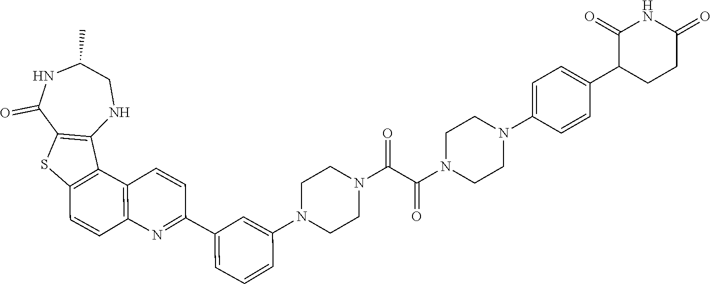 [SMILES: C[C@@H]1CNc2c(sc3ccc4nc(-c5cccc(N6CCN(C(=O)C(=O)N7CCN(c8ccc(C9CCC(=O)NC9=O)cc8)CC7)CC6)c5)ccc4c23)C(=O)N1] | Ex 13 |
| 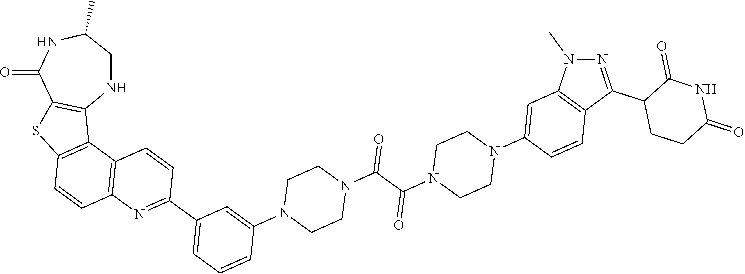 [SMILES: C[C@@H]1CNc2c(sc3ccc4nc(-c5cccc(N6CCN(C(=O)C(=O)N7CCN(c8ccc9c(C%10CCC(=O)NC%10=O)nn(C)c9c8)CC7)CC6)c5)ccc4c23)C(=O)N1] | Ex 14 |
| 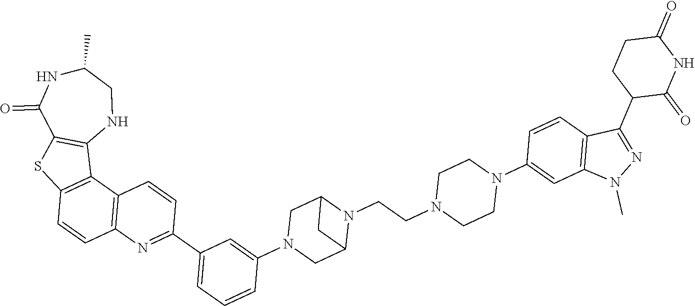 [SMILES: C[C@@H]1CNc2c(sc3ccc4nc(-c5cccc(N6CC7CC(C6)N7CCN6CCN(c7ccc8c(C9CCC(=O)NC9=O)nn(C)c8c7)CC6)c5)ccc4c23)C(=O)N1] | Ex 15 |
| 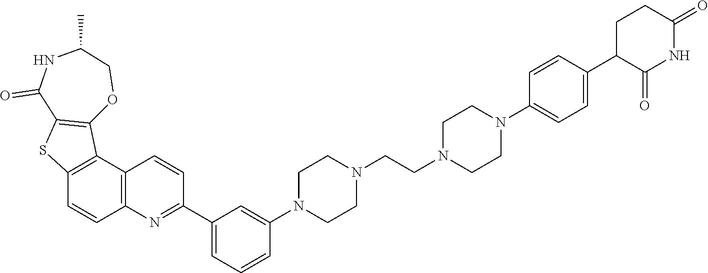 [SMILES: C[C@@H]1COc2c(sc3ccc4nc(-c5cccc(N6CCN(CCN7CCN(c8ccc(C9CCC(=O)NC9=O)cc8)CC7)CC6)c5)ccc4c23)C(=O)N1] | Ex 16 |
| 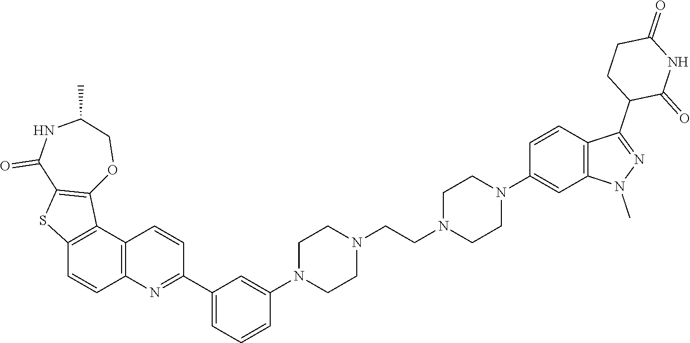 [SMILES: C[C@@H]1COc2c(sc3ccc4nc(-c5cccc(N6CCN(CCN7CCN(c8ccc9c(C%10CCC(=O)NC%10=O)nn(C)c9c8)CC7)CC6)c5)ccc4c23)C(=O)N1] | Ex 17 |
| 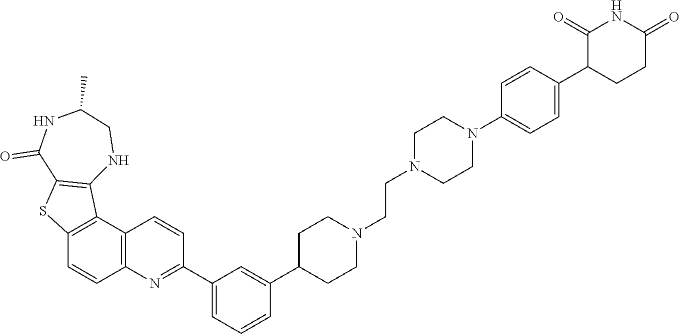 [SMILES: C[C@@H]1CNc2c(sc3ccc4nc(-c5cccc(C6CCN(CCN7CCN(c8ccc(C9CCC(=O)NC9=O)cc8)CC7)CC6)c5)ccc4c23)C(=O)N1] | Ex 18 |
| 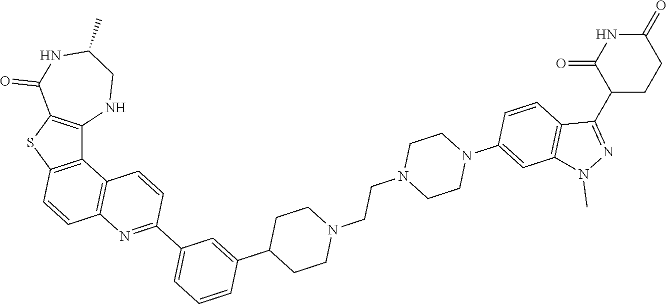 [SMILES: C[C@@H]1CNc2c(sc3ccc4nc(-c5cccc(C6CCN(CCN7CCN(c8ccc9c(C%10CCC(=O)NC%10=O)nn(C)c9c8)CC7)CC6)c5)ccc4c23)C(=O)N1] | Ex 19 |
| 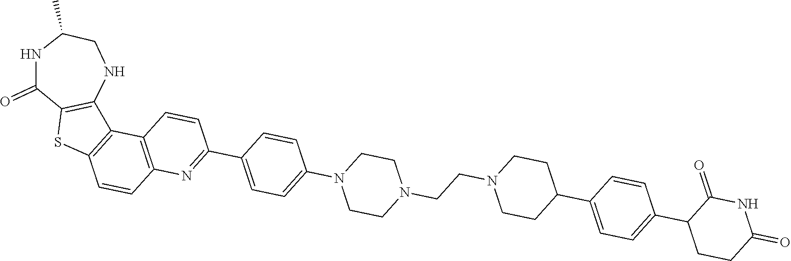 [SMILES: C[C@@H]1CNc2c(sc3ccc4nc(-c5ccc(N6CCN(CCN7CCC(c8ccc(C9CCC(=O)NC9=O)cc8)CC7)CC6)cc5)ccc4c23)C(=O)N1] | Ex 20 |
| 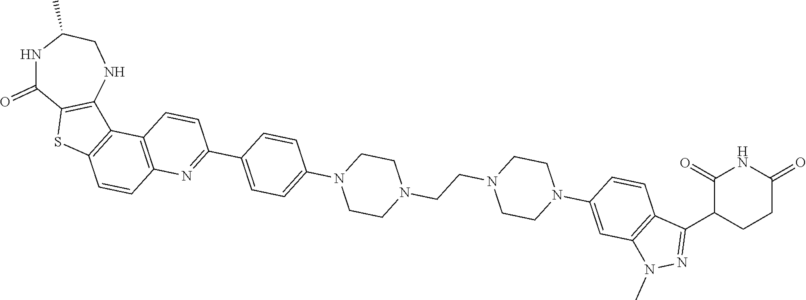 [SMILES: C[C@@H]1CNc2c(sc3ccc4nc(-c5ccc(N6CCN(CCN7CCN(c8ccc9c(C%10CCC(=O)NC%10=O)nn(C)c9c8)CC7)CC6)cc5)ccc4c23)C(=O)N1] | Ex 21 |
| 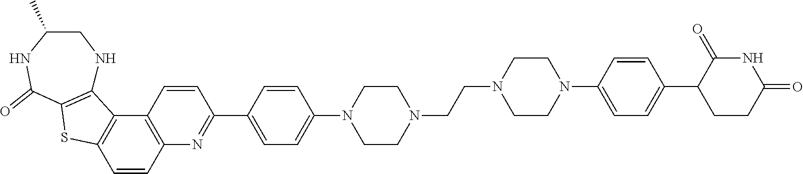 [SMILES: C[C@@H]1CNc2c(sc3ccc4nc(-c5ccc(N6CCN(CCN7CCN(c8ccc(C9CCC(=O)NC9=O)cc8)CC7)CC6)cc5)ccc4c23)C(=O)N1] | Ex 22 |
| 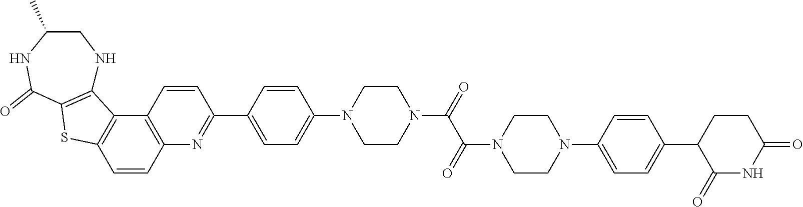 [SMILES: C[C@@H]1CNc2c(sc3ccc4nc(-c5ccc(N6CCN(C(=O)C(=O)N7CCN(c8ccc(C9CCC(=O)NC9=O)cc8)CC7)CC6)cc5)ccc4c23)C(=O)N1] | Ex 23 |
| 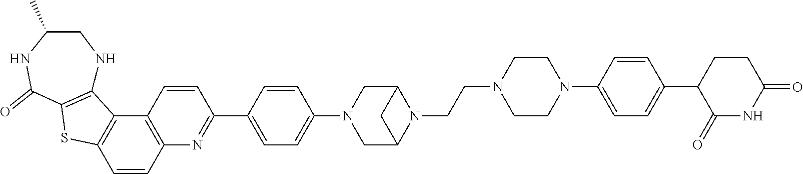 [SMILES: C[C@@H]1CNc2c(sc3ccc4nc(-c5ccc(N6CC7CC(C6)N7CCN6CCN(c7ccc(C8CCC(=O)NC8=O)cc7)CC6)cc5)ccc4c23)C(=O)N1] | Ex 24 |
| 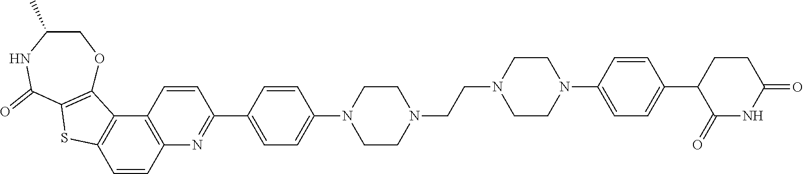 [SMILES: C[C@@H]1COc2c(sc3ccc4nc(-c5ccc(N6CCN(CCN7CCN(c8ccc(C9CCC(=O)NC9=O)cc8)CC7)CC6)cc5)ccc4c23)C(=O)N1] | Ex 25 |
| 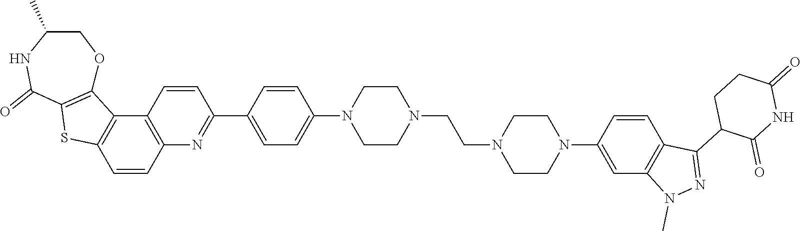 [SMILES: C[C@@H]1COc2c(sc3ccc4nc(-c5ccc(N6CCN(CCN7CCN(c8ccc9c(C%10CCC(=O)NC%10=O)nn(C)c9c8)CC7)CC6)cc5)ccc4c23)C(=O)N1] | Ex 26 |
| 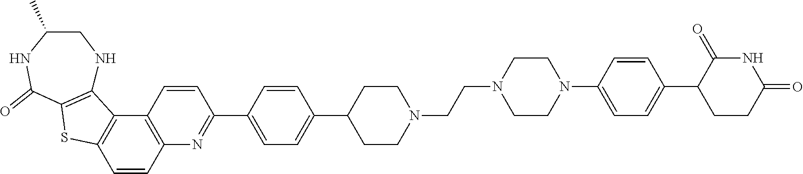 [SMILES: C[C@@H]1CNc2c(sc3ccc4nc(-c5ccc(C6CCN(CCN7CCN(c8ccc(C9CCC(=O)NC9=O)cc8)CC7)CC6)cc5)ccc4c23)C(=O)N1] | Ex 27 |
| 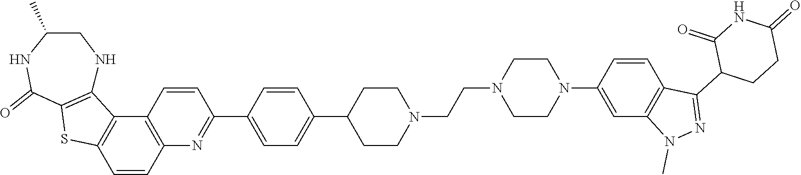 [SMILES: C[C@@H]1CNc2c(sc3ccc4nc(-c5ccc(C6CCN(CCN7CCN(c8ccc9c(C%10CCC(=O)NC%10=O)nn(C)c9c8)CC7)CC6)cc5)ccc4c23)C(=O)N1] | Ex 28 |
| 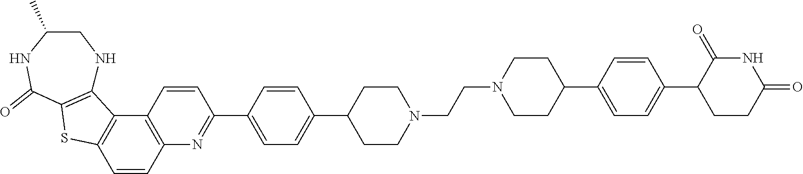 [SMILES: C[C@@H]1CNc2c(sc3ccc4nc(-c5ccc(C6CCN(CCN7CCC(c8ccc(C9CCC(=O)NC9=O)cc8)CC7)CC6)cc5)ccc4c23)C(=O)N1] | Ex 29 |
| 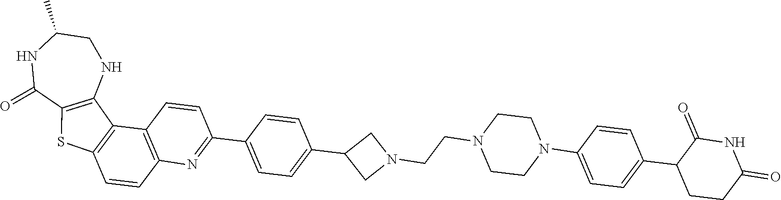 [SMILES: C[C@@H]1CNc2c(sc3ccc4nc(-c5ccc(C6CN(CCN7CCN(c8ccc(C9CCC(=O)NC9=O)cc8)CC7)C6)cc5)ccc4c23)C(=O)N1] | Ex 30 |
| 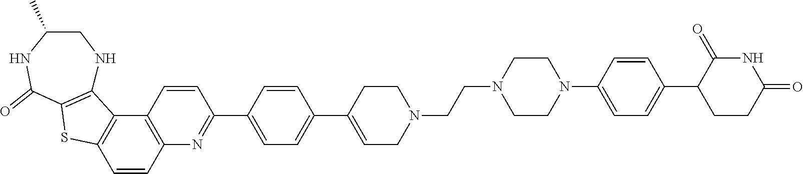 [SMILES: C[C@@H]1CNc2c(sc3ccc4nc(-c5ccc(C6=CCN(CCN7CCN(c8ccc(C9CCC(=O)NC9=O)cc8)CC7)CC6)cc5)ccc4c23)C(=O)N1] | Ex 31 |
| 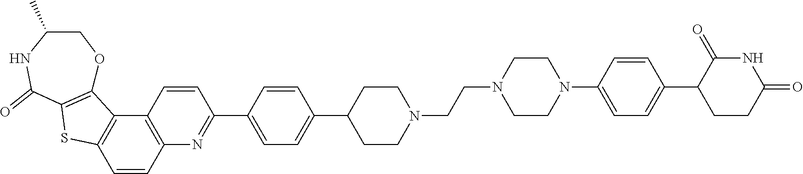 [SMILES: C[C@@H]1COc2c(sc3ccc4nc(-c5ccc(C6CCN(CCN7CCN(c8ccc(C9CCC(=O)NC9=O)cc8)CC7)CC6)cc5)ccc4c23)C(=O)N1] | Ex 32 |
| 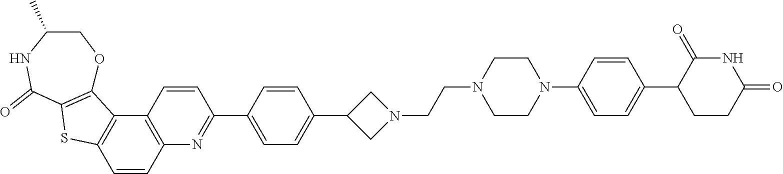 [SMILES: C[C@@H]1COc2c(sc3ccc4nc(-c5ccc(C6CN(CCN7CCN(c8ccc(C9CCC(=O)NC9=O)cc8)CC7)C6)cc5)ccc4c23)C(=O)N1] | Ex 33 |
| 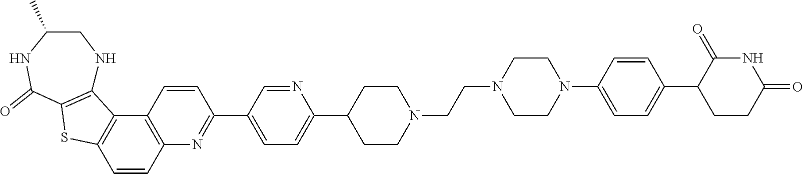 [SMILES: C[C@@H]1CNc2c(sc3ccc4nc(-c5ccc(C6CCN(CCN7CCN(c8ccc(C9CCC(=O)NC9=O)cc8)CC7)CC6)nc5)ccc4c23)C(=O)N1] | Ex 34 |
| 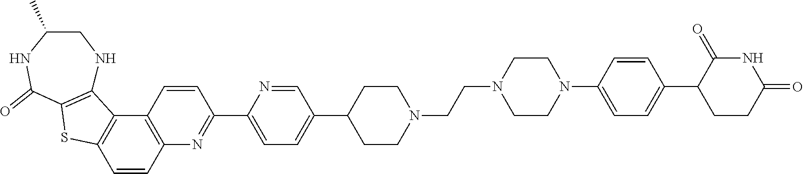 [SMILES: C[C@@H]1CNc2c(sc3ccc4nc(-c5ccc(C6CCN(CCN7CCN(c8ccc(C9CCC(=O)NC9=O)cc8)CC7)CC6)cn5)ccc4c23)C(=O)N1] | Ex 35 |
| 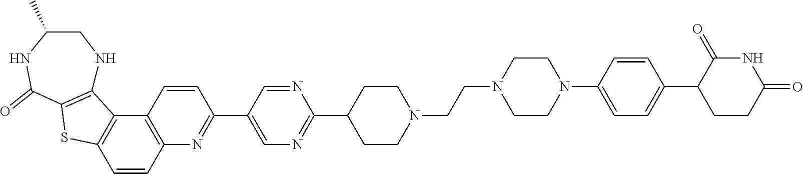 [SMILES: C[C@@H]1CNc2c(sc3ccc4nc(-c5cnc(C6CCN(CCN7CCN(c8ccc(C9CCC(=O)NC9=O)cc8)CC7)CC6)nc5)ccc4c23)C(=O)N1] | Ex 36 |
| 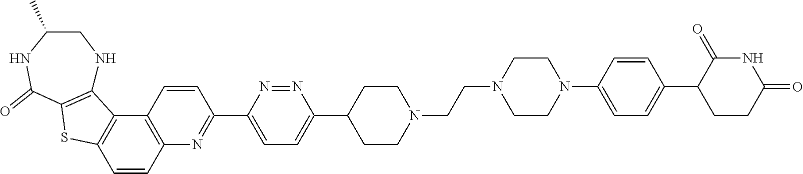 [SMILES: C[C@@H]1CNc2c(sc3ccc4nc(-c5ccc(C6CCN(CCN7CCN(c8ccc(C9CCC(=O)NC9=O)cc8)CC7)CC6)nn5)ccc4c23)C(=O)N1] | Ex 37 |
| 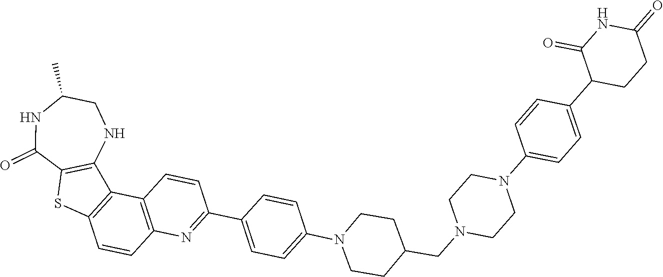 [SMILES: C[C@@H]1CNc2c(sc3ccc4nc(-c5ccc(N6CCC(CN7CCN(c8ccc(C9CCC(=O)NC9=O)cc8)CC7)CC6)cc5)ccc4c23)C(=O)N1] | Ex 38 |
| 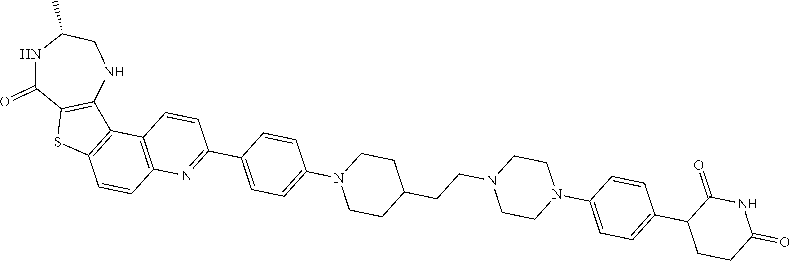 [SMILES: C[C@@H]1CNc2c(sc3ccc4nc(-c5ccc(N6CCC(CCN7CCN(c8ccc(C9CCC(=O)NC9=O)cc8)CC7)CC6)cc5)ccc4c23)C(=O)N1] | Ex 39 |
| 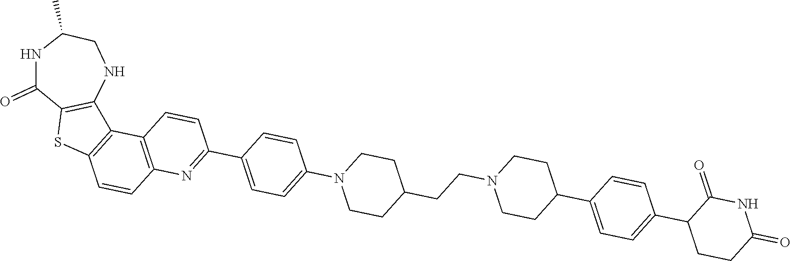 [SMILES: C[C@@H]1CNc2c(sc3ccc4nc(-c5ccc(N6CCC(CCN7CCC(c8ccc(C9CCC(=O)NC9=O)cc8)CC7)CC6)cc5)ccc4c23)C(=O)N1] | Ex 40 |
| 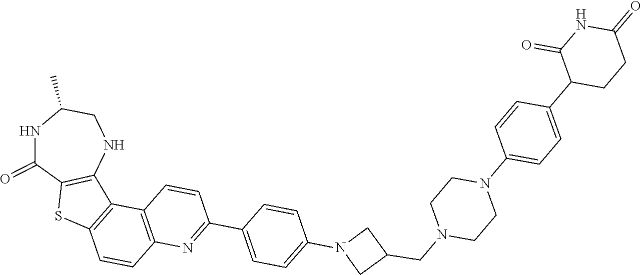 [SMILES: C[C@@H]1CNc2c(sc3ccc4nc(-c5ccc(N6CC(CN7CCN(c8ccc(C9CCC(=O)NC9=O)cc8)CC7)C6)cc5)ccc4c23)C(=O)N1] | Ex 41 |
| 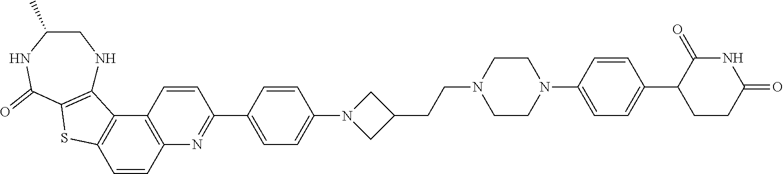 [SMILES: C[C@@H]1CNc2c(sc3ccc4nc(-c5ccc(N6CC(CCN7CCN(c8ccc(C9CCC(=O)NC9=O)cc8)CC7)C6)cc5)ccc4c23)C(=O)N1] | Ex 42 |
| 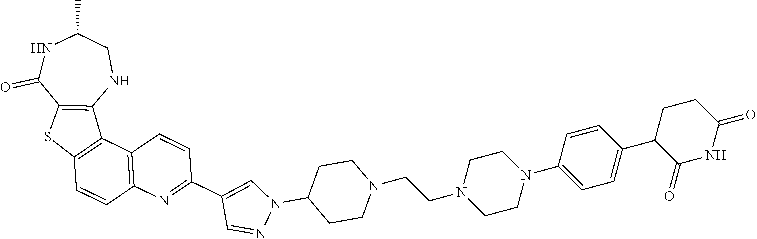 [SMILES: C[C@@H]1CNc2c(sc3ccc4nc(-c5cnn(C6CCN(CCN7CCN(c8ccc(C9CCC(=O)NC9=O)cc8)CC7)CC6)c5)ccc4c23)C(=O)N1] | Ex 43 |
| 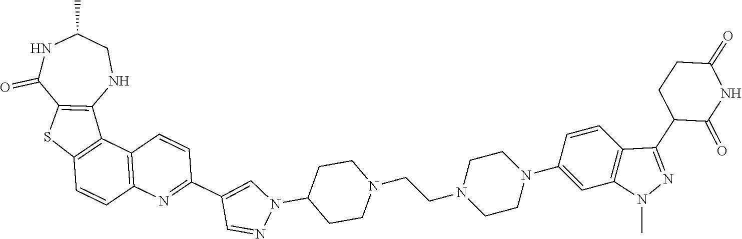 [SMILES: C[C@@H]1CNc2c(sc3ccc4nc(-c5cnn(C6CCN(CCN7CCN(c8ccc9c(C%10CCC(=O)NC%10=O)nn(C)c9c8)CC7)CC6)c5)ccc4c23)C(=O)N1] | Ex 44 |
| 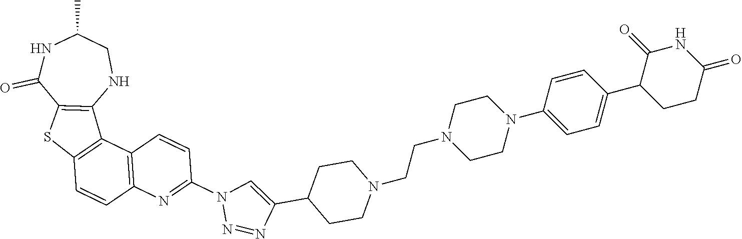 [SMILES: C[C@@H]1CNc2c(sc3ccc4nc(-n5cc(C6CCN(CCN7CCN(c8ccc(C9CCC(=O)NC9=O)cc8)CC7)CC6)nn5)ccc4c23)C(=O)N1] | Ex 45 |
| 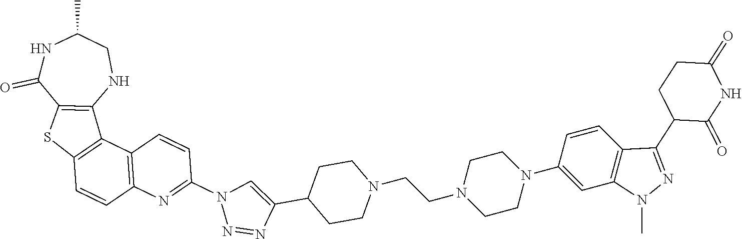 [SMILES: C[C@@H]1CNc2c(sc3ccc4nc(-n5cc(C6CCN(CCN7CCN(c8ccc9c(C%10CCC(=O)NC%10=O)nn(C)c9c8)CC7)CC6)nn5)ccc4c23)C(=O)N1] | Ex 46 |
| 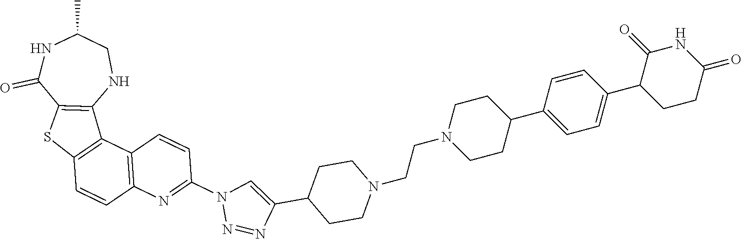 [SMILES: C[C@@H]1CNc2c(sc3ccc4nc(-n5cc(C6CCN(CCN7CCC(c8ccc(C9CCC(=O)NC9=O)cc8)CC7)CC6)nn5)ccc4c23)C(=O)N1] | Ex 47 |
| 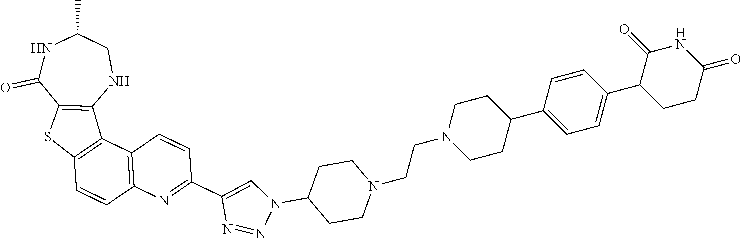 [SMILES: C[C@@H]1CNc2c(sc3ccc4nc(-c5cn(C6CCN(CCN7CCC(c8ccc(C9CCC(=O)NC9=O)cc8)CC7)CC6)nn5)ccc4c23)C(=O)N1] | Ex 48 |
| 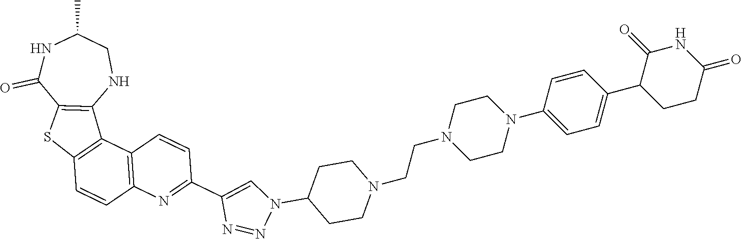 [SMILES: C[C@@H]1CNc2c(sc3ccc4nc(-c5cn(C6CCN(CCN7CCN(c8ccc(C9CCC(=O)NC9=O)cc8)CC7)CC6)nn5)ccc4c23)C(=O)N1] | Ex 49 |
| 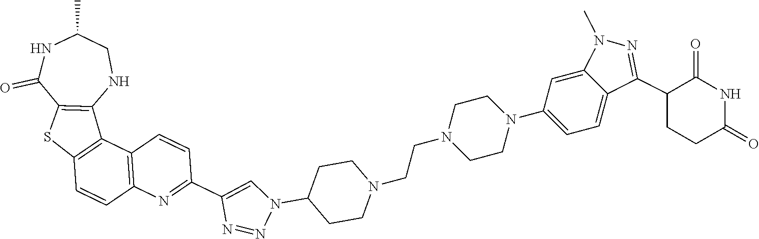 [SMILES: C[C@@H]1CNc2c(sc3ccc4nc(-c5cn(C6CCN(CCN7CCN(c8ccc9c(C%10CCC(=O)NC%10=O)nn(C)c9c8)CC7)CC6)nn5)ccc4c23)C(=O)N1] | Ex 50 |
| 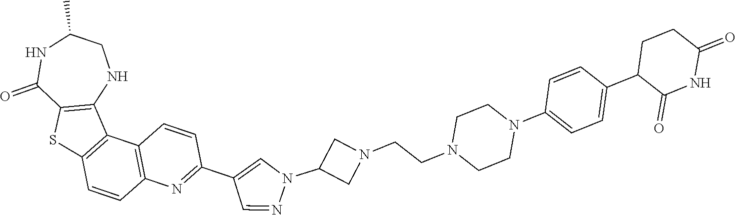 [SMILES: C[C@@H]1CNc2c(sc3ccc4nc(-c5cnn(C6CN(CCN7CCN(c8ccc(C9CCC(=O)NC9=O)cc8)CC7)C6)c5)ccc4c23)C(=O)N1] | Ex 51 |
| 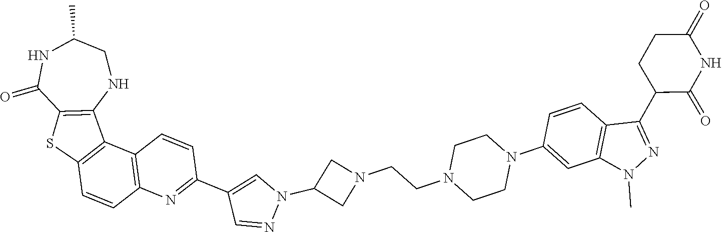 [SMILES: C[C@@H]1CNc2c(sc3ccc4nc(-c5cnn(C6CN(CCN7CCN(c8ccc9c(C%10CCC(=O)NC%10=O)nn(C)c9c8)CC7)C6)c5)ccc4c23)C(=O)N1] | Ex 52 |
| 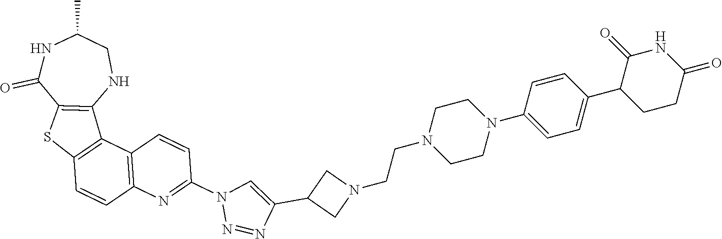 [SMILES: C[C@@H]1CNc2c(sc3ccc4nc(-n5cc(C6CN(CCN7CCN(c8ccc(C9CCC(=O)NC9=O)cc8)CC7)C6)nn5)ccc4c23)C(=O)N1] | Ex 53 |
| 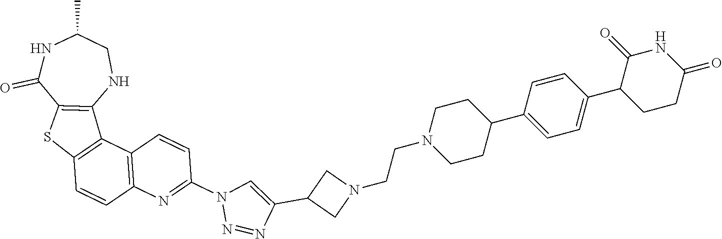 [SMILES: C[C@@H]1CNc2c(sc3ccc4nc(-n5cc(C6CN(CCN7CCC(c8ccc(C9CCC(=O)NC9=O)cc8)CC7)C6)nn5)ccc4c23)C(=O)N1] | Ex 54 |
| 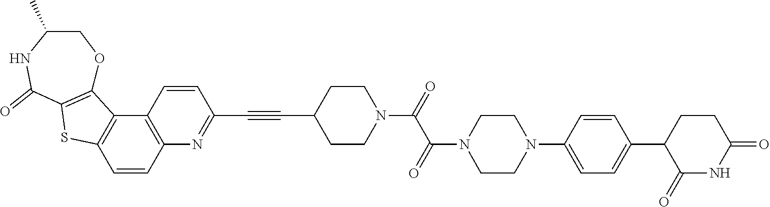 [SMILES: C[C@@H]1COc2c(sc3ccc4nc(C#CC5CCN(C(=O)C(=O)N6CCN(c7ccc(C8CCC(=O)NC8=O)cc7)CC6)CC5)ccc4c23)C(=O)N1] | Ex 55 |

or a pharmaceutically acceptable salt thereof.

In other embodiments of the disclosure, the pharmaceutical composition described herein comprises a compound described herein and a pharmaceutically acceptable excipient, carrier, or diluent.

In other embodiments of the disclosure, the method of degrading the activity of MK2, or a mutant thereof comprises contacting a biological sample with a compound of the present disclosure.

In another embodiment of the disclosure, the method of treating a disease, disorder, or condition mediated by MK2, or a mutant thereof, comprises administering to a patient in need thereof a compound or a composition of the present disclosure.

### 4. Uses, Formulation and Administration

### Pharmaceutically Acceptable Compositions

According to another embodiment, the present disclosure provides a composition comprising a compound described herein, or a pharmaceutically acceptable salt thereof, and a pharmaceutically acceptable carrier, adjuvant, or vehicle. In certain embodiments, the amount of compound in provided compositions is sufficient to measurably degrade MK2, or a mutant thereof, in a biological sample or in a patient. In certain embodiments, a provided composition is formulated for administration to a patient in need of such composition. In some embodiments, a provided composition is formulated for oral administration to a patient.

Compounds and compositions, according to a provided method, are administered using any amount and any route of administration effective for treating or lessening the severity of a disorder provided herein (i.e., an MK2-mediated disease or disorder). The exact amount required will vary from subject to subject, depending on the species, age, and general condition of the subject, the severity of the infection, the particular agent, its mode of administration, and the like. Compounds described herein may further be formulated in unit dosage form for ease of administration and uniformity of dosage.

Compositions provided herein may be administered orally, parenterally, by inhalation spray, topically, rectally, nasally, buccally, vaginally, intraperitoneally, intracisternally or via an implanted reservoir. In some embodiments, the compositions are administered orally, intraperitoneally or intravenously.

Sterile injectable forms of the compositions provided herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound provided herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In some embodiments, provided pharmaceutically acceptable compositions are formulated for oral administration. Such formulations may be administered with or without food. In some embodiments, pharmaceutically acceptable compositions provided herein are administered without food. In other embodiments, pharmaceutically acceptable compositions provided herein are administered with food. Pharmaceutically acceptable compositions provided herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, a provided compound is mixed with at least one inert, pharmaceutically acceptable excipient or carrier such as (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and/or (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, a provided compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that releases the active ingredient(s) only in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the provided compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents.

Alternatively, pharmaceutically acceptable compositions provided herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Compositions for rectal or vaginal administration may also be suppositories which can be prepared by mixing the compounds provided herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Pharmaceutically acceptable compositions provided herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing a compound described herein suspended or dissolved in one or more carriers. Carriers for topical administration of provided compounds include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing a compound described herein suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, including solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions provided herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Dosage forms for topical or transdermal administration of a compound described herein include ointments, pastes, creams, lotions, gels, powders, solutions, sprays, inhalants or patches. A compound may be admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Uses of Compounds and Pharmaceutically Acceptable Compositions

Compounds and compositions described herein are generally useful for the degradation of kinase activity of one or more enzymes and the treatment of diseases and disorders associated with MK2 degradation. Examples of kinases that are degraded by the compounds and compositions described herein and against which the methods described herein are useful include MK2, or a mutant thereof.

The activity of a compound utilized as a degrader of a MK2 kinase, or a mutant thereof, may be assayed in vitro, in vivo or in a cell line. In vitro assays include assays that determine inhibition of either the phosphorylation activity and/or the subsequent functional consequences, or ATPase activity of activated MK2 kinase, or a mutant thereof. Alternate in vitro assays quantitate the ability of a provided compound to bind to MK2. Degrader activity may be measured by radiolabeling the test compound prior to binding, isolating the compound/MK2 complex and determining the amount of radiolabel bound. Alternatively, degrader activity may be determined by running a competition experiment where test compounds are incubated with MK2 kinase bound to known radioligands. Detailed conditions for assaying a compound utilized in this disclosure as a degrader of MK2, or a mutant thereof, are set forth in the Examples, below.

According to one embodiment, the present disclosure relates to a method of degrading protein kinase activity in a biological sample comprising the step of contacting said biological sample with a compound provided herein, or a composition comprising said compound.

According to another embodiment, the present disclosure relates to a method of degrading MK2 kinase, or a mutant thereof, activity in a biological sample comprising the step of contacting said biological sample with a compound provided herein, or a composition comprising said compound. In certain embodiments, the present disclosure relates to a method of irreversibly degrading MK2 kinase, or a mutant thereof, activity in a biological sample comprising the step of contacting said biological sample with a compound provided herein, or a composition comprising said compound.

According to another embodiment, the present disclosure relates to a method of degrading MK2 kinase, or a mutant thereof, activity in a patient comprising the step of administering to said patient a compound provided herein, or a composition comprising said compound. According to certain embodiments, the present disclosure relates to a method of irreversibly degrading MK2 kinase, or a mutant thereof, activity in a patient comprising the step of administering to said patient a compound provided herein, or a composition comprising said compound. In other embodiments, the present disclosure provides a method for treating an MK2-mediated disease or disorder, in a patient in need thereof, comprising the step of administering to said patient a compound provided herein or pharmaceutically acceptable composition thereof. Such disorders are described in detail herein.

### MK2 Kinase

MAP kinase-activated protein kinase 2 (“MK2”) is an enzyme that in humans is encoded by the MAPKAPK2 gene. This gene encodes a member of the Ser/Thr protein kinase family. This kinase is regulated through direct phosphorylation by p38 MAP kinase. In conjunction with p38 MAP kinase, this kinase is known to be involved in many cellular processes including stress and inflammatory responses, nuclear export, gene expression regulation and cell proliferation. Heat shock protein HSP27 was shown to be one of the substrates of this kinase in vivo. Two transcript variants encoding two different isoforms have been found for this gene.

MK2 is a multi-domain protein consisting of an N-terminal proline-rich domain, a catalytic domain, an autoinhibitory domain and at the C-terminus a nuclear export signal (NES) and nuclear localization signal (NLS). Two isoforms of human MK2 have been characterized. One isoform consists of 400 amino acids and the other isoform 370 residues which is thought to be a splice variant missing the C-terminal NLS. MK2 is located in the nucleus of the cell and upon binding and phosphorylation by p38, the MK2 NES becomes functional and both kinases are co-transported out of the nucleus to the cytoplasm. Interestingly, transport of the MK2/p38 complex does not require catalytically active MK2, as the active site mutant, Asp207Ala, is still transported to the cytoplasm. Phosphorylation of human MK2 by p38 on residues T222, S272 and T334 is thought to activate the enzyme by inducing a conformational change of the autoinhibitory domain thus exposing the active site for substrate binding. Mutations of two autoinhibitory domain residues W332A and K326E in murine MK2 demonstrate an increase in basal activity and a C-terminal deletion of the autoinhibitory domain renders the enzyme constitutively active, providing additional evidence to the role of this domain in inhibition of MK2 activity.

Diseases or disorders associated with MK2 that are treated by compounds of the present disclosure include autoimmune disorders, chronic inflammatory disorders, acute inflammatory disorders, auto-inflammatory disorders, fibrotic disorders, metabolic disorders, neoplasias, or cardiovascular or cerebrovascular disorders. Thus, in some embodiments, the present disclosure provides a method for treating an MK2-mediated disease or disorder in a patient in need thereof, wherein said method comprises administering to said patient a therapeutically effective amount of a provided compound, or composition thereof. Such MK2-mediated diseases or disorders include, but are not limited to those described herein.

In some embodiments, the MK2-mediated disease or disorder is an autoimmune disorder, chronic and/or acute inflammatory disorder, and/or auto-inflammatory disorder. Exemplary autoimmune and/or inflammatory and/or auto-inflammatory disorders include: inflammatory bowel diseases (for example, ulcerative colitis or Crohn's disease), multiple sclerosis, psoriasis, arthritis, rheumatoid arthritis, osteoarthritis, juvenile arthritis, psoriatic arthritis, reactive arthritis, ankylosing spondylitis, cryopyrin associated periodic syndromes, Muckle-Wells syndrome, familial cold auto-inflammatory syndrome, neonatal-onset multisystem inflammatory disease, TNF receptor associated periodic syndrome, acute and chronic pancreatitis, atherosclerosis, gout, ankylosing spondylitis, fibrotic disorders (for example, hepatic fibrosis or idiopathic pulmonary fibrosis), nephropathy, sarcoidosis, scleroderma, anaphylaxis, diabetes (for example, diabetes mellitus type 1 or diabetes mellitus type 2), diabetic retinopathy, Still's disease, vasculitis, sarcoidosis, pulmonary inflammation, acute respiratory distress syndrome, wet and dry age-related macular degeneration, autoimmune hemolytic syndromes, autoimmune and inflammatory hepatitis, autoimmune neuropathy, autoimmune ovarian failure, autoimmune orchitis, autoimmune thrombocytopenia, silicone implant associated autoimmune disease, Sjogren's syndrome, familial Mediterranean fever, systemic lupus erythematosus, vasculitis syndromes (for example, temporal, Takayasu's and giant cell arteritis, Behçet's disease or Wegener's granulomatosis), vitiligo, secondary hematologic manifestation of autoimmune diseases (for example, anemias), drug-induced autoimmunity, Hashimoto's thyroiditis, hypophysitis, idiopathic thrombocytic pupura, metal-induced autoimmunity, myasthenia gravis, pemphigus, autoimmune deafness (for example, Meniere's disease), Goodpasture's syndrome, Graves' disease, HW-related autoimmune syndromes, Gullain-Barre disease, Addison's disease, anti-phospholipid syndrome, asthma, atopic dermatitis, Celiac disease, Cushing's syndrome, dermatomyositis, idiopathic adrenal adrenal atrophy, idiopathic thrombocytopenia, Kawasaki syndrome, Lambert-Eaton Syndrome, pernicious anemia, pollinosis, polyarteritis nodosa, primary biliary cirrhosis, primary sclerosing cholangitis, Raynaud's, Reiter's Syndrome, relapsing polychondritis, Schmidt's syndrome, thyrotoxidosis, sepsis, septic shock, endotoxic shock, exotoxin-induced toxic shock, gram negative sepsis, toxic shock syndrome, glomerulonephritis, peritonitis, interstitial cystitis, hyperoxia-induced inflammations, chronic obstructive pulmonary disease (COPD), vasculitis, graft vs. host reaction (for example, graft vs. host disease), allograft rejections (for example, acute allograft rejection or chronic allograft rejection), early transplantation rejection (for example, acute allograft rejection), reperfusion injury, pain (for example, acute pain, chronic pain, neuropathic pain, or fibromyalgia), chronic infections, meningitis, encephalitis, myocarditis, gingivitis, post surgical trauma, tissue injury, traumatic brain injury, enterocolitis, sinusitis, uveitis, ocular inflammation, optic neuritis, gastric ulcers, esophagitis, peritonitis, periodontitis, dermatomyositis, gastritis, myositis, polymyalgia, pneumonia and bronchitis.

In some embodiments, the MK2-mediated disease or disorder is a fibrotic disorder. Exemplary fibrotic disorders include systemic sclerosis/scleroderma, lupus nephritis, connective tissue disease, wound healing, surgical scarring, spinal cord injury, CNS scarring, acute lung injury, pulmonary fibrosis (for example, idiopathic pulmonary fibrosis or cystic fibrosis), chronic obstructive pulmonary disease, adult respiratory distress syndrome, acute lung injury, drug-induced lung injury, glomerulonephritis, chronic kidney disease (for example, diabetic nephropathy), hypertension-induced nephropathy, alimentary track or gastrointestinal fibrosis, renal fibrosis, hepatic or biliary fibrosis, liver fibrosis (for example, nonalcoholic steatohepatitis, hepatitis C, or hepatocellular carcinoma), cirrhosis (for example, primary biliary cirrhosis or cirrhosis due to fatty liver disease (for example, alcoholic and nonalcoholic steatosis)), radiation-induced fibrosis (for example, head and neck, gastrointestinal or pulmonary), primary sclerosing cholangitis, restenosis, cardiac fibrosis (for example, endomyocardial fibrosis or atrial fibrosis), opthalmic scarring, fibrosclerosis, fibrotic cancers, fibroids, fibroma, fibroadenomas, fibrosarcomas, transplant arteriopathy, keloid, mediastinal fibrosis, myelofibrosis, retroperitoneal fibrosis, progressive massive fibrosis, and nephrogenic systemic fibrosis.

In some embodiments, the MK2-mediated disease or disorder is a metabolic disorder. Exemplary metabolic disorders include obesity, steroid-resistance, glucose intolerance, and metabolic syndrome.

In some embodiments, the MK2-mediated disease or disorder is a neoplasia. Exemplary neoplasias include cancers. In some embodiments, exemplary neoplasias include angiogenesis disorders, multiple myeloma, leukemias (for example, acute lymphocytic leukemia, acute and chronic myelogenous leukemia, chronic lymphocytic leukemia, acute lymphoblastic leukemia, or promyelocytic leukemia), lymphomas (for example, B-cell lymphoma, T-cell lymphoma, mantle cell lymphoma, hairy cell lymphoma, Burkitt's lymphoma, mast cell tumors, Hodgkin's disease or non-Hodgkin's disease), myelodysplastic syndrome, fibrosarcoma, rhabdomyosarcoma; astrocytoma, neuroblastoma, glioma and schwannomas; melanoma, seminoma, teratocarcinoma, osteosarcoma, xenoderma pigmentosum, keratoctanthoma, thyroid follicular cancer, Kaposi's sarcoma, melanoma, teratoma, rhabdomyosarcoma, metastatic and bone disorders, as well as cancer of the bone, mouth/pharynx, esophagus, larynx, stomach, intestine, colon, rectum, lung (for example, non-small cell lung cancer or small cell lung cancer), liver, pancreas, nerve, brain (for example, glioma or glioblastoma multiforme), head and neck, throat, ovary, uterus, prostate, testis, bladder, kidney, breast, gall bladder, cervix, thyroid, prostate, and skin.

In some embodiments, the MK2-mediated disorder is a cardiovascular or cerebrovascular disorder. Exemplary cardiovascular disorders include atherosclerosis, restenosis of an atherosclerotic coronary artery, acute coronary syndrome, myocardial infarction, cardiac-allograft vasculopathy and stroke. Exemplary cerebrovascular diseases include central nervous system disorders with an inflammatory or apoptotic component, Alzheimer's disease, Parkinson's disease, Huntington's disease, amyotrophic lateral sclerosis, spinal cord injury, neuronal ischemia and peripheral neuropathy.

Diseases or disorders associated with MK2 that are treated by a compound provided herein include autoimmune disorders, chronic inflammatory disorders, acute inflammatory disorders, auto-inflammatory disorders, fibrotic disorders, metabolic disorders, neoplasias, or cardiovascular or cerebrovascular disorders. Thus, in some embodiments, the present disclosure provides a method for treating an MK2-mediated disease or disorder in a patient in need thereof, wherein said method comprises administering to said patient a composition comprising a therapeutically effective amount of a compound provided herein. Such MK2-mediated diseases or disorders include, but are not limited to those described herein.

In some embodiments, the MK2-mediated disease or disorder is an autoimmune disorder, chronic and/or acute inflammatory disorder, and/or auto-inflammatory disorder. Exemplary autoimmune and/or inflammatory and/or auto-inflammatory disorders include: inflammatory bowel diseases (for example, ulcerative colitis or Crohn's disease), multiple sclerosis, psoriasis, arthritis, rheumatoid arthritis, osteoarthritis, juvenile arthritis, psoriatic arthritis, reactive arthritis, ankylosing spondylitis, cryopyrin associated periodic syndromes, Muckle-Wells syndrome, familial cold auto-inflammatory syndrome, neonatal-onset multisystem inflammatory disease, TNF receptor associated periodic syndrome, acute and chronic pancreatitis, atherosclerosis, gout, ankylosing spondylitis, fibrotic disorders (for example, hepatic fibrosis or idiopathic pulmonary fibrosis), nephropathy, sarcoidosis, scleroderma, anaphylaxis, diabetes (for example, diabetes mellitus type 1 or diabetes mellitus type 2), diabetic retinopathy, Still's disease, vasculitis, sarcoidosis, pulmonary inflammation, acute respiratory distress syndrome, wet and dry age-related macular degeneration, autoimmune hemolytic syndromes, autoimmune and inflammatory hepatitis, autoimmune neuropathy, autoimmune ovarian failure, autoimmune orchitis, autoimmune thrombocytopenia, silicone implant associated autoimmune disease, Sjogren's syndrome, familial Mediterranean fever, systemic lupus erythematosus, vasculitis syndromes (for example, temporal, Takayasu's and giant cell arteritis, Behçet's disease or Wegener's granulomatosis), vitiligo, secondary hematologic manifestation of autoimmune diseases (for example, anemias), drug-induced autoimmunity, Hashimoto's thyroiditis, hypophysitis, idiopathic thrombocytic pupura, metal-induced autoimmunity, myasthenia gravis, pemphigus, autoimmune deafness (for example, Meniere's disease), Goodpasture's syndrome, Graves' disease, HW-related autoimmune syndromes, Gullain-Barre disease, Addison's disease, anti-phospholipid syndrome, asthma, atopic dermatitis, Celiac disease, Cushing's syndrome, dermatomyositis, idiopathic adrenal adrenal atrophy, idiopathic thrombocytopenia, Kawasaki syndrome, Lambert-Eaton Syndrome, pernicious anemia, pollinosis, polyarteritis nodosa, primary biliary cirrhosis, primary sclerosing cholangitis, Raynaud's, Reiter's Syndrome, relapsing polychondritis, Schmidt's syndrome, thyrotoxidosis, sepsis, septic shock, endotoxic shock, exotoxin-induced toxic shock, gram negative sepsis, toxic shock syndrome, glomerulonephritis, peritonitis, interstitial cystitis, hyperoxia-induced inflammations, chronic obstructive pulmonary disease (COPD), vasculitis, graft vs. host reaction (for example, graft vs. host disease), allograft rejections (for example, acute allograft rejection or chronic allograft rejection), early transplantation rejection (for example, acute allograft rejection), reperfusion injury, pain (for example, acute pain, chronic pain, neuropathic pain, or fibromyalgia), chronic infections, meningitis, encephalitis, myocarditis, gingivitis, post-surgical trauma, tissue injury, traumatic brain injury, enterocolitis, sinusitis, uveitis, ocular inflammation, optic neuritis, gastric ulcers, esophagitis, peritonitis, periodontitis, dermatomyositis, gastritis, myositis, polymyalgia, pneumonia and bronchitis,

In some embodiments, the MK2-mediated disease or disorder is a fibrotic disorder. Exemplary fibrotic disorders include systemic sclerosis/scleroderma, lupus nephritis, connective tissue disease, wound healing, surgical scarring, spinal cord injury, CNS scarring, acute lung injury, pulmonary fibrosis (for example, idiopathic pulmonary fibrosis or cystic fibrosis), chronic obstructive pulmonary disease, adult respiratory distress syndrome, acute lung injury, drug-induced lung injury, glomerulonephritis, chronic kidney disease (for example, diabetic nephropathy), hypertension-induced nephropathy, alimentary track or gastrointestinal fibrosis, renal fibrosis, hepatic or biliary fibrosis, liver fibrosis (for example, nonalcoholic steatohepatitis, hepatitis C, or hepatocellular carcinoma), cirrhosis (for example, primary biliary cirrhosis or cirrhosis due to fatty liver disease (for example, alcoholic and nonalcoholic steatosis)), radiation-induced fibrosis (for example, head and neck, gastrointestinal or pulmonary), primary sclerosing cholangitis, restenosis, cardiac fibrosis (for example, endomyocardial fibrosis or atrial fibrosis), opthalmic scarring, fibrosclerosis, fibrotic cancers, fibroids, fibroma, fibroadenomas, fibrosarcomas, transplant arteriopathy, keloid, mediastinal fibrosis, myelofibrosis, retroperitoneal fibrosis, progressive massive fibrosis, and nephrogenic systemic fibrosis.

In some embodiments, the MK2-mediated disease or disorder is a metabolic disorder. Exemplary metabolic disorders include obesity, steroid-resistance, glucose intolerance, and metabolic syndrome.

In some embodiments, the MK2-mediated disease or disorder is a neoplasia. Exemplary neoplasias include cancers. In some embodiments, exemplary neoplasias include angiogenesis disorders, multiple myeloma, leukemias (for example, acute lymphocytic leukemia, acute and chronic myelogenous leukemia, chronic lymphocytic leukemia, acute lymphoblastic leukemia, or promyelocytic leukemia), lymphomas (for example, B-cell lymphoma, T-cell lymphoma, mantle cell lymphoma, hairy cell lymphoma, Burkitt's lymphoma, mast cell tumors, Hodgkin's disease or non-Hodgkin's disease), myelodysplastic syndrome, fibrosarcoma, rhabdomyosarcoma; astrocytoma, neuroblastoma, glioma and schwannomas; melanoma, seminoma, teratocarcinoma, osteosarcoma, xenoderma pigmentosum, keratoctanthoma, thyroid follicular cancer, Kaposi's sarcoma, melanoma, teratoma, rhabdomyosarcoma, metastatic and bone disorders, as well as cancer of the bone, mouth/pharynx, esophagus, larynx, stomach, intestine, colon, rectum, lung (for example, non-small cell lung cancer or small cell lung cancer), liver, pancreas, nerve, brain (for example, glioma or glioblastoma multiforme), head and neck, throat, ovary, uterus, prostate, testis, bladder, kidney, breast, gall bladder, cervix, thyroid, prostate, and skin.

In some embodiments, the MK2-mediated disorder is a cardiovascular or cerebrovascular disorder. Exemplary cardiovascular disorders include atherosclerosis, restenosis of an atherosclerotic coronary artery, acute coronary syndrome, myocardial infarction, cardiac-allograft vasculopathy and stroke. Exemplary cerebrovascular diseases include central nervous system disorders with an inflammatory or apoptotic component, Alzheimer's disease, Parkinson's disease, Huntington's disease, amyotrophic lateral sclerosis, spinal cord injury, neuronal ischemia and peripheral neuropathy.

Accordingly, in some embodiments, provided formulations may be used to treat a MK2-mediated disease or disorder. In certain embodiments, the present disclosure provides methods of administering a provided formulation to human subjects.

Administration of provided formulations may be advantageous for the treatment, stabilization or lessening the severity or progression of one or more diseases and conditions associated with MK2 comprising the step of administering to the subject a provided formulation, as described herein. Such disorders or conditions include, among others, autoimmune diseases, inflammatory diseases, bone diseases, metabolic diseases, neurological and neurodegenerative diseases, cancer, cardiovascular diseases, allergies and asthma, Alzheimer's disease, and hormone-related diseases.

In some embodiments, the disease or disorder associated with MK2 is an autoimmune disease or disorder. In some embodiments, the disease or disorder associated with MK2 is an inflammatory disease or disorder. In some such embodiments, the inflammatory disease or disorder is selected from a chronic inflammatory disorder, an acute inflammatory disorder, or an auto-inflammatory disorder. In some embodiments, such autoimmune or inflammatory diseases and disorders are selected from rheumatoid arthritis, psoriatic arthritis, psoriasis, and ankylosing spondylitis.

In some embodiments, the present disclosure provides a method of preventing the progression of an autoimmune or inflammatory disease or disorder associated with MK2, comprising administering to a patient in need thereof a composition comprising a therapeutically effective amount of a compound provided herein. In some embodiments, such autoimmune or inflammatory diseases and disorders are selected from rheumatoid arthritis, psoriatic arthritis, psoriasis, and ankylosing spondylitis.

### Ankylosing Spondylitis

Ankylosing spondylitis (AS) is a chronic form of arthritis that primarily affects the spine, although other joints can become involved. A systemic inflammatory disease of indeterminate etiology, ankylosing spondylitis affects the axial spine (spondylitis), with sacroiliitis as its hallmark. The most common presenting symptom is chronic back pain and progressive spinal stiffness, a result of inflammation affecting the spine and sacroiliac joints (Feld et al. Axial disease in psoriatic arthritis and ankylosing spondylitis: a critical comparison. Nat Rev Rheumatol 2018; 14(6):363-71). In more advanced cases this inflammation can lead to ankylosis—new bone formation in the spine—causing sections of the spine to fuse in a fixed, immobile position.

Ankylosing spondylitis can also cause inflammation, pain, and stiffness in other areas of the body such as the shoulders, hips, ribs, heels, and small joints of the hands and feet. Sometimes the eyes can become involved (known as iritis or uveitis), and—rarely—the lungs and heart can be affected. The hallmark feature of ankylosing spondylitis is the involvement of the sacroiliac (SI) joints during the progression of the disease. The SI joints are located at the base of the spine, where the spine joins the pelvis.

Ankylosing spondylitis is typically diagnosed in people younger than 40 years and about 80% of patients develop first symptoms when they are younger than 30 years (Hanson et al. Genetics and the Causes of Ankylosing Spondylitis. Rheum Dis Clin North Am. 2017; 43(3):401-14). It is estimated that approximately 70% of patients with AS are males (de Winter et al. Prevalence of peripheral and extra-articular disease in ankylosing spondylitis versus non-radiographic axial spondyloarthritis: a meta-analysis. Arthritis Res Ther 2016; 18:196) Recent studies reported the prevalence of AS to range from 9 to 30 per 10,000 in the general population, depending on geographic area, study population or data source, case definition, and ascertainment methods. In general, there is a clear correlation between the prevalence of AS in a given population and the prevalence of HLA-B27 in that group, with the prevalence of AS being approximately 5 to 6 percent among people who are HLA-B27-positive (Reveille et al. The Epidemiology of Back Pain, Axial Spondyloarthritis and HLA-B27 in the United States. Am J Med Sci. 2013; 345(6): 431-6). Approximately 94% of individuals with AS are HLA-B27-positive (Brown et al. HLA class I associations of ankylosing spondylitis in the white population in the United Kingdom. Ann Rheum Dis. 1996; 55(4):268-70).

Although HLA-B27 is the largest single genetic contributor to disease pathophysiology, many other genetic loci, including those associated with the interleukin (IL)-17A pathway, have been associated with AS (Brown et al. Genetics of ankylosing spondylitis insights into pathogenesis. Nat Rev Rheumatol. 2016; 12(2):81-91; Costantino et al. Genetics and Functional Genomics of Spondyloarthritis. Front Immunol. 2018:9:2933). Chronic inflammation in AS is thought to be driven by CD4+ and/or CD8+T lymphocytes, including innate-like lymphocytes, and cytokines such as tumor necrosis factor (TNF)-α and IL-17A (Ranganathan et al. Macrophage Migration Inhibitory Factor Induces Inflammation and Predicts Spinal Progression in Ankylosing Spondylitis. Arthritis Rheumatol 2017; 69(9):1796-1806). Classification criteria for AS were proposed based on clinical grounds in the 1960s and later modified to include radiological criteria, known as the modified New York criteria for diagnosis of AS (van der Linden et al. Evaluation of Diagnostic Criteria for Ankylosing Spondylitis. A Proposal for Modification of the New York Criteria. Arthritis Rheum. 1984; 27(4):361-8). More recently, the Assessment of SpondyloArthritis international Society (ASAS) formulated classification criteria for axial spondyloarthritis (axSpA), of which AS is considered the prototype disease, based on imaging, clinical, and laboratory criteria (Rudwaleit et al. The development of Assessment of SpondyloArthritis international Society classification criteria for axial spondyloarthritis (part I): classification of paper patients by expert opinion including uncertainty appraisal. Ann Rheum Dis. 2009; 68(6):770-6; Rudwaleit et al. The development of Assessment of SpondyloArthritis international Society classification criteria for axial spondyloarthritis (part II): validation and final selection. Ann Rheum Dis 2009; 68(6):777-83). Disease classification of axSpA is established in persons with a history of back pain for 3 or more consecutive months before reaching 45 years of age, the presence of sacroiliitis confirmed on magnetic resonance imaging (MRI) or plain radiography, and with at least one clinical or laboratory finding that is characteristic of spondyloarthritis (SpA). Alternatively, persons with this history who have a positive test result for HLA-B27 and ≥2 clinical or laboratory features of SpA also fulfill the classification criteria for axSpA. Individuals with axSpA who have established radiographic evidence of sacroiliitis are considered to have met the definition for AS (Rudwaleit et al. The development of Assessment of SpondyloArthritis international Society classification criteria for axial spondyloarthritis (part I): classification of paper patients by expert opinion including uncertainty appraisal. Ann Rheum Dis. 2009; 68(6):770-6; Rudwaleit et al. The development of Assessment of SpondyloArthritis international Society classification criteria for axial spondyloarthritis (part II): validation and final selection. Ann Rheum Dis 2009; 68(6):777-83).

The treatment goal in patients with AS is to optimize long-term health-related quality of life and social participation through control of signs and symptoms, prevention of structural damage, normalization or preservation of function, avoidance of toxicities and minimization of comorbidities (Smolen et al. Treating axial spondyloarthritis and peripheral spondyloarthritis, especially psoriatic arthritis, to target: 2017 update of recommendations by an international task force. Ann Rheum Dis. 2018; 77(1):3-17). Current treatment guidelines for active AS (Bath Ankylosing Spondylitis Disease Activity Index [BASDAI] of at least 4, or Ankylosing Spondylitis Disease Activity Score—C-reactive protein [ASDAS-CRP] of at least 2.1) strongly recommend the use of nonsteroidal anti-inflammatory drugs (NSAIDs) and conditionally recommend their continuous use, based on very low-quality evidence (van der Heijde D, et al. 2016 update of the ASAS-EULAR management recommendations for axial spondyloarthritis. Ann Rheum Dis. 2017; 76(6):978-91). Tumor necrosis factor (TNF) blockers and anti-IL-17A monoclonal antibody (mAb) agents have become standard of care for patients who are unresponsive or intolerant to NSAIDs. Based on results of pivotal trials of currently approved biologics in AS, about 30% to 40% of patients treated with biologics do not achieve an Assessment of SpondyloArthritis International Society Response Criteria with an improvement of at least 20% (ASAS 20) and up to 64% of patients do not achieve an Assessment of SpondyloArthritis International Society Response Criteria with an improvement of at least 40% (ASAS 40) (Sieper et al. Secukinumab efficacy in anti-TNF-naive and anti-TNF-experienced subjects with active ankylosing spondylitis: results from the MEASURE 2 Study. Ann Rheum Dis. 2017; 76:571-75; Deodhar et al. Efficacy and Safety of Ixekizumab in the Treatment of Radiographic Axial Spondyloarthritis: Sixteen-Week Results From a Phase III Randomized, Double-Blind, Placebo-Controlled Trial in Patients With Prior Inadequate Response to or Intolerance of Tumor Necrosis Factor Inhibitors, Arthritis Rheumatol 2019; 71(4):599-611).

Although biologics can reduce inflammation and improve symptoms, there is only indirect evidence that currently available biologic TNF blockers influence spinal radiographic progression (Haroon et al. Effect of TNF-alpha inhibitor treatment on bone mineral density in patients with ankylosing spondylitis: A systematic review and meta-analysis. Semin Arthritis Rheum. 2014; 44(22):155-61; Maas et al. Reduction in Spinal Radiographic Progression in Ankylosing Spondylitis Patients Receiving Prolonged Treatment With Tumor Necrosis Factor Inhibitors. Arthritis Care Res (Hoboken). 2017; 69(7):1011-19; Molnar et al. TNF blockers inhibit spinal radiographic progression in ankylosing spondylitis by reducing disease activity: results from the Swiss Clinical Quality Management cohort. Ann Rheum Dis. 2018; 77(1):63-69), which continues to occur in spite of treatment (Poddubnyy et al. Physical Function and Spinal Mobility Remain Stable Despite Radiographic Spinal Progression in Patients with Ankylosing Spondylitis Treated with TNF-α Inhibitors for Up to 10 Years. J Rheumatol 2016; 43(12); 2142-8). Biologics require parenteral administration and are associated with development of autoantibodies, which may be neutralizing and limit drug effectiveness. In addition, profound TNF inhibition by currently available TNF-directed biologics is associated with increased risks of serious infections and malignancies.

In some embodiments, the present disclosure provides the recognition that AS patients who fail or cannot tolerate NSAIDs, and those who have also failed therapy with biologic agents, represent a patient population with high unmet medical need for whom there are currently no approved oral medications available to treat the underlying disease.

In some embodiments, the present disclosure provides a method for treating or lessening the severity of ankylosing spondylitis in a patient, comprising administering to the patient a composition comprising a compound provided herein. In some embodiments, a composition comprising a compound provided herein is administered to a subject who has radiologically confirmed AS. In some such embodiments, the subject has had an inadequate response to nonsteroidal anti-inflammatory drugs (NSAIDs).

In some embodiments, the term “treating or lessening the severity of ankylosing spondylitis” refers to the improvement of long-term health-related quality of life and social participation through one or more of (i) control of signs and symptoms of AS, (ii) prevention of structural damage, (iii) normalization or preservation of function, and (iv) avoidance of toxicities and minimization of comorbidities.

In some embodiments, the present disclosure provides a method of administering a composition comprising a compound provided herein to a subject who is HLA-B-27-positive. In some embodiments, provided methods comprise administering a composition comprising a compound provided herein to a subject in need thereof, wherein the subject is suffering from chronic inflammation associated with or mediated by one or more lymphocytes and/or cytokines. In some such embodiments, the one or more lymphocytes and/or cytokines is or are selected from CD4+T lymphocytes, CD8+T lymphocytes, innate-like lymphocytes, tumor necrosis factor (TNF)-α, and IL-17A.

In some embodiments, the present disclosure provides a method of administering a composition comprising a compound provided herein to a subject who satisfies the classification criteria for axial spondyloarthritis (axSpA). In some such embodiments, the classification criteria for axSpA is based on imaging, clinical, and laboratory criteria. In some embodiments, a subject has or is diagnosed with radiographic axSpA. In some embodiments, a subject has or is diagnosed with non-radiographic axSpA. Such subjects exhibit clinical signs and symptoms of SpA but does not exhibit characteristic radiographic changes on pelvic X-rays.

In some embodiments, a subject who satisfies the classification criteria for axSpA is a subject who has a history of back pain for 3 or more consecutive months before reaching 45 years of age, confirmed sacroiliitis, and at least one clinical or laboratory finding that is characteristic of spondyloarthritis (SpA). As used herein, “confirmed sacroiliitis” means sacroiliitis that is or has been confirmed on magnetic resonance imaging (MRI) or plain radiography. In some embodiments, a subject who satisfies the classification criteria for axSpA is a subject who has a positive test result for HLA-B27 and ≥2 clinical or laboratory features of SpA. In some embodiments, a subject suffering from AS is a subject who has axSpa and has established radiographic evidence of sacroiliitis.

In some embodiments, the present disclosure provides a method of preventing or slowing the progression of structural damage and/or preservation of function in a subject who is suffering from or has been diagnosed with ankylosing spondylitis. In some embodiments, a subject suffering from or diagnosed with ankylosing spondylitis exhibits one or more of the following criteria:
a. low back pain and stiffness for more than 3 months that improves with exercise, but is not relieved by rest;
b. limitation of motion of the lumbar spine in the sagittal and frontal planes;
c. limitation of chest expansion relative to normal values correlated for age and sex; and
d. sacroiliitis grade ≥2 bilaterally or grade 3 to 4 unilaterally.

In some embodiments, a subject has been diagnosed with AS according to the Modified New York Criteria for Ankylosing Spondylitis (1984). In some embodiments, a subject has symptoms of active AS based on a Bath Ankylosing Spondylitis Disease Activity Index (BASDAI) score ≥4. In some embodiments, a subject has a total Back Pain Numerical Rating Scales (NRS) score ≥4. In some embodiments, a subject meets one or more of the following criteria:
a. diagnosed with AS according to the Modified New York Criteria for Ankylosing Spondylitis (1984);
b. symptoms of active AS based on a BASDAI score 4; and
c. a total Back Pain Numerical Rating Scales (NRS) score ≥4.

In some embodiments, the present disclosure provides a method of treating AS in a subject, the method comprising:
administering to the subject a composition comprising a compound provided herein, wherein the subject experiences improvement or response in at least three of the following Assessment in SpondyloArthritis International Society (ASAS) criteria:
   a. patient global assessment of disease;
   b. total back pain;
   c. function; and
   d. inflammation.

In some embodiments, the subject experiences improvement or response in at least three of the ASAS criteria of at least 20% and a minimum of one unit on a scale of 0 to 10 and, for the remaining criterion, the subject experiences no worsening from baseline of no more than 20% and a minimum of one unit on a scale of 0 to 10. In some such embodiments, such improvement or response criteria are known as the “ASAS 20 improvement criteria.”

Accordingly, in some embodiments, the present disclosure provides a method of treating AS in a subject, the method comprising:
administering to the subject a composition comprising a compound provided herein, wherein the subject experiences improvement or response of at least 20% and a minimum of one unit in at least three of the following ASAS criteria:
   a. patient global assessment of disease (0 to 10 numerical rating scale);
   b. total back pain (0 to 10 numerical rating scale);
   c. function (assessed by Bath Ankylosing Spondylitis Functional Index (BASFI)); and
   d. inflammation (mean of numerical rating scales for Questions #5 and #6 on Bath Ankylosing Spondylitis Disease Activity Index (BASDAI)); and
wherein, for the remaining criterion, the subject experiences no worsening from baseline of greater than 20% and a minimum of one unit on a scale of 0 to 10.

In some embodiments, the present disclosure provides a method of improving disease activity (e.g., signs and symptoms of AS) in a subject who is suffering from or has been diagnosed with AS, the method comprising administering to the subject a composition comprising a compound provided herein, wherein disease activity is assessed by the ASAS 20 improvement criteria.

In some embodiments, the subject experiences improvement or response in at least three of the ASAS criteria of at least 40% and a minimum of two units on a scale of 0 to 10 and, for the remaining criterion, the subject experiences no worsening from baseline. In some embodiments, the subject experiences improvement or response in at least three of the ASAS criteria of at least 40% and a minimum of two units on a scale of 0 to 10 and, for the remaining criterion, the subject experiences no worsening from baseline of no more than 20% and a minimum of one unit on a scale of 0 to 10. In some such embodiments, such improvement or response criteria are known as the “ASAS 40 improvement criteria.”

Accordingly, in some embodiments, the present disclosure provides a method of treating AS in a subject, the method comprising:
administering to the subject a composition comprising a compound provided herein, wherein the subject experiences improvement or response of at least 40% and a minimum of two units in at least three of the following ASAS criteria:
   a. patient global assessment of disease (0 to 10 numerical rating scale);
   b. total back pain (0 to 10 numerical rating scale);
   c. function (assessed by Bath Ankylosing Spondylitis Functional Index (BASFI)); and
   d. inflammation (mean of numerical rating scales for Questions #5 and #6 on Bath Ankylosing Spondylitis Disease Activity Index (BASDAI)); and
wherein, for the remaining criterion, the subject experiences no worsening from baseline of greater than 20% and a minimum of one unit on a scale of 0 to 10.

In some embodiments, the present disclosure provides a method of improving disease activity (e.g., signs and symptoms of AS) in a subject who is suffering from or has been diagnosed with AS, the method comprising administering to the subject a composition comprising a compound provided herein, wherein disease activity is assessed by the ASAS 40 improvement criteria.

In some embodiments, the present disclosure provides a method of improving disease activity (e.g., signs and symptoms of AS) in a subject who is suffering from or has been diagnosed with AS, the method comprising administering to the subject a composition comprising a compound provided herein, wherein disease activity is assessed by the Ankylosing Spondylitis Disease Activity Score—C-reactive protein (ASDAS-CRP). In some embodiments, the subject achieves a ASDAS-CRP score of ≥1.1. In some such embodiments, the subject achieves a ASDAS-CRP score of ≥2.0. In some such embodiments, the subject achieves a ASDAS-CRP score of ≤1.3.

In some embodiments, the present disclosure provides a method of improving disease activity (e.g., signs and symptoms of AS) in a subject who is suffering from or has been diagnosed with AS, the method comprising administering to the subject a composition comprising a compound provided herein, wherein disease activity is assessed by the Bath Ankylosing Spondylitis Disease Activity Index (BASDAI).

In some embodiments, the present disclosure provides a method of improving physical function in a subject who is suffering from or has been diagnosed with AS, the method comprising administering to the subject a composition comprising a compound provided herein, wherein physical function is assessed by the Bath Ankylosing Spondylitis Functional Index (BASFI).

In some embodiments, the present disclosure provides a method of reducing spinal and sacroiliac joint inflammation in a subject who is suffering from or has been diagnosed with AS, the method comprising administering to the subject a composition comprising a compound provided herein, wherein spinal and sacroiliac joint inflammation is assessed by Spondylarthritis Research Consortium of Canada (SPARCC) MRI score of sacroiliac joints and spine.

In some embodiments, a subject suffering from or diagnosed with ankylosing spondylitis has failed therapy with at least 2 nonsteroidal anti-inflammatory drugs (NSAIDs). In some embodiments, a subject suffering from or diagnosed with ankylosing spondylitis has not received therapy selected from one or more of:
a. a cell depleting biologic agent such as an anti-CD20 antibody (e.g., rituximab), an anti-CD4 antibody, an anti-CD3 antibody, denosumab, an anti-IL-6 antibody (e.g., tocilizumab and sarilumab), and an anti-IL-23 antibody (e.g., ustekinuma) for at least 6 months prior to administration of a compound provided herein;
b. an oral corticosteroid (e.g., prednisone, etc.) in an amount greater than 10 mg/day systemically for at least 2 weeks prior to administration of a compound provided herein;
c. an intramuscular, intravenous, or intraarticular corticosteroid in any amount within at least 4 weeks of administration of a compound provided herein;
d. a vitamin K antagonist (e.g., warfarin);
e. isoniazid within at least 4 weeks of administration of a compound provided herein; and
f. any medication that is a substrate of one or more of the following transporters and has a narrow therapeutic index: p-glycoprotein (P-gp) (e.g., aliskiren, ambrisentan, colchicine, cyclosporine, dabigatran etexilate, digoxin, everolimus, fexofenadine, methotrexate, ranolazine, rivaroxaban, saxagliptin, sirolimus, sitagliptin, talinolol, ticagrelor, tolvaptan, etc.), breast cancer resistance protein (BCRP) (e.g., methotrexate, sulfasalazine, leflunomide, rosuvastatin, etc.), organic cation transporter 1 (OCT1) (e.g., metformin, gabapentin, pramipexole, tramadol, varenicline, etc.), organic anion transporting polypeptides 1B1 and 1B3 (OATP1B1 and OATP1B3, respectively) (e.g., ambrisentan, atorvastatin, ezetimibe, fluvastatin, glyburide, rosuvastatin, simvastatin acid, pitavastatin, pravastatin, repaglinide, telmisartan, valsartan, olmesartan, mycophenolic acid, etc.).

In some embodiments, a subject suffering from or diagnosed with ankylosing spondylitis has failed therapy with at least 2 nonsteroidal anti-inflammatory drugs (NSAIDs) and not more than 1 biological agent. In some embodiments, a subject who has failed therapy with not more than 1 biological agent is a subject who has had, for at least 12 weeks, an inadequate response and/or an unacceptable safety/tolerability to at least 1 dose of a biologic agent for AS (e.g., a TNF antagonist or IL-17A monoclonal antibody).

In some such embodiments, the patient is administered a composition comprising a therapeutically effective amount of a compound provided herein. In some embodiments, the patient is administered a unit dose of a compound provided herein.

In some embodiments, the present disclosure provides a use of a compound provided herein in the manufacture of a medicament for treating ankylosing spondylitis. In some embodiments, the present disclosure provides a compound described herein for use in treating ankylosing spondylitis.

Biomarkers of AS. In some embodiments, the present disclosure provides a method of administering to a subject a composition comprising a compound provided herein and monitoring the level of one or more biomarkers associated or correlated with AS. Pro-inflammatory cytokines and chemokines, including TNF-α, monocyte chemoattractant protein-1 (MCP-1), and IL-17A, have been shown to be increased in AS patients (Braun et al. Anti-tumour necrosis factor a therapy for ankylosing spondylitis: international experience. Ann Rheum Dis 2002; 61(Suppl III):iii51-iii60; West et al. Oncostatin M drives intestinal inflammation and predicts response to tumor necrosis factor-neutralizing therapy in patients with inflammatory bowel disease. Nat Med. 2017; 23(5)579-89; Romero-Sanchez et al. Serum monocyte chemotactic protein-1 concentrations distinguish patients with ankylosing spondylitis from patients with mechanical low back pain. J Spinal Disord Tech. 2011; 24(3):202-7). Additionally, increased serum levels of TNF-α have been correlated with increased CRP levels in AS patients (Wagner et al. Serum markers associated with clinical improvement in patients with ankylosing spondylitis treated with golimumab. Ann Rheum Dis. 2012; 71(5):674-80). Accordingly, in some embodiments, a biomarker associated or correlated with AS is selected from a pro-inflammatory cytokine or chemokine. In some such embodiments, a pro-inflammatory cytokine or chemokine is selected from TNF-α, monocyte chemoattractant protein-1 (MCP-1), and IL-17A. In some embodiments, the level of a pro-inflammatory cytokine or chemokine decreases over a period of time relative to a reference standard. In some such embodiments, a reference standard is the level of the pro-inflammatory cytokine or chemokine for a given subject or a given population prior to exposure to a compound provided herein.

In AS, several bone remodeling processes take place simultaneously: pathologic new bone formation in the form of syndesmophytes and bone loss in the form of bone erosion, osteolysis, and bone mineral density (BMD) loss leading to osteoporosis (Klingberg et al. Osteoporosis in ankylosing spondylitis—prevalence, risk factors and methods of assessment. Arthritis Res Ther 2012:14(3):R108). In some embodiments, a biomarker associated or correlated with AS is a bone formation marker. In some such embodiments, a bone formation marker is selected from procollagen type 1 N-terminal propeptide (P1NP) and bone resorption markers such as carboxy terminal cross-linked telopeptide of type 1 collagen (CTX-1). In some embodiments, the level of a bone formation marker decreases over a period of time relative to a reference standard. In some embodiments, the level of a bone formation marker increases over a period of time relative to a reference standard. In some embodiments, a reference standard is the level of the bone formation marker for a given subject or a given population prior to exposure to a compound provided herein.

Bone destruction is mediated by the recruitment of osteoclast precursors (OCPs) into the inflamed tissue and their differentiation into mature osteoclasts. TNF inhibition has resulted in sustained loss of circulating OCPs that can differentiate into osteoclasts (Lam et al. TNF-alpha induces osteoclastogenesis by direct stimulation of macrophages exposed to permissive levels of RANK ligand. J Clin Invest. 2000; 106(12):1481-8; Li et al. Systemic tumor necrosis factor alpha mediates an increase in peripheral CD11bhigh osteoclast precursors in tumor necrosis factor alpha-transgenic mice. Arthritis Rheum. 2004; 50(1):265-76). In some embodiments, a biomarker associated or correlated with AS is an osteoclast precursor (OCP). In some embodiments, the level of an osteoclast precursor decreases over a period of time relative to a reference standard. In some such embodiments, a reference standard is the level of the osteoclast precursor for a given subject or a given population prior to exposure to a compound provided herein.

In some embodiments, a biomarker associated or correlated with AS is a genetic marker. In some such embodiments, a genetic marker is selected from HLA-B27 and polygenic risk scores built using public AS data (see, e.g., Rostami et al. Prediction of Ankylosing Spondylitis in the HUNT Study by a Genetic Risk Score Combining 110 Single-nucleotide Polymorphisms of Genome-wide Significance. J Rheumatol 2019; 46:1-7).

### Rheumatoid Arthritis

Rheumatoid arthritis is a chronic autoimmune disorder in which the body's immune system attacks its own tissue, including joint linings, synovial tissues, cartilage and bone, causing painful swelling. The inflammation that results from immune system attacks results in the thickening of the synovium, the tissue that lines the insides of joints, leading to swelling and pain in and around the joints. Over long periods of time, the inflammation associated with rheumatoid arthritis can damage cartilage, the elastic tissue that covers the ends of bones in a joint, as well as the bones themselves. Over time, there is loss of cartilage, and the joint spacing between bones can become smaller. Joints can become loose, unstable, painful and lose their mobility. Joint deformity also can occur. Joint damage cannot be reversed, and because it can occur early, doctors recommend early diagnosis and aggressive treatment to control rheumatoid arthritis. In severe cases, rheumatoid arthritis attacks internal organs.

Patients with rheumatoid arthritis can be classified into distinct subsets, including lymphoid, myeloid and fibroid subsets. Dennis et al., “Synovial phenotypes in rheumatoid arthritis correlate with response to biologic therapeutics,” Arthritis Research & Therapy 2014, 16:R90, 1-18; Setiadi, et. al, “Synovial Subset-Derived Baseline Serum Biomarkers Segregate Rheumatoid Arthritis Patients into Subgroups with Distinct Serum Protein and Clinical Characteristics,” Abstract Number 1307, 2013 ACR/ARHP Annual Meeting.

In some embodiments, the present disclosure provides a method of treating rheumatoid arthritis in a patient, comprising administering to the patient a composition comprising a compound provided herein. In some such embodiments, the patient is administered a composition comprising a therapeutically effective amount of a compound provided herein. In some embodiments, the patient is administered a unit dose of a compound provided herein.

In some embodiments, the present disclosure provides a method of treating one or more of the lymphoid, myeloid and fibroid subsets of rheumatoid arthritis, comprising administering a composition comprising a compound provided herein to a patient in one or more subsets. Such subsets are classified by the presence of certain biomarkers which are detailed in Dennis et al., “Synovial phenotypes in rheumatoid arthritis correlate with response to biologic therapeutics,” Arthritis Research & Therapy 2014, 16:R90, 1-18; Setiadi, et. al, “Synovial Subset-Derived Baseline Serum Biomarkers Segregate Rheumatoid Arthritis Patients into Subgroups with Distinct Serum Protein and Clinical Characteristics,” Abstract Number 1307, 2013 ACR/ARHP Annual Meeting, each of which is hereby incorporated by reference.

In some embodiments, the present disclosure provides a method for treating or lessening the severity of rheumatoid arthritis in a patient, wherein the patient has one or more biomarkers for the lymphoid subset of rheumatoid arthritis, comprising administering to the patient a composition comprising a compound provided herein. Such biomarkers for the lymphoid subset of rheumatoid arthritis include, for example, high CXCL13 and low soluble ICAM1 expression levels. In some embodiments, the present disclosure provides a method for treating or lessening the severity of rheumatoid arthritis in a patient, wherein the patient has one or more biomarkers for the myeloid subset of rheumatoid arthritis, comprising administering to the patient a composition comprising a compound provided herein. In some embodiments, the present disclosure provides a method for treating or lessening the severity of rheumatoid arthritis in a patient, wherein the patient has one or more biomarkers for the fibroid subset of rheumatoid arthritis, comprising administering to the patient a composition comprising a compound provided herein. In some embodiments, the present disclosure provides a method for treating or lessening the severity of at least one subset of rheumatoid arthritis, comprising administering to the patient a composition comprising a compound provided herein. In some embodiments, the subset of rheumatoid arthritis is lymphoid. In some embodiments, the subset of rheumatoid arthritis is myeloid. In some embodiments, the subset of rheumatoid arthritis is fibroid.

In some embodiments, the present disclosure provides a use of a compound provided herein in the manufacture of a medicament for treating rheumatoid arthritis. In some embodiments, the present disclosure provides a compound described herein for use in treating rheumatoid arthritis.

### Psoriasis and Psoriatic Arthritis

Psoriasis is a chronic, inflammatory disease of the skin, scalp, nails, and joints that is characterized by a scaly rash that occurs most frequently on the elbows, knees, and scalp, but can cover much of the body. A normal skin cell matures and falls off the body's surface in 28 to 30 days, but a psoriatic skin cell takes only three to four days to mature and gathers at the surface, thus forming lesions.

Up to 30 percent of people with psoriasis also develop psoriatic arthritis. In most cases (though not always), the psoriasis will precede the arthritis, sometimes by many years. When arthritis symptoms occur with psoriasis, it is called psoriatic arthritis (PsA). In these cases, the joints at the end of the fingers are most commonly affected, causing inflammation and pain, but other joints like the wrists, knees, and ankles can also become involved. Symptoms in the fingernails and toenails range from small pits in the nails to nearly complete destruction and crumbling as seen in reactive arthritis or fungal infections.

About 20 percent of patients with PsA will develop spinal involvement, which is called psoriatic spondylitis. Inflammation of the spine can lead to complete fusion, as in ankylosing spondylitis (AS), or affect only certain areas such as the lower back or neck. Patients who are HLA-B27 positive are more likely than others to have their disease progress to the spine.

PsA and AS are considered genetically and clinically related because both are inflammatory rheumatic diseases linked to the HLA-B27 gene. HLA-B27 is a powerful predisposing gene associated with several rheumatic diseases. The gene itself does not cause disease, but can make people more susceptible. While a number of genes are linked to PsA, the highest predictive value is noted with HLA-B27.

In some embodiments, the present disclosure provides a method for treating or lessening the severity of psoriasis and/or psoriatic arthritis in a patient, comprising administering to the patient a composition comprising a compound provided herein. In some such embodiments, the patient is administered a composition comprising a therapeutically effective amount of a compound provided herein. In some embodiments, the patient is administered a unit dose of a compound provided herein.

In some embodiments, the present disclosure provides a use of a compound provided herein in the manufacture of a medicament for treating psoriasis and/or psoriatic arthritis. In some embodiments, the present disclosure provides a compound described herein for use in treating psoriasis and/or psoriatic arthritis.

### EXAMPLES

### General Information:

### LCMS Method 1.

Luna C18 (2) 50×3.0 mm, 3.0 um. Temperature: 45° C., Flow: 1.5 mL/min, run time: 2.5 min. Mobile phase conditions: Initial 95% H₂O+0.1% FA/5% MeCN+0.1% FA, then linear gradient to 95% MeCN+0.1% FA over 1.3 min. then hold for 1.2 min. at 95% MeCN+0.1% FA. MSD: ESI Positive

### LCMS Method 2.

SunFire C18 75×4.6 mm, 3.5 um. Temperature: 45° C., Flow: 1.5 mL/min, run time: 6.0 min. Mobile phase conditions: Initial 95% H₂O+0.1% FA/5% MeCN+0.1% FA, then linear gradient to 95% MeCN+0.1% FA over 4.0 min. then hold for 2.0 min. at 95% MeCN+0.1% FA. MSD: ESI Positive

### LCMS Method 3.

Column: HALO C18, 3.0×30 mm, 2.7 m particles; Mobile Phase A: water with 0.05% trifluoroacetic acid; Mobile Phase B: acetonitrile with 0.05% trifluoroacetic acid; Temperature: 40° C.; Gradient: 5% B to 100% B over 1.3 min, then a 0.50 min hold at 100% B; Flow: 1.5 mL/min

### Synthesis of Exemplary Compounds

### Methods of Preparation

Compounds exemplified below and intermediates used in the preparation of compounds exemplified below, can be prepared using procedures shown in the following examples and related procedures. The methods and conditions used in these examples, and the actual compounds prepared in these examples, are not meant to be limiting, but are meant to demonstrate how the compounds exemplified below can be prepared. Starting materials and reagents used in these examples, when not prepared by a procedure described herein, are generally either commercially available, or are reported in the chemical literature, or may be prepared by using procedures described in the chemical literature.

Abbreviations as used herein, are defined as follows: “1×” for once, “2×” for twice, “3×” for thrice, “° C.” for degrees Celsius, “equiv” for equivalent or equivalents, “g” for gram or grams, “mg” for milligram or milligrams, “L” for liter or liters, “mL” for milliliter or milliliters, “L” for microliter or microliters, “N” for normal, “M” for molar, “mmol” for millimole or millimoles, “min” for minute or minutes, “h” for hour or hours, “rt” for room temperature, “ON” for overnight, “RT” for retention time, “atm” for atmosphere, “psi” for pounds per square inch, “conc.” for concentrate, “sat” or “saturated” for saturated, “CVs” for column volumes, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “IRMS” for high resolution mass spectrometry, “LCMS” or “LC/MS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “nOe” for nuclear Overhauser effect spectroscopy, “¹H” for proton, “S” for delta, “s” for singlet, “d” for doublet, “t” for triplet, “q” for quartet, “m” for multiplet, “br” for broad, “MHz” for megahertz, and “a”, “p”, “R”, “S”, “E”, and “Z” are stereochemical designations familiar to one skilled in the art.

### LIST OF EXEMPLARY ABBREVIATIONS

| | |
|---|---|
| AcOH or HOAc | acetic acid |
| Aq | aqueous |
| pyAOP | ((7-Azabenzotriazol-1-yloxy)tripyrrolidinophosphonium |
| | hexafluorophosphate) |
| B(OMe)₃ | trimethyl borate |
| Boc | tert-butyloxycarbonyl |
| (Boc)₂O | Di-tert-butyldicarbonate |
| B₂pin₂ | Bis(pinacolato)diboron |
| BTEAC | benzyltriethylammonium chloride |
| Bu | butyl |
| CH₂Cl₂ | dichloromethane |
| CH₃I | methyl iodide |
| Cphos | 2-dicyclohexylphosphino-2′,6′-bis(N,N- |
| | dimethylamino)biphenyl |
| Cs₂CO₃ | cesium carbonate |
| Cu(OAc)₂ | copper acetate |
| CuBr₂ | copper (I) bromide |
| CV | Column volume |
| DCM | Dichloromethane |
| DCE | Dichloroethane |
| DMAP | 4-(dimethylamino)pyridine |
| DIEA/DIPEA/Hünig's Base | diisopropylethylamine |
| DMF | dimethyl formamide |
| DMSO | dimethyl sulfoxide |
| Et | Ethyl |
| Et₂O | diethyl ether |
| EtOAc | ethyl acetate |
| EtOH | ethanol |
| FA | formic acid |
| H₂ | hydrogen (gas) |
| H₂O | water |
| H₂SO₄ | sulfuric acid |
| Na₂SO₄ | Sodium sulfate |
| HATU | O-(7-azabenzotriazol-1-yl)-N,N,N′,N′-tetramethyluronium |
| | hexafluorophosphate |
| HCl | hydrochloric acid |
| i-Pr | isopropyl |
| K₂CO₃ | potassium carbonate |
| KOAc | potassium acetate |
| K₃PO₄ | potassium phosphate |
| LiOH | lithium hydroxide |
| Me | Methyl |
| MeI | methyl Iodide |
| MeCN | acetonitrile |
| MeOH | Methanol |
| MTBE | Methyl tert-butyl ether |
| N₂ | nitrogen (gas) |
| NaIO₄ | sodium periodate |
| Na₂SO₄ | sodium sulfate |
| NaBH(OAc)₃ | sodium triacetoxyborohydride |
| NaBH₃CN | sodium cyanoborohydride |
| NaHCO₃ | sodium bicarbonate |
| n-BuLi | n-butyllithium |
| NH₄Cl | ammonium chloride |
| NMI | N-Methylimidazole |
| Pd(OH)₂/C | palladium hydroxide on carbon |
| Pd(PPh₃)₄ | palladium tetrakis |
| Pd(PPh₃)₂Cl₂ | Bis(triphenylphosphine)palladium (II)dichloride |
| Pd/C | palladium on carbon |
| PCC | Pyridinium chlorochromate |
| Pd₂(dba)₃ | tris(dibenzylideneacetone)dipalladium(0) |
| Pd(dtbpf)Cl₂ | [1,1′-Bis(di-tert- |
| | butylphosphino)ferrocene]dichloropalladium(II) |
| PdCl₂(dppf) | [1,1′-bis(diphenylphosphino)-ferrocene]dichloropalladium(II) |
| PE | Petroleum ether |
| PF₆ | Hexafluorophosphate |
| Pin | Pinacolato |
| i-PrOH | Isopropyl alcohol |
| P(o-Tol)₃ | Tri(o-tolyl)phosphine |
| p-TsOH | p-Toluenesulfonic acid |
| Red-Al ® | Sodium bis(2-methoxyethoxy)aluminum dihydride, Sodium |
| | dihydrido-bis(2-methoxyethoxy)aluminate |
| Ruphos | 2-dicyclohexylphosphino-2′,6′-diisopropoxybiphenyl |
| Ruphos Pd G3 | (2-dicyclohexylphosphino-2′,6′-diisopropoxy-1,1′-biphenyl)[2- |
| | (2′-amino-1,1′-biphenyl)]palladium(II) methanesulfonate |
| Rochelle salt | Potassium sodium tartrate |
| rt | Room temperature |
| Sat. | Saturated |
| NaN₃ | Sodium azide |
| SiO₂ | silica oxide |
| t-BuOK | Potassium tert-butoxide |
| TCFH | Chloro-N,N,N′,N′-tetramethylformamidinium |
| | hexafluorophosphate |
| TEA | triethylamine |
| TFA | trifluoroacetic acid |
| TfOH | triflic acid |
| THF | tetrahydrofuran |
| Xphos | 2-dicyclohexylphosphino-2′,4′,6′-triisopropylbiphenyl |
| Xphos Pd G3 | 2-dicyclohexylphosphino-2′,4′,6′-triisopropyl-1,1′-biphenyl)[2- |
| | (2′-amino-1,1′-biphenyl)]palladium(II) methanesulfonate |
| XantPhos | 4,5-Bis(diphenylphosphino)-9,9-dimethylxanthene |
| ZnCl₂ | zinc chloride |

The compounds of the present invention may be synthesized by many methods available to those skilled in the art of organic chemistry (Smith, M. B., March's Advanced Organic Chemistry: Reactions, Mechanisms, and Structure, 7^{th} Edition (2013)). General synthetic schemes for preparing compounds of the present invention are described below. These schemes are illustrative and are not meant to limit the possible techniques one skilled in the art may use to prepare the compounds disclosed herein. Different methods to prepare the compounds of the present invention will be evident to those skilled in the art. Additionally, the various steps in the synthesis may be performed in an alternate sequence in order to give the desired compound or compounds.

Examples of compounds of the present invention prepared by methods described in the general schemes are given in the intermediates and examples section set out hereinafter. Example compounds are typically prepared as racemic mixtures. Preparation of homochiral examples may be carried out by techniques known to one skilled in the art. For example, homochiral compounds may be prepared by separation of racemic products by chiral phase preparative HPLC. Alternatively, the example compounds may be prepared by methods known to give enantiomerically enriched products. These include, but are not limited to, the incorporation of chiral auxiliary functionalities into racemic intermediates which serve to control the diastereoselectivity of transformations, providing enantio-enriched products upon cleavage of the chiral auxiliary.

Scheme 1 illustrates an approach to the synthesis of compounds exemplified by 8a. Intermediate 3 can be synthesized through Pd-catalyzed Suzuki cross-coupling (Miyaura, N. and Suzuki, A. Chemical Reviews, 95:2457-2483, 1995) of 1 (previously reported: Anderson et al., J. Med. Chem., 2007, 50, 2647-2654) and 2. The resulting ester 3 can be saponified to the desired acid 4 through treatment with a base, such as LiOH.

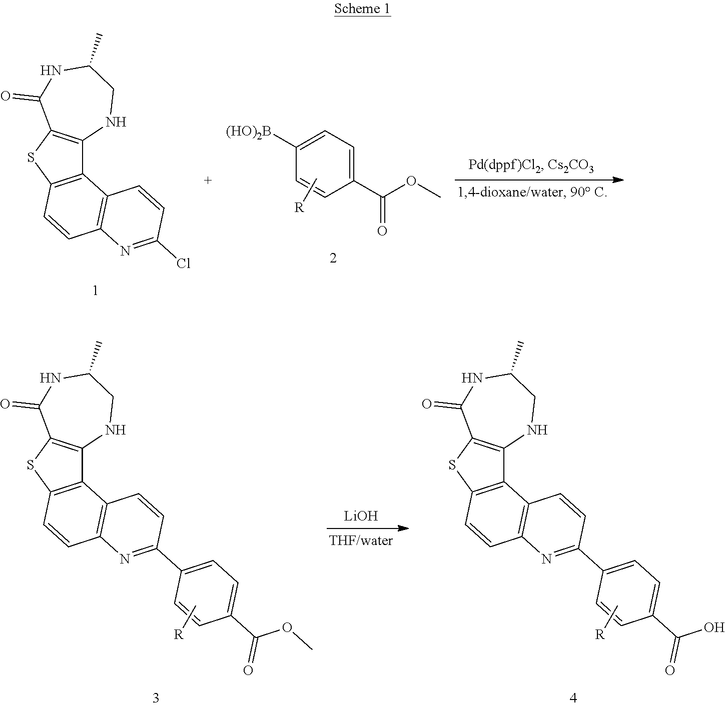

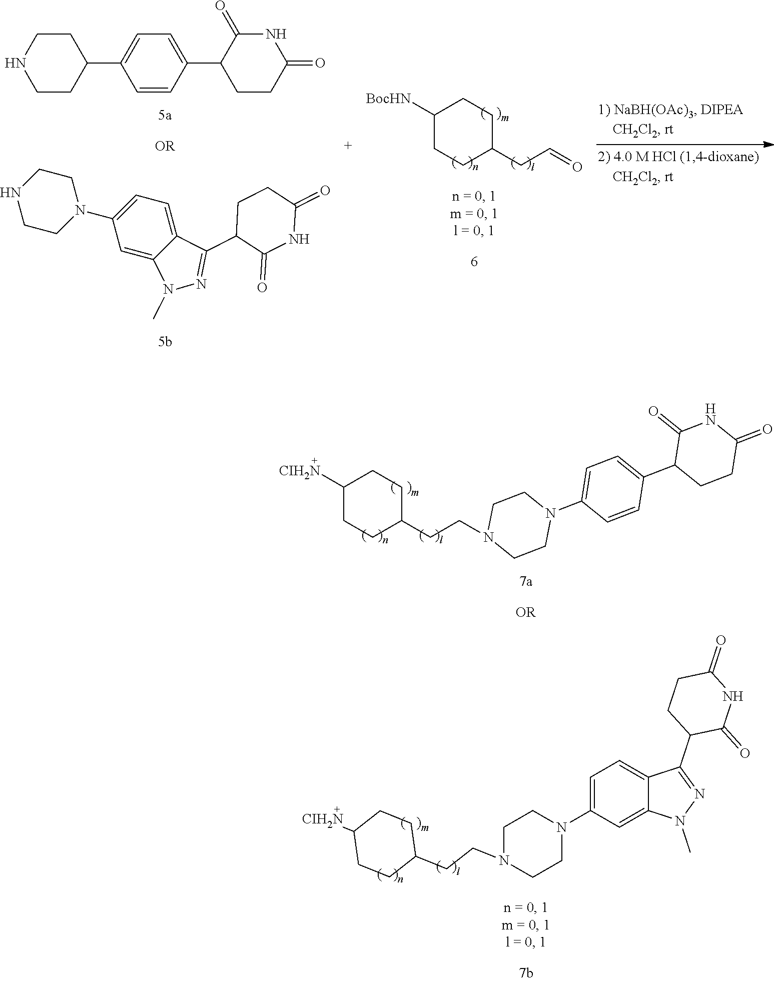

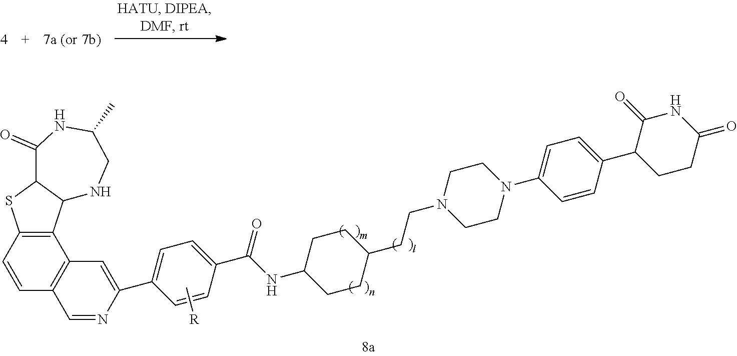

Synthesis of amine coupling partners 7a and 7b can be carried out through reductive amination (Afanasyev, O. I. et al. Chemical Reviews, 2019 11857-11911) of 5a or 5b with aldehyde 6 by treatment with NaBH(OAc)₃ in the presence of a base. Deprotection of the resulting hoc-protected amines can be carried out by treatment with acid, such as 4.0 M HCl in 1,4-dioxane generating 7a or 7b. Lastly, coupling of 4 with 7a (or 7b) through amide formation by a coupling reagent, such as HATU, in the presence of base yields compounds exemplified by 8a. Alternative amination conditions may also be used in this step. The route towards the preparation of compounds exemplified by 14a are shown in Scheme 2.

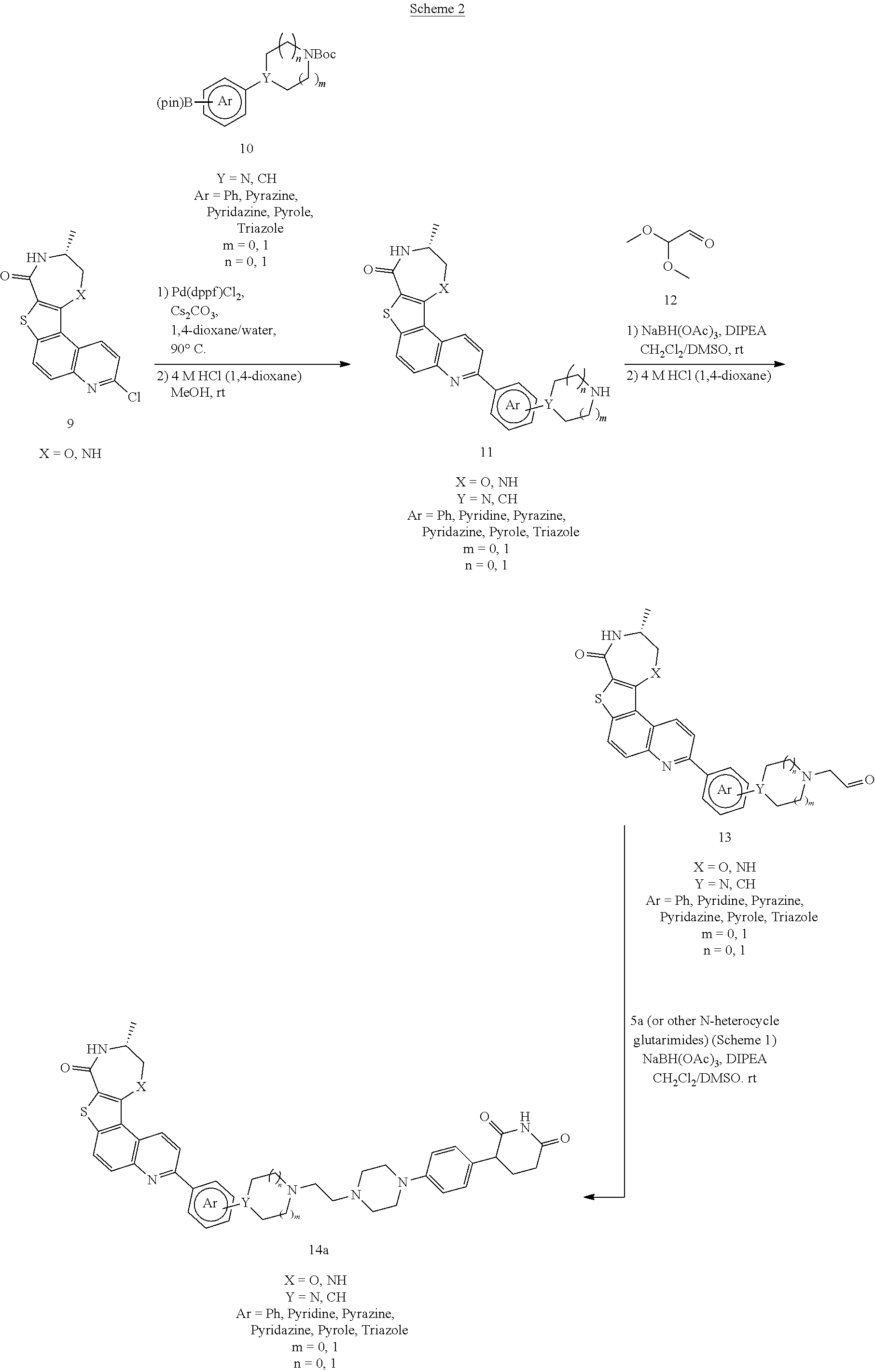

Pd-catalyzed Suzuki cross-coupling between compound 9 with a variety of aryl boronate esters (10) followed by acid-promoted boc-deprotection of the amine, affords compounds demonstrated by 11. Boronic acid, potassium trifluoroborate salt, or MIDA-boronate intermediates would also be functional under similar conditions. Aldehyde 13 can be synthesized by reductive amination with commercially available aldehyde 12, followed by acid-promoted deprotection of the resulting dimethoxy acetal. Alkylation with an analogous halogen-substituted acetal could also be employed. Reductive amination of 13 with intermediates akin to 5a or 5b would generate hetero-bifunctional compounds exemplified by 14a. Scheme 3 demonstrates the synthetic route employed for compounds which cannot be assembled through the double reductive amination strategy employed above.

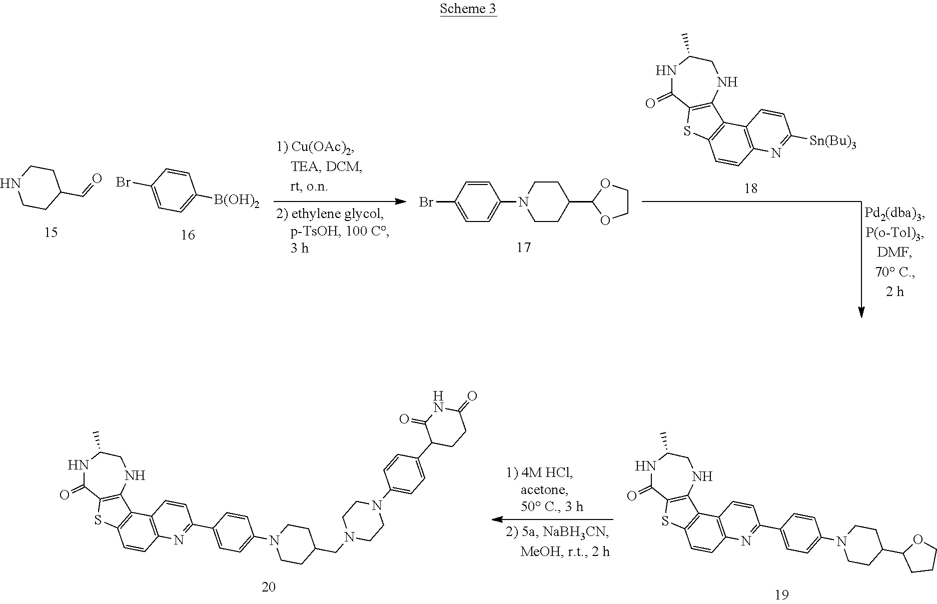

In this case, piperidine-4-carboxaldehyde 15 was coupled with 16 through a copper-catalyzed Chan-Lam coupling (West, M. J. et al. Chemical Reviews, 2019, 12491-12523). The resulting aldehyde was then protected by treatment with ethylene glycol and p-TsOH, furnishing intermediate 17. Coupling of aryl bromide 17 with stannane 18 was accomplished by a Pd-catalyzed Stille cross-coupling reaction. The synthesis of 20 was then accomplished by the acid-promoted deprotection of 19 followed by a reductive amination of the generated aldehyde with intermediate 5a. In other examples, the order of the above steps were changed, as shown in Scheme 4.

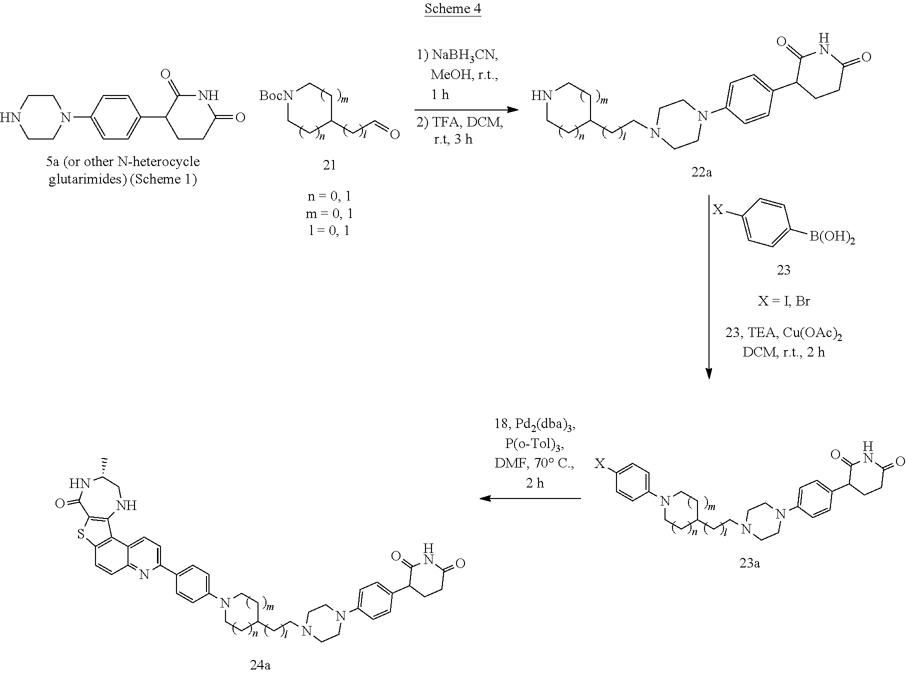

In these cases, intermediates like 5a (or other glutarimides) were first treated with aldehydes such as 21 in the presence of sodium cyanoborohydride, to afford compounds exemplified by 22a (after TFA-promoted deprotection of the boc-group). Chan-Lam coupling of 22a with 23 by treatment with copper(II) acetate and triethylamine at rt afforded 23a. Lastly, Stille-coupling of 23a with 18 catalyzed by Pd₂(dba)₃ and P(o-Tol)₃ in DMF afforded products exemplified by 24a. Schemes 5, 6 and 7 detail cases where compounds of type 11 (Scheme 2) cannot be accessed by a Suzuki cross-coupling.

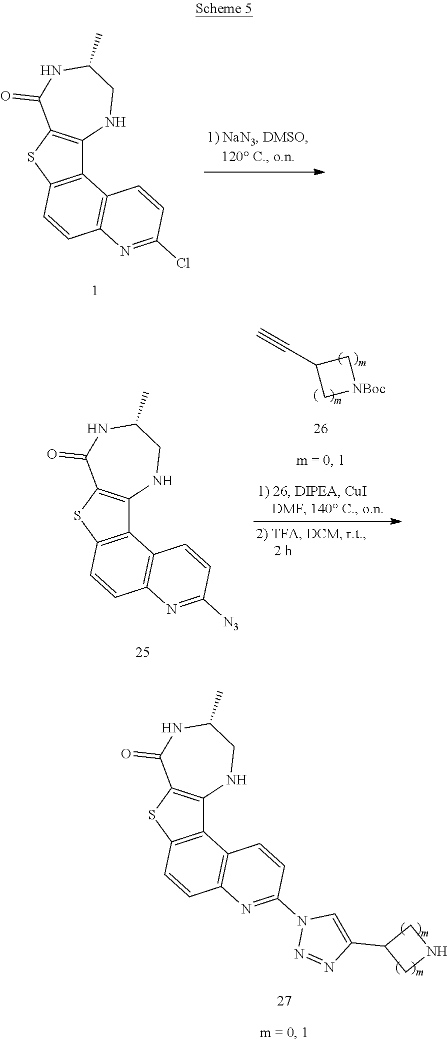

Scheme 5 illustrates an approach to access N-linked triazoles like 27. Treatment of aryl chloride 1 with NaN₃ in DMSO afforded azide 25. A copper-catalyzed Click reaction between azide 25 and alkyne 26, followed by TFA-promoted boc-deprotection afforded intermediate 27. Intermediate 27 was then subjected to the steps outlined in Scheme 2 to access fully elaborated heterobifunctional compounds exemplified by 14a.

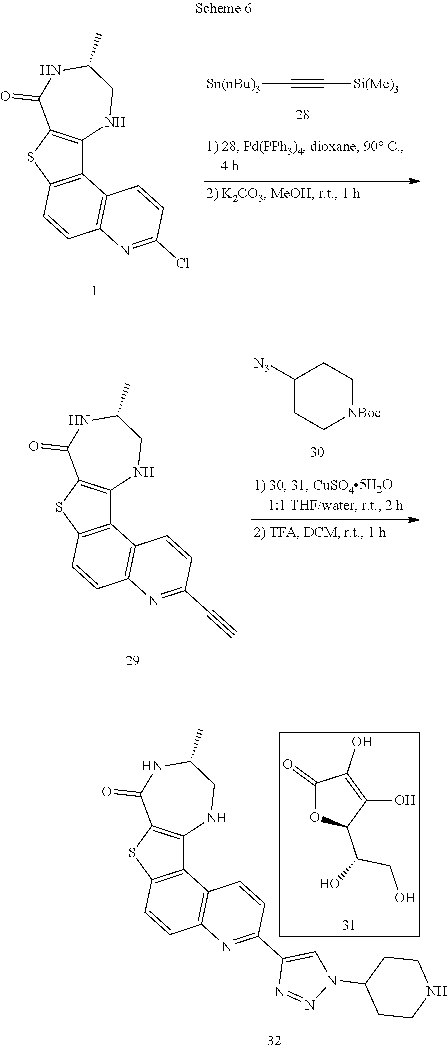

The corresponding C-linked triazoles were accessed through the route detailed in Scheme 6. In this case aryl chloride 1 was converted to alkyne 29 through a two-step procedure. A Stille cross-coupling was performed by treatment of 1 and stannane 28 with Pd(PPh₃)₄. The formed silyl-protected alkyne was then de-silylated by treatment with potassium carbonate in water, affording alkyne 29. A copper catalyzed Click reaction was the performed between alkyne 29 and azide 30 by CuSO₄·5H₂O and chiral tetraol 31. The resulting piperidinyl triazole was then deprotected by TFA in DCM, affording compound 32, which was then subjected to the steps outlined in Scheme 2 to access fully elaborated heterobifunctional compounds exemplified by 14a.

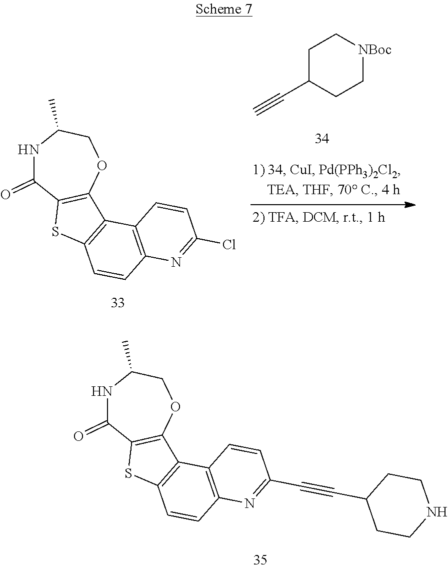 [SMILES: C[C@@H]1COc2c(sc3ccc4nc(Cl)ccc4c23)C(=O)N1 ; C#CC1CCN(C(=O)OC(C)(C)C)CC1 ; C[C@@H]1COc2c(sc3ccc4nc(C#CC5CCNCC5)ccc4c23)C(=O)N1]

Alkynyl-linked heterobifunctional compounds were accessed through a similar strategy. Sonogashira cross-coupling of aryl chloride 33 with alkyne 34 was accomplished by treatment with copper (I) iodide and Pd(PPh₃)₂Cl₂ and TEA. The generated boc-protected alkynyl piperidine was then deprotected by TFA and DCM, affording 35 which was then further elaborated by the procedures outline in scheme 2.

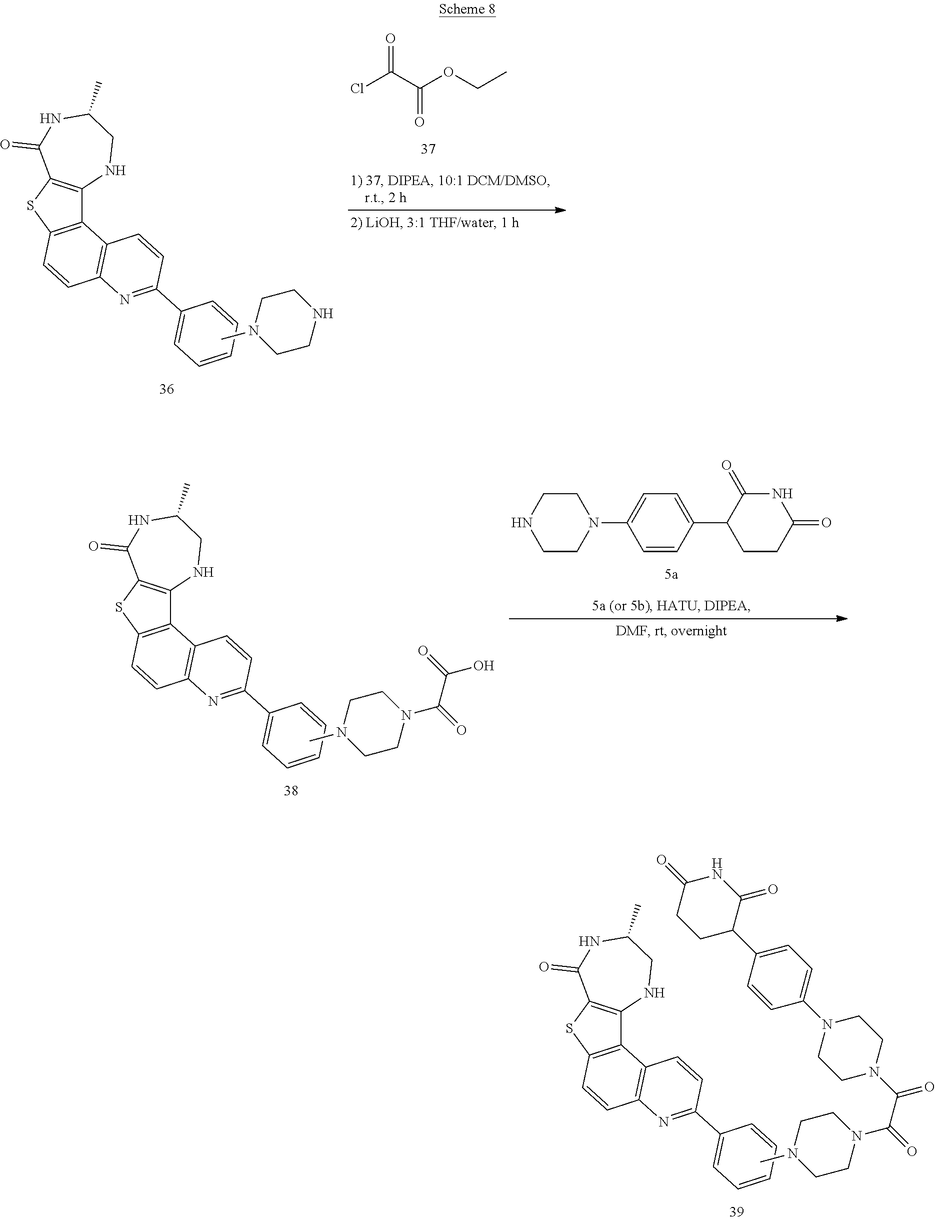

Bis-amide linked compounds exemplified by 39 were synthesized by a three-step sequence of amide formation, saponification, and amide coupling. Piperazinyl-substituted 36 was treated with ethyl chloroglyoxylate and DIPEA. The resulting ethyl ester was then saponified by with LiOH in 3:1 THF/water, furnishing free acid 38. Lastly, free acid 38 was coupled with glutarimides, such as 5a, with HATU and DIPEA, affording bis-amide compounds exemplified by 39.

Purification of intermediates and final products was carried out via either normal or reverse phase chromatography. Normal phase chromatography on an ISCO system was carried out using prepacked SiO₂ cartridges eluting with either gradients of hexanes and EtOAc or DCM and MeOH unless otherwise indicated. Reverse phase preparative HPLC or LCMS was carried out using C18 columns eluting with gradients of Solvent A (90% water, 10% MeOH, 0.1% TFA) and Solvent B (10% water, 90% MeOH, 0.1% TFA, UV 220 nm), or with gradients of Solvent A (95% water, 5% MeCN, 0.1% TFA) and Solvent B (5% water, 95% MeCN, 0.1% TFA, UV 220 nm), or with gradients of Solvent A (98% water, 2% MeCN, 0.05% TFA) and Solvent B (98% MeCN, 2% water, 0.05% TFA, UV 254 nm), or with gradients of Solvent A (95% water, 5% MeCN with 10 mM ammonium acetate) and Solvent B (95% MeCN, 5% water with 10 mM ammonium acetate).

In most examples, one of the following LC/MS conditions were utilized to determine purity:

### LC/MS Method 1

Luna C18 (2) 50×3.0 mm, 3.0 mm. Temperature: 45° C., Flow: 1.5 mL/min, run time: 2.5 min. Mobile phase conditions: Initial 95% H₂O+0.1% FA/5% CH₃CN+0.1% FA, then linear gradient to 95% CH3CN+0.1% FA over 1.3 min. then hold for 1.2 min. at 95% CH3CN+0.1% FA. MSD: ESI Positive

### LC/MS Method 2

SunFire C18 75×4.6 mm, 3.5 μm. Temperature: 45° C., Flow: 1.5 mL/min, run time: 6.0 min. Mobile phase conditions: Initial 95% H₂O+0.1% FA/5% CH₃CN+0.1% FA, then linear gradient to 95% CH₃CN+0.1% FA over 4.0 min. then hold for 2.0 min, at 95% CH₃CN+0.1% FA. MSD: ESI Positive

### LC/MS Method 3

Column: HALO C18, 3.0×30 mm, 2.7 μm particles; Mobile Phase A: H₂O with 0.05% trifluoroacetic acid; Mobile Phase B: CH₃CN with 0.05% trifluoroacetic acid; Temperature: 40° C.; Gradient: 5% B to 100% B over 1.3 min, then a 0.50 min hold at 100% B; Flow: 1.5 mL/min

### LC/MS Method 4

ACQUITY UPLC® BEH C18 1.7 M, 2.1×150 mm. Temperature: 25° C. Flow: 0.8 mL/min. Run time: 3.0 min. Mobile phase condition: Initially starting with 95% H₂O (0.1% formic acid) and 5% acetonitrile (0.1% formic acid), then linear gradient to 95% acetonitrile (0.1% formic acid) for 1.5 min. After holding the gradient for 0.5 min, it is back to with 95% H₂O (0.1% formic acid) and 5% acetonitrile (0.1% formic acid) for 0.1 min. Waters™ SQ Detector 2: ESI positive

### Example 11.1. Synthesis of Intermediate 1: 3-(4-(Piperazin-1-yl)phenyl)piperidine-2,6-dione

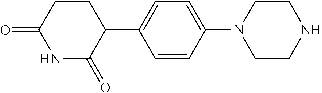 [SMILES: O=C1CCC(c2ccc(N3CCNCC3)cc2)C(=O)N1]

(2,6-Bis(benzyloxy)pyridin-3-yl)boronic acid. To a solution of 2,6-bis(benzyloxy)-3-bromopyridine (400.0 g, 1.08 mol) in THF (4.0 L) was added drop wise n-BuLi (2.5 M, 475.0 mL) at −70° C., then the solution was stirred at −70° C. for 0.5 h. After that, B(OMe)₃ (146.0 g, 1.40 mol, 159.0 mL) was added dropwise to the reaction solution at −70° C., and the reaction mixture was further stirred at −70° C. for 0.5 h. The reaction solution was poured into sat. aq. NH₄Cl (4.0 L). After the organic layer was separated, the aqueous phase was extracted with EtOAc (2×5.0 L). The combined organic layers were washed with brine (5.0 L), dried over Na₂SO₄, filtered and concentrated. The residue was triturated with (PE/EtOAc=10/1, 1.5 L) for 1 h and the solid was collected by filtration and dried under vacuum at 45° C. for 2 h to afford 3-(4-(piperazin-1-yl)phenyl)piperidine-2,6-dione (980.0 g, 64% yield) as a light blue solid. ¹H NMR (400 MHz, CDCl₃) δ ppm 8.05 (d, J=8.0 Hz, 1H), 7.27-7.44 (m, 10H), 6.48 (d, J=8.0 Hz, 1H), 5.93 (s, 2H), 5.45 (s, 2H), 5.38 (s, 2H).

tert-Butyl 4-(4-(2,6-bis(benzyloxy)pyridin-3-yl)phenyl)piperazine-1-carboxylate. A mixture of tert-butyl 4-(4-bromophenyl)piperazine-1-carboxylate (220.0 g, 645 mmol) and (2,6-bis(benzyloxy)pyridin-3-yl)boronic acid (261.0 g, 677 mmol), K₃PO₄ (645 mL, 1.29 mol 2.0 M aq.), Pd(PPh₃)₄(37.3 g, 32.2 mmol) in 1,4-dioxane (2.2 L) was degassed and purged with in-house nitrogen for three times. The mixture was then stirred at 90° C. for 16 h under N₂ atmosphere. After the reaction mixture was cooled to rt, it was filtered, and the filtrate was extracted with EtOAc (3×500 mL). The combined organic phase was washed with brine (500 mL), dried with Na₂SO₄(anhyd), filtered, and concentrated under vacuum. The residue was purified by column chromatography (PE/EtOAc=50/1 to 0/1) to afford tert-butyl 4-(4-(2,6-bis(benzyloxy)pyridin-3-yl)phenyl)piperazine-1-carboxylate (670.0 g) as a green solid. ¹H NMR (400 MHz, CDCl₃) δ ppm 7.59-7.61 (m, 1H), 7.50-7.53 (m, 2H), 7.34-7.43 (m, 10H), 6.96 (d, J=8.8 Hz, 2H), 6.70 (d, J=8.0 Hz, 1H), 5.44 (s, 2H), 5.37 (s, 2H), 3.61 (t, J=5.2 Hz, 4H), 3.18 (t, J=5.2 Hz, 4H), 1.51 (s, 9H).

tert-Butyl 4-(4-(2,6-dioxopiperidin-3-yl)phenyl)piperazine-1-carboxylate. A mixture of tert-butyl 4-(4-(2,6-bis(benzyloxy)pyridin-3-yl)phenyl)piperazine-1-carboxylate (85.0 g, 154 mmol) in THF (400.0 mL) and EtOH (400.0 mL) was added 10% Pd/C (20.0 g), 20% Pd(OH)₂/C (20.0 g) and AcOH (9.3 g, 154 mmol) under N₂ atmosphere. The suspension was degassed under vacuum and purged with hydrogen (H₂) several times. The resulted mixture was stirred at 50° C. under H₂ (40 psi) for 12 h. The reaction mixture was filtered and concentrated. The obtained crude product was triturated with EtOAc (100 mL) at 15° C. for 30 min to afford tert-butyl 4-(4-(2,6-dioxopiperidin-3-yl)phenyl)piperazine-1-carboxylate (220 g (100% purity) and 180 g of a second batch containing impurities. The 180 g crude material was purified by column chromatography (SiO₂, PE/EtOAc=20/1 to 0/1) to give 100 g product, tert-butyl 4-(4-(2,6-dioxopiperidin-3-yl)phenyl)piperazine-1-carboxylate (320 g, 80% average yield) was obtained as a white solid. ¹H NMR (400 MHz, CDCl₃) δ ppm 10.8 (s, 1H), 7.07 (d, J=8.8 Hz, 2H), 6.92 (d, J=8.8 Hz, 2H), 3.74 (dd, J=11.2 Hz, 6.4 Hz, 1H), 3.44-3.45 (m, 4H), 3.07 (t, J=6.4 Hz, 4H), 2.61-2.64 (m, 1H), 2.44-2.48 (m, 1H), 2.13 (m, 1H), 2.01-2.02 (m, 1H), 1.42 (s, 9H).

3-(4-(Piperazin-1-yl)phenyl)piperidine-2,6-dione. Two batches carried out: To a solution of tert-butyl 4-(4-(2,6-dioxopiperidin-3-yl)phenyl)piperazine-1-carboxylate (105 g, 281 mmol) in DCM (600 mL) cooled to 15° C. was added dropwise HCl/EtOAc (4 M, 500 mL). After addition, the mixture was stirred at 15° C. for 3 h. The two batches were combined and the reaction mixture was filtered and the filter cake was dried. The crude product was triturated with EtOAc (500 mL) at 15° C. for 30 min. 3-(4-(piperazin-1-yl)phenyl)piperidine-2,6-dione, 2 HCl (194 g, 560 mmol, quantitative yield) was obtained as a white solid. ¹H NMR (400 MHz, DMSO-d6) δ ppm 10.8 (s, 1H), 9.24 (s, 2H), 7.11 (d, J=8.4 Hz, 2H), 6.96 (d, J=8.8 Hz, 2H), 3.77 (q, J=5.2 Hz, 1H), 3.34-3.36 (m, 4H), 3.20 (s, 4H), 2.61-2.64 (m, 1H), 2.44-2.48 (m, 1H), 2.14 (m, 1H), 1.90-2.01 (m, 1H).

### Example 11.2. Synthesis of Intermediate 2: 3-(4-(Piperidin-4-yl)phenyl)piperidine-2,6-dione

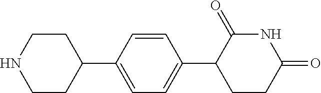 [SMILES: O=C1CCC(c2ccc(C3CCNCC3)cc2)C(=O)N1]

tert-Butyl 4-(4-(2,6-bis(benzyloxy)pyridin-3-yl)phenyl)piperidine-1-carboxylate. To a solution of tert-butyl 4-(4-bromophenyl)piperidine-1-carboxylate (2.0 g, 5.9 mmol), (2,6-dibenzyloxy-3-pyridyl)boronic acid (2.96 g, 8.8 mmol) and K₂CO₃ (1.6 g, 11.8 mmol) in 1,4-dioxane (20.0 mL) and water (2.0 mL) was added Pd(PPh₃)₄(497.0 mg, 0.6 mmol). The resulting solution was stirred at 50° C. for 3 h under an inert atmosphere. LCMS showed the reaction was completed. The resulting solution was diluted with water (50 mL), extracted with ethyl acetate (3×50 mL). The combined organic layers were washed with brine (2×30 mL), dried over anhydrous sodium sulfate, filtered, and concentrated under reduced pressure. The residue was purified by column chromatography on silica gel eluted with petroleum ether/ethyl acetate (3:1) to afford tert-butyl 4-[4-(2,6-dibenzyloxy-3-pyridyl)phenyl]piperidine-1-carboxylate (2.8 g, 86% yield) as a white solid. MS (ESI, m/z) [M+H]⁺ 551.

tert-Butyl 4-(4-(2,6-dioxopiperidin-3-yl)phenyl)piperidine-1-carboxylate. To a stirred solution of tert-butyl 4-[4-(2,6-dibenzyloxy-3-pyridyl)phenyl]piperidine-1-carboxylate (1.5 g, 2.7 mmol, 1.0 equiv) in ethanol (10.0 mL) and THF (10.0 mL) was added Pd/C (300.0 mg, 20% w/w), Pd(OH)₂/C (300.0 mg, 20% w/w) and AcOH (0.16 mL, 2.7 mmol, 1.0 equiv). The resulting solution was stirred for 18 h at 50° C. under hydrogen atmosphere at 40 psi. LCMS showed the reaction was completed. The solid was filtered out. The filtration was concentrated under reduced pressure and the residue purified by column chromatography on silica gel with petroleum ether/ethyl acetate (3:1) to afford tert-butyl 4-[4-(2,6-dioxo-3-piperidyl)phenyl]piperidine-1-carboxylate (850 mg, 83.7% yield) as a white solid. MS (ESI, m/z) [M+H]⁺ 373.

3-(4-(Piperidin-4-yl)phenyl)piperidine-2,6-dione. To a solution of tert-butyl 4-[4-(2,6-dioxo-3-piperidyl)phenyl]piperidine-1-carboxylate (850.0 mg, 2.3 mmol, 1.0 equiv) in HCl (20 mL, 2.0 M in EtOAc). The resulting solution was stirred at rt for 2 h. LCMS showed the reaction was completed. The resulting solution was concentrated under reduced pressure to afford crude 3-[4-(4-piperidyl)phenyl]piperidine-2,6-dione (800.0 mg) as a white solid. The crude product was used in the next step directly without further purification. MS (ESI, n z) [M+H]⁺ 273.

### Example 11.3. Synthesis of Intermediate 3: 3-(1-Methyl-6-(piperazin-1-yl)-1H-indazol-3-yl)piperidine-2,6-dione

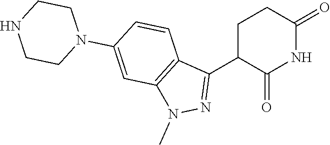 [SMILES: Cn1nc(C2CCC(=O)NC2=O)c2ccc(N3CCNCC3)cc21]

tert-Butyl 4-(3-(2,6-Bis(benzyloxy)pyridin-3-yl)-1-methyl-1H-indazol-6-yl)piperazine-1-carboxylate. A mixture of 6-bromo-3-(2,6-dibenzyloxy-3-pyridyl)-1-methyl-indazole (30.0 g, 60.0 mmol), tert-butyl piperazine-1-carboxylate (16.8 g, 89.9 mmol), RuPhos-Pd-G3 (10 g, 12.0 mmol) and Cs₂CO₃ (23.4 g, 71.9 mmol) in degassed 1,4-dioxane (150 mL) was heated to 75° C. for 18 h and then cooled to rt. The mixture was filtered through Celite and the filter cake washed with EtOAc (3×150 mL). The filtrate was concentrated under reduced pressure and the residue was purified by column chromatography on silica gel using a gradient of 0-40% EtOAc in hexanes to afford title compound tert-butyl 4-(3-(2,6-bis(benzyloxy)pyridin-3-yl)-1-methyl-1H-indazol-6-yl)piperazine-1-carboxylate (35.6 g, 98% yield) as a solid. MS (ESI, m/z) [M+H]⁺ 607.5.

tert-Butyl 4-(3-(2,6-dioxopiperidin-3-yl)-1-methyl-1H-indazol-6-yl)piperazine-1-carboxylate. A mixture of tert-butyl 4-[3-(2,6-dibenzyloxy-3-pyridyl)-1-methyl-indazol-6-yl]piperazine-1-carboxylate (35.6 g, 58.8 mmol) and Pearlman's catalyst (8.90 g, 25 wt. % loading) in EtOH (300 mL) and THE (300 mL) was subjected to hydrogenation (1.0 atm) at 50° C. for 10 h. The mixture was filtered through Celite and the filter cake washed with a 1:1 mixture of MeCN and MeOH (4×250 mL). The filtrate was concentrated under reduced pressure and the residue was purified by column chromatography on silica gel using a gradient of 0-100% EtOAc in hexanes to afford title compound tert-butyl 4-(3-(2,6-dioxopiperidin-3-yl)-1-methyl-1H-indazol-6-yl)piperazine-1-carboxylate (21.0 g, 84% yield) as a solid. MS (ESI, m/z) [M+H]⁺ 428.3.

3-(1-Methyl-6-(piperazin-1-yl)-1H-indazol-3-yl)piperidine-2,6-dione. To a solution of tert-butyl 4-[3-(2,6-dioxo-3-piperidyl)-1-methyl-indazol-6-yl]piperazine-1-carboxylate (21.0 g, 49.1 mmol) in 1,4-dioxane (150 mL) was added 4.0 N HCl in 1,4-dioxane (98.2 mL, 393 mmol) and the reaction mixture was stirred at rt for 20 h. Et₂O (250 mL) was added and the precipitate was collected by filtration, washed with Et₂O (3×30 mL), then dried under vacuum and lyophilized to afford the title compound 3-(1-methyl-6-(piperazin-1-yl)-1H-indazol-3-yl)piperidine-2,6-dione (17.6 g, 98% yield) as a solid. MS (ESI, m/z) [M+H]⁺ 328.2. ¹H NMR (500 MHz, DMSO-d₆) δ ppm 10.85 (s, 1H), 9.46 (s, 2H), 7.56 (d, J=8.9 Hz, 1H), 7.04-6.90 (m, 2H), 4.28 (dd, J=9.4, 5.0 Hz, 1H), 3.92 (s, 3H), 3.53-3.41 (m, 4H), 3.23 (s, 4H), 2.73-2.55 (m, 2H), 2.39-2.26 (m, 1H), 2.23-2.08 (m, 1H).

### Example 11.4. Synthesis of Intermediate 4: 3-(1-Methyl-7-(piperazin-1-yl)-1H-indazol-3-yl)piperidine-2,6-dione

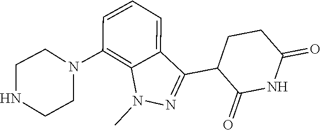 [SMILES: Cn1nc(C2CCC(=O)NC2=O)c2cccc(N3CCNCC3)c21]

7-Bromo-3-iodo-1-methyl-1H-indazole. Three batches carried out: to a solution of 7-bromo-3-iodo-1H-indazole (480 g, 1.49 mol) in THE (2.4 L) at 0° C. was added portion-wise t-BuOK (334 g, 2.97 mol). After the addition, the suspension was stirred at 0° C. for 1 h. Then a solution of CH₃I (422 g, 2.97 mol, 185 mL) in THE (400 mL) was added dropwise to the cooled (0° C.) reaction mixture. The suspension was then stirred at 25° C. for 3 h. TLC (PE/EtOAc=5/1, R_{f}=0.5) showed the reaction was completed. The mixtures of three reactions were combined and the resulting suspension was poured into water (10 L) and stirred for 10 min. The aqueous phase was extracted with EtOAc (5.0 L×1, then 3.0 L×1). The combined organic phase was washed with brine (3.0 L), dried with anhydrous Na₂SO₄, filtered, and concentrated in vacuum. The residue was purified by column chromatography on silica gel (PE/EtOAc=25/1, 5/1) to afford 7-bromo-3-iodo-1-methyl-1H-indazole (900 g, 60% yield) as a yellow solid. ¹H NMR (400 MHz, DMSO-d₆) δ ppm 7.72 (d, J=8.4 Hz, 1H), 7.49 (d, J=7.2 Hz, 1H), 7.11 (t, J=7.6 Hz, 1H), 4.34 (s, 3H).

3-(2,6-Bis(benzyloxy)pyridin-3-yl)-7-bromo-1-methyl-1H-indazole. Three batches carried out: to a solution of 7-bromo-3-iodo-1-methyl-1H-indazole (313 g, 929 mmol) in 1,4-dioxane (2.0 L) and H₂O (1.0 L) was added (2,6-bis(benzyloxy)pyridin-3-yl)boronic acid (389 g, 929 mmol, 80% purity), K₃PO₄ (493 g, 2.32 mol) and Pd(PPh₃)₄(21.5 g, 18.6 mmol). Then the suspension was purged with N₂ three times and stirred at 90° C. for 12 h. The three batches were combined for work-up and then the reaction mixture was poured into water (10 L) and stirred for 10 min. The aqueous phase was extracted with EtOAc (5 L×1, then 3 L×1). The combined organic phase was washed with brine (3 L), dried with anhydrous Na₂SO₄, filtered, and concentrated in vacuo. The residue was purified by column chromatography on silica gel (PE/EtOAc=25/1, 5/1). The purified residue was triturated with PE/EtOAc (2/1) at 25° C. for 3 h, then the solids were collected by vacuum filtration to afford 3-(2,6-bis(benzyloxy)pyridin-3-yl)-7-bromo-1-methyl-1H-indazole (920 g, 63% yield) as an off-white solid. ¹H NMR (400 MHz, DMSO-d₆) δ ppm 7.86 (d, J=8.4 Hz, 1H), 7.54 (dd, J=8.0, 0.8 Hz, 1H), 7.37 (dd, J=7.2, 0.8 Hz, 1H), 7.33 (m, 2H), 7.28 (m, 8H), 6.94 (t, J=7.6 Hz, 1H), 6.60 (d, J=7.6 Hz, 1H), 5.43 (s, 4H), 4.36 (s, 3H).

tert-Butyl 4-(3-(2,6-bis(benzyloxy)pyridin-3-yl)-1-methyl-1H-indazol-7-yl)piperazine-1-carboxylate. To a solution of 3-(2,6-bis(benzyloxy)pyridin-3-yl)-7-bromo-1-methyl-1H-indazole (100 g, 200 mmol,) and tert-butyl piperazine-1-carboxylate (55.8 g, 300 mmol) in 1,4-dioxane (700 mL) was added Cs₂CO₃ (130 g, 400 mmol), RuPhos (18.6 g, 40 mmol) and Pd₂(dba)₃ (18.3 g, 20 mmol), then the suspension was purged with N₂ three times and stirred at 110° C. for 12 h. TLC (PE/EtOAc=3/1, R_{f}=0.6) showed the reaction was completed. The reaction was cooled to 20° C. and filtered through a pad of celite. The filtrate was concentrated under vacuum and the residue was purified by silica gel chromatography (100-200 mesh silica gel, PE/EtOAc=20/1, 3/1) to give the product. The product was further purified by trituration with PE/EtOAc=(2/1, 200 mL) for 1 h. The solid was collected by filtration and dried under vacuum to afford tert-butyl 4-(3-(2,6-bis(benzyloxy)pyridin-3-yl)-1-methyl-1H-indazol-7-yl)piperazine-1-carboxylate (84 g, 67% yield) as a yellow solid. ¹H NMR (400 MHz DMSO-d₆) δ ppm 7.85 (d, J=8.4 Hz, 1H), 7.27-7.45 (m, 12H), 6.96-6.99 (m, 2H), 6.53 (d, J=8.0 Hz, 1H), 5.47 (s, 2H), 5.40 (s, 2H), 4.41 (s, 3H), 4.10-4.16 (m, 2H), 3.20-3.23 (m, 4H), 2.84-2.89 (m, 2H), 1.51 (s, 9H).

tert-Butyl 4-(3-(2,6-dioxopiperidin-3-yl)-1-methyl-1H-indazol-7-yl)piperazine-1-carboxylate. To a suspension of tert-butyl 4-(3-(2,6-bis(benzyloxy)pyridin-3-yl)-1-methyl-1H-indazol-7-yl)piperazine-1-carboxylate (42 g, 69.3 mmol) and AcOH (4.16 g, 69.3 mmol, 3.97 mL) in THF (210 mL) and EtOH (210 mL) was added 10% Pd/C (8.0 g) and 20% Pd(OH)₂ (8.0 g, 57 mmol), then the black suspension was purged with H₂ three times and stirred at 50° C. under 50 psi for 12 h. The suspension was filtered through a pad of celite and the filter cake was washed with hot THF (2 L). The filtrate was concentrated under vacuum at 45° C. to get the crude product. The crude material was purified by silica gel chromatography (100-200 mesh silica gel, 0% to 10% MeOH in DCM) to give a solid. The solid was further triturated with MTBE (50 mL) for 1 h. The solid was collected by filtration and dried under vacuum. tert-butyl 4-(3-(2,6-dioxopiperidin-3-yl)-1-methyl-1H-indazol-7-yl)piperazine-1-carboxylate (18.6 g, 30% yield) was obtained as a blue solid. MS (ESI, m/z) [M+H]⁺ 428.4.

3-(1-Methyl-7-(piperazin-1-yl)-1H-indazol-3-yl)piperidine-2,6-dione. To a solution of tert-butyl 4-(3-(2,6-dioxopiperidin-3-yl)-1-methyl-1H-indazol-7-yl)piperazine-1-carboxylate (18.6 g, 43.5 mmol) in DCM (420 mL) was added HCl/EtOAc (4.0 M, 93.0 mL), then the suspension was stirred at 20° C. for 2 h. The solid was collected by filtration and dried under vacuum at 45° C. for 2 h. The solid was suspended in MeCN (100 mL) and dried under vacuum at 45° C. for 2 h. The operation was repeated two more times. 3-(1-Methyl-7-(piperazin-1-yl)-1H-indazol-3-yl)piperidine-2,6-dione (16.5 g, 95% yield) was obtained as a light blue solid. ¹H NMR (400 MHz, DMSO-d₆) δ ppm 10.87 (s, 1H), 9.49-9.58 (m, 2H), 7.45 (d, J=6.8 Hz, 1H), 7.03-7.06 (m, 2H), 4.33-4.37 (m, 1H), 4.24 (s, 3H), 3.15-3.44 (m, 8H), 2.60-2.67 (m, 2H), 2.31-2.50 (m, 1H), 2.14-2.18 (m, 1H).

### Example 11.5. Synthesis of Intermediate 5: tert-Butyl (R)-3-chloro-10-methyl-8-oxo-10,11-dihydro-[1,4]oxazepino[7′,6′:4,5]thieno[3,2-f]quinoline-9(8H)-carboxylate

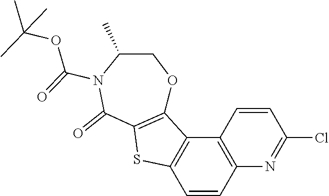 [SMILES: C[C@@H]1COc2c(sc3ccc4nc(Cl)ccc4c23)C(=O)N1C(=O)OC(C)(C)C]

Methyl 1-bromothieno[3,2-f]quinoline-2-carboxylate. To a solution of methyl 1-aminothieno[3,2-f]quinoline-2-carboxylate (30.0 g, 116.0 mmol) in acetonitrile (750 mL) was added tert-butyl nitrite (24.0 g, 232.0 mmol) drop wise at 0° C. under N₂. After the reaction mixture was stirred at 0° C. for 1 h, CuBr₂ (31.1 g, 139.0 mmol) was added at 0° C., and then the reaction mixture was stirred at 25° C. for 3 h under N₂. The mixture was concentrated and water (300 mL) was added, the mixture was extracted with DCM/i-PrOH (3/1, 600 mL×3). The combined organic layers were concentrated to give methyl 1-bromothieno[3,2-f]quinoline-2-carboxylate (34.0 g, 90.8% yield) as a yellow solid. MS (ESI+) [M+H]⁺ 321.9. ¹H NMR (400 MHz, DMSO-d₆) δ ppm 10.12 (d, J=8.4 Hz, 1H), 9.14-9.05 (m, 1H), 8.42 (d, J=8.4 Hz, 1H), 8.16 (d, J=8.4 Hz, 1H), 7.81-7.78 (m, 1H), 3.94 (s, 3H).

Methyl (R)-1-(2-aminopropoxy)thieno[3,2-f]quinoline-2-carboxylate. To a solution of methyl (R)-1-(2-((tert-butoxycarbonyl)amino)propoxy)thieno[3,2-f]quinoline-2-carboxylate (20.0 g, 48.0 mmol) in DCM (200 mL) was added TFA (67 mL, 875 mmol) drop wise at 0° C., then the reaction mixture was stirred at 20° C. for 3 h. The reaction mixture was concentrated, then added water (100 mL), extracted with petroleum ether (PE)/EtOAc (2:1, 15 mL×2). The aqueous phase was adjusted with sat. NaHCO₃ (aq.) to pH=7, the solid was slowly precipitated and filtered. The filtrate was concentrated under vacuum to afford methyl 1-[(2R)-2-aminopropoxy]thieno[3,2-f]quinoline-2-carboxylate (13.0 g, 85.6% yield) as a yellow solid. ¹H NMR (400 MHz, DMSO-d₆) δ ppm 9.20 (d, J=8.0 Hz, 1H), 9.01-9.00 (m, 1H), 8.31 (d, J=9.2 Hz, 1H), 8.23-8.10 (m, 3H), 7.74 (d, J=4.4 Hz, 1H), 4.43-4.31 (m, 2H), 3.93 (s, 3H), 3.92-3.90 (m, 1H), 1.36 (d, J=6.4 Hz, 3H).

(R)-10-Methyl-10,11-dihydro-[1,4]oxazepino[7′,6′:4,5]thieno[3,2-f]quinolin-8(9H)-one. To a solution of methyl (R)-1-(2-aminopropoxy)thieno[3,2-f]quinoline-2-carboxylate (13.0 g, 41.1 mmol) in methanol (400 mL) was added sodium methoxide (14.8 g, 82.2 mmol, 30 w/w % in MeOH) at 20° C., then the reaction mixture was stirred at 75° C. for 14 h. The reaction mixture was concentrated, then triturated with MTBE/water (1/1, 60 mL) for 30 mins. The mixture was filtered, and the filtrate was concentrated under vacuum to afford (R)-10-methyl-10,11-dihydro-[1,4]oxazepino[7′,6′:4,5]thieno[3,2-f]quinolin-8(9H)-one (9.9 g, 84.7% yield) as a yellow solid. ¹H NMR (400 MHz, DMSO-d₆) δ ppm 9.28 (dd, J=0.8 Hz, 8.0 Hz, 1H), 8.95 (dd, J=2.0 Hz, 4.4 Hz, 1H), 8.48 (d, J=4.0 Hz, 1H), 8.20 (d, J=9.2 Hz, 1H), 8.02 (d, J=8.8 Hz, 1H), 7.68 (q, J=4.4 Hz, 1H), 4.61 (s, 2H), 3.88-3.84 (m, 1H), 1.28 (d, J=6.8 Hz, 3H).

tert-Butyl (R)-10-methyl-8-oxo-10,11-dihydro-[1,4]oxazepino[7′,6′:4,5]thieno[3,2-f]quinoline-9(8H)-carboxylate. To a solution of (R)-10-methyl-10,11-dihydro-[1,4]oxazepino[7′,6′:4,5]thieno[3,2-f]quinolin-8(9H)-one (4.4 g, 15.5 mmol) and Boc₂O (6.8 g, 31.0 mmol) in THF (100.0 mL) was added DMAP (189 mg, 1.6 mmol). The resulting mixture was stirred at 60° C. for 2 h under nitrogen atmosphere. The resulting solution was concentrated under vacuum. The residue was purified by column chromatography on silica gel with ethyl acetate/petroleum ether (1:1) to afford tert-butyl (R)-10-methyl-8-oxo-10,11-dihydro-[1,4]oxazepino[7′,6′:4,5]thieno[3,2-f]quinoline-9(8H)-carboxylate (5.5 g, 93%) as an off-white solid. MS (ESI, m/z) [M+H]⁺ 385.1

tert-Butyl (R)-10-methyl-4-(11-oxidaneyl)-8-oxo-10,11-dihydro-414-[1,4]oxazepino[7′,6′:4,5]thieno[3,2-f]quinoline-9(8H)-carboxylate. To a solution of tert-butyl (R)-10-methyl-8-oxo-10,11-dihydro-[1,4]oxazepino[7′,6′:4,5]thieno[3,2-f]quinoline-9(8H)-carboxylate (5.0 g, 13.0 mmol) in DCM (120.0 mL) was added dropwise 3-chlorobenzoperoxoic acid (3.4 g, 19.5 mmol) at ice bath. The resulting mixture was stirred at 30° C. for 2 h. The resulting solution was diluted with water (100 mL), extracted with ethyl acetate (3×100 mL). The combined organic layers were washed by sodium carbonate (50 mL×3) and brine (50 mL×2), dried over sodium sulfate, filtered, and concentrated under reduced pressure to afford 5.2 g crude product. The crude product was used directly for the next step without further purification. MS (ESI, m/z) [M+H]⁺ 401.1.

tert-Butyl (R)-3-chloro-10-methyl-8-oxo-10,11-dihydro-[1,4]oxazepino[7′,6′:4,5]thieno[3,2-f]quinoline-9(8H)-carboxylate. To a solution of tert-butyl (R)-10-methyl-4-(11-oxidaneyl)-8-oxo-10,11-dihydro-414-[1,4]oxazepino[7′,6′:4,5]thieno[3,2-f]quinoline-9(8H)-carboxylate (5.2 g, 13.0 mmol) in DMF (60.0 mL) was added dropwise oxalyl dichloride (2.5 g, 19.5 mmol) at 0° C. The resulting mixture was allowed to warm up to rt and stirred overnight under nitrogen atmosphere. The reaction mixture was diluted with water (600 mL) and stirred for 10 min. The solid participated was collected by filtration, washed with petroleum ether (2×300 mL) and dried under vacuum to afford tert-butyl (R)-3-chloro-10-methyl-8-oxo-10,11-dihydro-[1,4]oxazepino[7′,6′:4,5]thieno[3,2-f]quinoline-9(8H)-carboxylate (5.0 g, 80% yield) as an off-white solid. MS (ESI, m/z) [M+H]⁺ 418.2

### Example 11.6. Synthesis of Intermediate 6: tert-Butyl (R)-3-bromo-10-methyl-8-oxo-10,11-dihydro-[1,4]oxazepino[7′,6′:4,5]thieno[3,2-f]quinoline-9(8H)-carboxylate

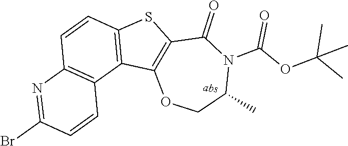 [SMILES: C[C@@H]1COc2c(sc3ccc4nc(Br)ccc4c23)C(=O)N1C(=O)OC(C)(C)C]

tert-Butyl (R)-10-methyl-8-oxo-10,11-dihydro-[1,4]oxazepino[7′,6′:4,5]thieno[3,2-f]quinoline-9(8H)-carboxylate. To a solution of (R)-10-methyl-10,11-dihydro-[1,4]oxazepino[7′,6′:4,5]thieno[3,2-f]quinolin-8(9H)-one (4.4 g, 15.5 mmol) and di-tert-butyl dicarbonate (6.8 g, 31.0 mmol) in THE (100 mL) was added DMAP (189 mg, 1.55 mmol). The resulting mixture was stirred under nitrogen atmosphere at 60° C. for 2 h. After concentrating under reduced pressure, the residue was purified by column chromatography on silica gel (eluted with ethyl acetate/petroleum ether, 1:1) to afford tert-butyl (R)-10-methyl-8-oxo-10,11-dihydro-[1,4]oxazepino[7′,6′:4,5]thieno[3,2-j]quinoline-9(8H)-carboxylate (5.5 g, 93% yield) as an off-white solid. MS (ESI, m/z) [M+H]⁺ 385.2.

(R)-9-(tert-Butoxycarbonyl)-10-methyl-8-oxo-8,9,10,11-tetrahydro-[1,4]oxazepino[7′,6′:4,5]thieno[3,2-f]quinoline 4-oxide. To a solution of tert-butyl (R)-10-methyl-8-oxo-10,11-dihydro-[1,4]oxazepino[7′,6′:4,5]thieno[3,2-f]quinoline-9(8H)-carboxylate (5.0 g, 13.0 mmol) in DCM (120 mL) was added dropwise 3-chlorobenzoperoxoic acid (3.4 g, 19.5 mmol) at 0° C. The resulted mixture was stirred at 30° C. for 2 h. After the reaction mixture was diluted with water (100 mL) and extracted with ethyl acetate (100 mL×3), the combined organic layers were washed with saturated aqueous sodium carbonate (50 mL×3) and brine (50 mL×2), dried over sodium sulfate, filtered and concentrated under reduced pressure to afford give (R)-9-(tert-butoxycarbonyl)-10-methyl-8-oxo-8,9,10,11-tetrahydro-[1,4]oxazepino[7′,6′:4,5]thieno[3,2-j]quinoline 4-oxide as a crude product (5.2 g), MS (ESI, m/z) [M+H]⁺ 401.3. The product was used directly for the next step without further purification.

tert-Butyl (R)-3-bromo-10-methyl-8-oxo-10,11-dihydro-[1,4]oxazepino[7′,6′:4,5]thieno[3,2-f]quinoline-9(8H)-carboxylate. To a solution of (R)-9-(tert-butoxycarbonyl)-10-methyl-8-oxo-8,9,10,11-tetrahydro-[1,4]oxazepino[7′,6′:4,5]thieno[3,2-f]quinoline 4-oxide (5.2 g, 13.0 mmol) in DMF (60 mL) was added dropwise phosphoryl bromide (5.6 g, 19.5 mmol) at 0° C. The resulted mixture was allowed to warm up to rt and continuously stirred under nitrogen atmosphere overnight. After the reaction was quenched with water (600 mL) at 0° C., the mixture was extracted with dichloromethane (3×100 mL). The combined organic layers were concentrated under reduced pressure, and the residue was purified by column chromatography on silica gel (eluted with ethyl acetate/dichloromethane, 1:2) to afford tert-butyl (R)-3-bromo-10-methyl-8-oxo-10,11-dihydro-[1,4]oxazepino[7′,6′:4,5]thieno[3,2-j]quinoline-9(8H)-carboxylate (4.5 g, 75% yield) as a white solid. ¹H NMR (400 MHz, DMSO-d₆) δ ppm 9.15 (d, J=8.8 Hz, 1H), 8.29 (d, J=9.2 Hz, 1H), 8.00 (d, J=9.2 Hz, 1H), 7.86 (d, J=8.8 Hz, 1H), 5.03-4.98 (m, 1H), 4.82-4.76 (m, 1H), 4.59-4.56 (m, 1H), 1.50 (s, 9H), 1.28 (d, J=6.8 Hz, 3H).

### Example 11.7. Synthesis of Intermediate 7: (R)-10-Methyl-3-(tributylstannyl)-9,10,11,12-tetrahydro-8H-[1,4]diazepino[5′,6′:4,5]thieno[3,2-f]quinolin-8-one

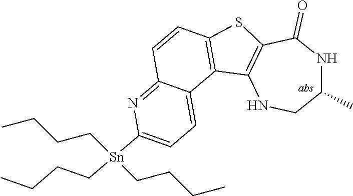 [SMILES: CCC[CH2][Sn]([CH2]CCC)([CH2]CCC)[c]1ccc2c(ccc3sc4c(c32)NC[C@@H](C)NC4=O)n1]

(R)-10-Methyl-3-(tributylstannyl)-9,10,11,12-tetrahydro-8H-[1,4]diazepino[5′,6′:4,5]thieno[3,2-f]quinolin-8-one. To a solution of (R)-3-chloro-10-methyl-9,10,11,12-tetrahydro-8H-[1,4]diazepino[5′,6′:4,5]thieno[3,2-f]quinolin-8-one (10.0 g, 31.4 mmol) and hexa-n-butylditin (22 g, 37.7 mmol) in DMF (200.0 mL) were added Pd(PPh₃)₂Cl₂ (1.1 g, 1.57 mmol) and LiCl (2.0 g, 47.2 mmol). The resulted mixture was stirred at 90° C. under nitrogen atmosphere overnight (16 h). After diluted with saturated aqueous potassium fluoride (1.0 L), the mixture was extracted with ethyl acetate (500 mL×5). The combined organic layer was washed with brine (300 mL×2), dried over anhydrous sodium sulfate, filtered, and concentrated under reduced pressure. The residue was purified by column chromatography on silica gel with ethyl acetate/petroleum ether (8:1) to afford (R)-10-methyl-3-(tributylstannyl)-9,10,11,12-tetrahydro-8H-[1,4]diazepino[5′,6′:4,5]thieno[3,2-f]quinolin-8-one (7.5 g, 41% yield) as a yellow solid. MS (ESI, m/z) [M+H]⁺ 574.2 ¹H NMR (400 MHz, DMSO-d₆) δ ppm 8.95 (d, J=8.4 Hz, 1H), 8.07 (s, 1H), 8.06 (d, J=9.2 Hz, 1H), 7.94 (d, J=9.2 Hz, 1H), 7.69-7.53 (m, 3H), 7.08 (t, J=5.2 Hz, 1H), 3.62-3.57 (m, 1H), 3.47-3.44 (m, 2H), 1.63-1.53 (m, 6H), 1.37-1.28 (m, 6H), 1.19-1.15 (m, 9H), 0.85 (t, J=7.2 Hz, 3H).

### Example 1: Synthesis of N-(1-(3-(2,6-dioxopiperidin-3-yl)-1-methyl-1H-indazol-6-yl)piperidin-4-yl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide

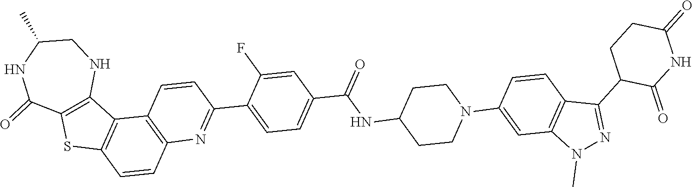 [SMILES: C[C@@H]1CNc2c(sc3ccc4nc(-c5ccc(C(=O)NC6CCN(c7ccc8c(C9CCC(=O)NC9=O)nn(C)c8c7)CC6)cc5F)ccc4c23)C(=O)N1]

(R)-3-fluoro-4-(10-methyl-8-oxo-9,10,11,12-tetrahydro-8H-[1,4]diazepino[5′,6′:4,5]thieno[3,2-f]quinolin-3-yl)benzoic acid. In a reaction tube, (R)-3-chloro-10-methyl-9,10,11,12-tetrahydro-8H-[1,4]diazepino[5′,6′:4,5]thieno[3,2-f]quinolin-8-one (2.0 g, 6.29 mmol) was first charged and followed by the addition of 4-borono-3-fluoro-benzoic acid (1.7 g, 9.44 mmol), Cs₂CO₃ (6.1 g, 18.9 mmol) and Pd(dppf)Cl₂ (0.51 g, 0.63 mmol). After the addition of 1,4-dioxane (45.0 mL) and water (15.0 mL) to the tube, the resulted mixture was degassed with N₂ for 20 min, capped and stirred at 90° C. for 18 h. After cooling to rt and filtering through a pad of celite and then washed by EtOAc (10 mL×2). The collected liquid was concentrated under vacuum, and the residue was purified by reverse-phase chromatography (RPC) using H₂O with 0.1% formic acid and acetonitrile as mobile phases to afford (R)-3-fluoro-4-(10-methyl-8-oxo-9,10,11,12-tetrahydro-8H-[1,4]diazepino[5′,6′:4,5]thieno[3,2-f]quinolin-3-yl)benzoic acid in a quantitative yield. MS (ESI, m/z) [M+H]⁺ 422.1.

N-(1-(3-(2,6-dioxopiperidin-3-yl)-1-methyl-1H-indazol-6-yl)piperidin-4-yl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide. To a round-bottom flask, 3-(1-methyl-6-(piperazin-1-yl)-1H-indazol-3-yl)piperidine-2,6-dione (121.5 mg, 0.36 mmol) was charged and followed by the addition of (R)-3-fluoro-4-(10-methyl-8-oxo-9,10,11,12-tetrahydro-8H-[1,4]diazepino[5′,6′:4,5]thieno[3,2-f]quinolin-3-yl)benzoic acid and DIPEA (0.52 mL, 2.97 mmol). After the addition of DMF (2.0 mL) to the flask, the reaction mixture was stirred at rt for 10 min. and then HATU (169.2 mg, 0.44 mmol) was added. The resulting mixture was stirred at rt for 1.0 h. LCMS indicated full conversion to the desired product. The reaction mixture was purified by a reverse-phase chromatography (RPC) using H₂O with 0.1% formic acid and acetonitrile as mobile phases to afford affording the desired product (30.1 mg, 13.4% yield) as a yellow solid. ¹H NMR (400 MHz, DMSO-d₆) δ ppm 1.20 (d, J=6.6 Hz, 3H), 1.70-1.82 (m, 2H), 1.91-1.99 (m, 2H), 2.13-2.21 (m, 1H), 2.25-2.36 (m, 1H), 2.55-2.65 (m, 2H), 2.93 (br, t, J=11.5 Hz, 2H), 3.45-3.52 (m, 2H), 3.58-3.66 (m, 1H), 3.86-3.94 (m, 5H), 4.02-4.12 (m, 1H), 4.26 (dd, J=9.2, 5.0 Hz, 1H), 6.89-6.93 (m, 1H), 6.93-6.99 (m, 1H), 7.18 (br t, J=5.0 Hz, 1H), 7.51 (d, J=9.0 Hz, 1H), 7.84-7.96 (m, 2H), 8.03-8.13 (m, 3H), 8.15-8.27 (m, 2H), 8.53 (d, J=7.6 Hz, 1H), 9.28 (d, J=8.8 Hz, 1H), 10.85 (s, 1H). MS (ESI, m/z) [M+H]⁺ 745.2.

### Example 2: Synthesis of N-(2-(4-(3-(2,6-dioxopiperidin-3-yl)-1-methyl-1H-indazol-6-yl)piperazin-1-yl)ethyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide

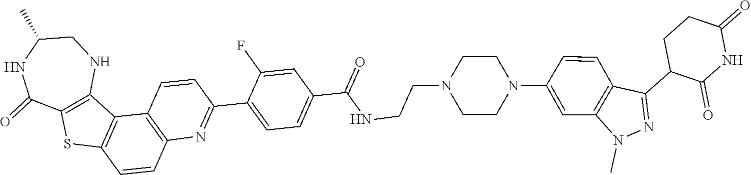 [SMILES: C[C@@H]1CNc2c(sc3ccc4nc(-c5ccc(C(=O)NCCN6CCN(c7ccc8c(C9CCC(=O)NC9=O)nn(C)c8c7)CC6)cc5F)ccc4c23)C(=O)N1]

tert-Butyl (2-(4-(3-(2,6-dioxopiperidin-3-yl)-1-methyl-1H-indazol-6-yl)piperazin-1-yl)ethyl)carbamate. To a round-bottomed flask, 3-(1-methyl-6-(piperazin-1-yl)-1H-indazol-3-yl)piperidine-2,6-dione dihydrochloride (0.2 g, 0.50 mmol) was charged and followed by the addition of tert-butyl (2-oxoethyl)carbamate (118.7 mg, 0.75 mmol) and DIPEA (0.17 mL, 0.99 mmol). After the addition of DCE (5.0 mL), the resulted mixture was stirred at 25° C. for 10 min., and then NaBH(OAc)₃ (189.6 mg, 0.89 mmol) was added. The reaction mixture was stirred overnight at rt for 16 hr. Additional NaBH(OAc)₃ (0.5 mmol) was added and stirred at rt for another 4.0 h. After evaporated the solvent, the residue was purified by reverse-phase chromatography (RPC) using H₂O with 0.1% formic acid and acetonitrile as mobile phases to afford the desired product as a white solid in a quantitative yield. MS (ESI+) [M+H]⁺ 471.2.

3-(6-(4-(2-Aminoethyl)piperazin-1-yl)-1-methyl-1H-indazol-3-yl)piperidine-2,6-dione; hydrochloride. In a round-bottom flask, tert-butyl N-[2-[4-[3-(2,6-dioxo-3-piperidyl)-1-methyl-indazol-6-yl]piperazin-1-yl]ethyl]carbamate (0.24 g mg, 0.51 mmol) and HCl (4.0 M) in dioxane (0.02 mL, 0.51 mmol) were added. The solution was stirred for 2.0 h at rt and LCMS spectra indicated complete conversion to the desired product. After the solvent was removed, the crude product was used to next step reaction without further purification. MS (ESI, m/z) [M+H]371.2.

N-(2-(4-(3-(2,6-Dioxopiperidin-3-yl)-1-methyl-1H-indazol-6-yl)piperazin-1-yl)ethyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide. In a round-bottom flask, 3-[6-[4-(2-aminoethyl)piperazin-1-yl]-1-methyl-indazol-3-yl]piperidine-2,6-dione; hydrochloride (0.58 g, 0.14 mmol) was charged, and followed by the addition of (R)-3-fluoro-4-(10-methyl-8-oxo-9,10,11,12-tetrahydro-8H-[1,4]diazepino[5′,6′:4,5]thieno[3,2-f]quinolin-3-yl)benzoic acid (0.05 g, 0.12 mmol) and DIPEA (0.21 mL, 1.19 mmol). After the addition of DMF (1.5 mL), the resulted mixture was stirred at rt for 10 min., and then HATU (0.68 g, 0.18 mmol) was added. The reaction was stirred at rt for 2.0 h, LCMS indicated a completed conversion to the desired product. The reaction mixture was purified by a reverse-phase chromatography (RPC) using H₂O with. 0.1% formic acid and acetonitrile as mobile phases to afford the desired product. ¹H NMR (400 MHz, DMSO-d₆) δ ppm 1.20 (d, J=6.8 Hz, 3H), 2.11-2.21 (m, 1H), 2.24-2.32 (m, 1H), 2.57-2.67 (m, 8H), 3.21-3.28 (m, 4H), 3.44-3.54 (m, 4H), 3.58-3.66 (m, 1H), 3.89 (s, 3H), 4.26 (dd, J=9.2, 5.0 Hz, 1H), 6.84-6.87 (m, 1H), 6.91-6.96 (m, 1H), 7.18 (br t, J=4.9 Hz, 1H), 7.50 (d, J=9.0 Hz, 1H), 7.82-7.93 (m, 2H), 8.02-8.13 (m, 3H), 8.15-8.27 (m, 2H), 8.69 (t, J=5.6 Hz, 1H), 9.28 (d, J=9.0 Hz, 1H), 10.85 (s, 1H). MS (ESI, m/z) [M+H]⁺ 774.2.

### Example 3: Synthesis of N-((trans)-3-((4-(3-(2,6-Dioxopiperidin-3-yl)-1-methyl-1H-indazol-6-yl)piperazin-1-yl)methyl)cyclobutyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide

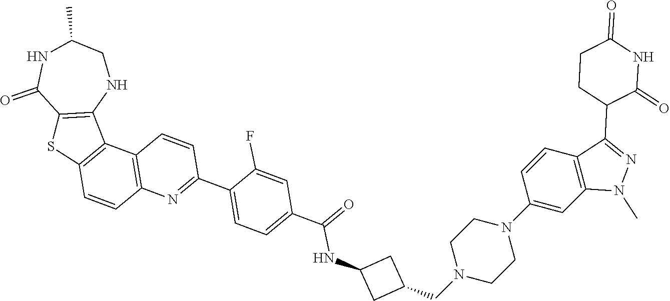 [SMILES: C[C@@H]1CNc2c(sc3ccc4nc(-c5ccc(C(=O)N[C@H]6C[C@H](CN7CCN(c8ccc9c(C%10CCC(=O)NC%10=O)nn(C)c9c8)CC7)C6)cc5F)ccc4c23)C(=O)N1]

tert-Butyl ((trans)-3-formylcyclobutyl)carbamate A solution of tert-butyl N-[3-(hydroxymethyl)cyclobutyl]carbamate (0.3 g, 1.49 mmol) and PCC (0.64 g, 2.98 mmol) in DCM (15.0 mL) was stirred at rt for 4 h. The mixture was filtered through a celite pad and washed the pad with DCM (10 mL×2). After evaporation of the solvent, the crude product was purified by normal-phase chromatography with EtOAc and heptane as mobile phases to afford the desired product as white solid (201.7 mg, 68% yield).

tert-Butyl ((trans)-3-((4-(3-(2,6-dioxopiperidin-3-yl)-1-methyl-1H-indazol-6-yl)piperazin-1-yl)methyl)cyclobutyl)carbamate. A suspension of 3-(1-methyl-6-piperazin-1-yl-indazol-3-yl)piperidine-2,6-dione; hydrochloride (0.30 g, 0.85 mmol), tert-butyl N-(3-formylcyclobutyl)carbamate (0.15 g 0.77 mmol), DIPEA (0.13 mL, 0.77 mmol) in DCM (8.0 mL mL) was sonicated for 5-10 min, and additional DMSO (1.0 mL) was added to the mixture. After 5 min, NaBH(OAc)₃ (0.41 g, 1.93 mmol) was added and the reaction was stirred at rt overnight. After concentrating under vacuum, the residue was purified by a reverse-phase chromatography (RPC) using H₂O with 0.1% formic acid and acetonitrile as mobile phases to afford the desired product (366.7 mg, yield=92%). MS (ESI, m/z) [M+H]⁺ 511.4.

3-(6-(4-(((trans)-3-aminocyclobutyl)methyl)piperazin-1-yl)-1-methyl-1H-indazol-3-yl)piperidine-2,6-dione. To a 100 mL of round-bottom flask are introduced tert-butyl N-[3-[[4-[3-(2,6-dioxo-3-piperidyl)-1-methyl-indazol-6-yl]piperazin-1-yl]methyl]cyclobutyl]carbamate (366.7 mg, 0.72 mmol) and HCl (4.0 N) in 1,4-dioxane (3.6 mL, 14.36 mmol), then the solution was stirred at rt for 2.0 h. The reaction mixture was diluted with MTBE. After filtration, the precipitates were recovered, washed by MTBE (5.0 mL×2), and dried under vacuum to afford the desired product (406 mg) as white solid. MS (ESI, m/z) [M+H]⁺ 411.2.

N-((trans)-3-((4-(3-(2,6-Dioxopiperidin-3-yl)-1-methyl-1H-indazol-6-yl)piperazin-1-yl)methyl)cyclobutyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide. To a solution of 3-[6-[4-[(3-aminocyclobutyl)methyl]piperazin-1-yl]-1-methyl-indazol-3-yl]piperidine-2,6-dione; dihydrochloride (0.21 g, 0.43 mmol) in DMF (2.0 mL), (R)-3-fluoro-4-(10-methyl-8-oxo-9,10,11,12-tetrahydro-8H-[1,4]diazepino[5′,6′:4,5]thieno[3,2-f]quinolin-3-yl)benzoic acid (0.15 g, 0.36 mmol) and DIPEA (0.62 mL, 3.56 mmol) were charged, and then sonicated and stirred for 5 min. After the addition of HATU (135.3 mg, 0.36 mmol), the reaction mixture was stirred at rt for 2.0 h. After concentrating under vacuum, the residue was purified by a reverse-phase chromatography (RPC) using H₂O with 0.1% formic acid and acetonitrile as mobile phases to afford the desired product (51 mg, 18% yield) as a yellow solid. ¹H NMR (400 MHz, DMSO-d₆) δ ppm 1.20 (d, J=6.8 Hz, 3H), 2.07-2.34 (m, 6H), 2.52-2.69 (m, 9H), 3.21-3.25 (m, 4H), 3.46-3.51 (m, 2H), 3.59-3.67 (m, 1H), 3.89 (s, 3H), 4.26 (dd, J=9.2, 5.0 Hz, 1H), 4.53 (q, J=7.8 Hz, 1H), 6.85 (d, J=1.5 Hz, 1H), 6.93 (dd, J=9.0, 1.7 Hz, 1H), 7.18 (t, J=5.1 Hz, 1H), 7.50 (d, J=9.0 Hz, 1H), 7.85-7.95 (m, 2H), 8.01-8.28 (m, 6H), 8.88 (d, J=7.1 Hz, 1H), 9.29 (d, J=9.0 Hz, 1H), 10.85 (s, 1H). MS (ESI, m/z) [M+H]⁺ 814.3.

### Example 4: Synthesis of N-(trans-3-(2-(4-(3-(2,6-Dioxopiperidin-3-yl)-1-methyl-1H-indazol-6-yl)piperazin-1-yl)ethyl)cyclobutyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide

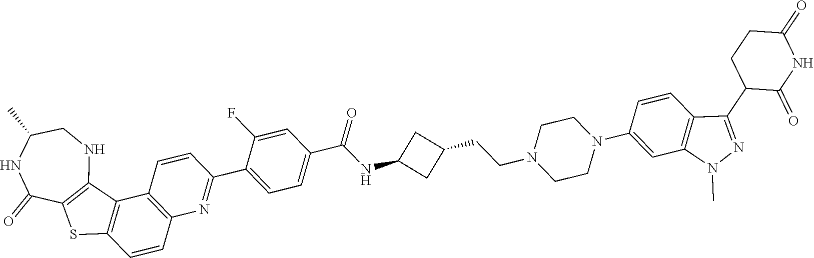 [SMILES: C[C@@H]1CNc2c(sc3ccc4nc(-c5ccc(C(=O)N[C@H]6C[C@H](CCN7CCN(c8ccc9c(C%10CCC(=O)NC%10=O)nn(C)c9c8)CC7)C6)cc5F)ccc4c23)C(=O)N1]

### tert-Butyl ((trans)-3-(2-(methoxy(methyl)amino)-2-oxoethyl)cyclobutyl)carbamate

In a solution of 2-[3-(tert-butoxycarbonylamino)cyclobutyl]acetic acid (250.0 mg, 1.09 mmol) in DCM (5.0 mL), N-methoxymethanamine hydrochloride (159.5 mg, 1.64 mmol) and DIPEA (0.95 mL, 5.45 mmol) were added. After the addition of HATU (621.9 mg, 1.64 mmol), the reaction mixture was stirred at rt overnight. The reaction mixture was poured into 3.0 mL of NaOH (1.0 M, aq) and stirred for 10 min. The organic layer was separated and was washed with HCl (2.0 mL, 1.0 M), water (2.0 mL×1), brine (2.0 mL×1) and dried over Na₂SO₄. After filtering and concentrated, the crude product was purified by chromatography using heptane and EtOAc and mobile phase to afford the desired product (283 mg, 95% yield) as a white solid.

tert-Butyl ((trans)-3-(2-oxoethyl)cyclobutyl)carbamate. Red-Al®60% in toluene (0.44 mL, 1.34 mmol) was added dropwise to a solution of tert-butyl (3-(2-(methoxy(methyl)amino]-2-oxo-ethyl)cyclobutyl)carbamate (280.7 mg, 1.03 mmol) in THF (5.0 mL) at −40° C. After stirring for 30 min at −40° C., the reaction mixture was continuously stirred overnight at rt. The reaction was quenched by adding EtOAc (4.0 mL) and Rochelle salt (4.0 mL, aq) and vigorously stirring for 1.0 hr. The organic layer was separated and was washed with brine (4.0 mL×1), water (4.0 mL×1), dried over Na₂SO₄. After filtering and concentrating, a colorless oil (259.7 mg) was obtained and used for the next step without further purification.

tert-butyl (3-(2-(4-(3-(2,6-dioxopiperidin-3-yl)-1-methyl-1H-indazol-6-yl)piperazin-1-yl)ethyl)cyclobutyl)carbamate. To a suspension of 3-(1-methyl-6-piperazin-1-yl-indazol-3-yl)piperidine-2,6-dione hydrochloride (329.2 mg, 0.90 mmol) in DCM (9.0 mL), tert-butyl N-[3-(2-oxoethyl)cyclobutyl]carbamate (193.0 mg, 0.90 mmol) and DIPEA (1.1 mL, 6.33 mmol) were added and then sonicated for 3 min. To the mixture, DMSO (1.0 mL) and NaBH(OAc)₃ (479.5 mg, 2.26 mmol) was charged, and the resulting mixture was stirred at rt overnight. After concentration under vacuum, the residue was purified by a reverse-phase chromatography (RPC) using H₂O with 0.1% formic acid and acetonitrile as mobile phases to afford the desired product (293.0 mg, 60% yield) as a tan foam.

3-(6-(4-(2-((trans)-3-aminocyclobutyl)ethyl)piperazin-1-yl)-1-methyl-1H-indazol-3-yl)piperidine-2,6-dione. In a round bottom flask, tert-butyl (3-(2-(methoxy(methyl)amino]-2-oxo-ethyl)cyclobutyl)carbamate (293.0 mg, 0.56 mmol) and 4.0 M HCl in 1,4-dioxane (0.41 mL, 11.17 mmol) were added. The resulting solution was stirred at rt for 2.0 h. To the reaction mixture, MTBE (5.0 mL) was added and the resulting precipitate was collected by filtration, affording the desired product (302.6 mg) as a white solid.

N-(trans-3-(2-(4-(3-(2,6-Dioxopiperidin-3-yl)-1-methyl-1H-indazol-6-yl)piperazin-1-yl)ethyl)cyclobutyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide To a solution of 3-[6-[4-[2-(3-aminocyclobutyl)ethyl]piperazin-1-yl]-1-methyl-indazol-3-yl]piperidine-2,6-dione dihydrochloride (177.1 mg, 0.36 mmol) in DMF (3.0 mL), (R)-3-fluoro-4-(10-methyl-8-oxo-9,10,11,12-tetrahydro-8H-[1,4]diazepino[5′,6′:4,5]thieno[3,2-f]quinolin-3-yl)benzoic acid (125.0 mg, 0.30 mmol) and DIPEA (0.52 mL, 2.97 mmol) in DMF was charged. After stirred for 5 min, HATU (112.8 mg, 0.30 mmol) was added and the resulting mixture was stirred at rt overnight. After the removal of solvent, the residue was purified by reverse-phase chromatography (RPC) using H₂O with 0.1% formic acid and acetonitrile as mobile phases to afford the desired product (18.6 mg, 7.5% yield) as a yellow solid. MS (ESI, m/z) [M+H]⁺ 822.4 ¹H NMR (400 MHz, DMSO-d₆) δ ppm 1.20 (d, J=6.6 Hz, 3H), 1.68-1.77 (m, 2H), 2.02-2.10 (m, 2H), 2.12-2.20 (m, 1H), 2.23-2.29 (m, 3H), 2.30-2.36 (m, 3H), 2.55-2.64 (m, 6H), 3.20-3.26 (m, 4H), 3.46-3.50 (m, 2H), 3.57-3.67 (m, 1H), 3.90 (s, 3H), 4.26 (dd, J=9.2, 5.0 Hz, 1H), 4.48-4.60 (m, 1H), 6.83-6.87 (m, 1H), 6.90 (dd, J=9.3, 1.0 Hz, 1H), 7.15-7.21 (m, 1H), 7.48-7.53 (m, 1H), 7.85-7.94 (m, 2H), 8.03-8.12 (m, 3H), 8.14 (s, 1H), 8.16-8.25 (m, 2H), 8.86 (d, J=7.1 Hz, 1H), 9.28 (d, J=8.8 Hz, 1H), 10.85 (s, 1H).

### Example 5: Synthesis of N-(trans-4-((4-(3-(2,6-dioxopiperidin-3-yl)-1-methyl-1H-indazol-6-yl)piperazin-1-yl)methyl)cyclohexyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide

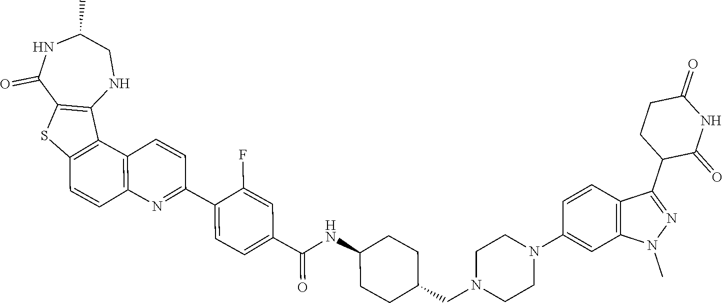 [SMILES: C[C@@H]1CNc2c(sc3ccc4nc(-c5ccc(C(=O)N[C@H]6CC[C@H](CN7CCN(c8ccc9c(C%10CCC(=O)NC%10=O)nn(C)c9c8)CC7)CC6)cc5F)ccc4c23)C(=O)N1]

tert-Butyl ((1R,4R)-4-((4-(3-(2,6-dioxopiperidin-3-yl)-1-methyl-1H-indazol-6-yl)piperazin-1-yl)methyl)cyclohexyl)carbamate. To a solution of 3-(1-methyl-6-piperazin-1-yl-indazol-3-yl)piperidine-2,6-dione;hydrochloride (660.0 mg, 1.81 mmol) in DCM (10.0 mL), DIPEA (1.44 mL, 8.25 mmol) and tert-butyl N-(4-formylcyclohexyl)carbamate (374.8 mg, 1.65 mmol) were charged. After the addition of NaBH(OAc)₃ (873.76 mg, 4.12 mmol), the resulting mixture was stirred at rt overnight. The solvent was evaporated under vacuum and the crude product was purified by a reverse-phase chromatography (RPC) using H₂O with 0.1% formic acid and acetonitrile as mobile phases to afford the desired product (735 mg, 76% yield) as an off-white solid. MS(ESI, m/z) [M−HCOOH+H]⁺ 539.4. ¹H NMR (400 MHz, DMSO-d6) δ ppm 0.82-0.98 (m, 2H), 1.06-1.22 (m, 2H), 1.37 (s, 9H), 1.42-1.50 (m, 1H), 1.78 (br d, J=10.5 Hz, 4H), 2.11-2.22 (m, 3H), 2.24-2.36 (m, 1H), 2.52-2.69 (m, 5H), 3.22 (br s, 6H), 3.89 (s, 3H), 4.25 (dd, J=9.2, 5.0 Hz, 1H), 6.70 (br d, J=7.6 Hz, 1H), 6.83 (d, J=1.5 Hz, 1H), 6.91 (dd, J=9.0, 2.0 Hz, 1H), 7.49 (d, J=9.0 Hz, 1H), 8.13 (s, 1H), 10.84 (s, 1H).

3-(6-(4-((4-Aminocyclohexyl)methyl)piperazin-1-yl)-1-methyl-1H-indazol-3-yl)piperidine-2,6-dione. To a solution of tert-butyl N-[4-[[4-[3-(2,6-dioxo-3-piperidyl)-1-methyl-indazol-6-yl]piperazin-1-yl]methyl]cyclohexyl]carbamate (735.0 mg, 1.36 mmol) in DCM (5.0 mL), a solution of 4.0 M HCl in 1,4 dioxane (5.0 mL, 20.5 mmol) was added and the resulting mixture was stirred at rt overnight. After evaporated the solvent under vacuum, the residue was further azeotropically dried with toluene (5.0 mL×2) to afford (700 mg, quantitative yield) as an off-white solid. MS (ESI, m/z) [M+H]⁺ 439.2. ¹H NMR (400 MHz, DMSO-d₆) δ ppm 1.01-1.15 (m, 2H), 1.37 (br d, J=10.5 Hz, 2H), 1.81 (br d, J=2.7 Hz, 1H), 1.97 (br d, J=11.0 Hz, 4H), 2.12-2.21 (m, 1H), 2.31 (dt, J=13.8, 4.5 Hz, 1H), 2.55-2.69 (m, 2H), 2.88-3.04 (m, 3H), 3.09-3.22 (m, 2H), 3.38 (br t, J=12.1 Hz, 2H), 3.60 (br s, 1H), 3.64-3.73 (m, 1H), 3.87 (br d, J=13.0 Hz, 2H), 3.92 (s, 3H), 4.28 (br dd, J=9.4, 5.0 Hz, 1H), 6.93-7.00 (m, 2H), 7.52-7.60 (m, 1H), 8.09 (br s, 3H), 8.14 (s, 1H), 10.68 (br d, J=1.5 Hz, 1H), 10.86 (s, 1H).

N-(trans-4-((4-(3-(2,6-dioxopiperidin-3-yl)-1-methyl-1H-indazol-6-yl)piperazin-1-yl)methyl)cyclohexyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide. To a solution of (R)-3-fluoro-4-(10-methyl-8-oxo-9,10,11,12-tetrahydro-8H-[1,4]diazepino[5′,6′:4,5]thieno[3,2-f]quinolin-3-yl)benzoic acid (145.0 mg, 0.34 mmol) in DMF (3.0 mL), DIPEA (0.42 mL, 2.41 mmol) was charged. After 10 min, HATU (196.2 mg, 0.52 mmol) was added, and then the resulting mixture was stirred at rt for 2.0 h. The mixture was purified by reverse-phase chromatography (RPC) using H₂O with 0.1% formic acid and acetonitrile as mobile phases to afford the desired product (75.0 mg, 52.4% yield) as a yellow solid. MS (ESI, m/z) [M+H]⁺ 842.5 ¹H NMR (400 MHz, DMSO-d₆) δ ppm 0.95-1.08 (m, 2H), 1.20 (d, J=6.6 Hz, 4H), 1.33-1.48 (m, 2H), 1.50-1.61 (m, 1H), 1.83-1.99 (m, 4H), 2.11-2.23 (m, 3H), 2.25-2.33 (m, 1H), 2.52-2.65 (m, 5H), 3.23 (br s, 4H), 3.45-3.55 (m, 2H), 3.62 (td, J=7.0, 3.9 Hz, 1H), 3.73-3.85 (m, 1H), 3.89 (s, 3H), 4.26 (dd, J=9.2, 5.3 Hz, 1H), 6.85 (br s, 1H), 6.93 (br d, J=8.8 Hz, 1H), 7.18 (br t, J=5.1 Hz, 1H), 7.50 (d, J=8.8 Hz, 1H), 7.82-7.95 (m, 2H), 8.02-8.12 (m, 3H), 8.17-8.25 (m, 2H), 8.39-8.51 (m, 1H), 9.28 (d, J=8.8 Hz, 1H), 10.85 (s, 1H).

### Example 6: Synthesis of N-(trans-4-(2-(4-(3-(2,6-Dioxopiperidin-3-yl)-1-methyl-1H-indazol-6-yl)piperazin-1-yl)ethyl)cyclohexyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide

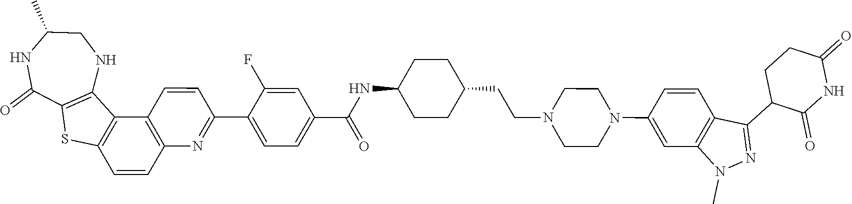 [SMILES: C[C@@H]1CNc2c(sc3ccc4nc(-c5ccc(C(=O)N[C@H]6CC[C@H](CCN7CCN(c8ccc9c(C%10CCC(=O)NC%10=O)nn(C)c9c8)CC7)CC6)cc5F)ccc4c23)C(=O)N1]

tert-Butyl ((1r,4r)-4-(2-(4-(3-(2,6-dioxopiperidin-3-yl)-1-methyl-1H-indazol-6-yl)piperazin-1-yl)ethyl)cyclohexyl)carbamate. To a round bottom flask with 3-(1-methyl-6-piperazin-1-yl-indazol-3-yl)piperidine-2,6-dione dihydrochloride (150.0 mg, 0.37 mmol) in DMSO (1.5 mL), tert-butyl N-[4-(2-oxoethyl)cyclohexyl]carbamate (90.4 mg, 0.37 mmol) and DIPEA (0.65 mL, 3.75 mmol) were charged. After stirring the mixture for 10 min at rt, NaBH(OAc)₃ (238.3 mg, 1.12 mmol) was added. The resulting mixture was continuously stirred overnight at rt. The DCM was evaporated under vacuum and the residue was purified by reverse-phase chromatography (RPC) using H₂O with 0.1% formic acid and acetonitrile as mobile phases to afford the desired product (130.0 mg, 63% yield). MS (ESI, m/z) [M+H]⁺ 553.2.

3-(6-(4-(2-((1r,4r)-4-aminocyclohexyl)ethyl)piperazin-1-yl)-1-methyl-1H-indazol-3-yl)piperidine-2,6-dione. To a round bottom flask charged with tert-butyl N-[4-[2-[4-[3-(2,6-dioxo-3-piperidyl)-1-methyl-indazol-6-yl]piperazin-1-yl]ethyl]cyclohexyl]carbamate (130.0 mg, 0.24 mmol), a solution of 4.0 M HCl in dioxane (0.06 mL, 0.24 mmol) was added. The reaction mixture was stirred for 2.0 h at rt. After evaporated the solvent under vacuum, the residue was further azeotropically dried with toluene (2.0 mL×2) to afford desired product as a white solid (120 mg, 93% yield) as a white solid. MS (ESI, m/z) [M+H]⁺ 453.2.

N-(trans-4-(2-(4-(3-(2,6-Dioxopiperidin-3-yl)-1-methyl-1H-indazol-6-yl)piperazin-1-yl)ethyl)cyclohexyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide. To a round bottom flask with 3-[6-[4-[2-(4-aminocyclohexyl)ethyl]piperazin-1-yl]-1-methyl-indazol-3-yl]piperidine-2,6-dione dihydrochloride (104.7 mg, 0.20 mmol) in DMF (2.0 mL), (R)-3-fluoro-4-(10-methyl-8-oxo-9,10,11,12-tetrahydro-8H-[1,4]diazepino[5′,6′:4,5]thieno[3,2-f]quinolin-3-yl)benzoic acid (70.0 mg, 0.17 mmol) and DIPEA (0.29 mL, 1.66 mmol) were added. After the mixture was stirred at rt for 10 min, HATU (94.7 mg, 0.25 mmol) was added. The reaction mixture was continuously stirred for 2.0 h at rt. The residue was purified by reverse-phase chromatography (RPC) using H₂O with 0.1% formic acid and acetonitrile as mobile phases to afford the desired product (25.9 mg, 17.8% yield) as a yellow solid. MS (ESI, m/z) [M+H]⁺ 856.3 ¹H NMR (400 MHz, DMSO-d₆) δ ppm 1.01-1.13 (m, 2H), 1.20 (d, J=6.8 Hz, 3H), 1.24-1.46 (m, 5H), 1.82 (br d, J=11.7 Hz, 2H), 1.90 (br d, J=9.4 Hz, 2H), 2.11-2.21 (m, 1H), 2.25-2.32 (m, 1H), 2.39 (br t, J=7.3 Hz, 2H), 2.52-2.57 (m, 4H), 2.58-2.65 (m, 2H), 3.19-3.26 (m, 4H), 3.45-3.51 (m, 2H), 3.57-3.67 (m, 1H), 3.73-3.83 (m, 1H), 3.89 (s, 3H), 4.26 (dd, J=9.2, 5.0 Hz, 1H), 6.83-6.86 (m, 1H), 6.92 (dd, J=9.0, 1.7 Hz, 1H), 7.18 (br t, J=4.9 Hz, 1H), 7.50 (d, J=8.8 Hz, 1H), 7.82-7.92 (m, 2H), 8.03-8.13 (m, 3H), 8.16-8.26 (m, 2H), 8.42 (d, J=7.8 Hz, 1H), 9.28 (d, J=8.8 Hz, 1H), 10.85 (s, 1H).

### Example 7: Synthesis of N-(1-(4-(2,6-Dioxopiperidin-3-yl)phenyl)piperidin-4-yl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide

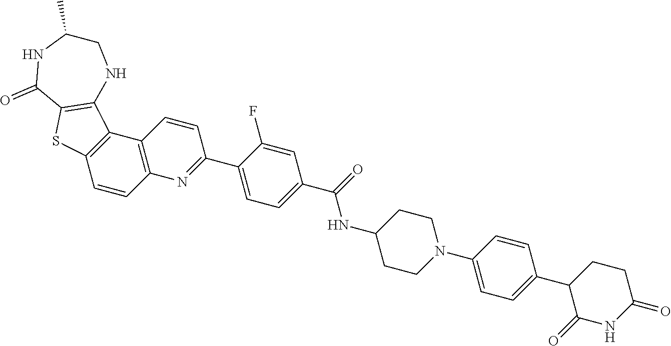 [SMILES: C[C@@H]1CNc2c(sc3ccc4nc(-c5ccc(C(=O)NC6CCN(c7ccc(C8CCC(=O)NC8=O)cc7)CC6)cc5F)ccc4c23)C(=O)N1]

tert-Butyl (1,5-dioxopentan-3-yl)carbamate. To a solution of tert-butyl N-(3,4-dihydroxycyclopentyl)carbamate (150.4 mg, 0.69 mmol) in THF (2.8 mL)/water (1.4 mL) at rt was added NaIO₄ (177.8 mg, 0.83 mmol). The resulting mixture was stirred at rt for 1 h. THF was removed under vacuum, and brine (5 mL) was added. The resulting aqueous solution was extracted with EtOAc (10 mL×3). The combined organic layers were washed with brine, dried over MgSO₄, filtered, and concentrated. The residue was dissolved in dichloromethane (10 mL), and MgSO₄ (3 g) was added. The mixture was stirred at rt over weekend. The mixture was filtered and concentrated to afford the product, tert-butyl (1,5-dioxopentan-3-yl)carbamate (156.6 mg, quantitative yield) as a yellow solid which was used without further purification.

tert-Butyl (1-(4-(2,6-dioxopiperidin-3-yl)phenyl)piperidin-4-yl)carbamate. To a solution of 3-(4-aminophenyl)piperidine-2,6-dione (159.0 mg, 0.78 mmol), which can be prepared according to methods described in WO2022012622 A1, and is hereby incorporated by reference in its entirety, and tert-butyl N-[3-tert-butyl (1,5-dioxopentan-3-yl)carbamate (202 mg, 0.94 mmol) in DCE (5 mL) at rt was added NaBH(OAc)₃ (396.3 mg, 1.87 mmol). The resulting solution was stirred for an hour. LCMS analysis showed incomplete conversion. More NaBH(OAc)₃ (176.9 mg, 0.83 mmol) was added, and the reaction was stirred for another 1.0 h. LCMS analysis still showed incomplete conversion. More NaBH(OAc)₃ (186.6 mg, 0.88 mmol) was added and the reaction was stirred for another 2 h. The reaction was quenched with saturated NH₄Cl (5.0 mL) and the mixture was extracted with dichloromethane (10 mL). The organic phases were separated, and the aqueous layer was further extracted with dichloromethane (10 mL×3). The combined organic layers were washed with brine, dried over MgSO₄, filtered, and concentrated. The residue was purified by reverse-phase chromatography (RPC) using H₂O with 0.1% formic acid and acetonitrile as mobile phases to afford the desired product, tert-Butyl (1-(4-(2,6-dioxopiperidin-3-yl)phenyl)piperidin-4-yl)carbamate (203 mg, 67% yield) as a tan solid. MS (ESI, m/z) [M+H]⁺ 388.4.

3-(4-(4-Aminopiperidin-1-yl)phenyl)piperidine-2,6-dione. tert-Butyl N-[1-[4-(2,6-dioxo-3-piperidyl)phenyl]-4-piperidyl]carbamate (201 mg, 0.52 mmol) was suspended in 4.0 M HCl in dioxane (5 mL, 20 mmol), the resulting mixture was stirred at rt for 2 h. The mixture was then concentrated under vacuum to afford the product, 3-(4-(4-aminopiperidin-1-yl)phenyl)piperidine-2,6-dione;trihydrochloride (215.8 mg, quant.) as a tan solid. MS (ESI, m/z) [M+H]⁺ 288.2.

N-(1-(4-(2,6-Dioxopiperidin-3-yl)phenyl)piperidin-4-yl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide. To a suspension of (R)-3-fluoro-4-(10-methyl-8-oxo-9,10,11,12-tetrahydro-8H-[1,4]diazepino[5′,6′:4,5]thieno[3,2-f]quinolin-3-yl)benzoic acid. (50 mg, 0.12 mmol) and 3-(4-(4-aminopiperidin-1-yl)phenyl)piperidine-2,6-dione trihydrochloride (51.6 mg, 0.13 mmol) in DMF (1 mL) at rt was added DIPEA (150 μL, 0.86 mmol). The resulting mixture was stirred for 7 min, then pyAOP (74 mg, 0.14 mmol) was added in one portion. The resulting mixture was stirred at rt for 1.0 h. After removed the solvent under vacuum, the residue was purified by reverse-phase chromatography (RPC) using H₂O with 0.1% formic acid and acetonitrile as mobile phases to afford the desired product, N-(1-(4-(2,6-dioxopiperidin-3-yl)phenyl)piperidin-4-yl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide (6.9 mg, 8% yield) as a yellow solid. MS (ESI, m/z) [M+H]⁺ 691.3 ¹H NMR (400 MHz, DMSO-d₆) δ ppm 10.78 (s, 1H), 9.28 (br d, J=8.8 Hz, 1H), 8.52 (br d, J=7.8 Hz, 1H), 8.15-8.29 (m, 2H), 8.01-8.14 (m, 3H), 7.84-7.95 (m, 2H), 7.14-7.23 (m, 1H), 7.06 (br d, J=8.6 Hz, 2H), 6.94 (br d, J=8.8 Hz, 2H), 3.95-4.10 (m, 1H), 3.69-3.84 (m, 3H), 3.58-3.67 (m, 1H), 3.45-3.52 (m, 2H), 2.80-2.89 (m, 2H), 2.09-2.20 (m, 1H), 1.98-2.05 (m, 1H), 1.85-1.94 (m, 2H), 1.64-1.77 (m, 2H), 1.23 (br s, 1H), 1.20 (br d, J=6.6 Hz, 3H), 1.15 (s, 1H).

### Example 8: Synthesis of N-(2-(4-(4-(2,6-Dioxopiperidin-3-yl)phenyl)piperazin-1-yl)ethyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide

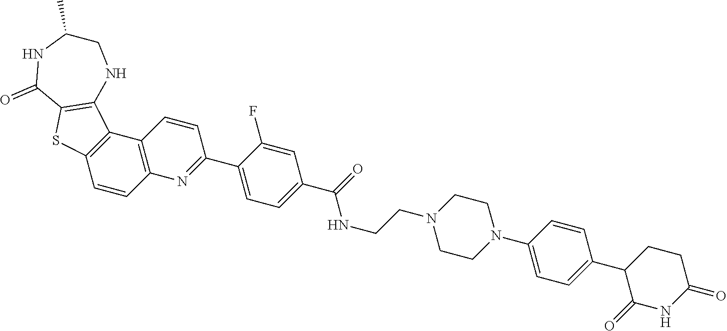 [SMILES: C[C@@H]1CNc2c(sc3ccc4nc(-c5ccc(C(=O)NCCN6CCN(c7ccc(C8CCC(=O)NC8=O)cc7)CC6)cc5F)ccc4c23)C(=O)N1]

tert-Butyl (2-(4-(4-(2,6-dioxopiperidin-3-yl)phenyl)piperazin-1-yl)ethyl)carbamate. To a solution of 3-(4-piperazin-1-ylphenyl)piperidine-2,6-dione;dihydrochloride (250 mg, 0.72 mmol) in DCM (5 mL) were added DIPEA (0.63 mL, 3.61 mmol) followed by tert-butyl N-(2-oxoethyl)carbamate (149 mg, 0.94 mmol) and NaBH(OAc)₃ (382 mg, 1.81 mmol) at rt. The reaction was then stirred at rt for 2 h. After the solvent was evaporated under vacuum, the residue was purified by reverse-phase chromatography (RPC) using H₂O with 0.1% formic acid and acetonitrile as mobile phases to afford the desired product, tert-butyl (2-(4-(4-(2,6-dioxopiperidin-3-yl)phenyl)piperazin-1-yl)ethyl)carbamate (190 mg, 63% yield), as an off-white solid. MS (ESI, m/z) [M+H]⁺ 417.4.

3-(4-(4-(2-Aminoethyl)piperazin-1-yl)phenyl)piperidine-2,6-dione. To a solution of tert-butyl (2-(4-(4-(2,6-dioxopiperidin-3-yl)phenyl)piperazin-1-yl)ethyl)carbamate (190 mg, 0.46 mmol) in DCM (3 mL) was added HCl (3 mL, 11.4 mmol) at rt. After 1.5 h, LCMS showed full conversion. The solvent was evaporated under vacuum, co-evaporated with MeCN (4×), DCM (2×) to afford the product, 3-(4-(4-(2-aminoethyl)piperazin-1-yl)phenyl)piperidine-2,6-dione dihydrochloride (170 mg, 96% yield) as an off-white solid. MS (ESI, m/z) [M+H]⁺ 317.2

N-(2-(4-(4-(2,6-Dioxopiperidin-3-yl)phenyl)piperazin-1-yl)ethyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide. To a solution of 3-[4-[4-(2-aminoethyl)piperazin-1-yl]phenyl]piperidine-2,6-dione dihydrochloride (167 mg, 0.4300 mmol) and (R)-3-fluoro-4-(10-methyl-8-oxo-9,10,11,12-tetrahydro-8H-[1,4]diazepino[5′,6′:4,5]thieno[3,2-f]quinolin-3-yl)benzoic acid (165 mg, 0.39 mmol) in DMF (2 mL) was added DIPEA (0.48 mL, 2.74 mmol) at rt. After 10 min, HATU (223.3 mg, 0.59 mmol) was added, and the reaction mixture was stirred at rt for 2 h. LCMS showed full conversion. The mixture was stirred at rt overnight. After the solvent was evaporated under vacuum, the residue was purified by reverse-phase chromatography (RPC) using H₂O with 0.1% formic acid and acetonitrile as mobile phases to afford the desired product, N-(2-(4-(4-(2,6-dioxopiperidin-3-yl)phenyl)piperazin-1-yl)ethyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide (53 mg, 19% yield) as a yellow solid. MS (ESI, m/z) [M+H]⁺ 720.3 ¹H NMR (400 MHz, DMSO-d₆) δ ppm 10.77 (s, 1H), 9.28 (d, J=8.8 Hz, 1H), 8.69 (t, J=5.7 Hz, 1H), 8.17-8.27 (m, 2H), 8.02-8.13 (m, 3H), 7.81-7.93 (m, 2H), 7.18 (br t, J=5.1 Hz, 1H), 7.05 (d, J=8.8 Hz, 2H), 6.90 (d, J=8.8 Hz, 2H), 3.73 (dd, J=11.0, 4.9 Hz, 1H), 3.62 (m, 1H), 3.43-3.52 (m, 4H), 3.11-3.20 (m, 4H), 2.54-2.65 (m, 7H), 2.44 (br d, J=4.4 Hz, 1H), 2.08-2.20 (m, 1H), 1.96-2.05 (m, 1H), 1.20 (d, J=6.6 Hz, 3H).

### Example 9: Synthesis of N-(trans-4-((4-(4-(2,6-Dioxopiperidin-3-yl)phenyl)piperazin-1-yl)methyl)cyclohexyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide

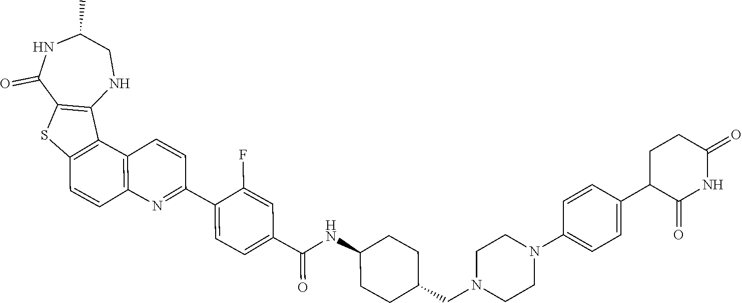 [SMILES: C[C@@H]1CNc2c(sc3ccc4nc(-c5ccc(C(=O)N[C@H]6CC[C@H](CN7CCN(c8ccc(C9CCC(=O)NC9=O)cc8)CC7)CC6)cc5F)ccc4c23)C(=O)N1]

tert-Butyl-N-[4-[[4-[4-(2,6-dioxo-3-piperidyl)phenyl]piperazin-1-yl]methyl]cyclohexyl]carbamate. To a stirred solution of 3-(4-piperazin-1-ylphenyl)piperidine-2,6-dione (200 mg, 0.73 mmol) and tert-butyl N-(4-formylcyclohexyl)carbamate (249 mg, 1.1 mmol) and TEA (0.2 mL, 1.15 mmol) and NaBH₃CN (92 mg, 1.44 mmol) was added MeOH (2 mL). The mixture was stirred at rt for 3 h. LC/MS showed full conversion to the desired product. The reaction was then quenched with water (30 mL) and extracted with EtOAc (30 mL×3). The combined organic layer was dried over Na₂SO₄, filtered, and concentrated. The residue was purified by silica gel column chromatography and eluted by PE/EtOAc (0-70%) to afford tert-butyl N-[4-[[4-[4-(2,6-dioxo-3-piperidyl)phenyl]piperazin-1-yl]methyl]cyclohexyl]carbamate (240 mg, 68% yield) as a white solid. MS (ESI, m/z) [M+H]⁺ 485.4.

3-[4-[4-[(4-Aminocyclohexyl)methyl]piperazin-1-yl]phenyl]piperidine-2,6-dione. To a stirred solution of tert-butyl N-[4-[[4-[4-(2,6-dioxo-3-piperidyl)phenyl]piperazin-1-yl]methyl]cyclohexyl]carbamate (240 mg, 0.50 mmol) was added HCl in EtOAc (10 mL, 0.50 M). The mixture was stirred at rt for 1 h. LC/MS showed full conversion to the expected product. After filtration, the crude product, 3-[4-[4-[(4-aminocyclohexyl)methyl]piperazin-1-yl]phenyl]piperidine-2,6-dione (180 mg) was obtained as a white solid. MS (ESI, m/z) [M+H]⁺ 385.1.

N-(trans-4-((4-(4-(2,6-Dioxopiperidin-3-yl)phenyl)piperazin-1-yl)methyl)cyclohexyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide. To a round-bottom flask was added 3-[4-[4-[(4-aminocyclohexyl)methyl]piperazin-1-yl]phenyl]piperidine-2,6-dione dihydrochloride (65 mg, 0.14 mmol) and (R)-3-fluoro-4-(10-methyl-8-oxo-9,10,11,12-tetrahydro-8H-[1,4]diazepino[5′,6′:4,5]thieno[3,2-f]quinolin-3-yl)benzoic acid (50 mg, 0.12 mmol) in DMF (1.2 mL). The reaction mixture was stirred at rt for 10 min and then HATU (45 mg, 0.12 mmol) was added. The reaction mixture was continuously stirred at rt for 1 h and then was directly purified by reverse-phase chromatography (RPC) using H₂O with 0.1% formic acid and acetonitrile as mobile phases to afford the desired product N-(trans-4-((4-(4-(2,6-dioxopiperidin-3-yl)phenyl)piperazin-1-yl)methyl)cyclohexyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide (29.3 mg, 30% yield) as a yellow solid. MS (ESI, m/z) [M+H]⁺ 788.3 ¹H NMR (400 MHz, DMSO-d₆) δ ppm 10.77 (s, 1H), 9.28 (d, J=8.8 Hz, 1H), 8.45 (d, J=7.8 Hz, 1H), 8.16-8.25 (m, 2H), 8.03-8.12 (m, 3H), 7.83-7.93 (m, 2H), 7.18 (br t, J=5.0 Hz, 1H), 7.05 (d, J=8.8 Hz, 2H), 6.89 (d, J=8.8 Hz, 2H), 3.76-3.85 (m, 1H), 3.73 (dd, J=11.0, 4.9 Hz, 1H), 3.58-3.66 (m, 1H), 3.44-3.52 (m, 2H), 3.08-3.15 (m, 4H), 2.58-2.66 (m, 1H), 2.42-2.49 (m, 5H), 2.17 (br d, J=7.1 Hz, 2H), 2.07-2.15 (m, 1H), 1.97-2.05 (m, 1H), 1.89 (br t, J=13.7 Hz, 4H), 1.46-1.60 (m, 1H), 1.39 (q, J=11.6 Hz, 2H), 1.20 (d, J=6.6 Hz, 3H), 0.92-1.07 (m, 2H).

### Example 10: Synthesis of N-((trans)-4-(2-(4-(4-(2,6-Dioxopiperidin-3-yl)phenyl)piperazin-1-yl)ethyl)cyclohexyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide

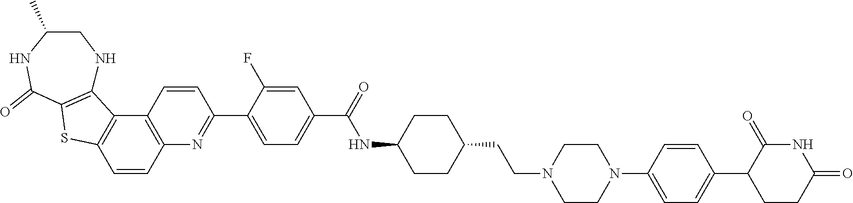 [SMILES: C[C@@H]1CNc2c(sc3ccc4nc(-c5ccc(C(=O)N[C@H]6CC[C@H](CCN7CCN(c8ccc(C9CCC(=O)NC9=O)cc8)CC7)CC6)cc5F)ccc4c23)C(=O)N1]

tert-Butyl (trans-4-(2-(4-(4-(2,6-dioxopiperidin-3-yl)phenyl)piperazin-1-yl)ethyl)cyclohexyl)carbamate. To a stirred solution of 3-(4-piperazin-1-ylphenyl)piperidine-2,6-dione (150 mg, 0.55 mmol) and tert-butyl-N-[4-(2-oxoethyl)cyclohexyl]carbamate (264.9 mg, 1.1 mmol) in MeOH (5 mL) was added ZnCl₂(2.0 M in 2-Me-THF, 0.1 mL 0.55 mmol) and NaBH₃CN (70.24 mg, 1.1 mmol). The mixture was stirred at 60° C. for 4 h. The reaction was quenched with H₂O (30 mL) and extracted with EtOAc (30 mL×3). The combined organic phase was dried over Na₂SO₄, filtered and concentrated. The residue was purified by silica gel column chromatography and using hexane/EtOAc (0-70%) as eluent to afford tert-butyl N-[4-[2-[4-[4-(2,6-dioxo-3-piperidyl)phenyl]piperazin-1-yl]ethyl]cyclohexyl]carbamate (220 mg, 80% yield) as a white solid. MS (ESI, m/z) [M+H]⁺ 499.4.

3-[4-[4-[2-(4-Aminocyclohexyl)ethyl]piperazin-1-yl]phenyl]piperidine-2,6-dione. To a stirred solution of tert-butyl N-[4-[2-[4-[4-(2,6-dioxo-3-piperidyl)phenyl]piperazin-1-yl]ethyl]cyclohexyl]carbamate (220 mg, 0.44 mmol) was added HCl in EtOAc (10 mL, 0.38 mmol). The mixture was stirred at rt for 1 h. The solvent was removed under vacuum to afford 3-[4-[4-[2-(4-aminocyclohexyl)ethyl]piperazin-1-yl]phenyl]piperidine-2,6-dione (160 mg, 90.9%) as a yellow solid. MS (ESI) [M+H]⁺ 399.3.

N-((trans)-4-(2-(4-(4-(2,6-Dioxopiperidin-3-yl)phenyl)piperazin-1-yl)ethyl)cyclohexyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide. To a solution of (R)-3-fluoro-4-(10-methyl-8-oxo-9,10,11,12-tetrahydro-8H-[1,4]diazepino[5′,6′:4,5]thieno[3,2-f]quinolin-3-yl)benzoic acid (100 mg, 0.24 mmol) in DMF (2.4 mL) was added 3-[4-[4-[2-(4-aminocyclohexyl)ethyl]piperazin-1-yl]phenyl]piperidine-2,6-dione;dihydrochloride (134 mg, 0.28 mmol). DIPEA (0.41 mL, 2.37 mmol) was then added and the reaction mixture was sonicated and stirred for 10 min. HATU (90 mg, 0.24 mmol) was then added and the reaction mixture was stirred at rt for 1 h. After removed the solvent under vacuum, the residue is purified by reverse-phase chromatography (RPC) using H₂O with 0.1% FA and acetonitrile as mobile phases to afford the product, N-((trans)-4-(2-(4-(4-(2,6-dioxopiperidin-3-yl)phenyl)piperazin-1-yl)ethyl)cyclohexyl)-3-fluoro-4-((R)-10-methyl-8-oxo-9,10,11,12-tetrahydro-8H-[1,4]diazepino[5′,6′:4,5]thieno[3,2-f]quinolin-3-yl)benzamide (58 mg, 0.072 mmol, 30% yield) as yellow solid after lyophilization. MS (ESI, m/z) [M+H]⁺ 802.4 ¹H NMR (400 MHz, DMSO-d6) δ ppm 10.77 (s, 1H), 9.28 (d, J=9.5 Hz, 1H), 8.42 (d, J=8.1 Hz, 1H), 8.13-8.25 (m, 3H), 8.07-8.12 (m, 2H), 8.02-8.07 (m, 2H), 7.81-7.93 (m, 2H), 7.18 (br t, J=5.0 Hz, 1H), 7.05 (d, J=8.8 Hz, 2H), 6.89 (d, J=8.8 Hz, 2H), 3.75-3.83 (m, 1H), 3.73 (dd, J=11.1, 4.5 Hz, 1H), 3.56-3.67 (m, 1H), 3.44-3.53 (m, 3H), 3.12 (br s, 5H), 2.57-2.66 (m, 1H), 2.37 (br d, J=0.7 Hz, 3H), 2.06-2.20 (m, 1H), 1.96-2.06 (m, 1H), 1.89 (br d, J=10.8 Hz, 2H), 1.81 (br d, J=10.8 Hz, 2H), 1.23-1.45 (m, 6H), 1.20 (d, J=6.6 Hz, 3H), 0.99-1.16 (m, 3H).

### Example 11: Synthesis of 3-(4-(4-(2-(4-(3-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperazin-1-yl)phenyl)piperidine-2,6-dione

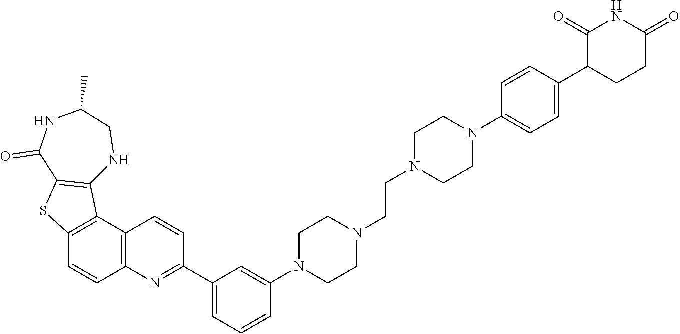 [SMILES: C[C@@H]1CNc2c(sc3ccc4nc(-c5cccc(N6CCN(CCN7CCN(c8ccc(C9CCC(=O)NC9=O)cc8)CC7)CC6)c5)ccc4c23)C(=O)N1]

(R)-10-Methyl-3-(3-(piperazin-1-yl)phenyl)-9,10,11,12-tetrahydro-8H-[1,4]diazepino[5′,6′:4,5]thieno[3,2-f]quinolin-8-one. 3-(4-tert-Butoxycarbonylpiperazinyl)phenylboronic acid pinacol ester (147 mg, 0.38 mmol), (R)-3-chloro-10-methyl-9,10,11,12-tetrahydro-8H-[1,4]diazepino[5′,6′:4,5]thieno[3,2-f]quinolin-8-one (100 mg, 0.31 mmol), cesium carbonate (308 mg, 0.94 mmol), and Pd(dppf)Cl₂ (34.5 mg, 0.05 mmol) were weighed out into 20 mL vial with a stir bar. The vial was sealed, evacuated and filled with nitrogen (×3). 1,4-Dioxane (3 mL) and water (0.3 mL) were then added. The reaction was heated to 100° C. and stirred overnight. The reaction was diluted with EtOAc and transferred to a separatory funnel containing water. The organic layer was removed and the aqueous layer was extracted with EtOAc (20 mL×3). The organic layers were dried over sodium sulfate, filtered, and concentrated. The crude was purified by normal phase column chromatography (0% to 5% MeOH in DCM). After the fractions were concentrated, the solid was then dissolved into a solution of DCM and TFA (4:1), and stirred at rt for 1 h. LC/MS indicated that the reaction was completed. After removing solvent under vacuum, the residue was purified by reverse-phase chromatography (RPC) using H₂O with 0.1% TFA and acetonitrile as mobile phases to afford the product (R)-10-methyl-3-(3-(piperazin-1-yl)phenyl)-9,10,11,12-tetrahydro-8H-[1,4]diazepino[5′,6′:4,5]thieno[3,2-f]quinolin-8-one trifluoro acetate salt (148 mg, 0.27 mmol, 86%) as an orange solid after lyophilization. MS (ESI, m/z) [M+H]⁺ 444.4

(R)-3-(3-(4-(2,2-dimethoxyethyl)piperazin-1-yl)phenyl)-10-methyl-9,10,11,12-tetrahydro-8H-[1,4]diazepino[5′,6′:4,5]thieno[3,2-f]quinolin-8-one. To a solution of (R)-10-methyl-3-(3-(piperazin-1-yl)phenyl)-9,10,11,12-tetrahydro-8H-[1,4]diazepino[5′,6′:4,5]thieno[3,2-f]quinolin-8-one trifluoro acetate salt (350 mg, 0.63 mmol) in CH₂Cl₂ (5 mL) was added DIPEA (0.55 mL, 3.14 mmol). The reaction mixture was stirred at rt for 10 min. Then, 2,2-dimethoxyacetaldehyde (110 μL, 0.75 mmol) and NaBH(OAc)₃ (332 mg, 1.57 mmol) were added. The reaction was stirred at rt overnight. LCMS showed completed conversion. After removing the solvent under vacuum, the residue is purified by reverse-phase chromatography (RPC) using H₂O with 0.1% FA and acetonitrile as mobile phases to afford the product, (R)-3-(3-(4-(2,2-dimethoxyethyl)piperazin-1-yl)phenyl)-10-methyl-9,10,11,12-tetrahydro-8H-[1,4]diazepino[5′,6′:4,5]thieno[3,2-f]quinolin-8-one formic acid salt (295 mg, 88% yield) as a yellow solid. MS (ESI, m/z) [M+H]⁺ 532.2.

(R)-2-(4-(3-(10-methyl-8-oxo-9,10,11,12-tetrahydro-8H-[1,4]diazepino[5′,6′:4,5]thieno[3,2-f]quinolin-3-yl)phenyl)piperazin-1-yl)acetaldehyde. To a solution of (R)-3-(3-(4-(2,2-dimethoxyethyl)piperazin-1-yl)phenyl)-10-methyl-9,10,11,12-tetrahydro-8H-[1,4]diazepino[5′,6′:4,5]thieno[3,2-f]quinolin-8-one formic acid salt (295 mg, 0.55 mmol) in 1,4-dioxane (2 mL) and water (0.25 mL) was added 4.0 M HCl in 1,4-dioxane (3 mL, 11.1 mmol). The reaction mixture was stirred at rt overnight. LCMS showed 50% conversion to the desired product. A solution of HCl (2.0 mL, 4.0 M in 1,4-dioxane) was added and then heated to 40° C. After 1.5 h, LCMS showed full conversion. The solvent was evaporated under vacuum, co-evaporated with MeCN (3×) and CH₂Cl₂ (2×) to afford (R)-2-(4-(3-(10-methyl-8-oxo-9,10,11,12-tetrahydro-8H-[1,4]diazepino[5′,6′:4,5]thieno[3,2-f]quinolin-3-yl)phenyl)piperazin-1-yl)acetaldehyde;dihydrochloride (300 mg, 97%) as a red solid. MS (ESI, m/z) [M+H]⁺ 488.2.

3-(4-(4-(2-(4-(3-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperazin-1-yl)phenyl)piperidine-2,6-dione. To a solution of (R)-2-(4-(3-(10-methyl-8-oxo-9,10,11,12-tetrahydro-8H-[1,4]diazepino[5′,6′:4,5]thieno[3,2-f]quinolin-3-yl)phenyl)piperazin-1-yl)acetaldehyde;dihydrochloride (160 mg, 0.29 mmol) and 3-(4-piperazin-1-ylphenyl)piperidine-2,6-dione dihydrochloride (119.0 mg, 0.34 mmol) in CH₂Cl₂ (2 mL) and DMSO (0.75 mL) was added DIPEA (0.4 mL, 2.29 mmol) under an inert atmosphere. The solution was stirred at rt for 10 min. Then, NaBH(OAc)₃ (182 mg, 0.86 mmol) was added, and the reaction mixture was stirred at rt overnight. After removed the solvent under vacuum, the residue is purified by reverse-phase chromatography (RPC) using H₂O with 0.1% FA and acetonitrile as mobile phases to afford the product, 3-(4-(4-(2-(4-(3-((R)-10-methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperazin-1-yl)phenyl)piperidine-2,6-dione (49 mg, 23% yield) as a yellow solid. MS (ESI, m/z) [M+H]⁺ 743.3. ¹H NMR (400 MHz, DMSO-d₆) δ ppm 10.77 (s, 1H), 9.21 (d, J=8.8 Hz, 1H), 8.22 (d, J=9.0 Hz, 1H), 8.14 (s, 1H), 8.08 (br d, J=4.4 Hz, 1H), 8.02 (d, J=8.8 Hz, 1H), 7.86 (s, 1H), 7.63-7.76 (m, 1H), 7.40 (t, J=7.9 Hz, 1H), 7.15 (br t, J=5.1 Hz, 1H), 7.01-7.12 (m, 3H), 6.89 (d, J=8.8 Hz, 2H), 3.72 (dd, J=10.9, 5.0 Hz, 1H), 3.62 (td, J=7.0, 3.2 Hz, 1H), 3.48 (br s, 3H), 3.13 (br s, 5H), 2.54-2.73 (m, 13H), 1.95-2.21 (m, 2H), 1.20 (d, J=6.8 Hz, 3H). Three protons not observed.

### Example 12: Synthesis of 3-(1-Methyl-6-(4-(2-(4-(3-((R)-10-methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperazin-1-yl)-1H-indazol-3-yl)piperidine-2,6-dione

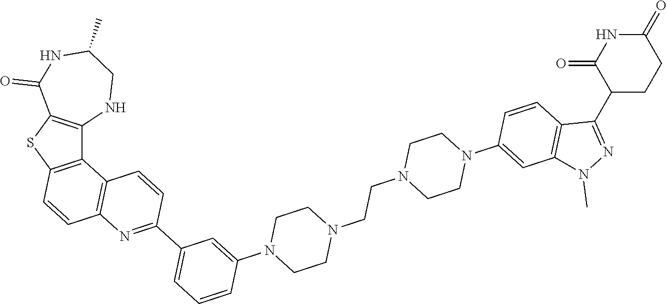 [SMILES: C[C@@H]1CNc2c(sc3ccc4nc(-c5cccc(N6CCN(CCN7CCN(c8ccc9c(C%10CCC(=O)NC%10=O)nn(C)c9c8)CC7)CC6)c5)ccc4c23)C(=O)N1]

3-(1-Methyl-6-(4-(2-(4-(3-((R)-10-methyl-8-oxo-9,10,11,12-tetrahydro-8H-[1,4]diazepino[5′,6′:4,5]thieno[3,2-(]quinolin-3-yl)phenyl)piperazin-1-yl)ethyl)piperazin-1-yl)-1H-indazol-3-yl)piperidine-2,6-dione. To a solution of (R)-2-(4-(3-(10-methyl-8-oxo-9,10,11,12-tetrahydro-8H-[1,4]diazepino[5′,6′:4,5]thieno[3,2-f]quinolin-3-yl)phenyl)piperazin-1-yl)acetaldehyde;dihydrochloride (160 mg, 0.29 mmol) and 3-(1-methyl-6-piperazin-1-yl-indazol-3-yl)piperidine-2,6-dione;hydrochloride (125 mg, 0.34 mmol) in CH₂Cl₂ (2 mL) and DMSO (0.75 mL) was added DIPEA (0.4 mL, 2.29 mmol) under inert atmosphere. The solution was stirred at rt for 10 min. Then, NaBH(OAc)₃ (182 mg, 0.86 mmol) was added, and the reaction mixture was stirred at rt overnight. After removing the solvent under vacuum, the residue is purified by reverse-phase chromatography (RPC) using H₂O with 0.1% FA and acetonitrile as mobile phases to afford the product, 3-(1-methyl-6-(4-(2-(4-(3-((R)-10-methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperazin-1-yl)-1H-indazol-3-yl)piperidine-2,6-dione (69 mg, 30% yield) as a yellow solid. MS (ESI, m/z) [M+H]⁺ 797.4 ¹H NMR (400 MHz, DMSO-d₆) δ ppm 10.85 (s, 1H), 9.22 (d, J=9.0 Hz, 1H), 8.22 (d, J=9.0 Hz, 1H), 8.14 (s, 1H), 8.08 (d, J=4.2 Hz, 1H), 8.02 (d, J=9.0 Hz, 1H), 7.86 (s, 1H), 7.63-7.74 (m, 1H), 7.50 (d, J=8.8 Hz, 1H), 7.40 (t, J=7.9 Hz, 1H), 7.15 (t, J=5.3 Hz, 1H), 7.10 (dd, J=8.1, 2.0 Hz, 1H), 6.93 (dd, J=8.9, 1.8 Hz, 1H), 6.85 (d, J=1.5 Hz, 1H), 4.26 (dd, J=9.0, 5.1 Hz, 1H), 3.89 (s, 3H), 3.58-3.68 (m, 1H), 3.43-3.52 (m, 3H), 3.22-3.26 (m, 5H), 2.56-2.73 (m, 14H), 2.27-2.37 (m, 1H), 2.11-2.23 (m, 1H), 1.20 (d, J=6.8 Hz, 3H).

### Example 13: Synthesis of 3-(4-(4-(2-(4-(3-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetyl)piperazin-1-yl)phenyl)piperidine-2,6-dione

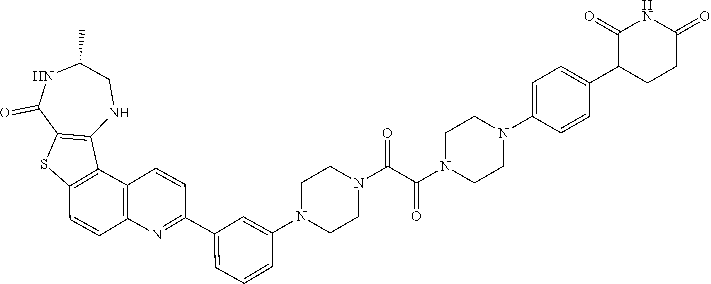 [SMILES: C[C@@H]1CNc2c(sc3ccc4nc(-c5cccc(N6CCN(C(=O)C(=O)N7CCN(c8ccc(C9CCC(=O)NC9=O)cc8)CC7)CC6)c5)ccc4c23)C(=O)N1]

(R)-2-(4-(3-(10-Methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetic acid. A 20 mL vial equipped with stir bar was filled with (R)-10-methyl-3-(3-(piperazin-1-yl)phenyl)-9,10,11,12-tetrahydro-8H-[1,4]diazepino[5′,6′:4,5]thieno[3,2-f]quinolin-8-one trifluoroacetate salt (100 mg, 0.18 mmol), DCM/DMSO (5 mL, 10:1) was then added followed by DIPEA (125 μL, 0.72 mmol) and ethyl 2-chloro-2-oxoacetate (21 μL, 0.19 mmol). The resulting mixture was allowed to stir at rt for 2 h after LCMS analysis revealed formation of the desired ethyl ester. The reaction mixture was concentrated and lithium hydroxide (21.5 mg, 0.9 mmol) was added follow by THF/water (5 mL, 3:2). After stirring at rt for 1.0 h, the reaction mixture was concentrated under vacuum. The residue was purified by reverse-phase chromatography (RPC) using H₂O with 0.1% TFA and acetonitrile as mobile phases to afford the product (R)-2-(4-(3-(10-methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetic acid trifluoroacetate salt (43 mg, 32% yield, 85% purity) as a red solid. MS (ESI, m/z) [M+H]⁺ 516.2.

3-(4-(4-(2-(4-(3-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetyl)piperazin-1-yl)phenyl)piperidine-2,6-dione. In a 2-dram vial, DMF (1.5 mL) was charged and followed by the addition of (R)-2-(4-(3-(10-methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetic acid (19.0 mg, 37 mmol), HATU (26.6 mg, 74 mmol) and 3-(4-(piperazin-1-yl)phenyl)piperidine-2,6-dione (11.2 mg, 41 mmol). To the resulting mixture, DIPEA (0.02 mL, 0.11 mmol) was added, and then the reaction was allowed to stir at rt for overnight. After the filtration of reaction mixture, the filtrate is purified by reverse-phase chromatography (RPC) using H₂O with 0.05% TFA and acetonitrile as mobile phases to afford 3-(4-(4-(2-(4-(3-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetyl)piperazin-1-yl)phenyl)piperidine-2,6-dione (20.9 mg, 72% yield). MS (ESI, m/z) [M+H]⁺ 771.4

### Example 14: Synthesis of 3-(1-Methyl-6-(4-(2-(4-(3-((R)-10-methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetyl)piperazin-1-yl)-1H-indazol-3-yl)piperidine-2,6-dione

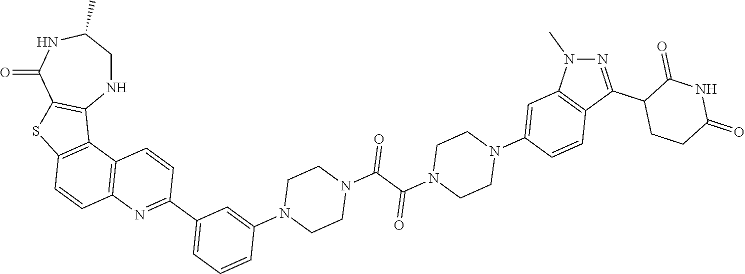 [SMILES: C[C@@H]1CNc2c(sc3ccc4nc(-c5cccc(N6CCN(C(=O)C(=O)N7CCN(c8ccc9c(C%10CCC(=O)NC%10=O)nn(C)c9c8)CC7)CC6)c5)ccc4c23)C(=O)N1]

3-(1-Methyl-6-(4-(2-(4-(3-((R)-10-methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetyl)piperazin-1-yl)-1H-indazol-3-yl)piperidine-2,6-dione. In a 2-dram vial, DMF (1.5 mL) was charged and followed by the addition of (R)-2-(4-(3-(10-methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetic acid (19.0 mg, 37 mmol), HATU (26.6 mg, 74 mmol) and 3-(1-methyl-6-(piperazin-1-yl)-1H-indazol-3-yl)piperidine-2,6-dione (13.4 mg, 41 mmol). To the resulting mixture, DIPEA (0.02 mL, 0.11 mmol) was added, and then the reaction was allowed to stir at rt for overnight. After the filtration of reaction mixture, the filtrate is purified by reverse-phase chromatography (RPC) using H₂O with 0.05% TFA and acetonitrile as mobile phases to afford 3-(1-Methyl-6-(4-(2-(4-(3-((R)-10-methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetyl)piperazin-1-yl)-1H-indazol-3-yl)piperidine-2,6-dione (17.9 mg, 59% yield). MS (ESI, m/z) [M+H]⁺ 825.5.

### Example 15: Synthesis of 3-(1-Methyl-6-(4-(2-(3-(3-((R)-10-methyl-8-oxo-9,10,11,12-tetrahydro-8H-[1,4]diazepino[5′,6′:4,5]thieno[3,2-f]quinolin-3-yl)phenyl)-3,6-diazabicyclo[3.1.1]heptan-6-yl)ethyl)piperazin-1-yl)-1H-indazol-3-yl)piperidine-2,6-dione

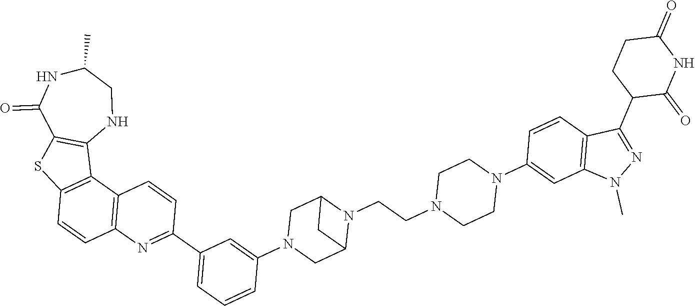 [SMILES: C[C@@H]1CNc2c(sc3ccc4nc(-c5cccc(N6CC7CC(C6)N7CCN6CCN(c7ccc8c(C9CCC(=O)NC9=O)nn(C)c8c7)CC6)c5)ccc4c23)C(=O)N1]

tert-Butyl 3-(3-bromophenyl)-3,6-diazabicyclo[3.1.1]heptane-6-carboxylate. To a stirred solution of tert-butyl 3,6-diazabicyclo[3.1.1]heptane-6-carboxylate (1.0 g, 5.0 mmol) and 1-bromo-3-iodo-benzene (2.1 g, 7.6 mmol) and Pd₂(dba)₃ (279.0 mg, 0.25 mmol) and XantPhos (241.0 mg, 0.50 mmol) in toluene (10.0 mL), t-BuONa (1.5 g, 15.1 mmol) was added. The resulting solution was stirred at 60° C. for 3 h under an inert atmosphere. After cooling down to rt, the reaction mixture was diluted with water (300 mL) and then extracted by ethyl acetate (100 mL×3). The combined organic layer was washed with brine (100 mL×2), dried over anhydrous sodium sulfate, filtered and concentrated under vacuum. The residue was purified by purified by silica gel column chromatography with petroleum ether/ethyl acetate (2:1) as mobile phases to afford tert-butyl 3-(3-bromophenyl)-3,6-diazabicyclo[3.1.1]heptane-6-carboxylate (1.5 g, 84%) as a yellow solid. MS (ESI, m/z) [M+H]⁺ 353.1

3-(3-Bromophenyl)-3,6-diazabicyclo[3.1.1]heptane. To a solution of tert-butyl 3-(3-bromophenyl)-3,6-diazabicyclo[3.1.1]heptane-6-carboxylate (1.5 g, 4.25 mmol) in DCM (20.0 mL), TFA (10.0 mL) was added. The resulted solution was stirred at rt for 2 hr. After concentrating under vacuum to remove solvent, the crude product, 3-(3-bromophenyl)-3,6-diazabicyclo[3.1.1]heptane (1.2 g) was obtained and used directly to the next step without further purification. MS (ESI, m/z) [M+H]⁺ 253.2.

3-(3-Bromophenyl)-6-(2,2-dimethoxyethyl)-3,6-diazabicyclo[3.1.1]heptane. To a solution of 3-(3-bromophenyl)-3,6-diazabicyclo[3.1.1]heptane (1.2 g, 4.74 mmol) and 2,2-dimethoxyacetaldehyde (0.71 g, 6.85 mmol) in MeOH (5.0 mL), NaBH₃CN (896 mg, 14.229 mmol, 3.00 equiv) was added. The resulted mixture was stirred at rt for 4 h. After concentrating under vacuum to remove the solvent, the residue was purified by reverse flash chromatography on silica gel with water (0.05% TFA)/MeCN (1.2), to afford 3-(3-bromophenyl)-6-(2,2-dimethoxyethyl)-3,6-diazabicyclo[3.1.1]heptane (1.4 g, 90%) as a white solid. MS (ESI, m/z) [M+H]⁺ 341.2.

6-(2,2-Dimethoxyethyl)-3-[3-(4,4,5,5-tetramethyl-1,3,2-dioxaborolan-2-yl)phenyl]-3,6-diazabicyclo[3.1.1]heptane. To a solution of 3-(3-bromophenyl)-6-(2,2-dimethoxyethyl)-3,6-diazabicyclo[3.1.1]heptane (700.0 mg, 2.05 mmol) and B₂pin₂ (628.0 mg, 2.47 mmol) and Pd(dppf)Cl₂ (167.0 mg, 0.21 mmol) in 1,4-dioxane (10.0 mL), KOAc (605.0 mg, 6.16 mmol) was added. The resulting solution was stirred at 80° C. for 6 h under an inert atmosphere. After cooling down to rt, the reaction mixture was diluted with water (100.0 mL) and extracted by ethyl acetate (100 mL×3). The combined organic layer was washed with brine (100 mL×2). The combined organic extracts were dried over anhydrous sodium sulfate, filtered, and concentrated under vacuum. The residue was purified by flash chromatography on silica gel with dichloromethane/methanol (15:1) to give 6-(2,2-dimethoxyethyl)-3-[3-(4,4,5,5-tetramethyl-1,3,2-dioxaborolan-2-yl)phenyl]-3,6-diazabicyclo[3.1.1]heptane (380 mg, 48%) as a red solid. MS (ESI, m/z) [M+H]⁺ 389.1 To a solution of 6-(2,2-dimethoxyethyl)-3-[3-(4,4,5,5-tetramethyl-1,3,2-dioxaborolan-2-yl)phenyl]-3,6-diazabicyclo[3.1.1]heptane (350.0 mg, 0.90 mmol) and (R)-3-chloro-10-methyl-9,10,11,12-tetrahydro-8H-[1,4]diazepino[5′,6′:4,5]thieno[3,2-f]quinolin-8-one (200.0 mg, 0.63 mmol) and Pd(dtbpf)Cl₂ (70.0 mg, 0.06 mmol) in 1,4-dioxane (5.0 mL) and water (1.0 mL), K₃PO₄ (268.0 mg, 1.26 mmol) was added. The resulting solution was stirred at 90° C. overnight under an inert atmosphere. After cooling down to rt, the reaction mixture was diluted with water (100 mL) and extracted by ethyl acetate (100 mL×3). The combined organic layer was washed with brine (100 mL×2), dried over anhydrous sodium sulfate, filtered, and concentrated under vacuum. The residue was purified by reverse-phase chromatography (RPC) using H₂O with 0.05% TFA and acetonitrile (MeCN) as mobile phases to afford (10R)-3-(3-(6-(2,2-dimethoxyethyl)-3,6-diazabicyclo[3.1.1]heptan-3-yl)phenyl)-10-methyl-9,10,11,12-tetrahydro-8H-[1,4]diazepino[5′,6′:4,5]thieno[3,2-f]quinolin-8-one (270 mg, 79% yield) as a red solid. MS (ESI, m/z) [M+H]⁺ 544.2.

2-(3-(3-((R)-10-methyl-8-oxo-9,10,11,12-tetrahydro-8H-[1,4]diazepino[5′,6′:4,5]thieno[3,2-f]quinolin-3-yl)phenyl)-3,6-diazabicyclo[3.1.1]heptan-6-yl)acetaldehyde. To a solution of (10R)-3-(3-(6-(2,2-dimethoxyethyl)-3,6-diazabicyclo[3.1.1]heptan-3-yl)phenyl)-10-methyl-9,10,11,12-tetrahydro-8H-[1,4]diazepino[5′,6′:4,5]thieno[3,2-f]quinolin-8-one (100.0 mg, 0.18 mmol) in 1,4-dioxane (1.0 mL) and water (0.50 mL), HCl (1.0 mL, 12.1 M) was added. The resulting solution was stirred at 40° C. for 24 h. After concentrating under vacuum, the crude product, 2-(3-(3-((R)-10-methyl-8-oxo-9,10,11,12-tetrahydro-8H-[1,4]diazepino[5′,6′:4,5]thieno[3,2-f]quinolin-3-yl)phenyl)-3,6-diazabicyclo[3.1.1]heptan-6-yl)acetaldehyde (80.0 mg) was obtained and used directly for the next step without further purification. MS (ESI, m/z) [M+H]⁺ 518.1.

3-(1-Methyl-6-(4-(2-(3-(3-((R)-10-methyl-8-oxo-9,10,11,12-tetrahydro-8H-[1,4]diazepino[5′,6′:4,5]thieno[3,2-f]quinolin-3-yl)phenyl)-3,6-diazabicyclo[3.1.1]heptan-6-yl)ethyl)piperazin-1-yl)-1H-indazol-3-yl)piperidine-2,6-dione. To a solution of 2-(3-(3-((R)-10-methyl-8-oxo-9,10,11,12-tetrahydro-8H-[1,4]diazepino[5′,6′:4,5]thieno[3,2-f]quinolin-3-yl)phenyl)-3,6-diazabicyclo[3.1.1]heptan-6-yl)acetaldehyde (91.0 mg, 0.18 mmol) and 3-(1-methyl-6-piperazin-1-yl-indazol-3-yl)piperidine-2,6-dione (90.0 mg, 0.27 mmol) in methanol (5.0 mL), NaBH₃CN (34.0 mg, 0.54 mmol) was added. The resulting solution was stirred at rt overnight. The reaction mixture was diluted with DMSO (1.0 mL) and purified by reverse-phase chromatography (RPC) using H₂O with 0.05% TFA and acetonitrile (MeCN) as mobile phases to afford the desired product, 3-(1-Methyl-6-(4-(2-(3-(3-((R)-10-methyl-8-oxo-9,10,11,12-tetrahydro-8H-[1,4]diazepino[5′,6′:4,5]thieno[3,2-f]quinolin-3-yl)phenyl)-3,6-diazabicyclo[3.1.1]heptan-6-yl)ethyl)piperazin-1-yl)-1H-indazol-3-yl)piperidine-2,6-dione (51.0 mg, 33% yield) as a red solid. MS (ESI, m/z) [M+H]⁺ 810.1 ¹H NMR (400 MHz, DMSO-d₆) δ ppm 9.25 (d, J=8.8 Hz, 1H), 8.25 (d, J=9.2 Hz, 1H), 8.17 (d, J=8.8 Hz, 1H), 8.05 (d, J=8.8 Hz, 1H), 7.76 (s, 1H), 7.67 (d, J=7.6 Hz, 1H), 7.60-7.49 (m, 2H), 7.04-6.93 (m, 3H), 4.72-4.63 (m, 2H), 4.28 (s, 1H), 3.95-3.90 (m, 6H), 3.65 (d, J=6.8 Hz, 1H), 3.52-3.39 (m, 13H), 2.96-2.90 (m, 1H), 2.72-2.62 (m, 3H), 2.30-2.20 (m, 2H), 2.17-2.11 (m, 2H), 1.22 (d, J=6.8 Hz, 3H).

### Example 16: Synthesis of 3-(4-(4-(2-(4-(3-((R)-10-Methyl-8-oxo-8,9,10,11-tetrahydro-[1,4]oxazepino[7′,6′:4,5]thieno[3,2-j]quinolin-3-yl)phenyl)piperazin-1-yl)ethyl)piperazin-1-yl)phenyl)piperidine-2,6-dione

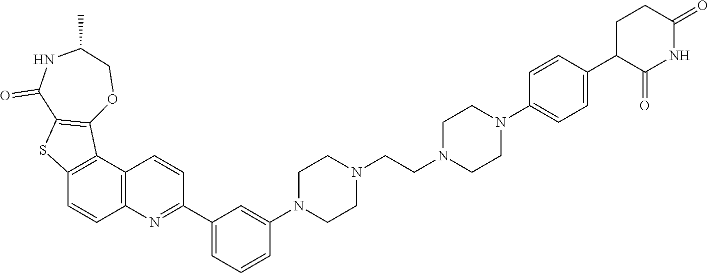 [SMILES: C[C@@H]1COc2c(sc3ccc4nc(-c5cccc(N6CCN(CCN7CCN(c8ccc(C9CCC(=O)NC9=O)cc8)CC7)CC6)c5)ccc4c23)C(=O)N1]

1-(3-Bromophenyl)-4-(2,2-dimethoxyethyl)piperazine. To a solution of NaBH₃CN (398 mg, 6.2 mmol) in methanol (10.0 mL) was added ZnCl₂ in THE (1.9 M, 7.9 mL, 2.1 mmol). After the mixture was stirred at rt for 10 min. 1-(3-bromophenyl)piperazine (500 mg, 2.1 mmol) and 2,2-dimethoxyacetaldehyde (431 mg, 4.2 mmol) were added. The resulting solution was stirred at rt overnight. LCMS showed the reaction was completed. The resulting solution was then concentrated under vacuum. The residue was purified by reverse phase column chromatography (0% to 10% MeCN in water w/ 0.1% TFA) to afford 1-(3-bromophenyl)-4-(2,2-dimethoxyethyl)piperazine (550 mg, 80%) as an off-white solid. MS (ESI, m/z) [M+H]⁺ 329.4.

1-(2,2-Dimethoxyethyl)-4-(3-(4,4,5,5-tetramethyl-1,3,2-dioxaborolan-2-yl)phenyl)piperazine. To a solution of 1-(3-bromophenyl)-4-(2,2-dimethoxyethyl)piperazine (550 mg, 1.7 mmol) in 1,4-dioxane (10 mL) were added B₂pin₂ (636 mg, 2.5 mmol), Pd(dppf)Cl₂ (68 mg, 0.08 mmol) and KOAc (491 mg, 5.1 mmol). The resulting solution was stirred at 100° C. overnight under a nitrogen atmosphere. LCMS showed the reaction was completed. The resulting solution was diluted with water (50.0 mL) and extracted with ethyl acetate (30 mL×3). The combined organic layer was washed with brine (30 mL×2), dried over anhydrous sodium sulfate, filtered, and concentrated under vacuum. The residue was purified by silica gel chromatography with ethyl acetate/petroleum ether (1:1) to afford 1-(2,2-dimethoxyethyl)-4-[3-(4,4,5,5-tetramethyl-1,3,2-dioxaborolan-2-yl)phenyl]piperazine (370 mg, 59%) as a yellow solid. MS (ESI, m/z) [M+H]⁺ 377.3.

tert-Butyl (R)-3-(3-(4-(2,2-dimethoxyethyl)piperazin-1-yl)phenyl)-10-methyl-8-oxo-10,11-dihydro-[1,4]oxazepino[7′,6′:4,5]thieno[3,2-f]quinoline-9(8H)-carboxylate. To a solution of 1-(2,2-dimethoxyethyl)-4-[3-(4,4,5,5-tetramethyl-1,3,2-dioxaborolan-2-yl)phenyl]piperazine (100 mg, 0.27 mmol) in 1,4-dioxane (3.0 mL) and water (0.5 mL) were added tert-butyl (R)-3-chloro-10-methyl-8-oxo-10,11-dihydro-[1,4]oxazepino[7′,6′:4,5]thieno[3,2-f]quinoline-9(8H)-carboxylate (166 mg, 0.398 mmol), Pd(PPh₃)₄(31 mg, 0.027 mmol) and K₂CO₃ (110 mg, 0.80 mmol). The resulting solution was stirred at 100° C. overnight under a nitrogen atmosphere. LCMS showed the reaction was completed. The resulting solution was diluted with water (50.0 mL) and extracted with ethyl acetate (30 mL×3). The combined organic layer was washed with brine (30 mL×2), dried over anhydrous sodium sulfate, filtered, and concentrated under vacuum. The residue was purified by silica gel chromatography with ethyl acetate/petroleum ether (1:1) to afford tert-butyl (R)-3-(3-(4-(2,2-dimethoxyethyl)piperazin-1-yl)phenyl)-10-methyl-8-oxo-10,11-dihydro-[1,4]oxazepino[7′,6′:4,5]thieno[3,2-f]quinoline-9(8H)-carboxylate (100 mg, 60% yield) as a yellow solid. MS (ESI, m/z) [M+H]⁺ 633.2.

tert-Butyl (R)-10-methyl-8-oxo-3-(3-(4-(2-oxoethyl)piperazin-1-yl)phenyl)-10,11-dihydro-[1,4]oxazepino[7′,6′:4,5]thieno[3,2-f]quinoline-9(8H)-carboxylate. To a solution of tert-butyl (R)-3-(3-(4-(2,2-dimethoxyethyl)piperazin-1-yl)phenyl)-10-methyl-8-oxo-10,11-dihydro-[1,4]oxazepino[7′,6′:4,5]thieno[3,2-f]quinoline-9(8H)-carboxylate (100 mg, 0.16 mmol) in 1,4-dioxane (5.0 mL) was added 4 M HCl (aq.) (10 mL). The resulting solution was stirred at 40° C. for 2 h. LCMS analysis showed the reaction was completed. The resulting solution was concentrated under vacuum to afford crude tert-butyl (R)-10-methyl-8-oxo-3-(3-(4-(2-oxoethyl)piperazin-1-yl)phenyl)-10,11-dihydro-[1,4]oxazepino[7′,6′:4,5]thieno[3,2-f]quinoline-9(8H)-carboxylate (80 mg). MS (ESI, m/z) [M+H]⁺ 487.1.

3-(4-(4-(2-(4-(3-((R)-10-Methyl-8-oxo-8,9,10,11-tetrahydro-[1,4]oxazepino[7′,6′:4,5]thieno[3,2-f]quinolin-3-yl)phenyl)piperazin-1-yl)ethyl)piperazin-1-yl)phenyl)piperidine-2,6-dione. In a 20 mL vials with the crude product from (R)-2-(4-(3-(10-methyl-8-oxo-8,9,10,11-tetrahydro-[1,4]oxazepino[7′,6′:4,5]thieno[3,2-f]quinolin-3-yl)phenyl)piperazin-1-yl)acetaldehyde (33.2 mg, 68 mmol), 1,2-dichloroethane (2 mL) and THE (1.0 mL) were charged to the vial. To the mixture, DIPEA (0.034 mL, 0.194 mmol), 3-(4-(piperazin-1-yl)phenyl)piperidine-2,6-dione (0.311 mL, 78 mmol) and NaBH(OAc)₃ (0.518 mL, 0.5 M in THE) were added. The resulting mixture was stirred at rt for overnight. After additional NaBH(OAc)₃ (0.272 mL, 0.5 M in THF) was added, the reaction was continuously stirred and heated at 50° C. for 4.0 h. After the removal the solvent, the residue was dissolved into 2.0 mL of DMSO and purified by reverse-phase chromatography (RPC) using H₂O with 0.1% formic acid and acetonitrile as mobile phases to afford the desired product 3-(4-(4-(2-(4-(3-((R)-10-methyl-8-oxo-8,9,10,11-tetrahydro-[1,4]oxazepino[7′,6′:4,5]thieno[3,2-f]quinolin-3-yl)phenyl)piperazin-1-yl)ethyl)piperazin-1-yl)phenyl)piperidine-2,6-dione, formic acid salt (4.0 mg, 7.4% yield) as light yellow solid. MS (ESI, m/z) [M+H]⁺ 744.5.

### Example 17: Synthesis of 3-(1-Methyl-6-(4-(2-(4-(3-((R)-10-methyl-8-oxo-8,9,10,11-tetrahydro-[1,4]oxazepino[7′,6′:4,5]thieno[3,2-f]quinolin-3-yl)phenyl)piperazin-1-yl)ethyl)piperazin-1-yl)-1H-indazol-3-yl)piperidine-2,6-dione

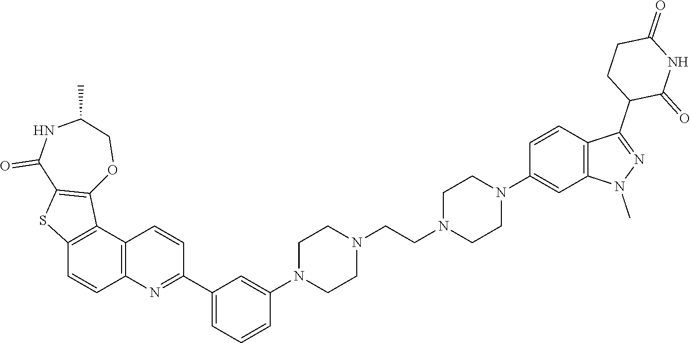 [SMILES: C[C@@H]1COc2c(sc3ccc4nc(-c5cccc(N6CCN(CCN7CCN(c8ccc9c(C%10CCC(=O)NC%10=O)nn(C)c9c8)CC7)CC6)c5)ccc4c23)C(=O)N1]

3-(1-Methyl-6-(4-(2-(4-(3-((R)-10-methyl-8-oxo-8,9,10,11-tetrahydro-[1,4]oxazepino[7′,6′:4,5]thieno[3,2-f]quinolin-3-yl)phenyl)piperazin-1-yl)ethyl)piperazin-1-yl)-1H-indazol-3-yl)piperidine-2,6-dione. To a solution of 3-(1-methyl-6-piperazin-1-yl-indazol-3-yl)piperidine-2,6-dione (67 mg, 0.21 mmol) in DCM (5.0 mL), was added TEA until the pH of the solution reached 9˜10. Then, tert-butyl (R)-10-methyl-8-oxo-3-(3-(4-(2-oxoethyl)piperazin-1-yl)phenyl)-10,11-dihydro-[1,4]oxazepino[7′,6′:4,5]thieno[3,2-f]quinoline-9(8H)-carboxylate (80 mg, 0.16 mmol) was added to the solution. The pH value of the solution was then adjusted to 5˜6 with HOAc, and then NaBH(OAc)₃ (130 mg, 0.62 mmol) was added and the resulting solution was stirred at rt for 2 h. LCMS showed the reaction was completed. After concentration to remove the solvent under vacuum, the residue was purified by reverse-phase chromatography (RPC) using H₂O with 0.05% TFA and acetonitrile with 0.05% TFA as mobile phases to afford the product, 3-(1-methyl-6-(4-(2-(4-(3-((R)-10-methyl-8-oxo-8,9,10,11-tetrahydro-[1,4]oxazepino[7′,6′:4,5]thieno[3,2-f]quinolin-3-yl)phenyl)piperazin-1-yl)ethyl)piperazin-1-yl)-1H-indazol-3-yl)piperidine-2,6-dione (33.9 mg, 20% yield) as an orange solid. MS (ESI) [M+H]⁺ 798. ¹H NMR (400 MHz, DMSO-d₆): δ ppm 9.35 (d, J=8.8 Hz, 1H), 8.29-8.22 (m, 2H), 8.10 (d, J=9.2 Hz, 1H), 7.91 (s, 1H), 7.77 (d, J=7.2 Hz, 1H), 7.58 (d, J=9.2 Hz, 1H), 7.49 (t, J=8.0 Hz, 1H), 7.21 (d, J=8.0 Hz, 1H), 7.00 (d, J=6.4 Hz, 1H), 4.62 (s, 2H), 4.30-4.27 (m, 1H), 3.92 (s, 3H), 3.87 (s, 1H), 3.80-3.50 (m, 18H), 2.80-2.50 (m, 4H), 2.38-2.10 (m, 2H), 1.30 (d, J=6.4 Hz, 3H). Three protons not observed. ¹⁹F NMR (376 MHz, DMSO-d): δ ppm ˜74.41.

### Example 18: Synthesis of 3-(4-(4-(2-(4-(3-((R)-10-Methyl-8-oxo-9,10,11,12-tetrahydro-8H-[1,4]diazepino[5′,6′:4,5]thieno[3,2-f]quinolin-3-yl)phenyl)piperidin-1-yl)ethyl)piperazin-1-yl)phenyl)piperidine-2,6-dione

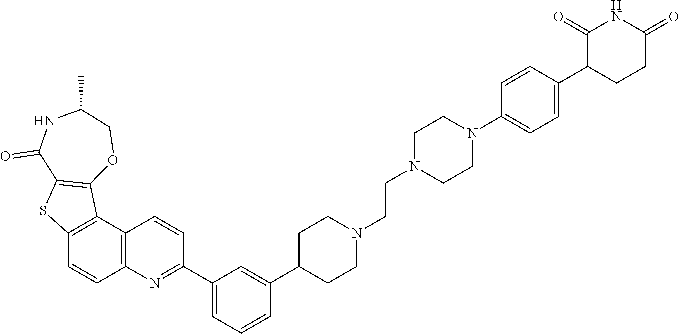 [SMILES: C[C@@H]1COc2c(sc3ccc4nc(-c5cccc(C6CCN(CCN7CCN(c8ccc(C9CCC(=O)NC9=O)cc8)CC7)CC6)c5)ccc4c23)C(=O)N1]

(R)-10-methyl-3-(3-(piperidin-4-yl)phenyl)-9,10,11,12-tetrahydro-8H-[1,4]diazepino[5′,6′:4,5]thieno[3,2-f]quinolin-8-one tert-Butyl 4-[3-(4,4,5,5-tetramethyl-1,3,2-dioxaborolan-2-yl)phenyl]piperidine-1-carboxylate (145 mg, 0.38 mmol), (R)-3-chloro-10-methyl-9,10,11,12-tetrahydro-8H-[1,4]diazepino[5′,6′:4,5]thieno[3,2-f]quinolin-8-one (100 mg, 0.31 mmol), cesium carbonate (306 mg, 0.94 mmol), and Pd(dppf)Cl₂ (23 mg, 0.03 mmol) were weighed into a 20 mL vial with a stir bar. The vial was evacuated and back-filled with nitrogen three times, and then 1,4-dioxane (3.1 mL) and water (0.3 mL) were added to the vial. The reaction was then heated at 100° C. for 2 h. LCMS analysis revealed that the reaction was completed. The reaction was then diluted with EtOAc washed with saturated aqueous NaHCO₃ and the aqueous layer was extracted with EtOAc (×3). The collected organic layers were dried over sodium sulfate, filtered, and concentrated. The residue was purified by normal phase column chromatography (0 to 10% MeOH in DCM). The collected fractions were concentrated, the residue was dissolved into 4:1 DCM/TFA and stirred for 1.5 h at rt. LCMS indicated that the deprotection was completed. After removing the solvent under vacuum, the residue was purified by reverse-phase chromatography (RPC) using H₂O with 0.05% TFA and acetonitrile with 0.05% TFA as mobile phases to afford tert-butyl (R)-3-(3-(4-(2,2-dimethoxyethyl)piperazin-1-yl)phenyl)-10-methyl-8-oxo-10,11-dihydro-[1,4]oxazepino[7′,6′:4,5]thieno[3,2-f]quinoline-9(8H)-carboxylate (151 mg, 86% yield). MS (ESI, m/z) [M+H]⁺ 443.4.

(R)-3-(3-(1-(2,2-dimethoxyethyl)piperidin-4-yl)phenyl)-10-methyl-9,10,11,12-tetrahydro-8H-[1,4]diazepino[5′,6′:4,5]thieno[3,2-f]quinolin-8-one. To a solution of (R)-10-methyl-3-(3-(piperidin-4-yl)phenyl)-9,10,11,12-tetrahydro-8H-[1,4]diazepino[5′,6′:4,5]thieno[3,2-f]quinolin-8-one-trifluoroacetic acid (349.4 mg, 0.63 mmol) in DCM (5.0 mL) were added DIPEA (0.55 mL, 3.14 mmol) and followed by the additions of 2,2-dimethoxyacetaldehyde (0.11 mL, 0.75 mmol) and NaBH(OAc)₃ (332.6 mg, 1.57 mmol) at rt. The resulting mixture was stirred for 2 h at rt and then the solvent was evaporated under vacuum. The crude product was purified by reverse-phase chromatography (RPC) using H₂O with 0.1% formic acid (FA) and methanol as mobile phases to afford the desired product (262.0 mg, 79% yield) as a yellow solid. MS (ESI, m/z) [M+H]⁺ 531.2.

(R)-2-(4-(3-(10-Methyl-8-oxo-9,10,11,12-tetrahydro-8H-[1,4]diazepino[5′,6′:4,5]thieno[3,2-f]quinolin-3-yl)phenyl)piperidin-1-yl)acetaldehyde. To a solution of (R)-3-(3-(1-(2,2-dimethoxyethyl)piperidin-4-yl)phenyl)-10-methyl-9,10,11,12-tetrahydro-8H-[1,4]diazepino[5′,6′:4,5]thieno[3,2-f]quinolin-8-one (260.0 mg, 0.49 mmol) in 1,4-dioxane (2.0 mL), water (0.25 mL) and HCl (3.0 mL, 12.3 mmol) were added at rt. The reaction mixture was heated at 40° C. for 2 h and then continuously stirred at rt overnight. After the removal of the solvent under vacuum, the residue was further azeotropically evaporated with MeCN (5.0 mL) and DCM (5.0 mL) to afford the crude product (280.0 mg) as a red solid, which was used for the next step reaction without further purification. MS (ESI, m/z) [M+H]⁺ 485.2

3-(4-(4-(2-(4-(3-((R)-10-Methyl-8-oxo-9,10,11,12-tetrahydro-8H-[1,4]diazepino[5′,6′:4,5]thieno[3,2-f]quinolin-3-yl)phenyl)piperidin-1-yl)ethyl)piperazin-1-yl)phenyl)piperidine-2,6-dione. Under the protection of inert atmosphere, to a solution of (R)-2-(4-(3-(10-methyl-8-oxo-9,10,11,12-tetrahydro-8H-[1,4]diazepino[5′,6′:4,5]thieno[3,2-f]quinolin-3-yl)phenyl)piperidin-1-yl)acetaldehyde hydrochloride (140.0 mg, 0.27 mmol) and 3-(4-piperazin-1-ylphenyl)piperidine-2,6-dione;dihydrochloride (102.3 mg, 0.30 mmol) in DCM (1.5 mL) and DMSO (0.5 mL), DIPEA (0.33 mL, 1.88 mmol) was added and the resulting mixture was stirred for 10 min at rt. After the addition of NaBH(OAc)₃ (142.4 mg, 0.67 mmol), the reaction mixture was stirred at rt overnight. The solvent was evaporated under vacuum and the crude product was purified by reverse-phase chromatography (RPC) using water with 0.1% formic acid and acetonitrile (MeCN) as mobile phases to afford the desired product (49.5 mg, 24% yield) as a yellow solid. MS (ESI, m/z) [M+H]⁺ 742.4. ¹H NMR (400 MHz, DMSO-d₆) δ ppm 1.20 (d, J=6.6 Hz, 3H), 1.72-1.88 (m, 4H), 1.96-2.06 (m, 1H), 2.08-2.22 (m, 3H), 2.44 (br d, J=4.4 Hz, 1H), 2.53-2.69 (m, 10H), 3.07-3.13 (m, 6H), 3.48 (br s, 2H), 3.61 (dt, J=7.0, 3.6 Hz, 1H), 3.73 (dd, J=11.0, 4.9 Hz, 1H), 6.89 (d, J=8.8 Hz, 2H), 7.05 (d, J=8.8 Hz, 2H), 7.16 (t, J=5.1 Hz, 1H), 7.40 (d, J=7.8 Hz, 1H), 7.47-7.52 (m, 1H), 8.01-8.06 (m, 1H), 8.08 (d, J=4.2 Hz, 1H), 8.10-8.15 (m, 2H), 8.17 (s, 1H), 8.19 (s, 1H), 8.25 (d, J=9.0 Hz, 1H), 9.23 (d, J=8.8 Hz, 1H), 10.77 (s, 1H).

### Example 19: Synthesis of 3-(1-Methyl-6-(4-(2-(4-(3-((R)-10-methyl-8-oxo-9,10,11,12-tetrahydro-8H-[1,4]diazepino[5′,6′:4,5]thieno[3,2-f]quinolin-3-yl)phenyl)piperidin-1-yl)ethyl)piperazin-1-yl)-1H-indazol-3-yl)piperidine-2,6-dione

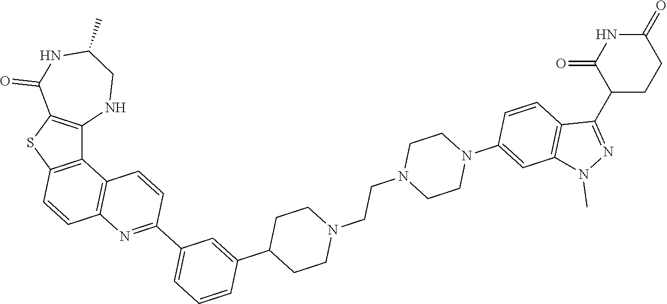 [SMILES: C[C@@H]1CNc2c(sc3ccc4nc(-c5cccc(C6CCN(CCN7CCN(c8ccc9c(C%10CCC(=O)NC%10=O)nn(C)c9c8)CC7)CC6)c5)ccc4c23)C(=O)N1]

3-(1-Methyl-6-(4-(2-(4-(3-((R)-10-methyl-8-oxo-9,10,11,12-tetrahydro-8H-[1,4]diazepino[5′,6′:4,5]thieno[3,2-f]quinolin-3-yl)phenyl)piperidin-1-yl)ethyl)piperazin-1-yl)-1H-indazol-3-yl)piperidine-2,6-dione. Under the protection of inert atmosphere, to a solution of (R)-2-(4-(3-(10-methyl-8-oxo-9,10,11,12-tetrahydro-8H-[1,4]diazepino[5′,6′:4,5]thieno[3,2-f]quinolin-3-yl)phenyl)piperidin-1-yl)acetaldehyde hydrochloride (140.0 mg, 0.27 mmol) and 3-(1-methyl-6-(piperazin-1-yl)-1H-indazol-3-yl)piperidine-2,6-dione dihydrochloride (102.3 mg, 0.30 mmol) in DCM (1.5 mL), DMSO (0.50 mL) and DIPEA (0.33 mL, 1.88 mmol) were added. After stirred at rt for 10 min, NaBH(OAc)₃ (142.4 mg, 0.67 mmol) was added, the resulting mixture was continuously stirred at rt overnight. The solvent was evaporated under vacuum and the crude product was purified by reverse-phase chromatography (RPC) using water with 0.1% formic acid and acetonitrile with 0.1% formic acid as mobile phases to afford the desired product (64 mg, 43.1% yield) as a yellow solid. MS (ESI, m/z) [M+H]⁺ 796.4. ¹H NMR (400 MHz, DMSO-d₆) δ ppm 1.20 (d, J=6.6 Hz, 3H), 1.74-1.89 (m, 4H), 2.13-2.24 (m, 3H), 2.25-2.34 (m, 1H), 2.55-2.68 (m, 11H), 3.12 (br d, J=11.5 Hz, 2H), 3.22-3.25 (m, 4H), 3.48 (br d, J=4.4 Hz, 2H), 3.62 (td, J=7.2, 3.4 Hz, 1H), 3.89 (s, 3H), 4.26 (dd, J=9.3, 5.1 Hz, 1H), 6.85 (d, J=1.7 Hz, 1H), 6.93 (dd, J=9.0, 1.7 Hz, 1H), 7.16 (t, J=5.3 Hz, 1H), 7.40 (d, J=7.8 Hz, 1H), 7.47-7.53 (m, 2H), 8.03 (d, J=9.0 Hz, 1H), 8.08 (d, J=4.2 Hz, 1H), 8.10-8.15 (m, 2H), 8.16 (s, 1H), 8.19-8.22 (m, 1H), 8.25 (d, J=9.0 Hz, 1H), 9.23 (d, J=9.0 Hz, 1H), 10.85 (s, 1H).

### Example 20: Synthesis of 3-(4-(1-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperidin-4-yl)phenyl)piperidine-2,6-dione

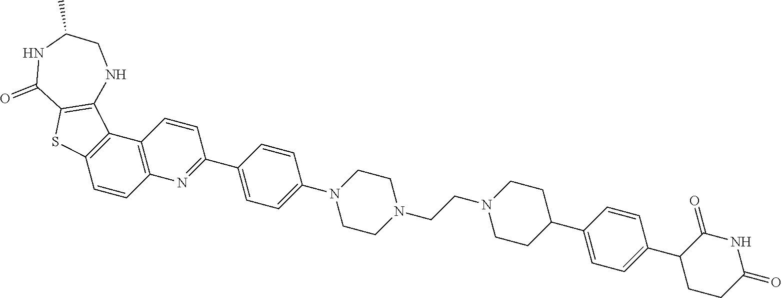 [SMILES: C[C@@H]1CNc2c(sc3ccc4nc(-c5ccc(N6CCN(CCN7CCC(c8ccc(C9CCC(=O)NC9=O)cc8)CC7)CC6)cc5)ccc4c23)C(=O)N1]

(R)-10-Methyl-3-(4-(piperazin-1-yl)phenyl)-9,10,11,12-tetrahydro-8H-[1,4]diazepino[5′,6′:4,5]thieno[3,2-f]quinolin-8-one. tert-Butyl 4-[4-(4,4,5,5-tetramethyl-1,3,2-dioxaborolan-2-yl)phenyl]piperazine-1-carboxylate (147 mg, 0.38 mmol), (R)-3-chloro-10-methyl-9,10,11,12-tetrahydro-8H-[1,4]diazepino[5′,6′:4,5]thieno[3,2-f]quinolin-8-one (100 mg, 0.31 mmol), cesium carbonate (307 mg, 0.94 mmol), and Pd(dppf)Cl₂ (35 mg, 0.05 mmol) were charged into 20 mL vial with a stir bar. The vial was sealed and repeated the evacuate-refill cycle with nitrogen three times, and then 1,4-dioxane (3 mL) and water (0.3 mL) were added. The resulting mixture was stirred and heated at 100° C. overnight. After cooling down to rt, the reaction was diluted with EtOAc and the aqueous layer was extracted with EtOAc (20 mL×3). The organic layers were dried over sodium sulfate, filtered, and concentrated. The residue was purified by silica gel column chromatography (0% to 5% MeOH in DCM) to afford the expected product which is further converted into TFA salt by stirring in 4:1 DCM:TFA at rt for 1 h. After removed the solvent, the residue was purified by reverse-phase chromatography (RPC) using water with 0.1% formic acid (FA) and acetonitrile (MeCN) as mobile phases to afford the desired product, (R)-10-Methyl-3-(4-(piperazin-1-yl)phenyl)-9,10,11,12-tetrahydro-8H-[1,4]diazepino[5′,6′:4,5]thieno[3,2-f]quinolin-8-one (60 mg, 43% yield), as an orange solid. MS (ESI, m/z) [M+H]⁺ 444.5.

(R)-3-(4-(4-(2,2-dimethoxyethyl)piperazin-1-yl)phenyl)-10-methyl-9,10,11,12-tetrahydro-8H-[1,4]diazepino[5′,6′:4,5]thieno[3,2-f]quinolin-8-one. To a solution of (R)-10-Methyl-3-(4-(piperazin-1-yl)phenyl)-9,10,11,12-tetrahydro-8H-[1,4]diazepino[5′,6′:4,5]thieno[3,2-f]quinolin-8-one dihydrochloride (120 mg, 0.23 mmol, 1.0 equiv.) and DIPEA (0.2 mL, 1.2 mmol) in CH₂Cl₂ (3 mL) and DMSO (1 mL) were added 2,2-dimethoxyacetaldehyde (42 μL, 0.28 mmol) and NaBH(OAc)₃ (148 mg, 0.70 mmol) at rt. The mixture was stirred at rt overnight. LCMS showed the reaction was completed. The reaction mixture was concentrated under vacuum, the residue was purified by reverse phase column chromatography (0% to 100% MeOH in water w/ 0.1% formic acid) to afford the product, (R)-10-methyl-3-(4-(piperazin-1-yl)phenyl)-9,10,11,12-tetrahydro-8H-[1,4]diazepino[5′,6′:4,5]thieno[3,2-f]quinolin-8-one (113 mg, 86% yield) as a red solid. MS (ESI, m/z) [M+2H]²⁺ 266.8.

(R)-2-(4-(4-(10-Methyl-8-oxo-9,10,11,12-tetrahydro-8H-[1,4]diazepino[5′,6′:4,5]thieno[3,2-f]quinolin-3-yl)phenyl)piperazin-1-yl)acetaldehyde. To a round-bottom flask containing (R)-10-methyl-3-(4-(piperazin-1-yl)phenyl)-9,10,11,12-tetrahydro-8H-[1,4]diazepino[5′,6′:4,5]thieno[3,2-f]quinolin-8-one (160 mg, 0.30 mmol) was added water (0.3 mL) and 4 M HCl in 1,4-dioxane (1.1 mL, 4.5 mmol) at rt. The mixture was stirred at 50° C. After 5 h, LCMS showed the reaction was completed. After removing the solvent under vacuum, the residue was washed by MeCN and dried under vacuum to afford (R)-2-(4-(4-(10-Methyl-8-oxo-9,10,11,12-tetrahydro-8H-[1,4]diazepino[5′,6′:4,5]thieno[3,2-f]quinolin-3-yl)phenyl)piperazin-1-yl)acetaldehyde hydrochloride (160 mg, quant.) as a yellow solid. MS (ESI, m/z) [M+2H]⁺ 243.8.

3-(4-(1-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperidin-4-yl)phenyl)piperidine-2,6-dione. (R)-2-(4-(4-(10-Methyl-8-oxo-9,10,11,12-tetrahydro-8H-[1,4]diazepino[5′,6′:4,5]thieno[3,2-f]quinolin-3-yl)phenyl)piperazin-1-yl)acetaldehyde (80.0 mg, 0.17 mmol) in methanol (1.0 mL) was added 3-[4-(4-piperidyl)phenyl]piperidine-2,6-dione (67 mg, 0.25 mmol) and NaBH₃CN (20 mg, 0.33 mmol). The resulting solution was stirred at rt for 18 h. LCMS showed the reaction was completed. The resulting solution was concentrated under vacuum and the residue was purified by reverse-phase chromatography (RPC) using water with 0.05% trifluoroacetic acid (TFA) and acetonitrile (MeCN) as mobile phases to afford 3-(4-(1-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperidin-4-yl)phenyl)piperidine-2,6-dione (2.5 mg, 2.0% yield) as a red solid. MS (ESI, m/z) [M+H]⁺ 742. ¹H NMR (400 MHz, DMSO-d₆) δ ppm 9.30-9.20 (m, 1H), 8.30-8.15 (m, 4H), 8.13-7.99 (m, 1H), 7.32-7.15 (m, 6H), 3.76-3.74 (m, 3H), 3.65-3.58 (m, 7H), 3.56-3.40 (m, 3H), 3.39-3.25 (m, 5H), 3.24-3.16 (m, 3H), 2.99-2.80 (m, 2H), 2.71-2.69 (m, 1H), 2.21-2.01 (m, 5H), 1.97-1.90 (m, 1H), 1.22-1.20 (m, 3H). ¹⁹F NMR (376 MHz, DMSO-d₆) δ −74.53.

### Example 21: Synthesis of 3-(1-Methyl-6-(4-(2-(4-(4-((R)-10-methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperazin-1-yl)-1H-indazol-3-yl)piperidine-2,6-dione

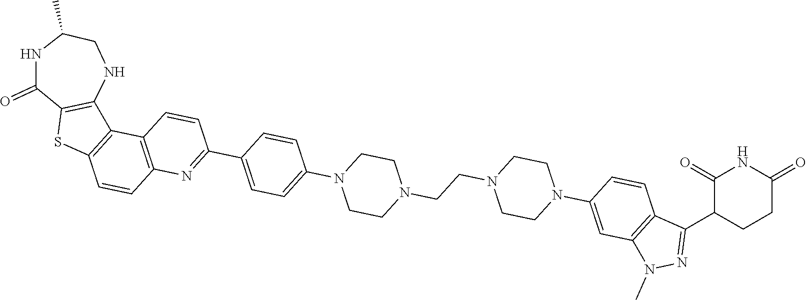 [SMILES: C[C@@H]1CNc2c(sc3ccc4nc(-c5ccc(N6CCN(CCN7CCN(c8ccc9c(C%10CCC(=O)NC%10=O)nn(C)c9c8)CC7)CC6)cc5)ccc4c23)C(=O)N1]

3-(1-Methyl-6-(4-(2-(4-(4-((R)-10-methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperazin-1-yl)-1H-indazol-3-yl)piperidine-2,6-dione. To a suspension of (R)-2-(4-(4-(10-methyl-8-oxo-9,10,11,12-tetrahydro-8H-[1,4]diazepino[5′,6′:4,5]thieno[3,2-f]quinolin-3-yl)phenyl)piperazin-1-yl)acetaldehyde hydrochloride (155 mg, 0.3 mmol) and DIPEA (360 μL, 2.1 mmol) in CH₂Cl₂ (2.3 mL) and DMSO (0.8 mL) were added 3-(1-methyl-6-piperazin-1-yl-indazol-3-yl)piperidine-2,6-dione hydrochloride (119 mg, 0.33 mmol) and NaBH(OAc)₃ (130 mg, 0.6 mmol) at rt. The reaction was then stirred at rt overnight. After removing the solvent under vacuum, the residue was purified by reverse phase column chromatography (0% to 100% MeCN in water w/ 0.1% formic acid) to afford 3-(1-methyl-6-(4-(2-(4-(4-((R)-10-methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperazin-1-yl)-1H-indazol-3-yl)piperidine-2,6-dione (22 mg, 9% yield) as a yellow solid. MS (ESI, m/z) [M+H]⁺ 797.3. ¹H NMR (400 MHz, DMSO-d₆) δ ppm 10.85 (s, 1H), 9.15 (d, J=9.0 Hz, 1H), 8.19 (d, J=9.0 Hz, 2H), 8.14 (d, J=9.3 Hz, 1H), 8.04-8.11 (m, 2H), 7.96 (d, J=9.0 Hz, 1H), 7.50 (d, J=8.8 Hz, 1H), 7.06-7.15 (m, 3H), 6.93 (br d, J=8.6 Hz, 1H), 6.85 (s, 1H), 4.26 (dd, J=9.0, 4.9 Hz, 1H), 3.89 (s, 3H), 3.57-3.65 (m, 1H), 3.44-3.50 (m, 2H), 3.20-3.25 (m, 4H), 2.58-2.65 (m, 10H), 2.53-2.58 (m, 4H), 2.24-2.32 (m, 1H), 2.10-2.20 (m, 1H), 1.20 (d, J=6.6 Hz, 3H).

### Example 22: Synthesis of 3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperazin-1-yl)phenyl)piperidine-2,6-dione

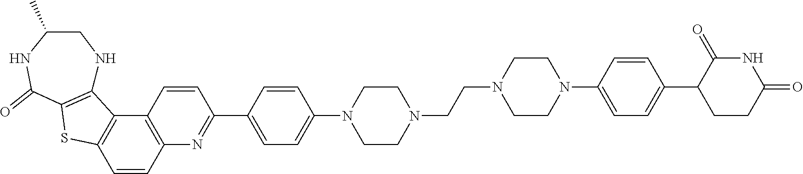 [SMILES: C[C@@H]1CNc2c(sc3ccc4nc(-c5ccc(N6CCN(CCN7CCN(c8ccc(C9CCC(=O)NC9=O)cc8)CC7)CC6)cc5)ccc4c23)C(=O)N1]

3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)ethyl)piperazin-1-yl)phenyl)piperidine-2,6-dione. To a suspension of (R)-2-(4-(4-(10-methyl-8-oxo-9,10,11,12-tetrahydro-8H-[1,4]diazepino[5′,6′:4,5]thieno[3,2-f]quinolin-3-yl)phenyl)piperazin-1-yl)acetaldehyde hydrochloride (160 mg, 0.31 mmol) and DIPEA (370 μL, 2.15 mmol) in CH₂Cl₂ (1.5 mL) and DMSO (0.5 mL) were added 3-(4-piperazin-1-ylphenyl)piperidine-2,6-dione dihydrochloride (117 mg, 0.34 mmol) and NaBH(OAc)₃ (130 mg, 0.61 mmol) at rt. The reaction was then stirred at rt overnight. After removed the solvent under vacuum, the residue was purified by reverse phase column chromatography (0% to 100% MeCN in water w/ 0.1% formic acid) to afford the product, 3-(4-(4-(2-(4-(4-((R)-10-Methyl-8-oxo-9,10,11,12-tetrahydro-8H-[1,4]diazepino[5′,6′:4,5]thieno[3,2-(]quinolin-3-yl)phenyl)piperazin-1-yl)ethyl)piperazin-1-yl)phenyl)piperidine-2,6-dione (37 mg, 16% yield) as a yellow solid. MS (ESI, m/z) [M+H]⁺ 743.3. ¹H NMR (400 MHz, DMSO-d₆) δ ppm 10.77 (s, 1H), 9.14 (d, J=9.0 Hz, 1H), 8.19 (d, J=8.8 Hz, 2H), 8.03-8.16 (m, 3H), 7.95 (d, J=8.8 Hz, 1H), 7.02-7.13 (m, 5H), 6.89 (d, J=8.6 Hz, 2H), 3.72 (dd, J=10.9, 4.8 Hz, 1H), 3.57-3.65 (m, 1H), 3.47 (br s, 2H), 3.25-3.28 (m, 3H), 3.08-3.15 (m, 4H), 2.55-2.64 (m, 9H), 2.54 (s, 4H), 2.42-2.47 (m, 2H), 2.08-2.20 (m, 1H), 1.96-2.05 (m, 1H), 1.20 (d, J=6.6 Hz, 3H).

### Example 23: Synthesis of 3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetyl)piperazin-1-yl)phenyl)piperidine-2,6-dione

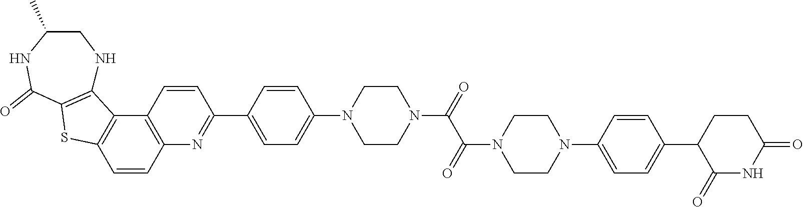 [SMILES: C[C@@H]1CNc2c(sc3ccc4nc(-c5ccc(N6CCN(C(=O)C(=O)N7CCN(c8ccc(C9CCC(=O)NC9=O)cc8)CC7)CC6)cc5)ccc4c23)C(=O)N1]

(R)-2-(4-(4-(10-Methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetic acid. A 20 mL vial equipped with stir bar was filled with (R)-10-methyl-3-(3-(piperazin-1-yl)phenyl)-9,10,11,12-tetrahydro-8H-[1,4]diazepino[5′,6′:4,5]thieno[3,2-f]quinolin-8-one trifluoroacetate salt (100 mg, 0.18 mmol). DCM/DMSO (5 mL, 10:1) was then added followed by DIPEA (125 μL, 0.72 mmol) and ethyl 2-chloro-2-oxoacetate (21 μL, 0.19 mmol). The resulting reaction was allowed to stir at rt for 2 h, LCMS indicated the reaction was completed. The reaction mixture was concentrated under vacuum, lithium hydroxide (21.5 mg, 0.9 mmol) was then added, followed by THF/water (5 mL, 3:2) and stirred for 1.0 h. After removed the solvent, the residue was purified by reverse phase column chromatography (0% to 100% MeCN in water w/ 0.1% TFA), affording the product (R)-2-(4-(3-(10-methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetic acid trifluoroacetate salt as a red solid.

3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetyl)piperazin-1-yl)phenyl)piperidine-2,6-dione. In a 2-dram vial, DMF (1.5 mL) was charged and followed by (R)-2-(4-(4-(10-methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetic acid (8.8 mg, 0.017 mmol) and HATU (0.068 mL, 0.034 mmol). To the resulting mixture, 3-(4-(piperazin-1-yl)phenyl)piperidine-2,6-dione (0.075 mL, 0.019 mmol) was added. After the addition of DIPEA (8.9 μl, 0.05 mmol), the reaction mixture was allowed to stir at rt for overnight. After the filtration of reaction mixture, the filtrate was purified by reverse-phase chromatography (RPC) using H₂O and acetonitrile (both with 0.05% TFA) as mobile phases to afford 3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)piperazin-1-yl)-2-oxoacetyl)piperazin-1-yl)phenyl)piperidine-2,6-dione (3.0 mg, 24% yield). LCMS Method 4: MS (ESI, m/z) [M+H]⁺ 771.5, r.t.: 1.21 min.

### Example 24: Synthesis of 3-(4-(4-(2-(3-(4-((R)-10-Methyl-8-oxo-9,10,11,12-tetrahydro-8H-[1,4]diazepino[5′,6′:4,5]thieno[3,2-f]quinolin-3-yl)phenyl)-3,6-diazabicyclo[3.1.1]heptan-6-yl)ethyl)piperazin-1-yl)phenyl)piperidine-2,6-dione

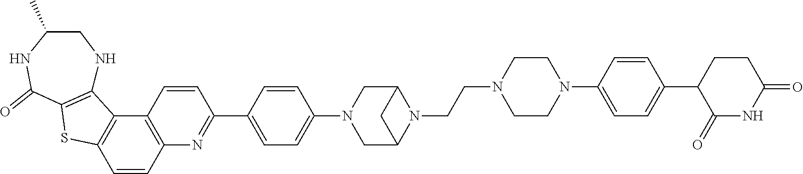 [SMILES: C[C@@H]1CNc2c(sc3ccc4nc(-c5ccc(N6CC7CC(C6)N7CCN6CCN(c7ccc(C8CCC(=O)NC8=O)cc7)CC6)cc5)ccc4c23)C(=O)N1]

tert-Butyl 3-(4-bromophenyl)-3,6-diazabicyclo[3.1.1]heptane-6-carboxylate. To a solution of tert-butyl 3,6-diazabicyclo[3.1.1]heptane-6-carboxylate (1.0 g, 5.0 mmol), 1-bromo-4-iodo-benzene (2.1 g, 7.6 mmol) and t-BuONa (968.0 mg, 10.1 mmol) in toluene (15.0 mL) was added XantPhos (258 mg, 0.5 mmol) and Pd₂(dba)₃ (520.0 mg, 0.5 mmol). The resulting solution was stirred at 60° C. for 18 h under a nitrogen atmosphere. LCMS showed the reaction was completed. The reaction was diluted with water (100 mL) and extracted with ethyl acetate (100 mL×3). The combined organic layer was washed with brine (50 mL×2), dried over anhydrous sodium sulfate, filtered, and concentrated under vacuum. The residue was purified by column chromatography on silica gel with ethyl acetate/petroleum ether (1:4) to afford the desired product tert-butyl 3-(4-bromophenyl)-3,6-diazabicyclo[3.1.1]heptane-6-carboxylate (1.6 g, 90.0%) as a yellow solid. MS (ESI, m/z) [M+H]⁺ 353.2.

3-(4-Bromophenyl)-3,6-diazabicyclo[3.1.1]heptane. To a solution of tert-butyl 3-(4-bromophenyl)-3,6-diazabicyclo[3.1.1]heptane-6-carboxylate (1.6 g, 4.5 mmol) in DCM (30.0 mL) was added dropwise TFA (10.0 mL). The resulting solution was stirred at rt for 1 h. LCMS showed the reaction was completed. The resulting solution was concentrated under vacuum. The residue was purified by reverse flash chromatography on silica gel with water (0.05% TFA)/MeCN (1:1) to afford 3-(4-bromophenyl)-3,6-diazabicyclo[3.1.1]heptane (1.1 g, 96.0%) as a white solid. MS (ESI, m/z) [M+H]⁺ 254.1.

3-(4-Bromophenyl)-6-(2,2-dimethoxyethyl)-3,6-diazabicyclo[3.1.1]heptane. To a solution of 3-(4-bromophenyl)-3,6-diazabicyclo[3.1.1]heptane (1.0 g, 4.0 mmol) in methanol (20.0 mL) was added 2,2-dimethoxyacetaldehyde (1.2 g, 11.9 mmol) and NaBH₃CN (756.0 mg, 11.9 mmol). The resulting solution was stirred at rt for 18 h. LCMS showed the reaction was completed. The resulting solution was concentrated under vacuum, and the residue was purified by reverse flash chromatography on silica gel with water (0.05% TFA)/MeCN (1:1) to afford the desired product, 3-(4-bromophenyl)-6-(2,2-dimethoxyethyl)-3,6-diazabicyclo[3.1.1]heptane (1.3 g, 96%) as a yellow oil. MS (ESI, m/z) [M+H]⁺ 341.3.

(10R)-3-(4-(6-(2,2-dimethoxyethyl)-3,6-diazabicyclo[3.1.1]heptan-3-yl)phenyl)-10-methyl-9,10,11,12-tetrahydro-8H-[1,4]diazepino[5′,6′:4,5]thieno[3,2-f]quinolin-8-one. To a solution of 3-(4-bromophenyl)-6-(2,2-dimethoxyethyl)-3,6-diazabicyclo[3.1.1]heptane (200.0 mg, 0.6 mmol), (R)-10-methyl-3-(tributylstannyl)-9,10,11,12-tetrahydro-8H-[1,4]diazepino[5′,6′:4,5]thieno[3,2-f]quinolin-8-one (503.0 mg, 0.9 mmol) in 1,4-dioxane (10.0 mL) was added XPhos Pd G₃ (50.0 mg, 0.05 mmol) and XPhos (50 mg, 0.10 mmol). The resulting solution was stirred at 90° C. for 5 h under a nitrogen atmosphere. LCMS showed the reaction was completed. The resulting solution was concentrated under vacuum, and the residue was by reverse flash chromatography on silica gel with water (0.05% TFA)/MeCN (3:2) to afford the desired product, (10R)-3-(4-(6-(2,2-dimethoxyethyl)-3,6-diazabicyclo[3.1.1]heptan-3-yl)phenyl)-10-methyl-9,10,11,12-tetrahydro-8H-[1,4]diazepino[5′,6′:4,5]thieno[3,2-f]quinolin-8-one (180.0 mg, 56.0%) as a red solid. MS (ESI, m/z) [M+H]⁺ 544.1.

2-(3-(4-((R)-10-Methyl-8-oxo-9,10,11,12-tetrahydro-8H-[1,4]diazepino[5′,6′:4,5]thieno[3,2-f]quinolin-3-yl)phenyl)-3,6-diazabicyclo[3.1.1]heptan-6-yl)acetaldehyde. To a solution of (10R)-3-(4-(6-(2,2-dimethoxyethyl)-3,6-diazabicyclo[3.1.1]heptan-3-yl)phenyl)-10-methyl-9,10,11,12-tetrahydro-8H-[1,4]diazepino[5′,6′:4,5]thieno[3,2-f]quinolin-8-one (90 mg, 0.170 mmol, 1.00 equiv) in 1,4-dioxane (2.0 mL) and water (1.0 mL) was added 4 M HCl in 1,4-dioxane (2.0 mL). The resulting solution was stirred at 40° C. for 18 h. LCMS showed the reaction was completed. The resulting solution was concentrated under vacuum to afford crude product which was used directly for the next step without further purification. MS (ESI, m % z) [M+H+18]⁺ 544.

3-(4-(4-(2-(3-(4-((R)-10-Methyl-8-oxo-9,10,11,12-tetrahydro-8H-[1,4]diazepino[5′,6′:4,5]thieno[3,2-f]quinolin-3-yl)phenyl)-3,6-diazabicyclo[3.1.1]heptan-6-yl)ethyl)piperazin-1-yl)phenyl)piperidine-2,6-dione. To a solution of 3-(4-piperazin-1-ylphenyl)piperidine-2,6-dione (49.0 mg, 0.18 mmol) in methanol (3.0 mL) was added 2-(3-(4-((R)-10-methyl-8-oxo-9,10,11,12-tetrahydro-8H-[1,4]diazepino[5′,6′:4,5]thieno[3,2-f]quinolin-3-yl)phenyl)-3,6-diazabicyclo[3.1.1]heptan-6-yl)acetaldehyde (90.0 mg, 0.18 mmol) and NaBH₃CN (34.0 mg, 0.5 mmol). The resulting solution was stirred at rt for 5 h and then concentrated under vacuum. The residue was dissolved into DMSO (2.0 mL) and filtered, the filtrate was purified by reverse-phase chromatography (RPC) using water and acetonitrile (MeCN) (both with 0.05% trifluoroacetic acid) as mobile phases to afford the desired product, 3-(4-(4-(2-(3-(4-((R)-10-Methyl-8-oxo-9,10,11,12-tetrahydro-8H-[1,4]diazepino[5′,6′:4,5]thieno[3,2-f]quinolin-3-yl)phenyl)-3,6-diazabicyclo[3.1.1]heptan-6-yl)ethyl)piperazin-1-yl)phenyl)piperidine-2,6-dione (46.0 mg, 32.0%) as a red solid. MS (ESI, m/z) [M+H]⁺ 755. ¹H NMR (400 MHz, DMSO-d₆) δ ppm 9.25 (d, J=9.2 Hz, 1H), 8.26 (d, J=8.0 Hz, 2H), 8.26-8.17 (m, 2H), 8.04 (d, J=9.2 Hz, 1H), 7.10 (d, J=8.0 Hz, 2H), 7.00 (d, J=9.2 Hz, 2H), 4.69-4.54 (m, 2H), 4.17-3.84 (m, 5H), 3.82-3.73 (m, 1H), 3.57-3.21 (m, 15H), 2.72-2.61 (m, 1H), 2.49-2.42 (m, 1H), 2.18-2.05 (m, 2H), 2.06-1.96 (m, 1H), 1.16 (d, J=6.8 Hz, 3H). ¹⁹F NMR (376 MHz, DMSO-d₆) δ −74.28.

### Example 25: Synthesis of 3-(4-(4-(2-(4-(4-((R)-10-Methyl-8-oxo-8,9,10,11-tetrahydro-[1,4]oxazepino[7′,6′:4,5]thieno[3,2-f]quinolin-3-yl)phenyl)piperazin-1-yl)ethyl)piperazin-1-yl)phenyl)piperidine-2,6-dione

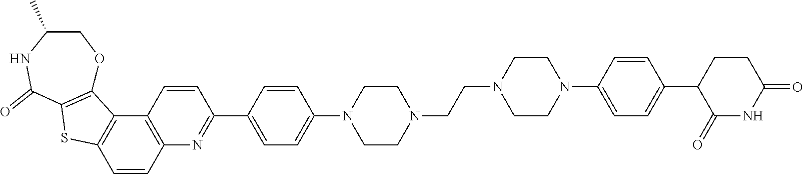 [SMILES: C[C@@H]1COc2c(sc3ccc4nc(-c5ccc(N6CCN(CCN7CCN(c8ccc(C9CCC(=O)NC9=O)cc8)CC7)CC6)cc5)ccc4c23)C(=O)N1]

1-(4-Bromophenyl)-4-(2,2-dimethoxyethyl)piperazine. To a solution of NaBH₃CN (796 mg, 12.44 mmol) in methanol (10.0 mL) was added 1.9 M ZnCl₂ in THF (15.8 mL, 4.14 mmol). The mixture was stirred at rt for 10 min. To the solution were added 1-(4-bromophenyl)piperazine (1.0 g, 4.14 mmol) and 2,2-dimethoxyacetaldehyde (520 mg, 5.01 mmol). The resulting solution was stirred at rt overnight. LCMS showed the reaction was completed. The resulting was concentrated under vacuum. The residue was purified by reverse flash with water (0.05% TFA)/MeCN(2:3) to afford 1-(4-bromophenyl)-4-(2,2-dimethoxyethyl)piperazine (1.1 g, 80%) as a colorless oil. LCMS (ESI, m/z) [M+H]⁺ 329.1

1-(2,2-Dimethoxyethyl)-4-[4-(4,4,5,5-tetramethyl-1,3,2-dioxaborolan-2-yl)phenyl]piperazine. To a solution of 1-(4-bromophenyl)-4-(2,2-dimethoxyethyl)piperazine (550 mg, 1.67 mmol) in 1,4-dioxane (10.0 mL) were added B₂pin₂ (636 mg, 2.50 mmol), Pd(dppf)Cl₂ (136 mg, 0.167 mmol) and KOAc (491 mg, 5.01 mmol). The resulting solution was stirred at 100° C. overnight under vacuum. LCMS showed the reaction was completed. The resulting solution was diluted with water (50.0 mL) and extracted with ethyl acetate (30 mL×3). The combined organic layer was washed with brine (30 mL×2), dried over anhydrous sodium sulfate, filtered and concentrated under vacuum. The residue was purified by silica gel chromatography with ethyl acetate/petroleum ether (1:1) to afford 1-(2,2-dimethoxyethyl)-4-[4-(4,4,5,5-tetramethyl-1,3,2-dioxaborolan-2-yl)phenyl]piperazine (370 mg, 59%) as a yellow solid. LCMS (ESI, m/z) [M+H]⁺ 377.

(R)-3-(4-(4-(2,2-Dimethoxyethyl)piperazin-1-yl)phenyl)-10-methyl-10,11-dihydro-[1,4]oxazepino[7′,6′:4,5]thieno[3,2-f]quinolin-8(9H)-one. To a solution of 1-(2,2-dimethoxyethyl)-4-[4-(4,4,5,5-tetramethyl-1,3,2-dioxaborolan-2-yl)phenyl]piperazine (100 mg, 0.265 mmol) in 1,4-dioxane (3.0 mL) and water (0.5 mL) were added (R)-3-(4-chlorophenyl)-10-methyl-10,11-dihydro-[1,4]oxazepino[7′,6′:4,5]thieno[3,2-f]quinolin-8(9H)-one (166 mg, 0.398 mmol), Pd(PPh₃)₄(31 mg, 0.027 mmol) and K₂CO₃ (110 mg, 0.795 mmol). The resulting solution was stirred at 100° C. overnight. LCMS showed the reaction was completed. The resulting solution was diluted with water (50.0 mL) and extracted with ethyl acetate (30 mL×3). The combined organic layer was washed with brine (30 mL×2), dried over anhydrous sodium sulfate, filtered, and concentrated under vacuum. The residue was purified by silica gel chromatography with ethyl acetate/petroleum ether (2:1) tert-butyl (R)-3-(4-(4-(2,2-dimethoxyethyl)piperazin-1-yl)phenyl)-10-methyl-8-oxo-10,11-dihydro-[1,4]oxazepino[7′,6′:4,5]thieno[3,2-f]quinoline-9(8H)-carboxylate (100 mg, 60%) as a yellow solid. LCMS (ESI, m/z) [M+H]⁺ 633.1.

(R)-2-(4-(4-(10-methyl-8-oxo-8,9,10,11-tetrahydro-[1,4]oxazepino[7′,6′:4,5]thieno[3,2-f]quinolin-3-yl)phenyl)piperazin-1-yl)acetaldehyde. To a solution of tert-butyl (R)-3-(4-(4-(2,2-dimethoxyethyl)piperazin-1-yl)phenyl)-10-methyl-8-oxo-10,11-dihydro-[1,4]oxazepino[7′,6′:4,5]thieno[3,2-f]quinoline-9(8H)-carboxylate (100 mg, 0.158 mmol) in 1,4-dioxane (4.0 mL) was added 4 M HCl (aq.) (10 mL). The resulting solution was stirred at 40° C. for 2 h. LCMS showed the reaction was completed. The resulting solution was concentrated under vacuum to afford crude (R)-2-(4-(4-(10-methyl-8-oxo-8,9,10,11-tetrahydro-[1,4]oxazepino[7′,6′:4,5]thieno[3,2-f]quinolin-3-yl)phenyl)piperazin-1-yl)acetaldehyde (80 mg) was used directly for next step without further purification. MS (ESI, m/z) [M+H]⁺ 487.2.

3-(4-(4-(2-(4-(4-((R)-10-Methyl-8-oxo-8,9,10,11-tetrahydro-[1,4]oxazepino[7′,6′:4,5]thieno[3,2-f]quinolin-3-yl)phenyl)piperazin-1-yl)ethyl)piperazin-1-yl)phenyl)piperidine-2,6-dione. To a solution of 3-(1-methyl-6-piperazin-1-yl-indazol-3-yl)piperidine-2,6-dione (67 mg, 0.21 mmol) in DCM (5.0 mL), the pH value of the solution was adjusted to 9˜10 with TEA. Then the (R)-2-(4-(4-(10-methyl-8-oxo-8,9,10,11-tetrahydro-[1,4]oxazepino[7′,6′:4,5]thieno[3,2-f]quinolin-3-yl)phenyl)piperazin-1-yl)acetaldehyde (80 mg, 0.164 mmol) was added the resulting solution and the pH value of the solution was then adjusted to 5˜6 with HOAc. Then the NaBH(OAc)₃ (130 mg, 0.62 mmol) was added the resulting solution and stirred at rt for 2 h. LCMS showed the reaction was completed. The resulting solution was concentrated under vacuum. The residue was purified by reverse-phase chromatography (RPC) using H₂O and acetonitrile (both with 0.05% TFA) as mobile phases to afford the desired product 3-(4-(4-(2-(4-(4-((R)-10-Methyl-8-oxo-8,9,10,11-tetrahydro-[1,4]oxazepino[7′,6′:4,5]thieno[3,2-f]quinolin-3-yl)phenyl)piperazin-1-yl)ethyl)piperazin-1-yl)phenyl)piperidine-2,6-dione (25.1 mg, 14% yield) as an orange solid. Analytic Conditions: Column: SPM20A C18 100 A Column 4.6*100 mm, 3.5 um; Mobile Phase A: Water/0.05% TFA, Mobile Phase B: Acetonitrile/0.05% TFA; Flow rate: 1.5000 mL/min; Gradient: 10% B to 40% B in 6 min; 254 nm; Rt: 4.751 min. MS (ESI, m/z) [M+H]⁺ 743. ¹H NMR (400 MHz, DMSO-d): δ ppm 9.33 (d, J=8.8 Hz, 1H), 8.23-8.18 (m, 4H), 8.07 (d, J=8.8 Hz, 1H), 7.20 (d, J=8.8 Hz, 2H), 7.12 (d, J=8.8 Hz, 2H), 6.99 (d, J=8.8 Hz, 2H), 4.62 (s, 2H), 3.90-3.70 (m, 15H), 3.61-3.42 (m, 7H), 2.72-2.60 (m, 1H), 2.45-2.43 (m, 1H), 2.25-2.10 (m, 1H), 2.10-1.95 (m, 1H), 1.30 (d, J=6.4 Hz, 3H).

### Example 26: Synthesis of 3-(1-Methyl-6-(4-(2-(4-(4-((R)-10-ethyl-8-oxo-8,9,10,11-tetrahydro-[1,4]oxazepino[7′,6′:4,5]thieno[3,2-f]quinolin-3-yl)phenyl)piperazin-1-yl)ethyl)piperazin-1-yl)-1H-indazol-3-yl)piperidine-2,6-dione

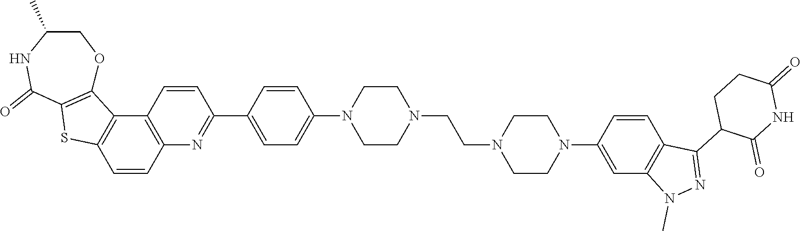 [SMILES: C[C@@H]1COc2c(sc3ccc4nc(-c5ccc(N6CCN(CCN7CCN(c8ccc9c(C%10CCC(=O)NC%10=O)nn(C)c9c8)CC7)CC6)cc5)ccc4c23)C(=O)N1]

3-(1-Methyl-6-(4-(2-(4-(4-((R)-10-ethyl-8-oxo-8,9,10,11-tetrahydro-[1,4]oxazepino[7′,6′:4,5]thieno[3,2-f]quinolin-3-yl)phenyl)piperazin-1-yl)ethyl)piperazin-1-yl)-1H-indazol-3-yl)piperidine-2,6-dione. A 20 mL vial equipped with stir bar was charged with (R)-2-(4-(4-(10-methyl-8-oxo-8,9,10,11-tetrahydro-[1,4]oxazepino[7′,6′:4,5]thieno[3,2-f]quinolin-3-yl)phenyl)piperazin-1-yl)acetaldehyde (36.5 mg, 0.075 mmol), 1,2-dichloroethane (1.0 mL) and THF (1.0 mL) were added. DIPEA (0.039 mL, 0.23 mmol) and 3-(1-methyl-6-(piperazin-1-yl)-1H-indazol-3-yl)piperidine-2,6-dione (0.36 mL, 0.090 mmol) were added, and followed by sodium triacetoxyborohydride (0.60 mL, 0.30 mmol). The resulting mixture was stirred at 23° C., overnight. Another 2 equiv. of NaBH(OAc)₃ was added, the reaction was heated at 50° C. and stirred for 4.0 h. After removal of the solvent, the residue was dissolved into 1.5 mL of DMSO. After filtration, the solution was purified by reverse phase preparatory HPLC (0% to 100% MeCN in water w/ 0.1% formic acid to afford the product, 3-(1-methyl-6-(4-(2-(4-(4-((R)-10-methyl-8-oxo-8,9,10,11-tetrahydro-[1,4]oxazepino[7′,6′:4,5]thieno[3,2-f]quinolin-3-yl)phenyl)piperazin-1-yl)ethyl)piperazin-1-yl)-1H-indazol-3-yl)piperidine-2,6-dione (2.0 mg, 3.0% yield). MS (ESI, m/z) [M+H]⁺ 798.5. ¹H NMR (400 MHz, CDCl₃) δ ppm 9.31-9.16 (m, 1H), 8.23-8.08 (m, 3H), 8.02-7.83 (m, 3H), 7.22-7.05 (m, 3H), 7.03-6.86 (m, 2H), 6.19-6.07 (m, 1H), 4.78-4.66 (m, 1H), 4.59-4.47 (m, 1H), 4.07-3.98 (m, 1H), 3.80-3.65 (m, 4H), 3.54-3.26 (m, 8H), 3.07-2.60 (m, 13H), 2.34-2.17 (m, 3H), 1.54-1.44 (m, 3H).

### Example 27: Synthesis of 3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)piperidin-1-yl)ethyl)piperazin-1-yl)phenyl)piperidine-2,6-dione

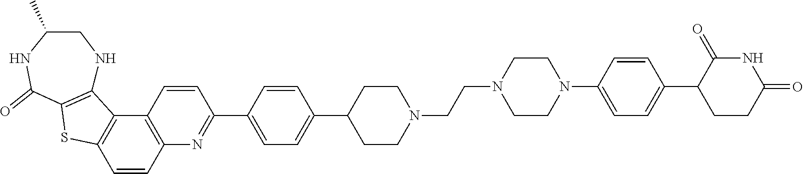 [SMILES: C[C@@H]1CNc2c(sc3ccc4nc(-c5ccc(C6CCN(CCN7CCN(c8ccc(C9CCC(=O)NC9=O)cc8)CC7)CC6)cc5)ccc4c23)C(=O)N1]

(R)-10-Methyl-3-(4-(piperidin-4-yl)phenyl)-9,10,11,12-tetrahydro-8H-[1,4]diazepino[5′,6′:4,5]thieno[3,2-f]quinolin-8-one. tert-Butyl 4-(4-(4,4,5,5-tetramethyl-1,3,2-dioxaborolan-2-yl)phenyl)piperidine-1-carboxylate (145 mg, 0.38 mmol), (R)-3-chloro-10-methyl-9,10,11,12-tetrahydro-8H-[1,4]diazepino[5′,6′:4,5]thieno[3,2-f]quinolin-8-one (100 mg, 0.31 mmol), cesium carbonate (306 mg, 0.94 mmol), and Pd(dppf)Cl₂ (23 mg, 0.03 mmol) were weighed into a 20 mL vial with a stir bar. The vial was evacuated and back-filled with nitrogen three times, 1,4-dioxane (3.1 mL) and water (0.3 mL) were added to the vial. The reaction was then heated at 100° C. for 2 h. LCMS analysis revealed that the reaction was completed. The reaction was then diluted with EtOAc and transferred to a separatory funnel containing saturated aqueous NaHCO₃. The organic layer was collected, and the aqueous layer was extracted with EtOAc (20 mL×3). The organic layers were dried over sodium sulfate, filtered, and concentrated under vacuum. The residue was purified by normal phase column chromatography (0 to 10% MeOH in DCM). After concentrating under vacuum, the solid was dissolved into 4:1 DCM/TFA and stirred for 1.5 h at rt. LCMS indicated that the deprotection was completed. After removed the solvent, the residue was purified using reverse phase column chromatography (0 to 100% MeCN in water w/ 0.1% TFA) to afford the product, (R)-10-methyl-3-(4-(piperidin-4-yl)phenyl)-9,10,11,12-tetrahydro-8H-[1,4]diazepino[5′,6′:4,5]thieno[3,2-f]quinolin-8-one (131 mg, 0.23 mmol, 75% yield). MS (ESI) [M+2H/2]⁺ 222.3.

(R)-3-(4-(1-(2,2-dimethoxyethyl)piperidin-4-yl)phenyl)-10-methyl-9,10,11,12-tetrahydro-8H-[1,4]diazepino[5′,6′:4,5]thieno[3,2-f]quinolin-8-one. To a solution of (R)-10-methyl-3-(4-(piperidin-4-yl)phenyl)-9,10,11,12-tetrahydro-8H-[1,4]diazepino[5′,6′:4,5]thieno[3,2-f]quinolin-8-one dihydrochloride (325 mg, 0.63 mmol) in DCM (3 mL) were added DIPEA (550 μL, 3.15 mmol) followed by 2,2-dimethoxyacetaldehyde (110 μL, 0.76 mmol) and NaBH(OAc)₃ (400 mg, 1.89 mmol) at rt. The reaction was stirred for 2 h at rt. LCMS indicated the reaction was completed. The reaction mixture was diluted with DCM and water. The aqueous phase was extracted with DCM (20 mL×3) and the combined organic layers were dried over sodium sulfate, filtered, and concentrated under vacuum. The residue was purified by reverse phase column chromatography (0% to 100% MeOH in water w/0.1% formic acid) to afford the product, (R)-3-(4-(1-(2,2-dimethoxyethyl)piperidin-4-yl)phenyl)-10-methyl-9,10,11,12-tetrahydro-8H-[1,4]diazepino[5′,6′:4,5]thieno[3,2-f]quinolin-8-one (170 mg, 0.32 mmol, 51% yield), as a yellow solid. MS (ESI) [M+H]⁺ 531.2.

(R)-2-(4-(4-(10-methyl-8-oxo-9,10,11,12-tetrahydro-8H-[1,4]diazepino[5′,6′:4,5]thieno[3,2-f]quinolin-3-yl)phenyl)piperidin-1-yl)acetaldehyde. To a solution of (R)-3-(4-(1-(2,2-dimethoxyethyl)piperidin-4-yl)phenyl)-10-methyl-9,10,11,12-tetrahydro-8H-[1,4]diazepino[5′,6′:4,5]thieno[3,2-f]quinolin-8-one (170 mg, 0.32 mmol) in 1,4-Dioxane (2 mL) and water (250 μL) was added HCl (3 mL, 12.8 mmol) at rt. The reaction mixture was stirred at rt overnight. LCMS indicated there was ˜85% conversion to the product. Additional HCl (2 mL, 8.5 mmol) was added and the reaction mixture was heated to 40° C. for 3 h. After removing the solvent, the solid was co-evaporated with MeCN (5 mL×3) and MTBE (5 mL×2) to afford the product, (R)-2-(4-(4-(10-methyl-8-oxo-9,10,11,12-tetrahydro-8H-[1,4]diazepino[5′,6′:4,5]thieno[3,2-(]quinolin-3-yl)phenyl)piperidin-1-yl)acetaldehyde hydrochloride (180 mg, quant. yield) as a red solid. MS (ESI) [M+H]⁺ 485.2.

3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)piperidin-1-yl)ethyl)piperazin-1-yl)phenyl)piperidine-2,6-dione. To a solution of (R)-2-(4-(4-(10-methyl-8-oxo-9,10,11,12-tetrahydro-8H-[1,4]diazepino[5′,6′:4,5]thieno[3,2-f]quinolin-3-yl)phenyl)piperidin-1-yl)acetaldehyde (90 mg, 0.17 mmol) and 3-(4-piperazin-1-ylphenyl)piperidine-2,6-dione dihydrochloride (65.8 mg, 0.19 mmol) in DCM (1.5 mL) and DMSO (0.5 mL) under a nitrogen atmosphere, was added DIPEA (210 μL, 1.21 mmol). The resulting solution was stirred at rt for 10 min. Then, NaBH(OAc)₃ (73 mg, 0.35 mmol) was added and the reaction mixture was stirred at rt overnight. LCMS analysis indicated the reaction was completed. The reaction mixture was concentrated under vacuum and purified by reverse phase column chromatography (0% to 100% MeCN in water w/ 0.1% formic acid) to the product, 3-(4-(4-(2-(4-(4-((R)-10-methyl-8-oxo-9,10,11,12-tetrahydro-8H-[1,4]diazepino[5′,6′:4,5]thieno[3,2-f]quinolin-3-yl)phenyl)piperidin-1-yl)ethyl)piperazin-1-yl)phenyl)piperidine-2,6-dione (24.8 mg, 19% yield) as a yellow solid. MS (ESI) [M+H]⁺ 742.3 ¹H NMR (400 MHz, DMSO-d₆) δ ppm 10.77 (s, 1H), 9.16-9.28 (m, 1H), 8.19-8.27 (m, 3H), 8.16 (s, 1H), 8.12 (d, J=9.0 Hz, 1H), 8.08 (br d, J=3.9 Hz, 1H), 8.00 (d, J=9.0 Hz, 1H), 7.45 (d, J=8.6 Hz, 2H), 7.15 (br t, J=4.9 Hz, 1H), 7.05 (d, J=8.8 Hz, 2H), 6.89 (d, J=8.8 Hz, 2H), 3.73 (dd, J=11.0, 4.9 Hz, 1H), 3.61 (td, J=6.8, 3.7 Hz, 1H), 3.48 (br s, 2H), 3.03-3.16 (m, 7H), 2.52-2.66 (m, 9H), 2.44 (br d, J=4.6 Hz, 1H), 2.07-2.23 (m, 3H), 1.95-2.05 (m, 1H), 1.67-1.87 (m, 4H), 1.20 (d, J=6.8 Hz, 3H), one proton not observed.

### Example 28: Synthesis of 3-(1-Methyl-6-(4-(2-(4-(4-((R)-10-methyl-8-oxo-9,10,11,12-tetrahydro-8H-[1,4]diazepino[5′,6′:4,5]thieno[3,2-f]quinolin-3-yl)phenyl)piperidin-1-yl)ethyl)piperazin-1-yl)-1H-indazol-3-yl)piperidine-2,6-dione

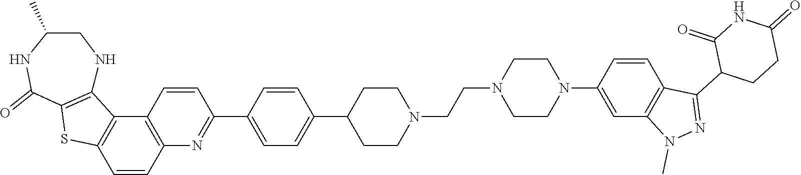 [SMILES: C[C@@H]1CNc2c(sc3ccc4nc(-c5ccc(C6CCN(CCN7CCN(c8ccc9c(C%10CCC(=O)NC%10=O)nn(C)c9c8)CC7)CC6)cc5)ccc4c23)C(=O)N1]

3-(1-Methyl-6-(4-(2-(4-(4-((R)-10-methyl-8-oxo-9,10,11,12-tetrahydro-8H-[1,4]diazepino[5′,6′:4,5]thieno[3,2-f]quinolin-3-yl)phenyl)piperidin-1-yl)ethyl)piperazin-1-yl)-1H-indazol-3-yl)piperidine-2,6-dione: A solution of (R)-2-(4-(4-(10-methyl-8-oxo-9,10,11,12-tetrahydro-8H-[1,4]diazepino[5′,6′:4,5]thieno[3,2-f]quinolin-3-yl)phenyl)piperidin-1-yl)acetaldehyde hydrochloride (97 mg, 0.19 mmol) and 3-(1-methyl-6-piperazin-1-yl-indazol-3-yl)piperidine-2,6-dione hydrochloride (74 mg, 0.20 mmol) in DCM (1.5 mL) and DMSO (0.5 mL) was prepared under a nitrogen atmosphere. To this solution, DIPEA (0.23 mL, 1.3 mmol) was added and the resulting solution was stirred for 10 min at rt. Then, NaBH(OAc)₃ (79 mg, 0.37 mmol) was added and the reaction mixture was stirred at rt overnight. The reaction was concentrated under vacuum and purified directly by reverse phase column chromatography (0% to 100% MeCN in water w/ 0.1% formic acid) to afford the product, 3-(1-methyl-6-(4-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)piperidin-1-yl)ethyl)piperazin-1-yl)-1H-indazol-3-yl)piperidine-2,6-dione (21 mg, 14% yield) as a yellow solid. MS (ESI) [M+H]⁺ 796.3 ¹H NMR (400 MHz, DMSO-d₆) δ ppm 10.85 (s, 1H), 9.14-9.28 (m, 1H), 8.24 (d, J=8.3 Hz, 2H), 8.21 (d, J=9.0 Hz, 1H), 8.12 (d, J=8.8 Hz, 1H), 8.08 (br d, J=4.2 Hz, 1H), 8.00 (d, J=8.8 Hz, 1H), 7.50 (d, J=9.0 Hz, 1H), 7.45 (d, J=8.6 Hz, 2H), 7.15 (br t, J=5.0 Hz, 1H), 6.93 (dd, J=9.0, 1.7 Hz, 1H), 6.85 (d, J=1.5 Hz, 1H), 4.26 (dd, J=9.0, 5.1 Hz, 1H), 3.89 (s, 3H), 3.61 (td, J=6.8, 3.7 Hz, 1H), 3.46-3.50 (m, 2H), 3.21-3.25 (m, 4H), 3.06 (br d, J=11.5 Hz, 2H), 2.52-2.65 (m, 11H), 2.25-2.36 (m, 1H), 2.05-2.21 (m, 3H), 1.66-1.86 (m, 4H), 1.20 (d, J=6.6 Hz, 3H).

### Example 29: Synthesis of 3-(4-(1-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)piperidin-1-yl)ethyl)piperidin-4-yl)phenyl)piperidine-2,6-dione

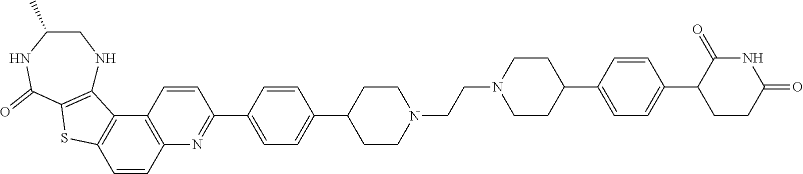 [SMILES: C[C@@H]1CNc2c(sc3ccc4nc(-c5ccc(C6CCN(CCN7CCC(c8ccc(C9CCC(=O)NC9=O)cc8)CC7)CC6)cc5)ccc4c23)C(=O)N1]

3-(4-(1-(2-(4-(4-((R)-10-Methyl-8-oxo-9,10,11,12-tetrahydro-8H-[1,4]diazepino[5′,6′:4,5]thieno[3,2-(]quinolin-3-yl)phenyl)piperidin-1-yl)ethyl)piperidin-4-yl)phenyl)piperidine-2,6-dione: To a solution of (R)-2-(4-(4-(10-methyl-8-oxo-9,10,11,12-tetrahydro-8H-[1,4]diazepino[5′,6′:4,5]thieno[3,2-f]quinolin-3-yl)phenyl)piperidin-1-yl)acetaldehyde (40 mg, 0.08 mmol) in methanol (1 mL) was added 3-(4-piperazin-1-ylphenyl)piperidine-2,6-dione (33 mg, 0.12 mmol) and NaBH₃CN (8 mg, 0.12 mmol). The resulting solution was stirred at rt for 2 h. LCMS indicated the reaction was completed. The resulting solution was concentrated under vacuum and the residue was purified by prep-HPLC (0 to 40% MeCN in water w/ 0.1% TFA) to afford the product, 3-(4-(1-(2-(4-(4-((R)-10-methyl-8-oxo-9,10,11,12-tetrahydro-8H-[1,4]diazepino[5′,6′:4,5]thieno[3,2-(]quinolin-3-yl)phenyl)piperidin-1-yl)ethyl)piperidin-4-yl)phenyl)piperidine-2,6-dione (4.9 mg, 8% yield) as a red solid. MS (ESI) [M+H]⁺ 741. ¹H NMR (400 MHz, DMSO-d₆) δ ppm 9.25 (d, J=9.0 Hz, 1H), 8.31-8.13 (m, 3H), 8.10-8.02 (m, 1H), 7.51 (d, J=8.0 Hz, 2H), 7.31-7.19 (m, 4H), 3.71-3.68 (m, 7H), 3.63-3.56 (m, 4H), 3.54-3.45 (m, 2H), 3.28-3.22 (m, 4H), 3.07-3.01 (m, 1H), 2.95-2.92 (m, 1H), 2.76-2.68 (m, 1H), 2.21-1.88 (m, 10H), 1.22 (d, J=6.8 Hz, 3H), 4 protons overlapping with residual solvent.

### Example 30: Synthesis of 3-(4-(4-(2-(3-(4-((R)-10-Methyl-8-oxo-9,10,11,12-tetrahydro-8H-[1,4]diazepino[5′,6′:4,5]thieno[3,2-f]quinolin-3-yl)phenyl)azetidin-1-yl)ethyl)piperazin-1-yl)phenyl)piperidine-2,6-dione

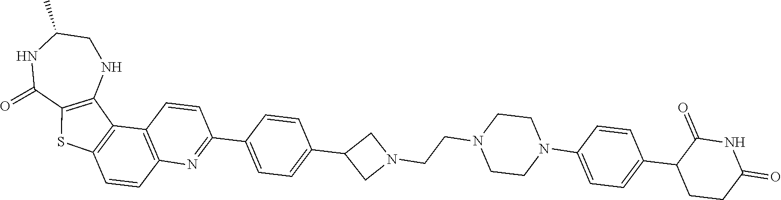 [SMILES: C[C@@H]1CNc2c(sc3ccc4nc(-c5ccc(C6CN(CCN7CCN(c8ccc(C9CCC(=O)NC9=O)cc8)CC7)C6)cc5)ccc4c23)C(=O)N1]

tert-Butyl 3-(4-(4,4,5,5-tetramethyl-1,3,2-dioxaborolan-2-yl)phenyl)azetidine-1-carboxylate. A 20 mL vial equipped with stir bar was charged with tert-butyl 3-(4-bromophenyl)azetidine-1-carboxylate (312 mg, 1.0 mmol), (bis)pinacolatodiboron (267 mg, 1.05 mmol), potassium acetate (196 mg, 2.0 mmol) and Pd(dppf)Cl₂ (73 mg, 0.1 mmol). The vial was evacuated and backfilled with nitrogen three times. 1,4-Dioxane (5.0 mL) was then added to the vial and the reaction was then heated and stirred at 80° C. overnight. The reaction mixture was filtered through a pad of silica gel, washed with 1:1 EtOAc/heptane. The filtrate was then concentrated under vacuum and used for the next step without further purification.

(R)-3-(4-(Azetidin-3-yl)phenyl)-10-methyl-9,10,11,12-tetrahydro-8H-[1,4]diazepino[5′,6′:4,5]thieno[3,2-f]quinolin-8-one. A 20 mL vial equipped with stir bar was charged with (R)-3-bromo-10-methyl-9,10,11,12-tetrahydro-8H-[1,4]diazepino[5′,6′:4,5]thieno[3,2-f]quinolin-8-one (290 mg, 0.8 mmol)), tert-butyl 3-(4-(4,4,5,5-tetramethyl-1,3,2-dioxaborolan-2-yl)phenyl)azetidine-1-carboxylate (60 wt. %, 555 mg), Pd(dppf)Cl₂ (58.5 mg, 8 mol %), and cesium carbonate (782 mg, 24 mmol). The vial was evacuated and backfilled with nitrogen three times. 1,4-Dioxane (3.6 mL) was then added and the reaction was stirred and heated at 100° C. overnight. The reaction mixture was concentrated under vacuum, treated with 4:1 DCM/TFA and allowed to stir for 4 h. LCMS indicated the reaction was completed. After removing the solvent, the residue was purified by reverse phase column chromatography (0% to 100% MeCN in water w/ 0.1% TFA) to afford the TFA salt of the product, (R)-3-(4-(azetidin-3-yl)phenyl)-10-methyl-9,10,11,12-tetrahydro-8H-[1,4]diazepino[5′,6′:4,5]thieno[3,2-f]quinolin-8-one (423 mg, 98% yield) as an orange solid. MS (ESI) [M+H]⁺ 415.4.

(R)-3-(4-(1-(2,2-Dimethoxyethyl)azetidin-3-yl)phenyl)-10-methyl-9,10,11,12-tetrahydro-8H-[1,4]diazepino[5′,6′:4,5]thieno[3,2-f]quinolin-8-one. A 20 mL vial equipped with stir bar was charged with (R)-3-(4-(azetidin-3-yl)phenyl)-10-methyl-9,10,11,12-tetrahydro-8H-[1,4]diazepino[5′,6′:4,5]thieno[3,2-f]quinolin-8-one trifluoroacetate (200 mg, 0.38 mmol). The material was then suspended in dichloromethane (3.4 mL) prior to the addition of DIPEA (264 μL, 1.5 mmol). The resulting mixture was allowed to stir for 5 minutes. A solution of 60 wt. % 2,2-dimethoxyacetaldehyde in water (86 μL) was then added and followed by sodium triacetoxyborohydride (160 mg, 0.76 mmol). The reaction was stirred at rt for 2 h, LCMS analysis revealed a full conversion to the desired product. The mixture was concentrated under vacuum and purified by reverse phase column chromatography (0% to 100% MeCN in water w/0.1% TFA) to afford the product, (R)-3-(4-(1-(2,2-dimethoxyethyl)azetidin-3-yl)phenyl)-10-methyl-9,10,11,12-tetrahydro-8H-[1,4]diazepino[5′,6′:4,5]thieno[3,2-f]quinolin-8-one (195 mg, 82% yield) as an orange solid. MS (ESI) [M+H]⁺ 503.4.

(R)-3-(4-(1-(2,2-Dihydroxyethyl)azetidin-3-yl)phenyl)-10-methyl-9,10,11,12-tetrahydro-8H-[1,4]diazepino[5′,6′:4,5]thieno[3,2-f]quinolin-8-one. A 20 mL vial equipped with stir was charged with (R)-3-(4-(1-(2,2-dimethoxyethyl)azetidin-3-yl)phenyl)-10-methyl-9,10,11,12-tetrahydro-8H-[1,4]diazepino[5′,6′:4,5]thieno[3,2-f]quinolin-8-one (154 mg, 0.25 mmol). Water (0.1 mL) was then added followed by 4 M HCl in dioxane (7.5 mmol, 1.9 mL). The reaction was heated and stirred at 50° C. for 4 h. The precipitate was filtered, and LCMS analysis revealed it was the product, (R)-3-(4-(1-(2,2-dihydroxyethyl)azetidin-3-yl)phenyl)-10-methyl-9,10,11,12-tetrahydro-8H-[1,4]diazepino[5′,6′:4,5]thieno[3,2-f]quinolin-8-one (quantitative yield), which was used directly in the next step without further purification. MS (ESI) [M+2H]/²⁺ 238.4.

3-(4-(4-(2-(3-(4-((R)-10-methyl-8-oxo-9,10,11,12-tetrahydro-8H-[1,4]diazepino[5′,6′:4,5]thieno[3,2-f]quinolin-3-yl)phenyl)azetidin-1-yl)ethyl)piperazin-1-yl)phenyl)piperidine-2,6-dione. A 20 mL vial equipped with a stir bar was charged with (R)-3-(4-(1-(2,2-Dihydroxyethyl)azetidin-3-yl)phenyl)-10-methyl-9,10,11,12-tetrahydro-8H-[1,4]diazepino[5′,6′:4,5]thieno[3,2-f]quinolin-8-one (119 mg, 0.25 mmol), 3-(4-(piperazin-1-yl)phenyl)piperidine-2,6-dione (68.3 mg, 0.25 mmol), and sodium triacetoxyborohydride (142 mg, 0.75 mmol). After added with DCM (1.25 mL) and DIPEA (2.5 mmol, 435 μL), the reaction mixture was stirred at 40° C. for 2 h. LCMS indicated full conversion to the desired product. The reaction mixture was then concentrated under vacuum, and the residue was purified by reverse phase column chromatography (0% to 100% MeCN in water w/ 0.1% TFA) to afford the product, 3-(4-(4-(2-(3-(4-((R)-10-methyl-8-oxo-9,10,11,12-tetrahydro-8H-[1,4]diazepino[5′,6′:4,5]thieno[3,2-f]quinolin-3-yl)phenyl)azetidin-1-yl)ethyl)piperazin-1-yl)phenyl)piperidine-2,6-dione (22 mg, 9% yield) as an orange solid. MS (ESI) [M+2H]/²⁺ 358.02. ¹H NMR (400 MHz, methanol-d₄) δ ppm 9.24-9.36 (m, 1H), 8.03-8.22 (m, 5H), 7.56-7.69 (m, 2H), 7.16-7.28 (m, 21H), 7.01-7.15 (m, 2H), 4.61-4.74 (m, 2H), 4.30-4.54 (m, 3H), 3.67-3.90 (m, 4H), 3.54-3.67 (m, 2H), 3.41-3.54 (m, 4H), 3.19-3.28 (m, 6H), 2.54-2.77 (m, 2H), 2.13-2.30 (m, 2H), 1.24-1.42 (m, 3H). Three exchangeable protons not observed due to the use of methanol-d₄.

### Example 31: Synthesis of 3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)-3,6-dihydropyridin-1(2H)-yl)ethyl)piperazin-1-yl)phenyl)piperidine-2,6-dione

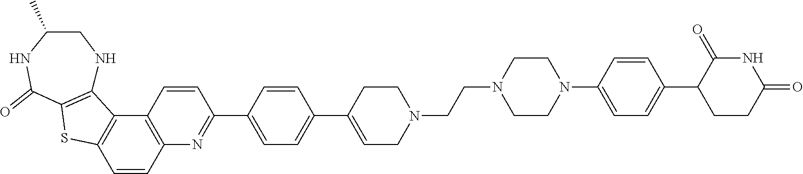 [SMILES: C[C@@H]1CNc2c(sc3ccc4nc(-c5ccc(C6=CCN(CCN7CCN(c8ccc(C9CCC(=O)NC9=O)cc8)CC7)CC6)cc5)ccc4c23)C(=O)N1]

4-(4-Bromophenyl)-1-(2,2-dimethoxyethyl)-1,2,3,6-tetrahydropyridine. To a solution of 4-(4-bromophenyl)-1,2,3,6-tetrahydropyridine hydrochloride (1.0 g, 3.64 mmol) and 2,2-dimethoxyacetaldehyde (379 mg, 3.64 mmol) in methanol (20.0 mL) was added NaBH₃CN (699 mg, 10.93 mmol). The resulting solution was stirred at rt for 3 h. LCMS indicated the reaction was completed. The reaction mixture was concentrated under vacuum, and the residue was purified by reverse phase column chromatography (0% to 100% MeCN in water w/ 0.1% TFA) to the product, 4-(4-bromophenyl)-1-(2,2-dimethoxyethyl)-1,2,3,6-tetrahydropyridine (1.1 g, 92% yield) as a white solid. MS (ESI) [M+H]⁺ 326.1.

1-(2,2-Dimethoxyethyl)-4-(4-(4,4,5,5-tetramethyl-1,3,2-dioxaborolan-2-yl)phenyl)-1,2,3,6-tetrahydropyridine. To a solution of 4-(4-bromophenyl)-1-(2,2-dimethoxyethyl)-1,2,3,6-tetrahydropyridine (600 mg, 1.84 mmol), B₂pin₂ (700 mg, 2.76 mmol) and KOAc (361 mg, 3.68 mmol) in 1,4-dioxane (8.0 mL) was added Pd(dppf)Cl₂ (150 mg, 0.184 mmol). The resulting solution was stirred at 90° C. for 4 h under a nitrogen atmosphere. LCMS indicated the reaction was completed. The resulting mixture was diluted with water (100 mL), extracted with ethyl acetate (100 mL×3). The combined organic layers were washed with brine (100 mL×2). The organic layers were then dried over anhydrous sodium sulfate, filtered and concentrated under vacuum. The residue was purified by silica gel column chromatography (petroleum ether/ethyl acetate; 1:2 ratio) to afford the product, 1-(2,2-dimethoxyethyl)-4-(4-(4,4,5,5-tetramethyl-1,3,2-dioxaborolan-2-yl)phenyl)-1,2,3,6-tetrahydropyridine (320 mg, 47% yield) as a yellow solid. MS (ESI) [M+H]⁺ 374.2.

(R)-3-(4-(1-(2,2-Dimethoxyethyl)-1,2,3,6-tetrahydropyridin-4-yl)phenyl)-10-methyl-9,10,11,12-tetrahydro-8H-[1,4]diazepino[5′,6′:4,5]thieno[3,2-f]quinolin-8-one. A mixture of 1-(2,2-dimethoxyethyl)-4-(4-(4,4,5,5-tetramethyl-1,3,2-dioxaborolan-2-yl)phenyl)-1,2,3,6-tetrahydropyridine (300 mg, 0.80 mmol), K₃PO₄ (340 mg, 1.60 mmol) and (R)-3-chloro-10-methyl-9,10,11,12-tetrahydro-8H-[1,4]diazepino[5′,6′:4,5]thieno[3,2-f]quinolin-8-one (255 mg, 0.80 mmol) in 1,4-dioxane (5.0 mL) and water (1.0 mL) was added Pd(PPh₃)₄(93 mg, 0.080 mmol). The resulting mixture was stirred at 90° C. for 18 h under a nitrogen atmosphere. LCMS showed the reaction went to completion. The reaction mixture was diluted with DMSO and then filtered. The filtrate was purified by reverse phase column chromatography (0% to 100% MeCN in water w/ 0.1% TFA) to afford the product, (R)-3-(4-(1-(2,2-dimethoxyethyl)-1,2,3,6-tetrahydropyridin-4-yl)phenyl)-10-methyl-9,10,11,12-tetrahydro-8H-[1,4]diazepino[5′,6′:4,5]thieno[3,2-f]quinolin-8-one (170 mg, 34% yield.) as an orange solid. MS (ESI) [M+H]⁺ 529.1.

(R)-2-(4-(4-(10-Methyl-8-oxo-9,10,11,12-tetrahydro-8H-[1,4]diazepino[5′,6′:4,5]thieno[3,2-f]quinolin-3-yl)phenyl)-3,6-dihydropyridin-1(2H)-yl)acetaldehyde. To a solution of (R)-3-(4-(1-(2,2-dimethoxyethyl)-1,2,3,6-tetrahydropyridin-4-yl)phenyl)-10-methyl-9,10,11,12-tetrahydro-8H-[1,4]diazepino[5′,6′:4,5]thieno[3,2-f]quinolin-8-one (150 mg, 0.28 mmol) in 1,4-dioxane (2.0 mL) and water (1.0 mL) was added 4 M HCl in 1,4-dioxane (2.0 mL). The resulting solution was stirred at 40° C. for 18 h. LCMS showed the reaction went to completion. The reaction mixture was concentrated under vacuum to afford the product, (R)-2-(4-(4-(10-methyl-8-oxo-9,10,11,12-tetrahydro-8H-[1,4]diazepino[5′,6′:4,5]thieno[3,2-f]quinolin-3-yl)phenyl)-3,6-dihydropyridin-1(2H)-yl)acetaldehyde, which was used directly in the next step without further purification. MS (ESI) [M+H₂O]⁺ 501.2.

3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)phenyl)-3,6-dihydropyridin-1(2H)-yl)ethyl)piperazin-1-yl)phenyl)piperidine-2,6-dione. To a solution of 3-(4-piperazin-1-ylphenyl)piperidine-2,6-dione (89 mg, 0.29 mmol) in methanol (4.0 mL), TEA was added at 0° C. to adjust the pH of the solution to 8˜10, and then (R)-2-(4-(4-(10-methyl-8-oxo-9,10,11,12-tetrahydro-8H-[1,4]diazepino[5′,6′:4,5]thieno[3,2-f]quinolin-3-yl)phenyl)-3,6-dihydropyridin-1(2H)-yl)acetaldehyde hydrochloride (150 mg, 0.29 mmol) was added. Still at 0° C., HOAc (0.04 mL, 0.58 mmol) was added to adjust the pH of the solution to 4˜6, NaBH₃CN (55 mg, 0.87 mmol) was then added, and the resulting solution was stirred at rt for 18 h, and LCMS showed the reaction went to completion. The resulting solution was concentrated under vacuum and the residue was purified by preparative HPLC (0% to 100% MeCN in water w/ 0.1% TFA), affording the product, 3-(4-(4-(2-(4-(4-((R)-10-methyl-8-oxo-9,10,11,12-tetrahydro-8H-[1,4]diazepino[5′,6′:4,5]thieno[3,2-f]quinolin-3-yl)phenyl)-3,6-dihydropyridin-1(2H)-yl)ethyl)piperazin-1-yl)phenyl)piperidine-2,6-dione (18.4 mg, 8% yield) as an orange solid. MS (ESI) [M+H]⁺ 741.2 ¹H NMR (400 MHz, DMSO-d₆) δ ppm 9.22 (d, J=8.8 Hz, 1H), 8.30 (dd, J=8.4, 4.8 Hz, 2H), 8.22 (d, J=8.8 Hz, 1H), 8.13 (d, J=8.8 Hz, 1H), 8.02 (d, J=8.8 Hz, 1H), 7.71 (d, J=8.4 Hz, 2H), 7.14-7.07 (m, 2H), 6.97 (d, J=8.8 Hz, 2H), 6.36 (s, 1H), 3.99 (s, 2H), 3.85-3.78 (m, 2H), 3.70-3.20 (m, 17H), 2.88 (s, 2H), 2.74-2.62 (m, 1H), 2.11 (d, J=9.5 Hz, 1H), 2.04-1.95 (m, 1H), 1.18 (d, J=6.8 Hz, 3H). Three protons not observed.

### Example 32: 3-(4-(4-(2-(4-(4-((R)-10-Methyl-8-oxo-8,9,10,11-tetrahydro-[1,4]oxazepino[7′,6′:4,5]thieno[3,2-f]quinolin-3-yl)phenyl)piperidin-1-yl)ethyl)piperazin-1-yl)phenyl)piperidine-2,6-dione

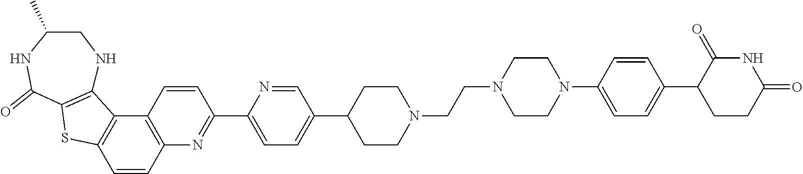 [SMILES: C[C@@H]1CNc2c(sc3ccc4nc(-c5ccc(C6CCN(CCN7CCN(c8ccc(C9CCC(=O)NC9=O)cc8)CC7)CC6)cn5)ccc4c23)C(=O)N1]

tert-butyl (R)-3-(4-(1-(tert-butoxycarbonyl)piperidin-4-yl)phenyl)-10-methyl-8-oxo-10,11-dihydro-[1,4]oxazepino[7′,6′:4,5]thieno[3,2-f]quinoline-9(8H)-carboxylate. To a solution of tert-butyl 4-[4-(4,4,5,5-tetramethyl-1,3,2-dioxaborolan-2-yl)phenyl]piperidine-1-carboxylate (300 mg, 0.77 mmol), tert-butyl (R)-3-chloro-10-methyl-8-oxo-10,11-dihydro-[1,4]oxazepino[7′,6′:4,5]thieno[3,2-f]quinoline-9(8H)-carboxylate (389 mg, 0.93 mmol) and K₂CO₃ (320 mg, 2.32 mmol) in 1,4-dioxane (20.0 mL) and water (2.0 mL) was added Pd(dppf)Cl₂ (63 mg, 0.080 mmol, 0.10). The reaction mixture was stirred at 100° C. overnight. LCMS showed the reaction went to completion. The resulting solution was cooled to rt and quenched with water (100 mL), extracted with ethyl acetate (50 mL×3). The organic layers were washed with brine (50 mL×2), dried over anhydrous sodium sulfate, filtered, and concentrated under vacuum. The residue was dissolved into ethyl acetate/petroleum ether (5.0 mL, 1:2) and filtered, The filtrate was concentrated, and the residue purified by reverse phase column chromatography (0% to 100% MeCN in water w/ 0.1% TFA) to afford the product, tert-butyl (R)-3-(4-(1-(tert-butoxycarbonyl)piperidin-4-yl)phenyl)-10-methyl-8-oxo-10,11-dihydro-[1,4]oxazepino[7′,6′:4,5]thieno[3,2-f]quinoline-9(8H)-carboxylate (250 mg, 50% yield) as a yellow solid. MS (ESI) [M+H]⁺ 643.3.

(R)-10-Methyl-3-(4-(piperidin-4-yl)phenyl)-10,11-dihydro-[1,4]oxazepino[7′,6′:4,5]thieno[3,2-f]quinolin-8(9H)-one. To a solution of tert-butyl (R)-3-(4-(1-(tert-butoxycarbonyl)piperidin-4-yl)phenyl)-10-methyl-8-oxo-10,11-dihydro-[1,4]oxazepino[7′,6′:4,5]thieno[3,2-f]quinoline-9(8H)-carboxylate (250 mg, 0.390 mmol) in DCM (4.0 mL) was added dropwise TFA (1.0 mL). The resulting mixture was stirred rt overnight. LCMS showed the reaction went to completion. The resulting solution was concentrated under reduced pressure to afford the product, (R)-10-methyl-3-(4-(piperidin-4-yl)phenyl)-10,11-dihydro-[1,4]oxazepino[7′,6′:4,5]thieno[3,2-f]quinolin-8(9H)-one (130 mg), which was used directly for the next step without further purification. MS (ESI) [M+H]⁺ 443.2

(R)-3-(4-(1-(2,2-Dimethoxyethyl)piperidin-4-yl)phenyl)-10-methyl-10,11-dihydro-[1,4]oxazepino[7′,6′:4,5]thieno[3,2-f]quinolin-8(9H)-one. To a solution of (R)-10-methyl-3-(4-(piperidin-4-yl)phenyl)-10,11-dihydro-[1,4]oxazepino[7′,6′:4,5]thieno[3,2-f]quinolin-8(9H)-one (130 mg, 0.29 mmol) in methanol (5.0 mL), 2,2-dimethoxyacetaldehyde (30 mg, 0.29 mmol) was added NaBH₃CN (71 mg, 1.17 mmol). The resulting solution was stirred at rt for 2 h. LCMS showed the reaction went to completion. The resulting solution was concentrated under vacuum and purified by reverse phase column chromatography (0% to 100% MeCN in water w/ 0.1% TFA) to afford the product, (R)-3-(4-(1-(2,2-dimethoxyethyl)piperidin-4-yl)phenyl)-10-methyl-10,11-dihydro-[1,4]oxazepino[7′,6′:4,5]thieno[3,2-f]quinolin-8(9H)-one (80 mg, 51% yield) as a brown solid. MS (ESI) [M+H]⁺ 532.4.

(R)-2-(4-(4-(10-Methyl-8-oxo-8,9,10,11-tetrahydro-[1,4]oxazepino[7′,6′:4,5]thieno[3,2-f]quinolin-3-yl)phenyl)piperidin-1-yl)acetaldehyde. To a solution of (R)-3-(4-(1-(2,2-dimethoxyethyl)piperidin-4-yl)phenyl)-10-methyl-10,11-dihydro-[1,4]oxazepino[7′,6′:4,5]thieno[3,2-f]quinolin-8(9H)-one (60 mg, 0.11 mmol) in 1,4-dioxane (6.0 mL) was added 4 M HCl in 1,4-dioxane (2.0 mL). The reaction mixture was stirred at rt overnight. LCMS showed the reaction went to completion. The resulting solution was concentrated under vacuum to the product, (R)-2-(4-(4-(10-methyl-8-oxo-8,9,10,11-tetrahydro-[1,4]oxazepino[7′,6′:4,5]thieno[3,2-f]quinolin-3-yl)phenyl)piperidin-1-yl)acetaldehyde (40 mg), which was used directly in the next step without further purification. MS (ESI) [M+H]⁺ 486.2.

3-(4-(4-(2-(4-(4-((R)-10-Methyl-8-oxo-8,9,10,11-tetrahydro-[1,4]oxazepino[7′,6′:4,5]thieno[3,2-f]quinolin-3-yl)phenyl)piperidin-1-yl)ethyl)piperazin-1-yl)phenyl)piperidine-2,6-dione. To a solution of (R)-2-(4-(4-(10-methyl-8-oxo-8,9,10,11-tetrahydro-[1,4]oxazepino[7′,6′:4,5]thieno[3,2-f]quinolin-3-yl)phenyl)piperidin-1-yl)acetaldehyde (20 mg, 0.040 mmol) in methanol (5.0 mL) was added TEA (0.5 mL). The mixture was stirred at 0° C. for 5 min. Then, 3-(4-piperazin-1-ylphenyl)piperidine-2,6-dione (11 mg, 0.040 mmol), CH₃COOH (1.0 mL) and NaBH₃CN (8 mg, 0.12 mmol), were added to the mixture. The resulting solution was stirred at rt for 2 h. LCMS showed the reaction went to completion. The reaction mixture was concentrated under vacuum, and the residue was purified by preparative HPLC (0% to 100% MeCN in water w/ 0.1% TFA) to afford the product, 3-(4-(4-(2-(4-(4-((R)-10-methyl-8-oxo-8,9,10,11-tetrahydro-[1,4]oxazepino[7′,6′:4,5]thieno[3,2-f]quinolin-3-yl)phenyl)piperidin-1-yl)ethyl)piperazin-1-yl)phenyl)piperidine-2,6-dione (1.4 mg, 4% yield.) as a yellow solid. MS (ESI) [M+H]⁺ 743.2 ¹H NMR (400 MHz, DMSO-d₆) δ ppm 9.34 (d, J=8.8 Hz, 1H), 8.29-8.21 (m, 4H), 8.08 (d, J=8.8 Hz, 1H), 7.48 (d, J=8.4 Hz, 2H), 7.14 (d, J=8.8 Hz, 2H), 7.01 (d, J=8.8 Hz, 2H), 4.63 (d, J=4.0 Hz, 2H), 3.98-3.15 (m, 18H), 3.05-2.95 (m, 1H), 2.75-2.65 (m, 1H), 2.20-2.13 (m, 3H), 2.05-1.95 (m, 3H), 1.30 (d, J=6.8 Hz, 3H). Three protons not observed.

### Example 33: Synthesis of 3-(4-(4-(2-(3-(4-((R)-10-Methyl-8-oxo-8,9,10,11-tetrahydro-[1,4]oxazepino[7′,6′:4,5]thieno[3,2-f]quinolin-3-yl)phenyl)azetidin-1-yl)ethyl)piperazin-1-yl)phenyl)piperidine-2,6-dione

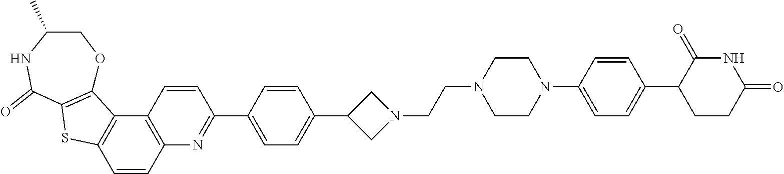 [SMILES: C[C@@H]1COc2c(sc3ccc4nc(-c5ccc(C6CN(CCN7CCN(c8ccc(C9CCC(=O)NC9=O)cc8)CC7)C6)cc5)ccc4c23)C(=O)N1]

tert-Butyl (R)-3-(4-(1-(tert-butoxycarbonyl)azetidin-3-yl)phenyl)-10-methyl-8-oxo-10,11-dihydro-[1,4]oxazepino[7′,6′:4,5]thieno[3,2-f]quinoline-9(8H)-carboxylate. To a solution of tert-butyl 3-[4-(4,4,5,5-tetramethyl-1,3,2-dioxaborolan-2-yl) phenyl]azetidine-1-carboxylate (1.6 mmol) in 1,4-dioxane (20.0 mL) and water (2.0 mL) was added tert-butyl (R)-3-chloro-10-methyl-8-oxo-10,11-dihydro-[1,4]oxazepino[7′,6′:4,5]thieno[3,2-f]quinoline-9(8H)-carboxylate (250 mg, 0.60 mmol), Pd(dppf)Cl₂ (51 mg, 0.060 mmol) and K₂CO₃ (260 mg, 1.89 mmol). The resulting solution was stirred at 100° C. for 3.0 h under a nitrogen atmosphere. LCMS showed the reaction went to completion. The reaction mixture was concentrated under vacuum, and the residue was purified by reverse phase column chromatography (0% to 100% MeCN in water w/ 0.1% ammonium formate) to afford the product, tert-butyl (R)-3-(4-(1-(tert-butoxycarbonyl)azetidin-3-yl)phenyl)-10-methyl-8-oxo-10,11-dihydro-[1,4]oxazepino[7′,6′:4,5]thieno[3,2-f]quinoline-9(8H)-carboxylate (240 mg 62% yield) as a yellow solid. MS (ESI) [M+H]⁺ 616.1.

(R)-3-(4-(Azetidin-3-yl)phenyl)-10-methyl-10,11-dihydro-[1,4]oxazepino[7′,6′:4,5]thieno[3,2-f]quinolin-8(9H)-one. To a solution of tert-butyl (R)-3-(4-(1-(tert-butoxycarbonyl)azetidin-3-yl)phenyl)-10-methyl-8-oxo-10,11-dihydro-[1,4]oxazepino[7′,6′:4,5]thieno[3,2-f]quinoline-9(8H)-carboxylate (240 mg, 0.39 mmol) in DCM (10.0 mL) was added TFA (2.0 mL), dropwise. The reaction mixture was stirred at rt for 3 h under a nitrogen atmosphere. LCMS showed the reaction went to completion. The reaction mixture was concentrated under vacuum to afford the product, (R)-3-(4-(azetidin-3-yl)phenyl)-10-methyl-10,11-dihydro-[1,4]oxazepino[7′,6′:4,5]thieno[3,2-f]quinolin-8(9H)-one, which was used directly in the next step without further purification. MS (ESI) [M+H]⁺ 416.3.

3-[4-[4-(2,2-Dimethoxyethyl) piperazin-1-yl]phenyl]piperidine-2,6-dione. To a solution of 3-(4-piperazin-1-ylphenyl) piperidine-2,6-dione (100 mg, 0.370 mmol) in methanol (5.0 mL), the pH value of the solution was adjusted to 9˜10 with TEA, and then the 2,2-dimethoxyacetaldehyde (45 mg, 0.440 mmol) was added and the pH value of the solution was adjusted to 5˜6 with AcOH, and followed by the addition of the NaBH₃CN (69 mg, 1.100 mmol). The resulting solution was stirred for 2 h at rt, LCMS showed the reaction was completed. After removing the solvent under vacuum, and the residue was purified by reverse flash chromatography with water (0.05% TFA)/MeCN (1:2) to give 3-[4-[4-(2,2-dimethoxyethyl) piperazin-1-yl]phenyl]piperidine-2,6-dione (80 mg, 60%) as a yellow oil. MS (ESI) [M+H]⁺ 362.3.

2-(4-(4-(2,6-Dioxopiperidin-3-yl)phenyl)piperazin-1-yl)acetaldehyde. To a solution of 3-[4-[4-(2,2-dimethoxyethyl) piperazin-1-yl]phenyl]piperidine-2,6-dione (80 mg, 0.22 mmol) in 1,4-dioxane (2.5 mL) was added 4 M HCl in 1,4-dioxane (2.5 mL). The reaction mixture was stirred at 40° C. for 3 h under a nitrogen atmosphere. LCMS showed the reaction went to completion. The resulting mixture was concentrated under vacuum to afford the product, 2-(4-(4-(2,6-dioxopiperidin-3-yl)phenyl)piperazin-1-yl)acetaldehyde (65 mg, 91% yield) as a yellow solid, which was used in the next step without further purification. MS (ESI) [M+H+H₂O]⁺ 334.2.

3-(4-(4-(2-(3-(4-((R)-10-Methyl-8-oxo-8,9,10,11-tetrahydro-[1,4]oxazepino[7′,6′:4,5]thieno[3,2-f]quinolin-3-yl)phenyl)azetidin-1-yl)ethyl)piperazin-1-yl)phenyl)piperidine-2,6-dione. A solution of (R)-3-(4-(azetidin-3-yl)phenyl)-10-methyl-10,11-dihydro-[1,4]oxazepino[7′,6′:4,5]thieno[3,2-f]quinolin-8(9H)-one (135 mg, 0.32 mmol) in methanol (5.0 mL) was prepared. The pH of the solution was adjusted to 9˜10 by TEA, and then the 2-[4-[4-(2,6-dioxo-3-piperidyl) phenyl]piperazin-1-yl]acetaldehyde (122 mg, 0.39 mmol) was added. The resulting solution was acidified with AcOH until a pH of 5˜6, and the NaBH₃CN (61 mg, 0.97 mmol) was added and stirred for at rt for 2 h, LCMS showed the reaction went to completion. The resulting mixture was then diluted with water (50 mL) and extracted with ethyl acetate (50 mL×3). The combined organic layers were washed with brine (50 mL×2), dried over anhydrous sodium sulfate, filtered and concentrated under reduced vacuum, the residue was purified by Prep-HPLC, to afford the product, 3-(4-(4-(2-(3-(4-((R)-10-methyl-8-oxo-8,9,10,11-tetrahydro-[1,4]oxazepino[7′,6′:4,5]thieno[3,2-f]quinolin-3-yl)phenyl)azetidin-1-yl)ethyl)piperazin-1-yl)phenyl)piperidine-2,6-dione (4.8 mg, 2% yield) as a yellow solid. MS (ESI) [M+H]⁺ 715.3 ¹H NMR (400 MHz, DMSO-d₆) δ ppm 10.80 (s, 1H), 9.31 (d, J=9.2 Hz, 1H), 8.49 (s, 1H), 8.38-8.20 (m, 4H), 8.07 (d, J=9.2 Hz, 1H), 7.53 (d, J=8.0 Hz, 2H), 7.05 (d, J=8.4 Hz, 2H), 6.88 (d, J=8.4 Hz, 2H), 4.63 (s, 2H), 3.86-3.71 (m, 6H), 3.26 (s, 3H), 3.10 (d, J=5.2 Hz, 4H), 2.80-2.30 (m, 8H), 2.20-1.95 (m, 2H), 1.29 (d, J=6.8 Hz, 3H).

### Example 34: Synthesis of 3-(4-(4-(2-(4-(5-((R)-10-Methyl-8-oxo-9,10,11,12-tetrahydro-8H-[1,4]diazepino[5′,6′:4,5]thieno[3,2-f]quinolin-3-yl)pyridin-2-yl)piperidin-1-yl)ethyl)piperazin-1-yl)phenyl)piperidine-2,6-dione

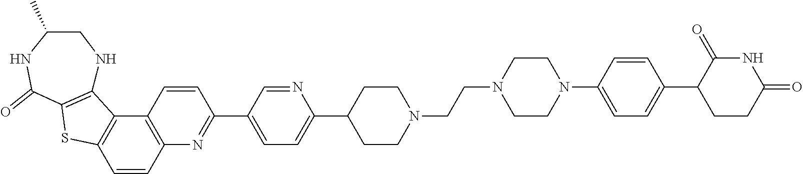 [SMILES: C[C@@H]1CNc2c(sc3ccc4nc(-c5ccc(C6CCN(CCN7CCN(c8ccc(C9CCC(=O)NC9=O)cc8)CC7)CC6)nc5)ccc4c23)C(=O)N1]

2-(1-(2,2-Dimethoxyethyl)piperidin-4-yl)pyridine. A solution of NaBH₃CN (796.0 mg, 12.4 mmol) in methanol (10.0 mL) was charged with 1.9 M ZnCl₂ in THE (15.8 mL, 4.14 mmol). The mixture was stirred at rt for 10 min. After the addition of 5-bromo-2-(piperidin-4-yl)pyridine (1.0 g, 4.14 mmol) and 2,2-dimethoxyacetaldehyde (1.2 g, 8.28 mmol), the reaction mixture was stirred at rt overnight. After removed the solvent under vacuum, the residue was purified by reverse phase chromatography with water and acetonitrile (MeCN) with 0.05% trifluoracetic acid (TFA) to afford 5-bromo-2-(1-(2,2-dimethoxyethyl)piperidin-4-yl)pyridine (750.0 mg, 55% yield) as a colorless oil. MS (ESI, m/z) [M+H]⁺ 329.1.

6-(1-(2,2-Dimethoxyethyl)piperidin-4-yl)pyridin-3-yl)boronic acid. To a solution of 5-bromo-2-(1-(2,2-dimethoxyethyl)piperidin-4-yl)pyridine (320 mg, 0.97 mmol) in 1,4-dioxane (5.0 mL) were charged with bis(pinacolato)diboron (494 mg, 1.9 mmol), Pd(dppf)Cl₂ (79.0 mg, 0.097 mmol) and KOAc (286 mg, 2.9 mmol). The resulting mixture was stirred at 80° C. overnight under inert atmosphere. After removed the solvent, the crude product was directly used for the next step without further purification. MS (ESI, m/z) [M+H]⁺ 295.2.

(R)-3-(6-(1-(2,2-Dimethoxyethyl)piperidin-4-yl)pyridin-3-yl)-10-methyl-9,10,11,12-tetrahydro-8H-[1,4]diazepino[5′,6′:4,5]thieno[3,2-f]quinolin-8-one. To a solution of (6-(1-(2,2-dimethoxyethyl)piperidin-4-yl)pyridin-3-yl)boronic acid (278 mg, 0.94 mmol) in 1,4-dioxane (5.0 mL) and water (0.5 mL) were added (R)-3-chloro-10-methyl-9,10,11,12-tetrahydro-8H-[1,4]diazepino[5′,6′:4,5]thieno[3,2-f]quinolin-8-one (200.0 mg, 0.63 mmol), Pd(PPh₃)₄(73.0 mg, 0.06 mmol) and K₂CO₃ (260.0 mg, 1.89 mmol). Under an inert atmosphere, the resulting solution was first stirred at 100° C. overnight and then continuously stirred overnight at rt. To the reaction mixture, water (50 mL) was added and then extracted with ethyl acetate (50 mL×3). The combined organic layer was washed with brine (50 mL×2), dried over anhydrous sodium sulfate, filtered and concentrated under vacuum. The residue was purified by prep-HPLC with petroleum ether/ethyl acetate (1:2) to afford (R)-3-(6-(1-(2,2-dimethoxyethyl)piperidin-4-yl)pyridin-3-yl)-10-methyl-9,10,11,12-tetrahydro-8H-[1,4]diazepino[5′,6′:4,5]thieno[3,2-f]quinolin-8-one (170.0 mg, 51% yield) as a light-yellow solid. MS (ESI, m/z) [M+H]⁺ 532.1.

(R)-2-(4-(5-(10-Methyl-8-oxo-9,10,11,12-tetrahydro-8H-[1,4]diazepino[5′,6′:4,5]thieno[3,2-f]quinolin-3-yl)pyridin-2-yl)piperidin-1-yl)acetaldehyde. To a solution of (R)-3-(6-(1-(2,2-dimethoxyethyl)piperidin-4-yl)pyridin-3-yl)-10-methyl-9,10,11,12-tetrahydro-8H-[1,4]diazepino[5′,6′:4,5]thieno[3,2-f]quinolin-8-one (170.0 mg, 0.32 mmol) in 1,4-dioxane (4.0 mL) and H₂O (1.0 mL) was added HCl 10.0 mL, conc.). The resulting solution was stirred at 40° C. for 2 hr, and then concentrated under vacuum to afford crude product, (R)-2-(4-(5-(10-methyl-8-oxo-9,10,11,12-tetrahydro-8H-[1,4]diazepino[5′,6′:4,5]thieno[3,2-f]quinolin-3-yl)pyridin-2-yl)piperidin-1-yl)acetaldehyde which was directly used for the next step without further purification. MS (ESI, m/z) [M+18]⁺ 504.3.

3-(4-(4-(2-(4-(5-((R)-10-Methyl-8-oxo-9,10,11,12-tetrahydro-8H-[1,4]diazepino[5′,6′:4,5]thieno[3,2-f]quinolin-3-yl)pyridin-2-yl)piperidin-1-yl)ethyl)piperazin-1-yl)phenyl)piperidine-2,6-dione. To a stirred a solution of (R)-2-(4-(5-(10-methyl-8-oxo-9,10,11,12-tetrahydro-8H-[1,4]diazepino[5′,6′:4,5]thieno[3,2-f]quinolin-3-yl)pyridin-2-yl)piperidin-1-yl)acetaldehyde (45.0 mg, 0.09 mmol) and 3-(4-piperazin-1-ylphenyl)piperidine-2,6-dione (38.0 mg, 0.14 mmol) in methanol (2.0 mL) was added NaBH₃CN (59.0 mg, 0.28 mmol). The resulting solution was stirred at rt overnight. After removed the solvent, the residue was purified by reverse-phase chromatography (RPC) using water and acetonitrile (MeCN) as mobile phases with NH₄HCO₃ (10 mmol/L) to afford the desired product; 3-(4-(4-(2-(4-(5-((R)-10-methyl-8-oxo-9,10,11,12-tetrahydro-8H-[1,4]diazepino[5′,6′:4,5]thieno[3,2-f]quinolin-3-yl)pyridin-2-yl)piperidin-1-yl)ethyl)piperazin-1-yl)phenyl)piperidine-2,6-dione (11.5 mg, 17.0% yield) as a yellow solid. MS (ESI, m/z) [M+H]⁺ 743.3 ¹H NMR (400 MHz, CF₃COOD-d₁): δ ppm 11.74 (s, 1H), 11.44 (s, 1H), 11.02 (d, J=8.8 Hz, 1H), 10.28 (d, J=9.2 Hz, 2H), 10.19 (d, J=9.2 Hz, 1H), 10.08 (d, J=8.4 Hz, 1H), 9.44 (d, J=8.4 Hz, 2H), 9.30 (d, J=8.4 Hz, 2H), 6.38-6.02 (m, 7H), 5.96-5.50 (m, 12H), 5.40-5.22 (m, 2H), 4.82-4.69 (m, 2H), 4.50-4.22 (m, 4H), 4.22-3.99 (m, 2H), 3.33-3.12 (d, J=6.8 Hz, 3H). ¹⁹F NMR (376 Hz, CF₃COOD-d₁) δ −76.40.

### Example 35: Synthesis of 3-(4-(4-(2-(4-(6-((R)-10-Methyl-8-oxo-9,10,11,12-tetrahydro-8H-[1,4]diazepino[5′,6′:4,5]thieno[3,2-f]quinolin-3-yl)pyridin-3-yl)piperidin-1-yl)ethyl)piperazin-1-yl)phenyl)piperidine-2,6-dione

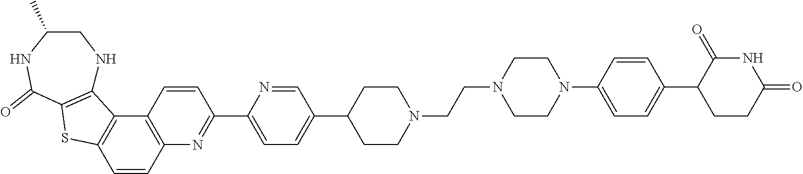 [SMILES: C[C@@H]1CNc2c(sc3ccc4nc(-c5ccc(C6CCN(CCN7CCN(c8ccc(C9CCC(=O)NC9=O)cc8)CC7)CC6)cn5)ccc4c23)C(=O)N1]

6-Chloro-1′-(2,2-dimethoxyethyl)-1′,2′,3′,6′-tetrahydro-3,4′-bipyridine. To a solution of 2-chloro-5-(1,2,3,6-tetrahydropyridin-4-yl)pyridine;hydrochloride (1.1 g, 4.76 mmol) in methanol (15.0 mL) was added 2,2-dimethoxyacetaldehyde (594.0 mg, 5.7 mmol) and NaBH₃CN (913.0 mg, 14.3 mmol). The resulting solution was stirred at rt for 3 h and concentrated to remove the solvent under vacuum. The residue was purified by reverse-phase chromatography (RPC) using water and acetonitrile (MeCN) as mobile phases with 0.05% trifluoracetic acid (TFA) to afford 2-chloro-5-[1-(2,2-dimethoxyethyl)-3,6-dihydro-2H-pyridin-4-yl]pyridine (1.3 g, 96%) as a white solid. MS (ESI, m/z) [M+H]⁺ 283.1.

(R)-3-(1′-(2,2-Dimethoxyethyl)-1′,2′,3′,6′-tetrahydro-[3,4′-bipyridin]-6-yl)-10-methyl-9,10,11,12-tetrahydro-8H-[1,4]diazepino[5′,6′:4,5]thieno[3,2-f]quinolin-8-one. To a solution of 6-chloro-1′-(2,2-dimethoxyethyl)-1′,2′,3′,6′-tetrahydro-3,4′-bipyridine (400.0 mg, 1.42 mmol) and (R)-10-methyl-3-(tributylstannyl)-9,10,11,12-tetrahydro-8H-[1,4]diazepino[5′,6′:4,5]thieno[3,2-f]quinolin-8-one (1.2 g, 2.1 mmol) in 1,4-dioxane (8.0 mL) was added XPhos Pd G₃ (120.0 mg, 0.142 mmol) and XPhos (120.0 mg, 0.28 mmol). The resulting solution was stirred at 90° C. for 6 h under an inert atmosphere. After removing the solvent under vacuum, the residue was purified by reverse-phase chromatography (RPC) using water and acetonitrile (MeCN) as mobile phases with 0.05% trifluoracetic acid (TFA) to afford the desired product, (R)-3-(1′-(2,2-dimethoxyethyl)-1′,2′,3′,6′-tetrahydro-[3,4′-bipyridin]-6-yl)-10-methyl-9,10,11,12-tetrahydro-8H-[1,4]diazepino[5′,6′:4,5]thieno[3,2-f]quinolin-8-one (260.0 mg, 35% yield) as a red solid. MS (ESI, m/z) [M+H]⁺ 530.2.

(R)-3-(5-(1-(2,2-Dimethoxyethyl)piperidin-4-yl)pyridin-2-yl)-10-methyl-9,10,11,12-tetrahydro-8H-[1,4]diazepino[5′,6′:4,5]thieno[3,2-f]quinolin-8-one. To a solution of (R)-3-(1′-(2,2-dimethoxyethyl)-1′,2′,3′,6′-tetrahydro-[3,4′-bipyridin]-6-yl)-10-methyl-9,10,11,12-tetrahydro-8H-[1,4]diazepino[5′,6′:4,5]thieno[3,2-f]quinolin-8-one (230.0 mg, 0.43 mmol) in methanol (10.0 mL) and EtOAc (10.0 mL) was added Pd/C (230.0 mg). The resulted suspension was stirred at rt for 6 h under a hydrogen atmosphere (1 atm). After filtration, the solid was washed by methanol (30 mL×2). The filtrate was concentrated under reduced pressure to afford crude (R)-3-(5-(1-(2,2-dimethoxyethyl)piperidin-4-yl)pyridin-2-yl)-10-methyl-9,10,11,12-tetrahydro-8H-[1,4]diazepino[5′,6′:4,5]thieno[3,2-f]quinolin-8-one (100.0 mg) which was used directly for the next step without further purification. MS (ESI, m/z) [M+H]⁺ 532.1.

(R)-2-(4-(6-(10-Methyl-8-oxo-9,10,11,12-tetrahydro-8H-[1,4]diazepino[5′,6′:4,5]thieno[3,2-f]quinolin-3-yl)pyridin-3-yl)piperidin-1-yl)acetaldehyde. To a solution of (R)-3-(5-(1-(2,2-dimethoxyethyl)piperidin-4-yl)pyridin-2-yl)-10-methyl-9,10,11,12-tetrahydro-8H-[1,4]diazepino[5′,6′:4,5]thieno[3,2-f]quinolin-8-one (100 mg, 0.188 mmol) in 1,4-dioxane (2.0 mL) and water (1.0 mL) was added 4.0 M HCl in 1,4-dioxane (2.0 mL). The resulting solution was stirred at 40° C. for 18 h, and then concentrated under reduced pressure to afford the crude product which was used directly for the next step without further purification. MS (ESI, m/z) [M+H+18]⁺ 504.3.

3-(4-(4-(2-(4-(6-((R)-10-Methyl-8-oxo-9,10,11,12-tetrahydro-8H-[1,4]diazepino[5′,6′:4,5]thieno[3,2-f]quinolin-3-yl)pyridin-3-yl)piperidin-1-yl)ethyl)piperazin-1-yl)phenyl)piperidine-2,6-dione. To a solution of 3-(4-piperazin-1-ylphenyl)piperidine-2,6-dione (53.0 mg, 0.20 mmol) in methanol (4.0 mL) was added (R)-2-(4-(6-(10-methyl-8-oxo-9,10,11,12-tetrahydro-8H-[1,4]diazepino[5′,6′:4,5]thieno[3,2-f]quinolin-3-yl)pyridin-3-yl)piperidin-1-yl)acetaldehyde (95.0 mg, 0.20 mmol) and NaBH₃CN (37.0 mg, 0.59 mmol). The resulted solution was stirred at rt for 5 h, and then concentrated under reduced pressure. The residue was dissolved with DMSO (2.0 mL). After the filtration, the filtrate was purified by reverse-phase chromatography (RPC) using water and acetonitrile (MeCN) as mobile phases with 0.05% trifluoroacetic acid (TFA) to afford the desired product; 3-(4-(4-(2-(4-(6-((R)-10-Methyl-8-oxo-9,10,11,12-tetrahydro-8H-[1,4]diazepino[5′,6′:4,5]thieno[3,2-f]quinolin-3-yl)pyridin-3-yl)piperidin-1-yl)ethyl)piperazin-1-yl)phenyl)piperidine-2,6-dione (44.8 mg, 28% yield) as an orange solid. MS (ESI, m/z) [M+H]⁺ 743.3 ¹H NMR (400 MHz, DMSO-d₆) δ ppm 10.79 (s, 1H), 9.32 (d, J=9.2 Hz, 1H), 8.72 (d, J=2.4 Hz, 1H), 8.62 (dd, J=8.8, 4.0 Hz, 2H), 8.18 (d, J=8.8 Hz, 1H), 8.10 (d, J=4.4 Hz, 1H), 8.05 (d, J=8.8 Hz, 1H), 7.95 (dd, J=8.4, 2.4 Hz, 1H), 7.13 (d, J=8.8 Hz, 3H), 7.00 (d, J=8.8 Hz, 2H), 3.82-2.90 (m, 22H), 2.72-2.59 (m, 1H), 2.21-2.13 (m, 3H), 2.10-1.96 (m, 3H), 1.21 (d, J=6.8 Hz, 3H). ¹⁹F NMR (376 MHz, DMSO-d₆) δ ppm ˜74.44.

### Example 36: Synthesis of 3-(4-(4-(2-(4-(5-((R)-10-Methyl-8-oxo-9,10,11,12-tetrahydro-8H-[1,4]diazepino[5′,6′:4,5]thieno[3,2-f]quinolin-3-yl)pyrimidin-2-yl)piperidin-1-yl)ethyl)piperazin-1-yl)phenyl)piperidine-2,6-dione

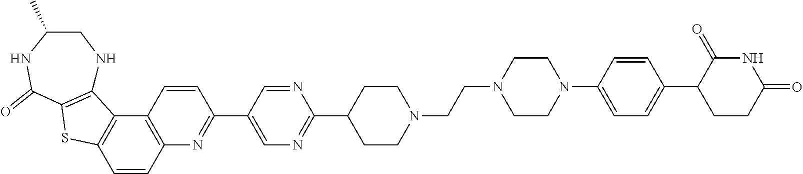 [SMILES: C[C@@H]1CNc2c(sc3ccc4nc(-c5cnc(C6CCN(CCN7CCN(c8ccc(C9CCC(=O)NC9=O)cc8)CC7)CC6)nc5)ccc4c23)C(=O)N1]

5-Bromo-2-(1-(2,2-dimethoxyethyl)piperidin-4-yl)pyrimidine. To a solution of NaBH₃CN (342.0 mg, 5.34 mmol) and ZnCl₂ (364.0 mg, 2.67 mmol) was added methanol (10.0 mL) and stirred at rt for 10 min. After the addition of 5-bromo-2-(4-piperidyl)pyrimidine (500.0 mg, 2.07 mmol) and 2,2-dimethoxyacetaldehyde (283.0 mg, 2.72 mmol), the resulting solution was stirred at rt for 4 h and then concentrated under reduced pressure. The residue was purified by reverse-phase chromatography (RPC) using water and acetonitrile (MeCN) as mobile phases with 0.05% trifluoroacetic acid (TFA) to afford the desired product, 5-bromo-2-[1-(2,2-dimethoxyethyl)-4-piperidyl]pyrimidine (600.0 mg, 88% yield) as a yellow liquid. MS (ESI, m/z) [M+H]⁺ 330.2.

(R)-3-(2-(1-(2,2-Dimethoxyethyl)piperidin-4-yl)pyrimidin-5-yl)-10-methyl-9,10,11,12-tetrahydro-8H-[1,4]diazepino[5′,6′:4,5]thieno[3,2-f]quinolin-8-one. To a solution of 5-bromo-2-(1-(2,2-dimethoxyethyl)piperidin-4-yl)pyrimidine (300.0 mg, 0.91 mmol) and (R)-10-methyl-3-(tributylstannyl)-9,10,11,12-tetrahydro-8H-[1,4]diazepino[5′,6′:4,5]thieno[3,2-f]quinolin-8-one (1.05 g, 1.83 mmol) in 1,4-dioxane (3.0 mL) was added Pd(PPh₃)₄(70.0 mg, 0.08 mmol). The resulting solution was stirred at 90° C. for 5 h under an inert atmosphere. After removing the solvent under vacuum, the residue was purified by reverse-phase chromatography (RPC) using water and acetonitrile (MeCN) as mobile phases with 0.05% trifluoroacetic acid (TFA) to afford the desired product, (R)-3-(2-(1-(2,2-Dimethoxyethyl)piperidin-4-yl)pyrimidin-5-yl)-10-methyl-9,10,11,12-tetrahydro-8H-[1,4]diazepino[5′,6′:4,5]thieno[3,2-f]quinolin-8-one (250.0 mg, 51% yield) as a yellow solid. MS (ESI, m/z) [M+H]⁺ 533.1.

(R)-2-(4-(5-(10-Methyl-8-oxo-9,10,11,12-tetrahydro-8H-[1,4]diazepino[5′,6′:4,5]thieno[3,2-f]quinolin-3-yl)pyrimidin-2-yl)piperidin-1-yl)acetaldehyde. To a solution of (R)-3-(2-(1-(2,2-Dimethoxyethyl)piperidin-4-yl)pyrimidin-5-yl)-10-methyl-9,10,11,12-tetrahydro-8H-[1,4]diazepino[5′,6′:4,5]thieno[3,2-f]quinolin-8-one (100.0 mg, 0.190 mmol) in 1,4-dioxane (1.0 mL) and water (0.50 mL) was added HCl (conc., 1.0 mL). The resulting solution was stirred at 40° C. for 4 h. After concentration under reduce pressure to remove the solvent, the crude product; (R)-2-(4-(5-(10-methyl-8-oxo-9,10,11,12-tetrahydro-8H-[1,4]diazepino[5′,6′:4,5]thieno[3,2-f]quinolin-3-yl)pyrimidin-2-yl)piperidin-1-yl)acetaldehyde was obtained as a red solid (80 mg, 87% yield), which was used directly for the next step without further purification. MS (ESI, m/z) [M+18]⁺ 533.4.

3-(4-(4-(2-(4-(5-((R)-10-Methyl-8-oxo-9,10,11,12-tetrahydro-8H-[1,4]diazepino[5′,6′:4,5]thieno[3,2-f]quinolin-3-yl)pyrimidin-2-yl)piperidin-1-yl)ethyl)piperazin-1-yl)phenyl)piperidine-2,6-dione. To a solution of (R)-2-(4-(5-(10-Methyl-8-oxo-9,10,11,12-tetrahydro-8H-[1,4]diazepino[5′,6′:4,5]thieno[3,2-f]quinolin-3-yl)pyrimidin-2-yl)piperidin-1-yl)acetaldehyde (90.0 mg, 0.18 mmol) and 3-(4-piperazin-1-ylphenyl)piperidine-2,6-dione (76.0 mg, 0.28 mmol) in methanol (2.0 mL) was added NaBH₃CN (35.0 mg, 0.56 mmol). The resulting solution was stirred at rt overnight and then concentrated under reduced pressure. The residue was purified by reverse-phase chromatography (RPC) using water and acetonitrile (MeCN) as mobile phases with 0.05% trifluoroacetic acid (TFA) to afford the desired product, 3-(4-(4-(2-(4-(5-((R)-10-Methyl-8-oxo-9,10,11,12-tetrahydro-8H-[1,4]diazepino[5′,6′:4,5]thieno[3,2-f]quinolin-3-yl)pyrimidin-2-yl)piperidin-1-yl)ethyl)piperazin-1-yl)phenyl)piperidine-2,6-dione (5.3 mg, 3.0% yield) as a yellow solid. MS (ESI, m/z) [M+H]⁺ 744.3 ¹H NMR (400 MHz, DMSO-d₆) δ ppm 9.56 (d, J=10.9 Hz, 1H), 9.30 (d, J=8.8 Hz, 1H), 8.32 (s, 4H), 8.20 (d, J=8.8 Hz, 1H), 8.10 (d, J=9.2 Hz, 1H), 7.08 (d, J=8.4 Hz, 1H), 6.92 (d, J=8.4 Hz, 1H), 3.91 (s, 3H), 3.66 (s, 4H), 3.54-3.38 (m, 4H), 3.17-2.83 (m, 6H), 2.66 (s, 6H), 2.19-2.07 (m, 6H), 1.22 (d, J=6.8 Hz, 3H).

### Example 37: Synthesis of 3-(4-(4-(2-(4-(6-((R)-10-Methyl-8-oxo-9,10,11,12-tetrahydro-8H-[1,4]diazepino[5′,6′:4,5]thieno[3,2-f]quinolin-3-yl)pyridazin-3-yl)piperidin-1-yl)ethyl)piperazin-1-yl)phenyl)piperidine-2,6-dione

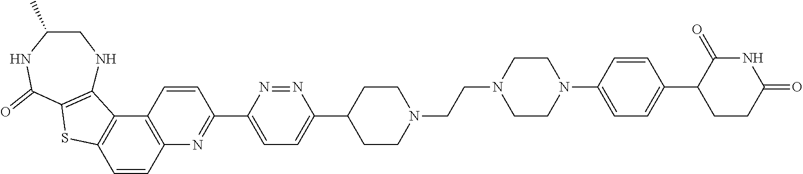 [SMILES: C[C@@H]1CNc2c(sc3ccc4nc(-c5ccc(C6CCN(CCN7CCN(c8ccc(C9CCC(=O)NC9=O)cc8)CC7)CC6)nn5)ccc4c23)C(=O)N1]

tert-Butyl 4-(6-chloropyridazin-3-yl)-3,6-dihydropyridine-1(2H)-carboxylate. To a solution of 3,6-dichloropyridazine (2.0 g, 13.4 mmol), tert-butyl 4-(4,4,5,5-tetramethyl-1,3,2-dioxaborolan-2-yl)-3,6-dihydro-2H-pyridine-1-carboxylate (3.3 g, 10.7 mmol) and K₂CO₃ (3.7 g, 26.85 mmol) in 1,4-dioxane (20.0 mL) and water (4.0 mL) was added Pd(dppf)Cl₂ (596.0 mg, 0.73 mmol). The resulting solution was stirred at 90° C. for 2 h under a nitrogen atmosphere. After cooling down to rt, the solution was diluted with water (200 mL) and then extracted by ethyl acetate (200 mL×3). The combined organic layer was washed by brine (100 mL×2), dried over anhydrous sodium sulfate, filtered, and concentrated under reduced pressure. The residue was purified by column chromatography on silica gel with ethyl acetate/petroleum ether (1:2) to afford the product, tert-butyl 4-(6-chloropyridazin-3-yl)-3,6-dihydro-2H-pyridine-1-carboxylate (2.0 g, 50% yield) as a white solid. MS (ESI, m/z) [M+H]⁺ 296.2.

3-Chloro-6-(1,2,3,6-tetrahydropyridin-4-yl)pyridazine. To a solution of tert-butyl 4-(6-chloropyridazin-3-yl)-3,6-dihydro-2H-pyridine-1-carboxylate (2.0 g, 6.76 mmol) in EtOAc (50.0 mL) was added dropwise with HCl (50.0 mL, 2.0 M aq). The resulting solution was stirred at rt for 5 h. After filtrated, the collected solid was washed by EtOAc (50 mL×2) and dried to afford 3-chloro-6-(1,2,3,6-tetrahydropyridin-4-yl)pyridazine;hydrochloride (1.4 g, 91% yield) as a white solid. MS (ESI, m/z) [M+H]⁺ 196.4.

3-Chloro-6-(1-(2,2-dimethoxyethyl)-1,2,3,6-tetrahydropyridin-4-yl)pyridazine. To a solution of 3-chloro-6-(1,2,3,6-tetrahydropyridin-4-yl)pyridazine;hydrochloride (1.4 g, 6.2 mmol) in methanol (40.0 mL) was added 2,2-dimethoxyacetaldehyde (1.3 g, 7.4 mmol) and NaBH₃CN (1.19 g, 18.6 mmol). The resulting solution was stirred at rt for 2 h and then concentrated under reduced pressure. The residue purified by reverse-phase chromatography (RPC) using water and acetonitrile (MeCN) as mobile phases with 0.05% trifluoroacetic acid (TFA) to afford the desired product; 3-chloro-6-[1-(2,2-dimethoxyethyl)-3,6-dihydro-2H-pyridin-4-yl]pyridazine (1.5 g, 85% yield) as a white solid. MS (ESI, m/z) [M+H]⁺ 284.3.

(R)-3-(6-(1-(2,2-Dimethoxyethyl)-1,2,3,6-tetrahydropyridin-4-yl)pyridazin-3-yl)-10-methyl-9,10,11,12-tetrahydro-8H-[1,4]diazepino[5′,6′:4,5]thieno[3,2-f]quinolin-8-one. To a solution of 3-chloro-6-[1-(2,2-dimethoxyethyl)-3,6-dihydro-2H-pyridin-4-yl]pyridazine (400.0 mg, 1.4 mmol), (R)-10-methyl-3-(tributylstannyl)-9,10,11,12-tetrahydro-8H-[1,4]diazepino[5′,6′:4,5]thieno[3,2-f]quinolin-8-one (1.2 g, 2.12 mmol) in 1,4-dioxane (6.0 mL) was added Pd(PPh₃)₄(162.0 mg, 0.14 mmol). The resulting solution was stirred at 90° C. for 5 h under a nitrogen atmosphere. After cooling down to rt, diluted with DMSO (2.0 mL) and filtered, the filtrate was purified by reverse-phase chromatography (RPC) using water and acetonitrile (MeCN) as mobile phases with 0.05% trifluoroacetic acid (TFA) to afford (R)-3-(6-(1-(2,2-Dimethoxyethyl)-1,2,3,6-tetrahydropyridin-4-yl)pyridazin-3-yl)-10-methyl-9,10,11,12-tetrahydro-8H-[1,4]diazepino[5′,6′:4,5]thieno[3,2-f]quinolin-8-one (240.0 mg, 32% yield) as a red solid. MS (ESI, m/z) [M+H]⁺ 531.3.

(R)-2-(4-(6-(10-Methyl-8-oxo-9,10,11,12-tetrahydro-8H-[1,4]diazepino[5′,6′:4,5]thieno[3,2-f]quinolin-3-yl)pyridazin-3-yl)-3,6-dihydropyridin-1(2H)-yl)acetaldehyde. To a solution (R)-3-(6-(1-(2,2-Dimethoxyethyl)-1,2,3,6-tetrahydropyridin-4-yl)pyridazin-3-yl)-10-methyl-9,10,11,12-tetrahydro-8H-[1,4]diazepino[5′,6′:4,5]thieno[3,2-f]quinolin-8-one (120.0 mg, 0.23 mmol) in 1,4-dioxane (2.0 mL) and water (1.0 mL) was added 4.0 M HCl in 1,4-dioxane (2.0 mL). The resulting solution was stirred at 40° C. for 18 h. After concentrated to remove the solvent under vacuum, the crude product; (R)-2-(4-(6-(10-methyl-8-oxo-9,10,11,12-tetrahydro-8H-[1,4]diazepino[5′,6′:4,5]thieno[3,2-f]quinolin-3-yl)pyridazin-3-yl)-3,6-dihydropyridin-1(2H)-yl)acetaldehyde was obtained and used directly for the next step without further purification. MS (ESI, m/z) [M+18]⁺ 503.1.

3-(4-(4-(2-(4-(6-((R)-10-Methyl-8-oxo-9,10,11,12-tetrahydro-8H-[1,4]diazepino[5′,6′:4,5]thieno[3,2-f]quinolin-3-yl)pyridazin-3-yl)piperidin-1-yl)ethyl)piperazin-1-yl)phenyl)piperidine-2,6-dione. To a solution of 3-(4-piperazin-1-ylphenyl)piperidine-2,6-dione;hydrochloride (64.0 mg, 0.21 mmol) in methanol (3.0 mL) was added (R)-2-(4-(6-(10-Methyl-8-oxo-9,10,11,12-tetrahydro-8H-[1,4]diazepino[5′,6′:4,5]thieno[3,2-f]quinolin-3-yl)pyridazin-3-yl)-3,6-dihydropyridin-1(2H)-yl)acetaldehyde (100.0 mg, 0.21 mmol) and NaBH₃CN (40.0 mg, 0.63 mmol). The resulting solution was stirred at rt for 5 h. After diluted with DMSO (2.0 mL) and filtered out, the filtrate was purified by reverse-phase chromatography (RPC) using water and acetonitrile (MeCN) as mobile phases with 0.05% trifluoroacetic acid (TFA) to afford the desired product, 3-(4-(4-(2-(4-(6-((R)-10-Methyl-8-oxo-9,10,11,12-tetrahydro-8H-[1,4]diazepino[5′,6′:4,5]thieno[3,2-f]quinolin-3-yl)pyridazin-3-yl)piperidin-1-yl)ethyl)piperazin-1-yl)phenyl)piperidine-2,6-dione (4.6 mg, 3.0% yield) as a red solid. MS (ESI, m/z) [M+H]⁺ 744.4 ¹H NMR (400 MHz, DMSO-d₆) δ ppm 10.80 (s, 1H), 9.40 (d, J=9.2 Hz, 1H), 8.84-8.72 (m, 2H), 8.22 (d, J=8.8 Hz, 1H), 8.14 (d, J=4.4 Hz, 1H), 8.12-8.05 (m, 1H), 7.91 (d, J=8.8 Hz, 1H), 7.22 (s, 1H), 7.12 (d, J=8.4 Hz, 2H), 6.99 (d, J=8.4 Hz, 2H), 3.80-3.00 (m, 21H), 2.72-2.59 (m, 1H), 2.46 (m, 1H), 2.37-1.93 (m, 6H), 1.21 (d, J=6.7 Hz, 3H). ¹⁹F NMR (376 MHz, DMSO-d₆) δ ppm ˜74.17.

### Example 38: Synthesis of 3-(4-(4-((1-(4-((R)-10-Methyl-8-oxo-9,10,11,12-tetrahydro-8H-[1,4]diazepino[5′,6′:4,5]thieno[3,2-f]quinolin-3-yl)phenyl)piperidin-4-yl)methyl)piperazin-1-yl)phenyl)piperidine-2,6-dione

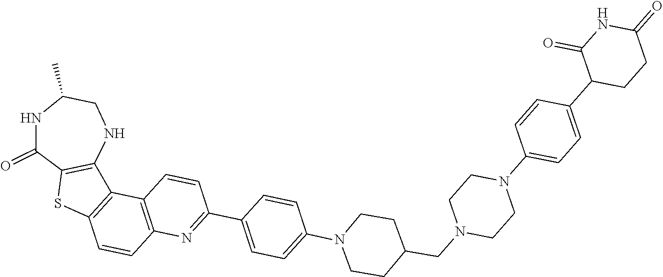 [SMILES: C[C@@H]1CNc2c(sc3ccc4nc(-c5ccc(N6CCC(CN7CCN(c8ccc(C9CCC(=O)NC9=O)cc8)CC7)CC6)cc5)ccc4c23)C(=O)N1]

Piperidine-4-carbaldehyde. To a solution of tert-butyl 4-formylpiperidine-1-carboxylate (1.0 g, 4.69 mmol) in DCM (20.0 mL) was added dropwise with TFA (5.0 mL). The reaction mixture was stirred at rt for 2 h. After concentrating under reduced pressure, the crude product; piperidine-4-carbaldehyde (600 mg) was obtained and used directly for the next step without further purification. MS (ESI, m/z) [M+H]⁺ 114.2.

1-(4-Bromophenyl)piperidine-4-carbaldehyde. To solution of piperidine-4-carbaldehyde;2,2,2-trifluoroacetic acid (500.0 mg, 2.2 mmol), TEA (1.5 mL, 8.8 mmol) and (4-bromophenyl)boronic acid (663.0 mg, 3.3 mmol) in dichloromethane (10.0 mL) was added Cu(OAc)₂ (401.0 mg, 2.2 mmol). The reaction mixture was stirred at rt overnight. The reaction mixture was diluted with water (50 mL) and extracted with dichloromethane (50 mL×3). The combined organic layer was washed with brine (50 mL×2), dried over anhydrous sodium sulfate, filtered, and concentrated under reduced pressure. The residue was purified by reverse-phase chromatography (RPC) using water and acetonitrile (MeCN) as mobile phases with 0.05% trifluoroacetic acid (TFA) to afford the desired product; 1-(4-bromophenyl)piperidine-4-carbaldehyde (200 mg, 34% yield) as an off-white solid. MS (ESI, m/z) [M+H]⁺ 268.2.

1-(4-Bromophenyl)-4-(1,3-dioxolan-2-yl)piperidine. To solution of 1-(4-bromophenyl)piperidine-4-carbaldehyde (60 mg, 0.22 mmol) and ethylene glycol (42.0 mg, 0.67 mmol) in toluene (2.0 mL) was added p-TsOH (4.0 mg, 0.022 mmol). The resulting solution was stirred for 3 h at 100° C. under a nitrogen atmosphere. The reaction mixture was diluted with water (20.0 mL) and extracted by ethyl acetate (20 mL×3). The combined organic layer was washed with brine (20 mL×2), dried over anhydrous sodium sulfate, filtered, and concentrated under reduced pressure. After removed solvent under vacuum, the residue was purified by prep-TLC (HPLC) with petroleum ether/ethyl acetate (2:1) to afford the product, 1-(4-bromophenyl)-4-(1,3-dioxolan (60 mg, 86% yield) as off-white solid. MS (ESI, m/z) [M+H]⁺ 312.3.

(R)-3-(4-(4-(1,3-Dioxolan-2-yl)piperidin-1-yl)phenyl)-10-methyl-9,10,11,12-tetrahydro-8H-[1,4]diazepino[5′,6′:4,5]thieno[3,2-f]quinolin-8-one. To solution of 1-(4-bromophenyl)-4-(1,3-dioxolan-2-yl)piperidine (45.0 mg, 0.14 mmol) and (R)-10-methyl-3-(tributylstannyl)-9,10,11,12-tetrahydro-8H-[1,4]diazepino[5′,6′:4,5]thieno[3,2-f]quinolin-8-one (82.0 mg, 0.14 mmol) in DMF (2.0 mL) was added Pd₂(dba)₃ (15.0 mg, 0.014 mmol) and P(o-Tol.)₃ (9.0 mg, 0.03 mmol). The reaction mixture was stirred at 80° C. for 3 h under a nitrogen atmosphere. After diluted with DMSO (2.0 mL), the resulting solution was purified by reverse-phase chromatography (RPC) using water and acetonitrile (CH₃CN) as mobile phases with 0.05% trifluoroacetic acid (TFA) to afford the desired product, (R)-3-(4-(4-(1,3-dioxolan-2-yl)piperidin-1-yl)phenyl)-10-methyl-9,10,11,12-tetrahydro-8H-[1,4]diazepino[5′,6′:4,5]thieno[3,2-(]quinolin-8-one (30.0 mg, 40% yield) as an off-white solid. MS (ESI, m/z) [M+H]⁺ 515.4.

(R)-1-(4-(10-Methyl-8-oxo-9,10,11,12-tetrahydro-8H-[1,4]diazepino[5′,6′:4,5]thieno[3,2-f]quinolin-3-yl)phenyl)piperidine-4-carbaldehyde. To solution of (R)-3-(4-(4-(1,3-dioxolan-2-yl)piperidin-1-yl)phenyl)-10-methyl-9,10,11,12-tetrahydro-8H-[1,4]diazepino[5′,6′:4,5]thieno[3,2-f]quinolin-8-one (30.0 mg, 0.06 mmol) in acetone (1.0 mL) was added 4.0 M HCl in 1,4-dioxane (1.0 mL). The reaction mixture was stirred at 50° C. for 3 h under a nitrogen atmosphere. After concentrating under vacuum, the crude product, (R)-1-(4-(10-methyl-8-oxo-9,10,11,12-tetrahydro-8H-[1,4]diazepino[5′,6′:4,5]thieno[3,2-f]quinolin-3-yl)phenyl)piperidine-4-carbaldehyde (20.0 mg, 73% yield), was obtained and used directly for the next step without further purification. MS (ESI, m/z) [M+H]⁺ 471.3.

3-(4-(4-((1-(4-((R)-10-Methyl-8-oxo-9,10,11,12-tetrahydro-8H-[1,4]diazepino[5′,6′:4,5]thieno[3,2-f]quinolin-3-yl)phenyl)piperidin-4-yl)methyl)piperazin-1-yl)phenyl)piperidine-2,6-dione. To solution of 3-(4-piperazin-1-ylphenyl)piperidine-2,6-dione hydrochloride salt (13.0 mg, 0.04 mmol) and (R)-1-(4-(10-methyl-8-oxo-9,10,11,12-tetrahydro-8H-[1,4]diazepino[5′,6′:4,5]thieno[3,2-f]quinolin-3-yl)phenyl)piperidine-4-carbaldehyde (20.0 mg, 0.04 mmol) in methanol (2.0 mL) was added NaBH₃CN (8.0 mg, 0.12 mmol). The resulting mixture was stirred at rt for 2 h. After diluted with water (2.0 mL) and filtered, the collected solid was washed by water (2.0 mL), and then purified by reverse-phase chromatography (RPC) using water and acetonitrile (MeCN) as mobile phases with 0.05% trifluoroacetic acid (TFA) to afford the desired product, 3-(4-(4-((1-(4-((R)-10-Methyl-8-oxo-9,10,11,12-tetrahydro-8H-[1,4]diazepino[5′,6′:4,5]thieno[3,2-f]quinolin-3-yl)phenyl)piperidin-4-yl)methyl)piperazin-1-yl)phenyl)piperidine-2,6-dione (2.9 mg, 9.0% yield) as a light-yellow solid. MS (ESI, m/z) [M+H]⁺ 728.3 ¹H NMR (400 MHz, DMSO-d₆) δ ppm 10.8 (s, 1H), 9.14 (d, J=9.0 Hz, 1H), 8.38 (s, 1H), 8.26-8.05 (m, 4H), 7.95 (d, J=8.8 Hz, 1H), 7.18-7.01 (m, 5H), 6.90 (d, J=8.4 Hz, 2H), 3.88 (d, J=12.4 Hz, 2H), 3.75-3.69 (m, 1H), 3.64-3.58 (m, 1H), 3.51-3.40 (m, 2H), 3.11 (s, 4H), 2.89-2.75 (m, 2H), 2.70-2.58 (m, 2H), 2.49-2.41 (m, 1H), 2.35-1.99 (m, 5H), 1.90-1.72 (m, 3H), 1.31-1.15 (m, 6H).

### Example 39: Synthesis of 3-(4-(4-(2-(1-(4-((R)-10-methyl-8-oxo-9,10,11,12-tetrahydro-8H-[1,4]diazepino[5′,6′:4,5]thieno[3,2-f]quinolin-3-yl)phenyl)piperidin-4-yl)ethyl)piperazin-1-yl)phenyl)piperidine-2,6-dione

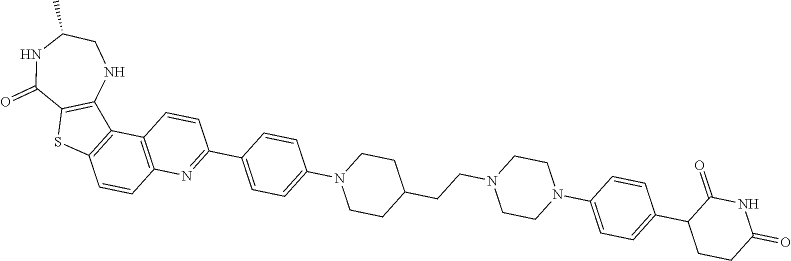 [SMILES: C[C@@H]1CNc2c(sc3ccc4nc(-c5ccc(N6CCC(CCN7CCN(c8ccc(C9CCC(=O)NC9=O)cc8)CC7)CC6)cc5)ccc4c23)C(=O)N1]

tert-Butyl 4-(2-(4-(4-(2,6-dioxopiperidin-3-yl)phenyl)piperazin-1-yl)ethyl)piperidine-1-carboxylate. To a solution of 3-(4-piperazin-1-ylphenyl) piperidine-2,6-dione (100.0 mg, 0.37 mmol) in methanol (10.0 mL), the pH value of the solution was adjusted to 9˜10 with triethyl amine (TEA) and the tert-butyl 4-(2-oxoethyl) piperidine-1-carboxylate (100.0 mg, 0.44 mmol) was added. After the pH value of the solution was adjusted to 5˜6 with acetic acid (AcOH), NaBH₃CN (69.0 mg, 1.1 mmol) was added and stirred at rt for 1 h. The reaction mixture was concentrated under reduced pressure and the residue by reverse-phase chromatography (RPC) using water and acetonitrile (MeCN) as mobile phases with NH₄HCO₃ (10 mmol/L) to afford the desired product, tert-butyl 4-[2-[4-[4-(2,6-dioxo-3-piperidyl) phenyl]piperazin-1-yl]ethyl]piperidine-1-carboxylate (120.0 mg, 67% yield) as a yellow solid. MS (ESI, m/z) [M+H−56]⁺ 429.3.

3-(4-(4-(2-(Piperidin-4-yl)ethyl)piperazin-1-yl)phenyl)piperidine-2,6-dione. To a solution of tert-butyl 4-[2-[4-[4-(2,6-dioxo-3-piperidyl) phenyl]piperazin-1-yl]ethyl]piperidine-1-carboxylate (120.0 mg, 0.25 mmol) in DCM (5.0 mL) was added dropwise with TFA (1.0 mL). The reaction mixture was stirred at rt for 3 h under a nitrogen atmosphere. The reaction mixture was concentrated under reduced pressure to afford the crude product which was used directly for the next step without further purification. MS (ESI, m/z) (M+H)⁺ 385.2.

3-(4-(4-(2-(1-(4-Iodophenyl)piperidin-4-yl)ethyl)piperazin-1-yl)phenyl)piperidine-2,6-dione. To a solution of 3-[4-[4-[2-(4-piperidyl) ethyl]piperazin-1-yl]phenyl]piperidine-2,6-dione (70.0 mg, 0.18 mmol) and TEA (35.0 mg, 0.27 mmol) in DCM (10.0 mL) was added (4-iodophenyl) boronic acid (54.0 mg, 0.22 mmol) and Cu(OAc)₂ (33.0 mg, 0.18 mmol). The reaction mixture was stirred at rt for 2 h under an inert atmosphere. After concentrating under reduced pressure to remove the solvent, and the residue was purified by reverse-phase chromatography (RPC) using water and acetonitrile (MeCN) as mobile phases with 0.05% trifluoroacetic acid (TFA) to afford the desired product, 3-(4-(4-(2-(1-(4-iodophenyl)piperidin-4-yl)ethyl)piperazin-1-yl)phenyl)piperidine-2,6-dione (35.0 mg, 33% yield) as a yellow solid. MS (ESI, m/z) (M+H)⁺ 587.4.

3-(4-(4-(2-(1-(4-((R)-10-methyl-8-oxo-9,10,11,12-tetrahydro-8H-[1,4]diazepino[5′,6′:4,5]thieno[3,2-f]quinolin-3-yl)phenyl)piperidin-4-yl)ethyl)piperazin-1-yl)phenyl)piperidine-2,6-dione. To a solution of 3-(4-(4-(2-(1-(4-iodophenyl)piperidin-4-yl)ethyl)piperazin-1-yl)phenyl)piperidine-2,6-dione (25.0 mg, 0.04 mmol) in DMF (5.0 mL) was added (R)-10-methyl-3-(tributylstannyl)-9,10,11,12-tetrahydro-8H-[1,4]diazepino[5′,6′:4,5]thieno[3,2-f]quinolin-8-one (29.0 mg, 0.05 mmol), P(o-Tol)₃ (5.0 mg, 0.016 mmol) and Pd₂(dba)₃ (9.0 mg, 0.008 mmol). The reaction mixture was stirred at 70° C. for 2 h under a nitrogen atmosphere. The reaction mixture was diluted with DMSO (2.0 mL) and then purified by reverse-phase chromatography (RPC) using water and acetonitrile (MeCN) as mobile phases with 0.1% trifluoroacetic acid (TFA) to afford the desired product; 3-(4-(4-(2-(1-(4-((R)-10-methyl-8-oxo-9,10,11,12-tetrahydro-8H-[1,4]diazepino[5′,6′:4,5]thieno[3,2-f]quinolin-3-yl)phenyl)piperidin-4-yl)ethyl)piperazin-1-yl)phenyl)piperidine-2,6-dione (8.6 mg, 27% yield) as a brown solid. MS (ESI, m/z) (M+H)⁺ 742.3 ¹H NMR (400 MHz, DMSO-d₆) δ ppm 10.77 (s, 1H), 9.14 (d, J=9.0 Hz, 1H), 8.21-8.03 (m, 5H), 7.95 (d, J=8.8 Hz, 1H), 7.15-7.02 (m, 5H), 6.90 (d, J=8.2 Hz, 2H), 3.86 (d, J=12.2 Hz, 2H), 3.78-3.70 (m, 1H), 3.66-3.55 (m, 2H), 3.50-3.40 (m, 2H), 3.18-3.13 (m, 5H), 2.88-2.40 (m, 8H), 2.20-2.12 (m, 1H), 1.96-2.05 (m, 1H), 1.80 (d, J=12.4 Hz, 2H), 1.62-1.45 (m, 3H), 1.40-1.13 (m, 5H).

### Example 40: Synthesis of 3-(4-(1-(2-(1-(4-((R)-10-Methyl-8-oxo-9,10,11,12-tetrahydro-8H-[1,4]diazepino[5′,6′:4,5]thieno[3,2-f]quinolin-3-yl)phenyl)piperidin-4-yl)ethyl)piperidin-4-yl)phenyl)piperidine-2,6-dione

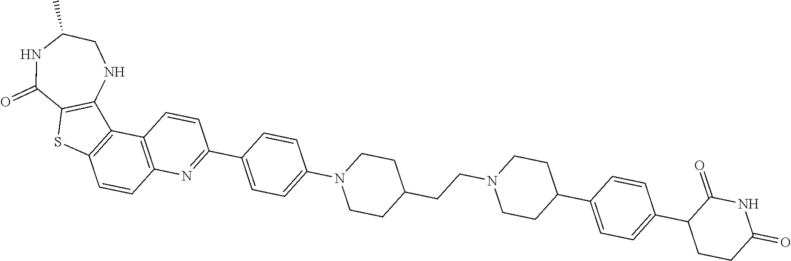 [SMILES: C[C@@H]1CNc2c(sc3ccc4nc(-c5ccc(N6CCC(CCN7CCC(c8ccc(C9CCC(=O)NC9=O)cc8)CC7)CC6)cc5)ccc4c23)C(=O)N1]

tert-Butyl 4-(2-(4-(4-(2,6-dioxopiperidin-3-yl)phenyl)piperidin-1-yl)ethyl)piperidine-1-carboxylate. To a solution of 3-[4-(4-piperidyl)phenyl]piperidine-2,6-dione (200.0 mg, 0.73 mmol) in methanol (3.0 mL) was added tert-butyl 4-(2-oxoethyl)piperidine-1-carboxylate (250.0 mg, 1.1 mmol) and NaBH₃CN (92.0 mg, 1.46 mmol). The resulting solution was stirred at rt overnight. The reaction mixture was diluted with water (50 mL), extracted with ethyl acetate (3×50 mL). The combined organic layers were washed with brine (30 mL×2), dried over anhydrous sodium sulfate, filtered, and concentrated under reduced pressure to afford the crude product, tert-butyl 4-[2-[4-[4-(2,6-dioxo-3-piperidyl)phenyl]-1-piperidyl]ethyl]piperidine-1-carboxylate (320.0 mg) as a white solid. It was directly used in the next step without further purification. MS (ESI, m/z) [M+H]⁺ 484.3.

3-(4-(1-(2-(Piperidin-4-yl)ethyl)piperidin-4-yl)phenyl)piperidine-2,6-dione. To a solution of tert-butyl 4-[2-[4-[4-(2,6-dioxo-3-piperidyl)phenyl]-1-piperidyl]ethyl]piperidine-1-carboxylate (320.0 mg, 0.66 mmol) in DCM (5.0 mL) was added dropwise with TFA (2.5 mL). The resulting solution was stirred at rt for 2 h. The reaction mixture was concentrated under reduced pressure, and the residue was purified by reverse-phase chromatography (RPC) using water and acetonitrile (MeCN) as mobile phases with 0.05% trifluoroacetic acid (TFA) to afford the desired product, 3-[4-[1-[2-(4-piperidyl)ethyl]-4-piperidyl]phenyl]piperidine-2,6-dione (270.0 mg) as a yellow solid. MS (ESI, m/z) [M+H]⁺ 384.2.

3-(4-(1-(2-(1-(4-Iodophenyl)piperidin-4-yl)ethyl)piperidin-4-yl)phenyl)piperidine-2,6-dione. To a solution of 3-[4-[1-[2-(4-piperidyl)ethyl]-4-piperidyl]phenyl]piperidine-2,6-dione (220.0 mg, 0.57 mmol) in DCM (5.0 mL) was added (4-iodophenyl)boronic acid (213.0 mg, 0.86 mmol), Cu(OAc)₂ (91.0 mg, 0.57 mmol) and TEA (148.0 mg, 1.15 mmol). The resulting solution was stirred at rt for 2 h. The reaction mixture was concentrated under reduced pressure, and the residue was purified by reverse-phase chromatography (RPC) using water and acetonitrile (MeCN) as mobile phases with 0.05% trifluoroacetic acid (TFA) to afford the desired product; 3-[4-[1-[2-[1-(4-iodophenyl)-4-piperidyl]ethyl]-4-piperidyl]phenyl]piperidine-2,6-dione (40.0 mg, 12% yield) as a white solid. MS (ESI, m/z) [M+H]⁺ 586.2.

3-(4-(1-(2-(1-(4-((R)-10-Methyl-8-oxo-9,10,11,12-tetrahydro-8H-[1,4]diazepino[5′,6′:4,5]thieno[3,2-f]quinolin-3-yl)phenyl)piperidin-4-yl)ethyl)piperidin-4-yl)phenyl)piperidine-2,6-dione. To a solution of 3-[4-[1-[2-[1-(4-iodophenyl)-4-piperidyl]ethyl]-4-piperidyl]phenyl]piperidine-2,6-dione (40.0 mg, 0.07 mmol) and (R)-10-methyl-3-(tributylstannyl)-9,10,11,12-tetrahydro-8H-[1,4]diazepino[5′,6′:4,5]thieno[3,2-f]quinolin-8-one (39.0 mg, 0.07 mmol) in 1,4-dioxane (1.0 mL) was added Pd(PPh₃)₄(11.0 mg, 0.01 mmol). The resulting solution was stirred at 70° C. for 6 h under a nitrogen atmosphere. After cooling down rt and filtrated, the filtrate was purified by reverse-phase chromatography (RPC) by using water and acetonitrile (MeCN) as mobile phases with 0.05% trifluoroacetic acid (TFA) to afford the desired product, 3-(4-(1-(2-(1-(4-((R)-10-Methyl-8-oxo-9,10,11,12-tetrahydro-8H-[1,4]diazepino[5′,6′:4,5]thieno[3,2-f]quinolin-3-yl)phenyl)piperidin-4-yl)ethyl)piperidin-4-yl)phenyl)piperidine-2,6-dione (8.4 mg, 16% yield) as a reddish oil. MS (ESI, m/z) [M+H]⁺ 741.2 ¹H NMR (400 MHz, DMSO-d₆) δ ppm 9.72 (s, 1H), 9.28 (d, J=8.9 Hz, 1H), 8.76-8.68 (m, 1H), 8.60-8.30 (m, 3H), 8.28 (d, J=8.9 Hz, 1H), 8.15 (d, J=8.8 Hz, 1H), 8.10-8.00 (m, 2H), 7.60-7.38 (m, 2H), 7.35-7.29 (m, 2H), 7.28-7.20 (m, 2H), 7.19-7.00 (m, 1H), 4.21-4.12 (m, 1H), 3.64-3.61 (m, 2H), 3.50 (d, J=5.6 Hz, 2H), 3.31-3.29 (m, 3H), 3.17-2.92 (m, 5H), 2.92-2.76 (m, 4H), 2.47-2.39 (m, 1H), 2.24-2.15 (m, 1H), 2.03 (d, J=13.7 Hz, 2H), 1.98-1.80 (m, 4H), 1.71-1.57 (m, 3H), 1.5-1.18 (m, 6H). ¹⁹F NMR (376 MHz, DMSO-d₆) δ ppm ˜74.91.

### Example 41: Synthesis of 3-(4-(4-((1-(4-((R)-10-Methyl-8-oxo-9,10,11,12-tetrahydro-8H-[1,4]diazepino[5′,6′:4,5]thieno[3,2-f]quinolin-3-yl)phenyl)azetidin-3-yl)methyl)piperazin-1-yl)phenyl)piperidine-2,6-dione

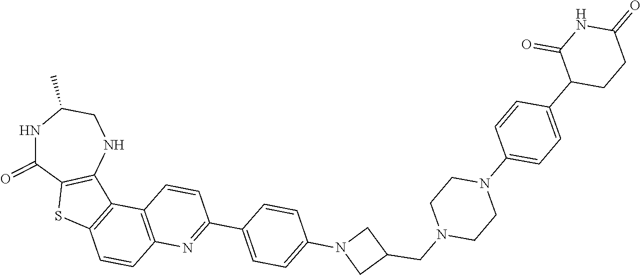 [SMILES: C[C@@H]1CNc2c(sc3ccc4nc(-c5ccc(N6CC(CN7CCN(c8ccc(C9CCC(=O)NC9=O)cc8)CC7)C6)cc5)ccc4c23)C(=O)N1]

tert-Butyl 3-((4-(4-(2,6-dioxopiperidin-3-yl)phenyl)piperazin-1-yl)methyl)azetidine-1-carboxylate. To a solution of 3-(4-piperazin-1-ylphenyl) piperidine-2,6-dione (100.0 mg, 0.37 mmol) in methanol (5.0 mL), the pH value of the solution was adjusted to 9˜10 with triethyl amine (TEA), and then tert-butyl 3-formylazetidine-1-carboxylate (102.0 mg, 0.55 mmol) was added and the pH value of the solution was further adjusted to 5˜6 with acetic acid (AcOH). After the addition of NaBH₃CN (69.0 mg, 1.11 mmol). The resulting solution was stirred at rt for 1 h, and then was diluted with water (50 mL), extracted with dichloromethane (50 mL×3). The combined organic layer was washed with brine (50 mL×2), dried over anhydrous sodium sulfate, filtered, and concentrated under reduced pressure. The residue was purified by flash chromatography on silica gel with dichloromethane/methanol (10:1) to afford the desired product, tert-butyl 3-[[4-[4-(2,6-dioxo-3-piperidyl)phenyl]piperazin-1-yl]methyl]azetidine-1-carboxylate (120.0 mg, 74% yield) as a colorless oil. MS (ESI, m/z) [M+H]⁺ 443.2.

3-(4-(4-(Azetidin-3-ylmethyl)piperazin-1-yl)phenyl)piperidine-2,6-dione. To a solution of tert-butyl 3-[[4-[4-(2,6-dioxo-3-piperidyl)phenyl]piperazin-1-yl]methyl]azetidine-1-carboxylate (120.0 mg, 0.27 mmol) in DCM (5.0 mL) was added TFA (1.0 mL). The reaction mixture was stirred at rt overnight. After concentrated under reduced pressure, the crude product was obtained and used directly for the next step without further purification. MS (ESI, m/z) [M+H]⁺ 343.1.

3-(4-(4-((1-(4-Bromophenyl)azetidin-3-yl)methyl)piperazin-1-yl)phenyl)piperidine-2,6-dione. To a solution of 3-[4-[4-(azetidin-3-ylmethyl)piperazin-1-yl]phenyl]piperidine-2,6-dione (100.0 mg, 0.29 mmol) and TEA (44.0 mg, 0.44 mmol) in DCM (10.0 mL) was added (4-bromophenyl)boronic acid (88.0 mg, 0.44 mmol) and Cu(OAc)₂ (46.0 mg, 0.29 mmol), the resulting solution was stirred at rt for 2 h under an inert atmosphere. The reaction mixture was diluted with water (30 mL) and extracted by dichloromethane (40 mL×4). The combined organic layer was washed with brine (30 mL×2), dried over anhydrous sodium sulfate, filtered, and concentrated under reduced pressure. The residue was directly used for the next step without further purification. MS (ESI, m/z) [M+H]⁺ 498.3.

3-(4-(4-((1-(4-((R)-10-Methyl-8-oxo-9,10,11,12-tetrahydro-8H-[1,4]diazepino[5′,6′:4,5]thieno[3,2-f]quinolin-3-yl)phenyl)azetidin-3-yl)methyl)piperazin-1-yl)phenyl)piperidine-2,6-dione. To a solution of 3-[4-[4-[[1-(4-bromophenyl)azetidin-3-yl]methyl]piperazin-1-yl]phenyl]piperidine-2,6-dione (60.0 mg, 0.120 mmol) and (R)-10-methyl-3-(tributylstannyl)-9,10,11,12-tetrahydro-8H-[1,4]diazepino[5′,6′:4,5]thieno[3,2-f]quinolin-8-one (69.0 mg, 0.12 mmol) in 1,4-dioxane (2.5 mL) was added Pd₂(dba)₃ (12.0 mg, 0.010 mmol) and P(o-Tol.)₃ (7.0 mg, 0.02 mmol). The mixture was stirred at 60° C. for 2 h under a nitrogen atmosphere. After cooling down to rt, the reaction mixture was concentrated under reduced pressure, and the residue was purified by reverse-phase chromatography (RPC) using water and acetonitrile (MeCN) as mobile phases with 0.05% trifluoroacetic acid (TFA) to afford the desired product, 3-(4-(4-((1-(4-((R)-10-Methyl-8-oxo-9,10,11,12-tetrahydro-8H-[1,4]diazepino[5′,6′:4,5]thieno[3,2-f]quinolin-3-yl)phenyl)azetidin-3-yl)methyl)piperazin-1-yl)phenyl)piperidine-2,6-dione (3.0 mg, 3% yield) as a yellow solid. MS (ESI, m/z) [M+H]⁺ 700.3 ¹H NMR (400 MHz, DMSO-d₆) δ ppm 10.77 (s, 1H), 9.12 (d, J=8.0 Hz, 1H), 8.17 (d, J=8.4 Hz, 2H), 8.10 (d, J=8.8 Hz, 1H), 8.10-8.02 (m, 2H), 7.94 (d, J=8.8 Hz, 1H), 7.10 (t, J=5.2 Hz, 1H), 7.05 (d, J=8.4 Hz, 2H), 6.89 (d, J=8.1 Hz, 2H), 6.56 (d, J=8.4 Hz, 2H), 4.04 (t, J=7.6 Hz, 2H), 3.73 (dd, J=11.2, 4.8 Hz, 1H), 3.63-3.56 (m, 4H), 3.46 (s, 3H), 3.12 (s, 4H), 3.01 (s, 1H), 2.70-2.57 (m, 3H), 2.54 (s, 4H), 2.51-2.41 (m, 1H), 2.12 (dd, J=12.4, 8.4 Hz, 1H), 2.01 (dd, J=13.2, 5.2 Hz, 1H), 1.20 (d, J=6.8 Hz, 3H).

### Example 42: Synthesis of 3-(4-(4-(2-(1-(4-((R)-10-Methyl-8-oxo-9,10,11,12-tetrahydro-8H-[1,4]diazepino[5′,6′:4,5]thieno[3,2-f]quinolin-3-yl)phenyl)azetidin-3-yl)ethyl)piperazin-1-yl)phenyl)piperidine-2,6-dione

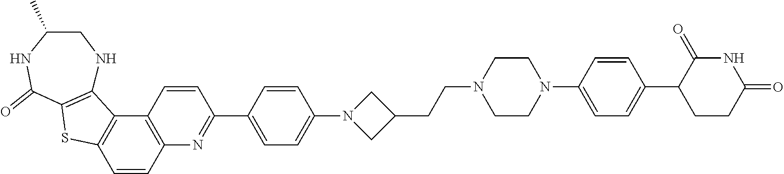 [SMILES: C[C@@H]1CNc2c(sc3ccc4nc(-c5ccc(N6CC(CCN7CCN(c8ccc(C9CCC(=O)NC9=O)cc8)CC7)C6)cc5)ccc4c23)C(=O)N1]

tert-Butyl 3-(2-(4-(4-(2,6-dioxopiperidin-3-yl)phenyl)piperazin-1-yl)ethyl)azetidine-1-carboxylate. To a solution of 3-(4-piperazin-1-ylphenyl) piperidine-2,6-dione (100.0 mg, 0.37 mmol) in methanol (10.0 mL), the pH for the solution was adjusted to 9˜10 with triethylamine (TEA), and then tert-butyl 3-(2-oxoethyl) azetidine-1-carboxylate (87.0 mg, 0.44 mmol) was added. After the pH for reaction mixture was adjusted to 5˜6 with acetic acid (AcOH) and the NaBH₃CN (69.0 mg, 1.10 mmol) was added, the reaction mixture was stirred at rt for 1 h. After removed the solvent under vacuum, the residue was purified by reverse-phase chromatography (RPC) using water and acetonitrile (MeCN) with NH₄HCO₃ (10 mmol/L) to afford the desired product, tert-butyl 3-[2-[4-[4-(2,6-dioxo-3-piperidyl) phenyl]piperazin-1-yl]ethyl]azetidine-1-carboxylate (120.0 mg, 71% yield) as an off-white solid. MS (ESI, m/z) [M+H−56]⁺ 401.1.

3-(4-(4-(2-(Azetidin-3-yl)ethyl)piperazin-1-yl)phenyl)piperidine-2,6-dione To a solution of tert-butyl 3-[2-[4-[4-(2,6-dioxo-3-piperidyl) phenyl]piperazin-1-yl]ethyl]azetidine-1-carboxylate (120.0 mg, 0.26 mmol) in DCM (5.0 mL) was added with TFA (1.0 mL). The resulting solution was stirred at rt for 3 h under a nitrogen atmosphere. The reaction mixture was concentrated under reduced pressure to afford the crude product. It was directly used for the next step without further purification. MS (ESI, m/z) [M+H]⁺ 357.2.

3-(4-(4-(2-(1-(4-Bromophenyl)azetidin-3-yl)ethyl)piperazin-1-yl)phenyl)piperidine-2,6-dione. To a solution of 3-[4-[4-[2-(azetidin-3-yl) ethyl]piperazin-1-yl]phenyl]piperidine-2,6-dione (80.0 mg, 0.22 mmol) and TEA (43.0 mg, 0.34 mmol) in DCM (10.0 mL) was added by (4-bromophenyl) boronic acid (54.0 mg, 0.27 mmol) and Cu(OAc)₂ (40.0 mg, 0.22 mmol). The resulting solution was stirred at rt for 2 h under an inert atmosphere. After removing the solvent under vacuum, the residue was purified by reverse-phase chromatography (RPC) using water and acetonitrile (MeCN) as mobile phases with 0.05% trifluoroacetic acid (TFA) to afford the desired product, 3-[4-[4-[2-[1-(4-bromophenyl) azetidin-3-yl]ethyl]piperazin-1-yl]phenyl]piperidine-2,6-dione (40.0 mg, 35% yield) as a yellow solid. MS (ESI, m/z) [M+H]⁺ 511.3.

3-(4-(4-(2-(1-(4-((R)-10-Methyl-8-oxo-9,10,11,12-tetrahydro-8H-[1,4]diazepino[5′,6′:4,5]thieno[3,2-f]quinolin-3-yl)phenyl)azetidin-3-yl)ethyl)piperazin-1-yl)phenyl)piperidine-2,6-dione. To a solution of 3-(4-(4-(2-(1-(4-bromophenyl)azetidin-3-yl)ethyl)piperazin-1-yl)phenyl)piperidine-2,6-dione (40.0 mg, 0.08 mmol) in DMF (2.0 mL) was added (R)-10-methyl-3-(tributylstannyl)-9,10,11,12-tetrahydro-8H-[1,4]diazepino[5′,6′:4,5]thieno[3,2-f]quinolin-8-one (53 mg, 0.09 mmol), P(o-Tol.)₃ (10.0 mg, 0.03 mmol) and Pd₂(dba)₃ (16.0 mg, 0.02 mmol). The resulting mixture was stirred at 60° C. for 2 h under a nitrogen atmosphere. After cooling down to rt, diluted with DMSO (2.0 mL) and filtered, the filtrate was purified by reverse-phase chromatography (RPC) using water and acetonitrile (MeCN) as mobile phases with 0.05% trifluoroacetic acid (TFA) to afford the desired product, 3-(4-(4-(2-(1-(4-((R)-10-methyl-8-oxo-9,10,11,12-tetrahydro-8H-[1,4]diazepino[5′,6′:4,5]thieno[3,2-f]quinolin-3-yl)phenyl)azetidin-3-yl)ethyl)piperazin-1-yl)phenyl)piperidine-2,6-dione (2.5 mg, 4.0% yield) as a brown solid. MS (ESI, m/z) [M+H]⁺ 714.25 ¹H NMR (400 MHz, MeOD-d₁) δ ppm 9.31 (t, J=8.4 Hz, 2H), 8.18-8.05 (m, 4H), 7.99 (d, J=8.4 Hz, 2H), 7.17 (d, J=8.0 Hz, 2H), 7.02 (d, J=8.4 Hz, 2H), 6.86 (d, J=8.4 Hz, 2H), 4.08-4.03 (m, 1H), 3.90-3.30 (m, 15H), 3.15-3.00 (m, 1H), 2.72-2.44 (m, 2H), 2.25-2.18 (m, 3H), 1.42-1.25 (m, 5H).

### Example 43: Synthesis of 3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)-1H-pyrazol-1-yl)piperidin-1-yl)ethyl)piperazin-1-yl)phenyl)piperidine-2,6-dione

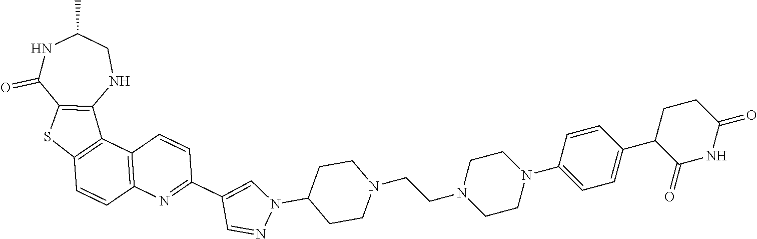 [SMILES: C[C@@H]1CNc2c(sc3ccc4nc(-c5cnn(C6CCN(CCN7CCN(c8ccc(C9CCC(=O)NC9=O)cc8)CC7)CC6)c5)ccc4c23)C(=O)N1]

tert-Butyl 4-(3-(4,4,5,5-tetramethyl-1,3,2-dioxaborolan-2-yl)-1H-pyrazol-1-yl)piperidine-1-carboxylate. To a solution of tert-butyl 4-(4-bromopyrazol-1-yl)piperidine-1-carboxylate (500.0 mg, 1.51 mmol), B₂pin₂ (576.0 mg, 2.27 mmol) and KOAc (297.0 mg, 3.03 mmol) in 1,4-dioxane (10.0 mL) was added Pd(dppf)Cl₂.DCM (247.0 mg, 0.30 mmol). The resulting mixture was stirred at 80° C. for 4 h under a nitrogen atmosphere. After removed the solvent under vacuum, the obtained crude product was directly used for the next step without further purification. MS (ESI, m/z) [(M+H]⁺ 378.3.

tert-Butyl (R)-4-(4-(10-methyl-8-oxo-9,10,11,12-tetrahydro-8H-[1,4]diazepino[5′,6′:4,5]thieno[3,2-f]quinolin-3-yl)-1H-pyrazol-1-yl)piperidine-1-carboxylate. To a solution of (R)-3-chloro-10-methyl-9,10,11,12-tetrahydro-8H-[1,4]diazepino[5′,6′:4,5]thieno[3,2-f]quinolin-8-one (300.0 mg, 0.94 mmol), tert-butyl 4-(4-methylpyrazol-1-yl)piperidine-1-carboxylate (375.0 mg, 1.42 mmol) and Cs₂CO₃ (306.0 mg, 0.94 mmol) in 1,4-dioxane (5.0 mL) and water (0.5 mL) was added Pd(dppf)Cl₂ (155.0 mg, 0.19 mmol). The resulting mixture was stirred at 100° C. for 4 h under a nitrogen atmosphere. The reaction mixture was then quenched with water (20.0 mL) and extracted with ethyl acetate (20.0 mL×3). The combined organic layers were washed with brine, dried over sodium sulfate, filtered, and concentrated under reduced pressure. The residue was purified by column chromatography on silica gel with petroleum ether/ethyl acetate (1:1) to afford the desired product, tert-Butyl (R)-4-(4-(10-methyl-8-oxo-9,10,11,12-tetrahydro-8H-[1,4]diazepino[5′,6′:4,5]thieno[3,2-f]quinolin-3-yl)-1H-pyrazol-1-yl)piperidine-1-carboxylate (400.0 mg, 80% yield) as a yellow solid. MS (ESI, m/z) [M+H]⁺ 533.2.

(R)-10-Methyl-3-(1-(piperidin-4-yl)-1H-pyrazol-4-yl)-9,10,11,12-tetrahydro-8H-[1,4]diazepino[5′,6′:4,5]thieno[3,2-f]quinolin-8-one. To a solution of tert-butyl (R)-4-(4-(10-methyl-8-oxo-9,10,11,12-tetrahydro-8H-[1,4]diazepino[5′,6′:4,5]thieno[3,2-f]quinolin-3-yl)-1H-pyrazol-1-yl)piperidine-1-carboxylate (400.0 mg, 0.75 mmol) in 1,4-dioxane (5.0 mL) was added dropwise with HCl 4.0 M in 1,4-dioxane (5 mL). The resulting mixture was stirred for 1 h at rt. The reaction mixture was concentrated under reduced pressure to afford the crude product, (R)-10-methyl-3-(1-(piperidin-4-yl)-1H-pyrazol-4-yl)-9,10,11,12-tetrahydro-8H-[1,4]diazepino[5′,6′:4,5]thieno[3,2-f]quinolin-8-one (300.0 mg) as a yellow solid. MS (ESI, m/z) [M+H]⁺ 433.2. It is used directly for the next step without further purification.

(R)-3-(1-(1-(2,2-Dimethoxyethyl)piperidin-4-yl)-1H-pyrazol-4-yl)-10-methyl-9,10,11,12-tetrahydro-8H-[1,4]diazepino[5′,6′:4,5]thieno[3,2-f]quinolin-8-one. To a solution of (R)-10-methyl-3-(1-(piperidin-4-yl)-1H-pyrazol-4-yl)-9,10,11,12-tetrahydro-8H-[1,4]diazepino[5′,6′:4,5]thieno[3,2-f]quinolin-8-one (300.0 mg, 0.69 mmol) in methanol (5.0 mL), the pH value of the solution was adjusted to 9˜10 with triethyl amine (TEA), and then followed by the addition of the 2,2-dimethoxyacetaldehyde (72.0 mg, 0.69 mmol). After the pH value of the solution was further adjusted to 5˜6 with acetic acid (AcOH), the NaBH₃CN (133.0 mg, 2.08 mmol) was added, and the resulting mixture was stirred at rt for 1 h. After removing the solvent under reduced pressure, and the residue was purified by reverse-phase chromatography (RPC) using water and acetonitrile (MeCN) as mobile phases with 0.05% trifluoroacetic acid (TFA) to afford the desired product, (R)-3-(1-(1-(2,2-dimethoxyethyl)piperidin-4-yl)-1H-pyrazol-4-yl)-10-methyl-9,10,11,12-tetrahydro-8H-[1,4]diazepino[5′,6′:4,5]thieno[3,2-f]quinolin-8-one (210.0 mg, 58% yield) as a yellow solid. MS (ESI, m/z) [M+H]⁺ 521.2.

(R)-2-(4-(4-(10-Methyl-8-oxo-9,10,11,12-tetrahydro-8H-[1,4]diazepino[5′,6′:4,5]thieno[3,2-f]quinolin-3-yl)-1H-pyrazol-1-yl)piperidin-1-yl)acetaldehyde. To a solution of (R)-3-(1-(1-(2,2-dimethoxyethyl)piperidin-4-yl)-1H-pyrazol-4-yl)-10-methyl-9,10,11,12-tetrahydro-8H-[1,4]diazepino[5′,6′:4,5]thieno[3,2-f]quinolin-8-one (190.0 mg, 0.36 mmol) 1,4-dioxane (5.0 mL) was added HCl (5.0 mL, 4.0 M in 1,4-dioxane). The resulting solution was stirred at 50° C. for 2 h, concentrated under reduced pressure to afford the crude product, (R)-2-(4-(4-(10-methyl-8-oxo-9,10,11,12-tetrahydro-8H-[1,4]diazepino[5′,6′:4,5]thieno[3,2-(]quinolin-3-yl)-1H-pyrazol-1-yl)piperidin-1-yl)acetaldehyde (110.0 mg) as a red solid, which was used directly for the next step without further purification. MS (ESI, m/z) [M+H]⁺ 493.3.

3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)-1H-pyrazol-1-yl)piperidin-1-yl)ethyl)piperazin-1-yl)phenyl)piperidine-2,6-dione. To a solution of 3-(4-piperazin-1-ylphenyl)piperidine-2,6-dione (29.0 mg, 0.110 mmol) in methanol (3.0 mL), the pH value for the solution was adjusted to 9˜10 with triethyl amine (TEA), and then the (R)-2-(4-(4-(10-methyl-8-oxo-9,10,11,12-tetrahydro-8H-[1,4]diazepino[5′,6′:4,5]thieno[3,2-f]quinolin-3-yl)-1H-pyrazol-1-yl)piperidin-1-yl)acetaldehyde (50.0 mg, 0.11 mmol) was added. The pH value for the resulting solution was further adjusted to 5˜6 with acetic acid (AcOH). After the addition of the NaBH₃CN (20.0 mg, 0.32 mmol), the resulting mixture was stirred at rt for 1 h. The reaction mixture was then concentrated under reduced pressure, and the residue was purified by reverse-phase chromatography (RPC) using water and acetonitrile (MeCN) as mobile phases with NH₄HCO₃ (10 mmol/L) to afford the desired product, 3-(4-(4-(2-(4-(4-((R)-10-Methyl-8-oxo-9,10,11,12-tetrahydro-8H-[1,4]diazepino[5′,6′:4,5]thieno[3,2-(]quinolin-3-yl)-1H-pyrazol-1-yl)piperidin-1-yl)ethyl)piperazin-1-yl)phenyl)piperidine-2,6-dion (10.2 mg, 13% yield) as a yellow solid. MS (ESI, m/z) [M+H]⁺ 732.3 ¹H NMR (300 MHz, DMSO-d₆) δ ppm 10.76 (s, 1H), 9.11 (d, J=9.0 Hz, 1H), 8.58 (s, 1H), 8.21 (s, 1H), 8.12-8.01 (m, 2H), 7.91 (t, J=9.3 Hz, 2H), 7.15-7.00 (m, 3H), 6.89 (d, J=8.4 Hz, 2H), 4.32-4.18 (m, 1H), 3.81-3.70 (m, 1H), 3.69-3.61 (m, 1H), 3.49-3.40 (m, 2H), 3.21-2.99 (m, 7H), 2.73-2.52 (m, 9H), 2.29-1.98 (m, 8H), 1.20 (d, J=6.9 Hz, 3H).

### Example 44: Synthesis of 3-(1-Methyl-6-(4-(2-(4-(4-((R)-10-methyl-8-oxo-9,10,11,12-tetrahydro-8H-[1,4]diazepino[5′,6′:4,5]thieno[3,2-f]quinolin-3-yl)-1H-pyrazol-1-yl)piperidin-1-yl)ethyl)piperazin-1-yl)-1H-indazol-3-yl)piperidine-2,6-dione

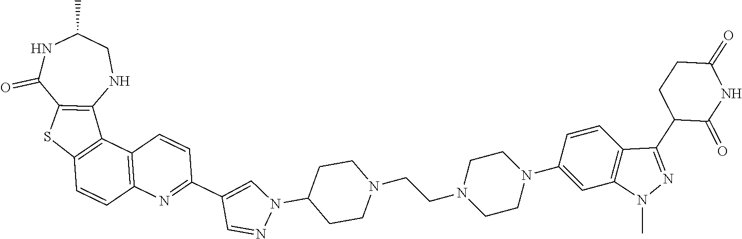 [SMILES: C[C@@H]1CNc2c(sc3ccc4nc(-c5cnn(C6CCN(CCN7CCN(c8ccc9c(C%10CCC(=O)NC%10=O)nn(C)c9c8)CC7)CC6)c5)ccc4c23)C(=O)N1]

3-(1-Methyl-6-(4-(2-(4-(4-((R)-10-methyl-8-oxo-9,10,11,12-tetrahydro-8H-[1,4]diazepino[5′,6′:4,5]thieno[3,2-f]quinolin-3-yl)-1H-pyrazol-1-yl)piperidin-1-yl)ethyl)piperazin-1-yl)-1H-indazol-3-yl)piperidine-2,6-dione. To solution of 3-(1-methyl-6-piperazin-1-yl-indazol-3-yl)piperidine-2,6-dione hydrochloride (115.0 mg, 0.32 mmol) and (R)-2-(4-(4-(10-methyl-8-oxo-9,10,11,12-tetrahydro-8H-[1,4]diazepino[5′,6′:4,5]thieno[3,2-f]quinolin-3-yl)-1H-pyrazol-1-yl)piperidin-1-yl)acetaldehyde (150.0 mg, 0.32 mmol) in methanol (5.0 mL) was added NaBH₃CN (61.0 mg, 0.95 mmol). The reaction mixture was stirred at rt for 1 h and then diluted with water (10 mL). After filtration, the solid was washed with water, dissolved into DMSO (2.0 mL), and then purified by reverse-phase chromatography (RPC) using water and acetonitrile (MeCN) as mobile phases with 0.05% trifluoroacetic acid (TFA) to afford the desired product, 3-(1-methyl-6-(4-(2-(4-(4-((R)-10-methyl-8-oxo-9,10,11,12-tetrahydro-8H-[1,4]diazepino[5′,6′:4,5]thieno[3,2-f]quinolin-3-yl)-1H-pyrazol-1-yl)piperidin-1-yl)ethyl)piperazin-1-yl)-1H-indazol-3-yl)piperidine-2,6-dione (75.2 mg, 30% yield) as an orange solid. MS (ESI, m/z) [M+H]⁺ 787.2 ¹H NMR (400 MHz, MeOD-d₁) δ ppm 9.49 (d, J=9.2 Hz, 1H), 8.73 (s, 1H), 8.43 (s, 1H), 8.27 (d, J=9.2 Hz, 1H), 8.17 (d, J=8.8 Hz, 1H), 8.12 (s, 1H), 7.61 (d, J=9.2 Hz, 1H), 7.01 (d, J=9.2 Hz, 1H), 6.92 (s, 1H), 4.68-4.64 (m, 1H), 4.32-4.29 (m, 1H), 3.96 (s, 1H), 3.80-3.68 (m, 3H), 3.68-3.60 (m, 2H), 3.58-3.45 (m, 4H), 3.43-3.25 (m, 4H), 3.18-3.10 (m, 2H), 2.72-2.68 (m, 2H), 2.52-2.40 (m, 5H), 2.32-2.27 (m, 1H), 1.43 (d, J=6.8 Hz, 3H).

### Example 45: Synthesis of 3-(4-(4-(2-(4-(1-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)ethyl)piperazin-1-yl)phenyl)piperidine-2,6-dione

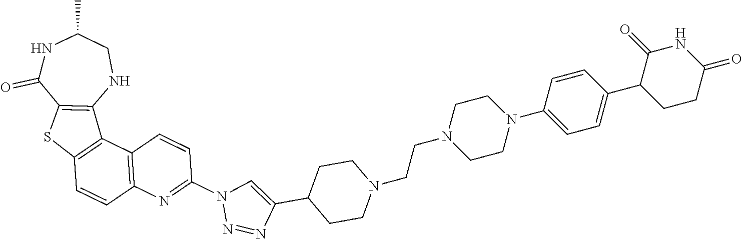 [SMILES: C[C@@H]1CNc2c(sc3ccc4nc(-n5cc(C6CCN(CCN7CCN(c8ccc(C9CCC(=O)NC9=O)cc8)CC7)CC6)nn5)ccc4c23)C(=O)N1]

(R)-3-Azido-10-methyl-9,10,11,12-tetrahydro-8H-[1,4]diazepino[5′,6′:4,5]thieno[3,2-f]quinolin-8-one. To a solution of (R)-3-chloro-10-methyl-9,10,11,12-tetrahydro-8H-[1,4]diazepino[5′,6′:4,5]thieno[3,2-f]quinolin-8-one (1.0 g, 3.15 mmol) in DMSO (10.0 mL) was added NaN₃ (409.0 mg, 6.29 mmol). The resulting solution was stirred at 120° C. overnight under nitrogen atmosphere. The reaction mixture was diluted with water (100.0 mL), and the participates was collected by filtration and washed with water (50 mL×2). The collected solid was dried under vacuum to afford the crude product, (R)-3-azido-10-methyl-9,10,11,12-tetrahydro-8H-[1,4]diazepino[5′,6′:4,5]thieno[3,2-f]quinolin-8-one (900.0 mg). It was used directly for the next step without further purification. MS (ESI, m/z) [M+H]⁺ 325.

tert-Butyl (R)-4-(1-(10-methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidine-1-carboxylate. To a solution of (R)-3-azido-10-methyl-9,10,11,12-tetrahydro-8H-[1,4]diazepino[5′,6′:4,5]thieno[3,2-f]quinolin-8-one (1.0 g, 3.08 mmol), tert-butyl 4-ethynylpiperidine-1-carboxylate (900.0 mg, 4.62 mmol) and DIPEA (1.2 g, 9.25 mmol) in DMF (20.0 mL) was added CuI (58.0 mg, 0.92 mmol). The resulting solution was stirred at 140° C. overnight under a nitrogen atmosphere. After cooling down to rt, the reaction mixture was diluted with ethyl acetate (20.0 mL). After filtration and concentration under reduced pressure, the residue was purified by reverse-phase chromatography (RPC) using water and acetonitrile (MeCN) as mobile phases with 0.05% trifluoroacetic acid (TFA) to afford the desired product, tert-butyl (R)-4-(1-(10-methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidine-1-carboxylate (970.0 mg, 58% yield) as a yellow solid. MS (ESI, m/z) [M+H]⁺ 534.3.

(R)-10-Methyl-3-(4-(piperidin-4-yl)-1H-1,2,3-triazol-1-yl)-9,10,11,12-tetrahydro-8H-[1,4]diazepino[5′,6′:4,5]thieno[3,2-f]quinolin-8-one. To a solution of tert-butyl (R)-4-(1-(10-methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidine-1-carboxylate (100.0 mg, 0.19 mmol) in DCM (2.5 mL) was added trifluoroacetic acid (0.5 mL). The resulting solution was stirred at rt for 2 h. After concentrating under reduced pressure, the residue was purified by reverse-phase chromatography (RPC) using water and acetonitrile (MeCN) as mobile phases with 0.05% trifluoroacetic acid (TFA) to afford the desired product, (R)-10-methyl-3-(4-(piperidin-4-yl)-1H-1,2,3-triazol-1-yl)-9,10,11,12-tetrahydro-8H-[1,4]diazepino[5′,6′:4,5]thieno[3,2-f]quinolin-8-one (6.2 mg, 8% yield) as a yellow solid. MS (ESI, m/z) [M+H]⁻ 434.1.

(R)-3-(4-(1-(2,2-Dimethoxyethyl)piperidin-4-yl)-1H-1,2,3-triazol-1-yl)-10-methyl-9,10,11,12-tetrahydro-8H-[1,4]diazepino[5′,6′:4,5]thieno[3,2-f]quinolin-8-one. To a solution of (R)-10-methyl-3-(4-(piperidin-4-yl)-1H-1,2,3-triazol-1-yl)-9,10,11,12-tetrahydro-8H-[1,4]diazepino[5′,6′:4,5]thieno[3,2-f]quinolin-8-one (500.0 mg, 1.15 mmol) and 2,2-dimethoxyacetaldehyde (120.0 mg, 1.15 mmol) in methanol (5.0 mL) was added NaBH₃CN (295.0 mg, 4.61 mmol). The resulting solution was stirred at rt for 1 h and diluted with water (5.0 mL). It was extracted with dichloromethane (10 mL×2). The combined organic layer was dried over anhydrous sodium sulfate, filtered, and concentrated under reduced pressure to afford the crude product (500.0 mg). It was used directly for the next step without further purification. MS (ESI, m/z) [M+H]⁺ 522.3.

(R)-2-(4-(1-(10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)acetaldehyde. To a solution of (R)-3-(4-(1-(2,2-Dimethoxyethyl)piperidin-4-yl)-1H-1,2,3-triazol-1-yl)-10-methyl-9,10,11,12-tetrahydro-8H-[1,4]diazepino[5′,6′:4,5]thieno[3,2-f]quinolin-8-one (180.0 mg, 0.35 mmol) in 1,4-dioxane (5.0 mL) was added with HCl (5.0 mL, 6.0 M aq.). The resulting solution was stirred at 40° C. overnight. After cooling down to rt, the reaction mixture was concentrated under reduced pressure to afford the crude product, (R)-2-(4-(1-(10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)acetaldehyde (120.0 mg) as a yellow solid. It was used directly for the next step without further purification. MS (ESI, m/z) [M+H+18]⁺ 494.3.

3-(4-(4-(2-(4-(1-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)ethyl)piperazin-1-yl)phenyl)piperidine-2,6-dione. To a solution of (R)-2-(4-(1-(10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)acetaldehyde (120.0 mg, 0.25 mmol) in methanol (2.0 mL) was added 3-(4-piperazin-1-ylphenyl)piperidine-2,6-dione (69.0 mg, 0.25 mmol) and NaBH₃CN (65.0 mg, 1.01 mmol). The resulting solution was stirred at rt for 1.0 h. The reaction mixture was concentrated under reduced pressure, and the residue was purified by reverse-phase chromatography (RPC) using water and acetonitrile (MeCN) as mobile phases with 0.05% trifluoroacetic acid (TFA) to afford the desired product, 3-(4-(4-(2-(4-(1-((R)-10-methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)ethyl)piperazin-1-yl)phenyl)piperidine-2,6-dione (20.5 mg, 11% yield) as a yellow solid. MS (ESI, m/z) [M+H]⁺ 733.25 ¹H NMR (400 MHz, DMSO-d₆) δ ppm 9.48 (d, J=8.8 Hz, 1H), 8.96 (s, 1H), 8.36 (d, J=9.2 Hz, 1H), 8.27 (d, J=8.8 Hz, 1H), 7.99 (d, J=9.2 Hz, 1H), 7.11 (d, J=8.4 Hz, 2H), 6.98 (d, J=8.8 Hz, 2H), 3.76 (m, 1H), 3.66-3.60 (m, 2H), 3.57-3.45 (m, 8H), 3.30-3.15 (s, 8H), 2.70-2.62 (m, 1H), 2.50-2.45 (m, 1H), 2.39-2.28 (m, 3H), 2.21-1.95 (m, 5H), 1.20 (d, J=6.8 Hz, 3H).

### Example 46: Synthesis of 3-(1-Methyl-6-(4-(2-(4-(1-((R)-10-methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)ethyl)piperazin-1-yl)-1H-indazol-3-yl)piperidine-2,6-dione

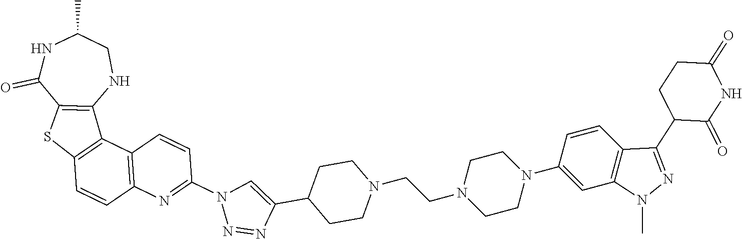 [SMILES: C[C@@H]1CNc2c(sc3ccc4nc(-n5cc(C6CCN(CCN7CCN(c8ccc9c(C%10CCC(=O)NC%10=O)nn(C)c9c8)CC7)CC6)nn5)ccc4c23)C(=O)N1]

3-(1-Methyl-6-(4-(2-(4-(1-((R)-10-methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)ethyl)piperazin-1-yl)-1H-indazol-3-yl)piperidine-2,6-dione. To a solution of (R)-2-(4-(1-(10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)acetaldehyde (60.0 mg, 0.13 mmol) in methanol (2.0 mL) was added 3-(1-methyl-6-piperazin-1-yl-indazol-3-yl)piperidine-2,6-dione (41.0 mg, 0.13 mmol). The resulting solution was stirred at rt for 1 h. After the addition of NaBH₃CN (31.0 mg, 0.50 mmol), the resulting mixture was continuously stirred at rt for 1 h. After removing the solvent reduced pressure, and the residue was purified by reverse-phase chromatography (RPC) using water and acetonitrile (MeCN) as mobile phases with 0.05% trifluoroacetic acid (TFA) to afford the desired product, 3-(1-methyl-6-(4-(2-(4-(1-((R)-10-methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)ethyl)piperazin-1-yl)-1H-indazol-3-yl)piperidine-2,6-dione (5.0 mg, 5% yield) as a yellow solid. LCMS (ESI, m/z) [M+H]⁺ 789.2 ¹H NMR (400 MHz, DMSO-d₆) δ ppm 9.47 (d, J=9.2 Hz, 1H), 8.95 (s, 1H), 8.36 (d, J=9.2 Hz, 1H), 8.27 (d, J=8.8 Hz, 1H), 7.99 (d, J=8.8 Hz, 1H), 7.55 (d, J=8.8 Hz, 1H), 7.02-6.90 (m, 2H), 4.32-4.24 (m, 1H), 3.92 (s, 3H), 3.68-3.55 (m, 5H), 3.31-3.03 (m, 9H), 2.70-2.55 (m, 3H), 2.38-2.24 (m, 4H), 2.20-2.03 (m, 4H), 1.28-1.15 (m, 6H).

### Example 47: Synthesis of 3-(4-(1-(2-(4-(1-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)ethyl)piperidin-4-yl)phenyl)piperidine-2,6-dione

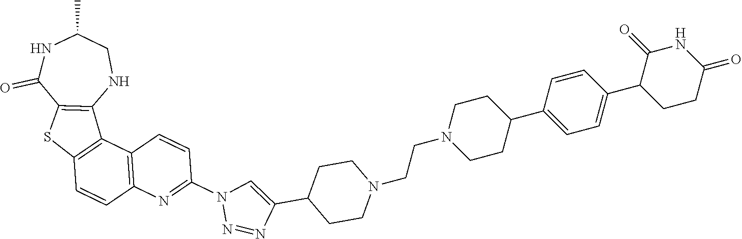 [SMILES: C[C@@H]1CNc2c(sc3ccc4nc(-n5cc(C6CCN(CCN7CCC(c8ccc(C9CCC(=O)NC9=O)cc8)CC7)CC6)nn5)ccc4c23)C(=O)N1]

3-(4-(1-(2-(4-(1-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)ethyl)piperidin-4-yl)phenyl)piperidine-2,6-dione. To a solution of (R)-2-(4-(1-(10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)acetaldehyde (120.0 mg, 0.25 mmol) in methanol (10.0 mL) was added 3-[4-(4-piperidyl)phenyl]piperidine-2,6-dione (68.0 mg, 0.25 mmol), the resulting solution was stirred at rt for 1 h, and then the NaBH₃CN (63.0 mg, 0.99 mmol) was added. The reaction mixture was continuously stirred at rt for 0.5 h. After concentrating to remove the solvent under reduced pressure, the residue was purified by reverse-phase chromatography (RPC) using water and acetonitrile (MeCN) as mobile phases with 0.05% trifluoroacetic acid (TFA) to afford the desired product, 3-(4-(1-(2-(4-(1-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)piperidin-1-yl)ethyl)piperidin-4-yl)phenyl)piperidine-2,6-dione (8.0 mg, 4.0% yield) as a yellow solid. MS (ESI, m/z) [M+H]⁺ 732.3 ¹H NMR (400 MHz, DMSO-d₆) δ ppm 10.82 (s, 1H), 9.46 (d, J=9.2 Hz, 1H), 8.80 (s, 1H), 8.34 (d, J=9.2 Hz, 1H), 8.25 (d, J=8.8 Hz, 1H), 8.15 (d, J=4.8 Hz, 1H), 7.99 (d, J=9.2 Hz, 1H), 7.25-7.08 (m, 5H), 3.84-3.78 (m, 1H), 3.62 (s, 1H), 3.49 (t, J=6.4 Hz, 2H), 3.02 (t, J=12.0 Hz, 4H), 2.87-2.77 (m, 2H), 2.72-2.59 (m, 2H), 2.53 (s, 2H), 2.47-2.43 (m, 1H), 2.21-1.98 (m, 9H), 1.82-1.58 (m, 6H), 1.20 (d, J=6.8 Hz, 3H).

### Example 48: Synthesis of 3-(4-(1-(2-(4-(4-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)ethyl)piperidin-4-yl)phenyl)piperidine-2,6-dione

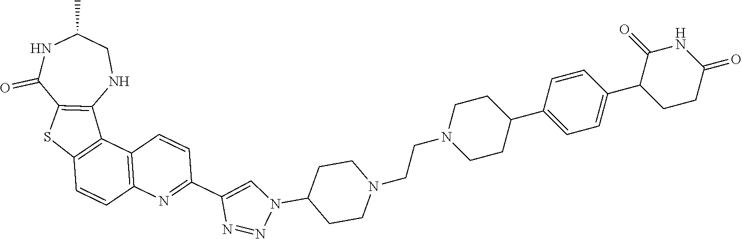 [SMILES: C[C@@H]1CNc2c(sc3ccc4nc(-c5cn(C6CCN(CCN7CCC(c8ccc(C9CCC(=O)NC9=O)cc8)CC7)CC6)nn5)ccc4c23)C(=O)N1]

3-(4-(1-(2-(4-(4-((R)-10-Methyl-8-oxo-9,10,11,12-tetrahydro-8H-[1,4]diazepino[5′,6′:4,5]thieno[3,2-(]quinolin-3-yl)-1H-1,2,3-triazol-1-yl)piperidin-1-yl)ethyl)piperidin-4-yl)phenyl)piperidine-2,6-dione

(R)-10-Methyl-3-((trimethylsilyl)ethynyl)-9,10,11,12-tetrahydro-8H-[1,4]diazepino[5′,6′:4,5]thieno[3,2-f]quinolin-8-one. To a solution of (R)-3-chloro-10-methyl-9,10,11,12-tetrahydro-8H-[1,4]diazepino[5′,6′:4,5]thieno[3,2-f]quinolin-8-one (300.0 mg, 0.94 mmol) and trimethyl(2-tributylstannylethynyl)silane (548.0 mg, 1.42 mmol) in 1,4-dioxane (5.0 mL) was added Pd(PPh₃)₄(160.0 mg, 0.19 mmol). The resulting solution was stirred at 90° C. for 4 h under a nitrogen atmosphere. After cooling down rt, the reaction mixture was diluted with 10.0 mL of petroleum ether/ethyl acetate (1:1). After filtered, the collected solid was washed by petroleum ether/ethyl acetate (1:1), and then dried down under reduced pressure to provide the crude product, (R)-10-methyl-3-((trimethylsilyl)ethynyl)-9,10,11,12-tetrahydro-8H-[1,4]diazepino[5′,6′:4,5]thieno[3,2-(]quinolin-8-one (300.0 mg) as a yellow solid. It was used directly to the next step without further purification. MS (ESI, m/z) [M+H]⁺ 380.2.

(R)-3-Ethynyl-10-methyl-9,10,11,12-tetrahydro-8H-[1,4]diazepino[5′,6′:4,5]thieno[3,2-f]quinolin-8-one. To a solution of (R)-10-Methyl-3-((trimethylsilyl)ethynyl)-9,10,11,12-tetrahydro-8H-[1,4]diazepino[5′,6′:4,5]thieno[3,2-f]quinolin-8-one (290.0 mg, 0.76 mmol) in methanol (5.0 mL) was added K₂CO₃ (210.0 mg, 1.53 mmol). The resulting mixture was stirred at rt for 1 h, and then diluted with water (10.0 mL). After filtration, the solid washed by water, the filtrate was concentrated under reduced pressure to afford the crude product; (R)-3-ethynyl-10-methyl-9,10,11,12-tetrahydro-8H-[1,4]diazepino[5′,6′:4,5]thieno[3,2-f]quinolin-8-one (220.0 mg) as a brown solid. It was used directly to the next step without further purification. MS (ESI, m/z) [M+H]⁺ 308.1.

tert-Butyl (R)-4-(4-(10-methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidine-1-carboxylate. To a solution of (R)-3-ethynyl-10-methyl-9,10,11,12-tetrahydro-8H-[1,4]diazepino[5′,6′:4,5]thieno[3,2-f]quinolin-8-one (80.0 mg, 0.26 mmol), tert-butyl 4-azidopiperidine-1-carboxylate (58.0 mg, 0.26 mmol) in THE (2.0 mL) and water (2.0 mL) was added CuSO₄·5H₂O (19.0 mg, 0.08 mmol) and (5R)-[(1S)-1,2-dihydroxyethyl]-3,4-dihydroxyfuran-2(5H)-one (27.0 mg, 0.16 mmol). The resulting solution was stirred at rt for 2 h and diluted with water (2.0 mL). After filtrated, the solid was washed by water and dried down under reduced pressure to afford the crude product, tert-butyl (R)-4-(4-(10-methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidine-1-carboxylate (100.0 mg) as a brown solid. It was used directly for the next step without further purification. MS (ESI, m/z) [(M+H]⁺ 534.3.

(R)-10-Methyl-3-(1-(piperidin-4-yl)-1H-1,2,3-triazol-4-yl)-9,10,11,12-tetrahydro-8H-[1,4]diazepino[5′,6′:4,5]thieno[3,2-f]quinolin-8-one. To a solution of (R)-4-(4-(10-methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidine-1-carboxylate (100.0 mg, 0.188 mmol) in DCM (2.0 mL), trifluoroacetic acid (0.5 mL) was added dropwise to the solution. After stirring at rt for 1 h, the reaction mixture was concentrated under reduced pressure, and the residue was purified by reverse-phase chromatography (RPC) using water and acetonitrile (MeCN) as mobile phases with 0.05% trifluoroacetic acid (TFA) to afford the desired product, (R)-10-methyl-3-(1-(piperidin-4-yl)-1H-1,2,3-triazol-4-yl)-9,10,11,12-tetrahydro-8H-[1,4]diazepino[5′,6′:4,5]thieno[3,2-f]quinolin-8-one (60.0 mg, 74% yield) as an orange solid. MS (ESI, m/z) [(M+H]⁺ 434.2 ¹H NMR (400 MHz, DMSO-d₆+D₂O) δ ppm 9.28 (d, J=9.0 Hz, 1H), 8.89 (s, 1H), 8.30 (d, J=9.0 Hz, 1H), 8.16 (d, J=9.0 Hz, 1H), 7.96 (d, J=9.0 Hz, 1H), 5.03-4.89 (m, 1H), 3.53-3.44 (m, 5H), 3.24-3.11 (m, 2H), 2.50-2.20 (m, 4H), 1.20 (d, J=6.9 Hz, 3H).

(R)-3-(1-(1-(2,2-Dimethoxyethyl)piperidin-4-yl)-1H-1,2,3-triazol-4-yl)-10-methyl-9,10,11,12-tetrahydro-8H-[1,4]diazepino[5′,6′:4,5]thieno[3,2-f]quinolin-8-one. To a solution of (R)-10-methyl-3-(1-(piperidin-4-yl)-1H-1,2,3-triazol-4-yl)-9,10,11,12-tetrahydro-8H-[1,4]diazepino[5′,6′:4,5]thieno[3,2-(]quinolin-8-one (60.0 mg, 0.14 mmol) in methanol (2.0 mL), triethyl amine (TEA) was added to adjust the pH value for the solution to 9˜10, and followed by 2,2-dimethoxyacetaldehyde (15.9 mg, 0.15 mmol). The pH value for the reaction mixture was further adjusted to 5˜6 with acetic acid, and then the NaBH₃CN (27.0 mg, 0.42 mmol) was added. The resulting mixture was stirred at rt for 1 h, and then concentrated under reduced pressure. The residue was purified by reverse-phase chromatography (RPC) using water and acetonitrile (MeCN) as mobile phases with 0.05% trifluoroacetic acid (TFA) to afford the desired product, (R)-3-(1-(1-(2,2-dimethoxyethyl)piperidin-4-yl)-1H-1,2,3-triazol-4-yl)-10-methyl-9,10,11,12-tetrahydro-8H-[1,4]diazepino[5′,6′:4,5]thieno[3,2-f]quinolin-8-one (60.0 mg, 83% yield) as a red solid. MS (ESI, m/z) [M+H]⁺ 522.1.

(R)-2-(4-(4-(10-methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)acetaldehyde. To a solution of (R)-3-(1-(1-(2,2-Dimethoxyethyl)piperidin-4-yl)-1H-1,2,3-triazol-4-yl)-10-methyl-9,10,11,12-tetrahydro-8H-[1,4]diazepino[5′,6′:4,5]thieno[3,2-f]quinolin-8-one (35.0 mg, 0.07 mmol) in 1,4-dioxane (1.0 mL) was added by HCl (1.0 mL, 4.0 M in 1,4-dioxane). The resulting solution was stirred at 40° C. overnight, and then concentrated under reduced pressure to afford the crude product, (R)-2-(4-(4-(10-methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)acetaldehyde (30.0 mg, 94% yield) as a yellow solid. MS (ESI, m/z) [M+H+18]⁺ 494.1. It was used directly for the next step without further purification.

3-(4-(1-(2-(4-(4-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)ethyl)piperidin-4-yl)phenyl)piperidine-2,6-dione. To a solution of 3-[4-(4-piperidyl)phenyl]piperidine-2,6-dione (13.0 mg, 0.05 mmol) in methanol (1.0 mL), the pH value for the solution was adjusted to 9˜10 with triethyl amine (TEA) and then added with (R)-2-(4-(4-(10-methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)acetaldehyde (20.0 mg, 0.04 mmol). The pH value for the resulting mixture was further adjusted to 5˜6 with acetic acid, and then NaBH₃CN (11.0 mg, 0.16 mmol) was added. The resulting mixture was stirred at rt for 1 h, and then concentrated under reduced pressure. The residue was purified by reverse-phase chromatography (RPC) using water and acetonitrile (MeCN) as mobile phases with 0.05% trifluoroacetic acid (TFA) to afford the desired product, 3-(4-(1-(2-(4-(4-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)ethyl)piperidin-4-yl)phenyl)piperidine-2,6-dione (3.0 mg, 9% yield) as an orange solid. MS (ESI, m/z)[M+H]⁺ 732.5. ¹H NMR (400 MHz, DMSO-d₆) δ ppm 9.28 (d, J=9.2 Hz, 1H), 8.92 (s, 1H), 8.31 (d, J=9.2 Hz, 1H), 8.16 (d, J=8.8 Hz, 1H), 7.96 (d, J=8.8 Hz, 1H), 7.33-7.04 (m, 5H), 4.92 (s, 1H), 3.86-3.79 (m, 1H), 3.69-3.56 (m, 5H), 3.21-3.02 (m, 6H), 2.91-2.78 (m, 2H), 2.73-2.62 (m, 2H), 2.45 (s, 4H), 2.24-2.10 (m, 2H), 2.08-2.00 (m, 4H), 2.00-1.84 (m, 3H), 1.20 (d, J=6.8 Hz, 3H).

### Example 49: Synthesis of 3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)ethyl)piperazin-1-yl)phenyl)piperidine-2,6-dione

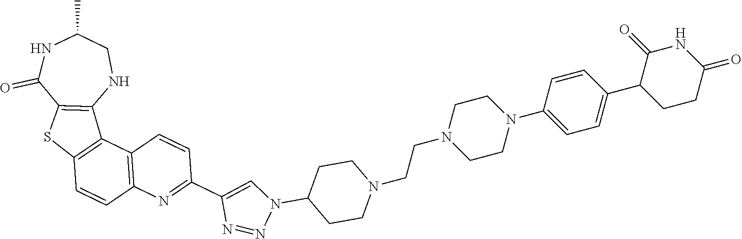 [SMILES: C[C@@H]1CNc2c(sc3ccc4nc(-c5cn(C6CCN(CCN7CCN(c8ccc(C9CCC(=O)NC9=O)cc8)CC7)CC6)nn5)ccc4c23)C(=O)N1]

3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)ethyl)piperazin-1-yl)phenyl)piperidine-2,6-dione: To a solution of 3-(4-piperazin-1-ylphenyl)piperidine-2,6-dione (13.0 mg, 0.05 mmol) in methanol (1.0 mL) was added by (R)-2-(4-(4-(10-methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)acetaldehyde (20.0 mg, 0.04 mmol) and stirred at rt for 0.5 h. After charged with NaBH₃CN (8.0 mg, 0.13 mmol), the resulting mixture was stirred at rt for 1 h, and then concentrated under reduced pressure. The residue was purified by reverse-phase chromatography (RPC) using water and acetonitrile (MeCN) as mobile phases with 0.05% trifluoroacetic acid (TFA) to afford the desired product, 3-(4-(4-(2-(4-(4-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)ethyl)piperazin-1-yl)phenyl)piperidine-2,6-dione (5.5 mg, 17% yield) as an orange solid. MS (ESI, m/z) [M+H]⁺ 733.4 ¹H NMR (300 MHz, DMSO-d₆) δ ppm 10.79 (s, 1H), 9.29 (d, J=9.0 Hz, 1H), 8.92 (s, 1H), 8.29 (d, J=8.7 Hz, 1H), 8.18-8.06 (m, 2H), 7.94 (d, J=8.7 Hz, 1H), 7.16-7.06 (m, 3H), 6.97 (d, J=8.7 Hz, 2H), 4.89 (s, 1H), 3.44-3.12 (m, 18H), 2.73-2.56 (m, 2H), 2.39 (s, 4H), 2.21-2.06 (m, 1H), 2.05-1.93 (m, 1H), 1.26-1.14 (m, 4H).

### Example 50: Synthesis of 3-(1-Methyl-6-(4-(2-(4-(4-((R)-10-methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)ethyl)piperazin-1-yl)-1H-indazol-3-yl)piperidine-2,6-dione

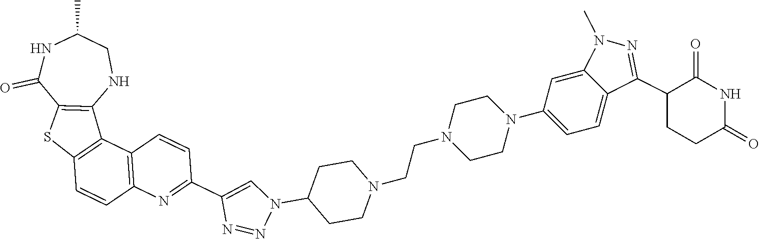 [SMILES: C[C@@H]1CNc2c(sc3ccc4nc(-c5cn(C6CCN(CCN7CCN(c8ccc9c(C%10CCC(=O)NC%10=O)nn(C)c9c8)CC7)CC6)nn5)ccc4c23)C(=O)N1]

3-(1-Methyl-6-(4-(2-(4-(4-((R)-10-methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)ethyl)piperazin-1-yl)-1H-indazol-3-yl)piperidine-2,6-dione. To a solution of 3-(1-methyl-6-piperazin-1-yl-indazol-3-yl)piperidine-2,6-dione (15.0 mg, 0.05 mmol) in methanol (1.0 mL) was added with triethyl amine (TEA) until the pH value for the solution to 9-10, and followed by the addition of (R)-2-(4-(4-(10-methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)acetaldehyde (20.0 mg, 0.04 mmol). The pH for resulting mixture was then adjusted to 5-6 by acetic acid. After NaBH₃CN (8.0 mg, 0.12 mmol) was added, the mixture was continuously stirred at rt for 1 h, and then concentrated under reduced pressure. The residue was purified by reverse-phase chromatography (RPC) using water and acetonitrile (MeCN) as mobile phases with 0.05% trifluoroacetic acid (TFA) to afford the desired product, 3-(1-methyl-6-(4-(2-(4-(4-((R)-10-methyl-8-oxo-9,10,11,12-tetrahydro-8H-[1,4]diazepino[5′,6′:4,5]thieno[3,2-f]quinolin-3-yl)-1H-1,2,3-triazol-1-yl)piperidin-1-yl)ethyl)piperazin-1-yl)-1H-indazol-3-yl)piperidine-2,6-dione (4.1 mg, 11% yield) as orange solid. MS (ESI, m/z) [M+H]⁺ 787.4 ¹H NMR (400 MHz, DMSO-d₆) δ ppm 10.90 (s, 1H), 9.29 (d, J=8.8 Hz, 1H), 8.93 (s, 1H), 8.31 (d, J=8.8 Hz, 1H), 8.16 (d, J=8.8 Hz, 11H), 8.10 (d, J=4.0 Hz, 1H), 7.96 (d, J=9.2 Hz, 1H), 7.56 (d, J=8.8 Hz, 1H), 7.15 (s, 1H), 6.98 (d, J=8.8 Hz, 1H), 6.96 (s, 1H), 4.98-4.82 (m, 1H), 4.27 (dd, J=9.2, 5.2 Hz, 1H), 3.93 (s, 3H), 3.60-3.10 (m, 18H), 2.76-2.60 (m, 3H), 2.50-2.30 (m, 4H), 2.22-2.10 (m, 1H), 1.21 (d, J=6.4 Hz, 3H).

### Example 51: Synthesis of 3-(4-(4-(2-(3-(4-((R)-10-Methyl-8-oxo-9,10,11,12-tetrahydro-8H-[1,4]diazepino[5′,6′:4,5]thieno[3,2-f]quinolin-3-yl)-1H-pyrazol-1-yl)azetidin-1-yl)ethyl)piperazin-1-yl)phenyl)piperidine-2,6-dione

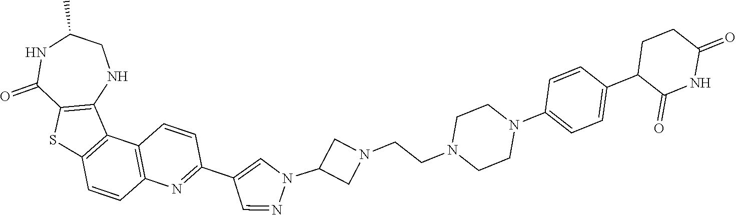 [SMILES: C[C@@H]1CNc2c(sc3ccc4nc(-c5cnn(C6CN(CCN7CCN(c8ccc(C9CCC(=O)NC9=O)cc8)CC7)C6)c5)ccc4c23)C(=O)N1]

tert-Butyl 3-(4-(4,4,5,5-tetramethyl-1,3,2-dioxaborolan-2-yl)-1H-pyrazol-1-yl)azetidine-1-carboxylate. To a solution of tert-butyl 3-(4-bromopyrazol-1-yl) azetidine-1-carboxylate (500 mg, 1.65 mmol) in 1,4-dioxane (20.0 mL) was added B₂pin₂ (504 mg, 1.99 mmol), Pd(dppf)Cl₂ (135 mg, 0.17 mmol) and KOAc (487 mg, 4.96 mmol). The resulting solution was stirred at 80° C. under nitrogen atmosphere for 2 h. LCMS showed the reaction went to completion. The reaction mixture was concentrated under vacuum, the residue was used directly in the next step without further purification. MS (ESI) [M+H-tBu]⁺ 294.2.

tert-Butyl (R)-3-(4-(10-methyl-8-oxo-9,10,11,12-tetrahydro-8H-[1,4]diazepino[5′,6′:4,5]thieno[3,2-f]quinolin-3-yl)-1H-pyrazol-1-yl)azetidine-1-carboxylate. To a solution of (R)-3-chloro-10-methyl-9,10,11,12-tetrahydro-8H-[1,4]diazepino[5′,6′:4,5]thieno[3,2-f]quinolin-8-one (400 mg, 1.25 mmol) and tert-butyl 3-[4-(4,4,5,5-tetramethyl-1,3,2-dioxaborolan-2-yl) pyrazol-1-yl]azetidine-1-carboxylate in 1,4-dioxane (15.0 mL) and water (1.5 mL) was added Cs₂CO₃ (1.2 g, 3.77 mmol) and Pd(dppf)Cl₂ (103 mg, 0.13 mmol). The resulting mixture was stirred at 100° C. under nitrogen atmosphere overnight. LCMS showed the reaction went to completion. The reaction mixture was concentrated under reduced pressure and diluted with DMF (10 mL). The precipitate was removed by filtration, and the filtrate was purified by reverse phase column chromatography (0% to 100% MeCN in 10 mM aqueous ammonium formate) to afford the product, tert-butyl (R)-3-(4-(10-methyl-8-oxo-9,10,11,12-tetrahydro-8H-[1,4]diazepino[5′,6′:4,5]thieno[3,2-f]quinolin-3-yl)-1H-pyrazol-1-yl)azetidine-1-carboxylate (450 mg, 71% yield) as a yellow oil. MS (ESI) [M+H]⁺ 505.3.

(R)-3-(1-(Azetidin-3-yl)-1H-pyrazol-4-yl)-10-methyl-9,10,11,12-tetrahydro-8H-[1,4]diazepino[5′,6′:4,5]thieno[3,2-f]quinolin-8-one. To a solution of tert-butyl (R)-3-(4-(10-methyl-8-oxo-9,10,11,12-tetrahydro-8H-[1,4]diazepino[5′,6′:4,5]thieno[3,2-f]quinolin-3-yl)-1H-pyrazol-1-yl)azetidine-1-carboxylate (400 mg, 0.52 mmol) in DCM (10.0 mL) was added TFA (2.0 mL). The reaction mixture was stirred at rt for 3 h under nitrogen atmosphere. LCMS showed the reaction went to completion. The resulting mixture was concentrated under reduced pressure, and the crude residue was used directly in the next step without further purification. MS (ESI) [M+H]⁺ 405.1.

(R)-3-(1-(1-(2,2-Dimethoxyethyl)azetidin-3-yl)-1H-pyrazol-4-yl)-10-methyl-9,10,11,12-tetrahydro-8H-[1,4]diazepino[5′,6′:4,5]thieno[3,2-f]quinolin-8-one. To a solution of (R)-3-(1-(azetidin-3-yl)-1H-pyrazol-4-yl)-10-methyl-9,10,11,12-tetrahydro-8H-[1,4]diazepino[5′,6′:4,5]thieno[3,2-f]quinolin-8-one (300 mg, 0.74 mmol) in methanol (10.0 mL) was added TEA to adjust the pH of the resulting solution to 9˜10. After the 2,2-dimethoxyacetaldehyde (92 mg, 0.89 mmol) was added, the resulting mixture was acidified to pH 5˜6 with AcOH, and charged with NaBH₃CN (140 mg, 2.23 mmol). The resulting solution was stirred for 1 h at rt, LCMS showed the reaction went to completion. The reaction mixture was diluted with water (10 mL) and extracted with EtOAc (20 mL×2). The combined organic layers were washed with brine, dried over anhydrous sodium sulfate, and concentrated under reduced pressure. The residue was purified by reverse phase column chromatography (0% to 100% MeCN in water w/ 0.1% TFA) to afford the product, (R)-3-(1-(1-(2,2-dimethoxyethyl)azetidin-3-yl)-1H-pyrazol-4-yl)-10-methyl-9,10,11,12-tetrahydro-8H-[1,4]diazepino[5′,6′:4,5]thieno[3,2-f]quinolin-8-one (200 mg, 0.41 mmol, 55% yield), as a yellow solid. MS (ESI) [M+H]⁺ 493.1.

(R)-2-(3-(4-(10-Methyl-8-oxo-9,10,11,12-tetrahydro-8H-[1,4]diazepino[5′,6′:4,5]thieno[3,2-f]quinolin-3-yl)-1H-pyrazol-1-yl)azetidin-1-yl)acetaldehyde. To a solution of (R)-3-(1-(1-(2,2-dimethoxyethyl)azetidin-3-yl)-1H-pyrazol-4-yl)-10-methyl-9,10,11,12-tetrahydro-8H-[1,4]diazepino[5′,6′:4,5]thieno[3,2-f]quinolin-8-one (100 mg, 0.21 mmol) in 1,4-dioxane (3.0 mL) was added 6M HCl (aq.) (3.0 mL). The reaction mixture was stirred at 40° C. for 6 h under a nitrogen atmosphere. LCMS showed the reaction went to completion. The reaction mixture was concentrated under reduced pressure to afford the product, (R)-2-(3-(4-(10-methyl-8-oxo-9,10,11,12-tetrahydro-8H-[1,4]diazepino[5′,6′:4,5]thieno[3,2-f]quinolin-3-yl)-1H-pyrazol-1-yl)azetidin-1-yl)acetaldehyde (90 mg, 0.20 mmol, 96% yield), as a yellow solid. It was used directly in the next step without further purification. MS (ESI) [M+H+H₂O]⁺ 465.2.

3-(4-(4-(2-(3-(4-((R)-10-Methyl-8-oxo-9,10,11,12-tetrahydro-8H-[1,4]diazepino[5′,6′:4,5]thieno[3,2-f]quinolin-3-yl)-1H-pyrazol-1-yl)azetidin-1-yl)ethyl)piperazin-1-yl)phenyl)piperidine-2,6-dione. A solution of (R)-2-(3-(4-(10-methyl-8-oxo-9,10,11,12-tetrahydro-8H-[1,4]diazepino[5′,6′:4,5]thieno[3,2-f]quinolin-3-yl)-1H-pyrazol-1-yl)azetidin-1-yl)acetaldehyde (40 mg, 0.09 mmol) in methanol (3.0 mL) was added with TEA to adjust the pH of the solution to 9˜10, and then charged with 3-(4-(piperazin-1-yl)phenyl)piperidine-2,6-dione (30 mg, 0.11 mmol). The resulting solution was then acidified with AcOH to pH 5˜6. After NaBH₃CN (16 mg, 0.27 mmol) was added, the resulting solution was stirred for 1 h at rt, and LCMS showed the reaction went to completion. The reaction mixture was diluted with water (10 mL) and extracted with EtOAc (20 mL×2). The combined organic layers were washed with brine, dried over anhydrous sodium sulfate, and concentrated under reduced pressure. The residue was purified by reverse phase HPLC to afford the product, 3-(4-(4-(2-(3-(4-((R)-10-methyl-8-oxo-9,10,11,12-tetrahydro-8H-[1,4]diazepino[5′,6′:4,5]thieno[3,2-f]quinolin-3-yl)-1H-pyrazol-1-yl)azetidin-1-yl)ethyl)piperazin-1-yl)phenyl)piperidine-2,6-dione (9 mg, 13% yield), as a yellow solid. MS (ESI) [M+H]⁺ 704.2 ¹H NMR (300 MHz, DMSO-d₆) δ ppm 10.76 (s, 1H), 9.12 (d, J=9.0 Hz, 1H), 8.68 (s, 1H), 8.27 (s, 1H), 8.07 (d, J=9.0 Hz, 2H), 7.96-7.88 (m, 2H), 7.09-7.03 (m, 3H), 6.89 (d, J=8.7 Hz, 2H), 5.10-5.00 (m, 1H), 3.85-3.70 (m, 3H), 3.68-3.55 (m, 1H), 3.50-3.48 (m, 4H), 3.20-3.05 (m, 4H), 2.76-2.35 (m, 10H), 2.32-1.90 (m, 2H), 1.20 (d, J=6.6 Hz, 3H).

### Example 52: Synthesis of 3-(1-Methyl-6-(4-(2-(3-(4-((R)-10-methyl-8-oxo-9,10,11,12-tetrahydro-8H-[1,4]diazepino[5′,6′:4,5]thieno[3,2-f]quinolin-3-yl)-1H-pyrazol-1-yl)azetidin-1-yl)ethyl)piperazin-1-yl)-1H-indazol-3-yl)piperidine-2,6-dione

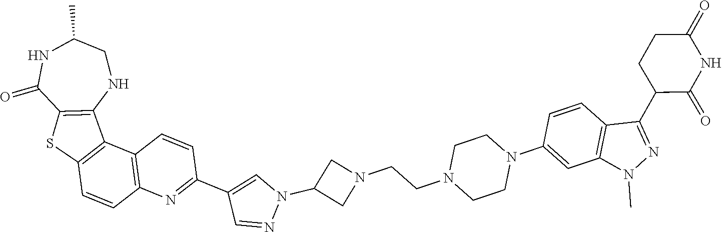 [SMILES: C[C@@H]1CNc2c(sc3ccc4nc(-c5cnn(C6CN(CCN7CCN(c8ccc9c(C%10CCC(=O)NC%10=O)nn(C)c9c8)CC7)C6)c5)ccc4c23)C(=O)N1]

3-(1-Methyl-6-(4-(2-(3-(4-((R)-10-methyl-8-oxo-9,10,11,12-tetrahydro-8H-[1,4]diazepino[5′,6′:4,5]thieno[3,2-f]quinolin-3-yl)-1H-pyrazol-1-yl)azetidin-1-yl)ethyl)piperazin-1-yl)-1H-indazol-3-yl)piperidine-2,6-dione. A solution of (R)-2-(3-(4-(10-methyl-8-oxo-9,10,11,12-tetrahydro-8H-[1,4]diazepino[5′,6′:4,5]thieno[3,2-f]quinolin-3-yl)-1H-pyrazol-1-yl)azetidin-1-yl)acetaldehyde (40 mg, 0.09 mmol) in methanol (3.0 mL) was charged with TEA to adjust the pH of the solution to 9˜10. Then, 3-(1-methyl-6-piperazin-1-yl-indazol-3-yl) piperidine-2,6-dione (35 mg, 0.11 mmol) was added. The resulting solution was then acidified with AcOH to pH 5˜6. After NaBH₃CN (16 mg, 0.27 mmol) was added, the resulting solution was stirred for 1 h at rt. LCMS showed the reaction went to completion, The reaction mixture was diluted with water (10 mL) and extracted with EtOAc (20 mL×2). The combined organic layers were washed with brine, dried over anhydrous sodium sulfate, and concentrated under reduced pressure. The residue was purified by preparative HPLC to afford the product, 3-(4-(4-(2-(3-(4-((R)-10-methyl-8-oxo-9,10,11,12-tetrahydro-8H-[1,4]diazepino[5′,6′:4,5]thieno[3,2-f]quinolin-3-yl)-1H-pyrazol-1-yl)azetidin-1-yl)ethyl)piperazin-1-yl)phenyl)piperidine-2,6-dione (7.2 mg, 10% yield) as a yellow solid. MS (ESI) [M+H]⁺ 758.3 ¹H NMR (300 MHz, DMSO-d₆) δ ppm 10.84 (s, 1H), 9.12 (d, J=8.7 Hz, 1H), 8.68 (s, 1H), 8.28 (s, 1H), 8.07 (d, J=9.0 Hz, 1H), 8.05 (s, 1H), 8.00-7.88 (m, 2H), 7.50 (d, J=9.0 Hz, 1H), 7.09 (s, 1H), 6.92 (d, J=9.0 Hz, 1H), 6.84 (s, 1H), 5.09 (t, J=6.6 Hz, 1H), 4.26 (t, J=4.5 Hz, 1H), 3.89 (s, 3H), 3.79 (t, J=6.6 Hz, 2H), 3.69-3.10 (m, 9H), 2.82-2.05 (m, 12H), 1.20 (d, J=6.6 Hz, 3H).

### Example 53: Synthesis of 3-(4-(4-(2-(3-(1-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)azetidin-1-yl)ethyl)piperazin-1-yl)phenyl)piperidine-2,6-dione

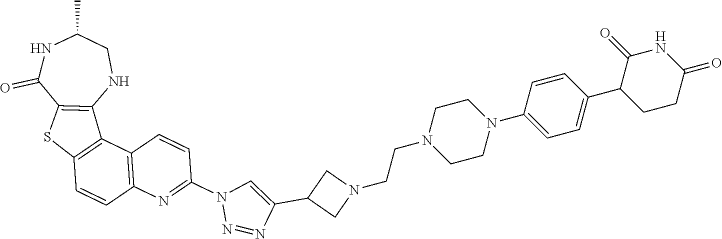 [SMILES: C[C@@H]1CNc2c(sc3ccc4nc(-n5cc(C6CN(CCN7CCN(c8ccc(C9CCC(=O)NC9=O)cc8)CC7)C6)nn5)ccc4c23)C(=O)N1]

(R)-3-Azido-10-methyl-9,10,11,12-tetrahydro-8H-[1,4]diazepino[5′,6′:4,5]thieno[3,2-f]quinolin-8-one: To a solution of (R)-3-chloro-10-methyl-9,10,11,12-tetrahydro-8H-[1,4]diazepino[5′,6′:4,5]thieno[3,2-f]quinolin-8-one (1.0 g, 3.15 mmol) in DMSO (10.0 mL) was added NaN₃ (409 mg, 6.29 mmol). The resulting solution was stirred at 120° C. overnight under a nitrogen atmosphere. LCMS showed the reaction was completed. The reaction mixture was diluted with water (100 mL). The precipitate was collected by filtration and washed with water (50 mL×2). The collected solid was dried under vacuum to afford the product, (R)-3-azido-10-methyl-9,10,11,12-tetrahydro-8H-[1,4]diazepino[5′,6′:4,5]thieno[3,2-f]quinolin-8-one (900 mg, 2.77 mmol, 88% yield). The crude product was used directly in the next step without further purification. MS (ESI) [M+H]⁺ 325.1.

tert-Butyl (R)-3-(1-(10-methyl-8-oxo-9,10,11,12-tetrahydro-8H-[1,4]diazepino[5′,6′:4,5]thieno[3,2-f]quinolin-3-yl)-1H-1,2,3-triazol-4-yl)azetidine-1-carboxylate. To a solution of (R)-3-azido-10-methyl-9,10,11,12-tetrahydro-8H-[1,4]diazepino[5′,6′:4,5]thieno[3,2-(]quinolin-8-one (400 mg, 1.23 mmol), tert-butyl 3-ethynylazetidine-1-carboxylate (335 mg, 1.85 mmol) and DIPEA (478 mg, 3.7 mmol) in DMF (5.0 mL) was added CuI (23 mg, 0.12 mmol). The resulting solution was stirred at 140° C. overnight under a nitrogen atmosphere. LCMS showed the reaction went to completion. The reaction mixture was diluted with DMF (5.0 mL). The precipitate was removed by filtration, and the filtrate was concentrated and purified by reverse phase column chromatography (0% to 100% MeCN in water w/ 0.1% TFA) to afford the product, tert-butyl (R)-3-(1-(10-methyl-8-oxo-9,10,11,12-tetrahydro-8H-[1,4]diazepino[5′,6′:4,5]thieno[3,2-f]quinolin-3-yl)-1H-1,2,3-triazol-4-yl)azetidine-1-carboxylate (300 mg, 48% yield), as an off-white solid. MS (ESI) [M+H]⁺ 506.3.

(R)-3-(4-(Azetidin-3-yl)-1H-1,2,3-triazol-1-yl)-10-methyl-9,10,11,12-tetrahydro-8H-[1,4]diazepino[5′,6′:4,5]thieno[3,2-f]quinolin-8-one. To a solution of tert-butyl (R)-3-(1-(10-methyl-8-oxo-9,10,11,12-tetrahydro-8H-[1,4]diazepino[5′,6′:4,5]thieno[3,2-(]quinolin-3-yl)-1H-1,2,3-triazol-4-yl)azetidine-1-carboxylate (30 mg, 0.06 mmol, 1.0) in DCM (2.5 mL) was added TFA (0.5 mL). The mixture was stirred at rt for 2 h. LCMS showed the reaction went to completion. The reaction mixture was concentrated under reduced pressure and purified by preparative HPLC (0% to 100% MeOH in water w/ 01.% TFA) to the product, (R)-3-(4-(azetidin-3-yl)-1H-1,2,3-triazol-1-yl)-10-methyl-9,10,11,12-tetrahydro-8H-[1,4]diazepino[5′,6′:4,5]thieno[3,2-f]quinolin-8-one (6.0 mg, 0.15 mmol, 24% yield) as a yellow solid. MS (ESI) [M+H]⁺ 406.4.

(R)-3-(4-(1-(2,2-Dimethoxyethyl)azetidin-3-yl)-1H-1,2,3-triazol-1-yl)-10-methyl-9,10,11,12-tetrahydro-8H-[1,4]diazepino[5′,6′:4,5]thieno[3,2-f]quinolin-8-one. To a solution of (R)-3-(4-(azetidin-3-yl)-1H-1,2,3-triazol-1-yl)-10-methyl-9,10,11,12-tetrahydro-8H-[1,4]diazepino[5′,6′:4,5]thieno[3,2-f]quinolin-8-one (150 mg, 0.37 mmol) and 2,2-dimethoxyacetaldehyde (77 mg, 0.74 mmol) in methanol (5.0 mL) was added NaBH₃CN (95 mg, 1.48 mmol). The resulting solution was stirred at rt for 1 h. LCMS showed the reaction went to completion. The resulting solution was diluted with water (50 mL), extracted with dichloromethane (50 mL×3). The combined organic layers were dried over anhydrous sodium sulfate, filtered, and concentrated under reduced pressure. The residue was used directly in the next step without further purification. MS (ESI) [M+H]⁺ 494.3.

(R)-2-(3-(1-(10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)azetidin-1-yl)acetaldehyde. To a solution of (R)-3-(4-(1-(2,2-dimethoxyethyl)azetidin-3-yl)-1H-1,2,3-triazol-1-yl)-10-methyl-9,10,11,12-tetrahydro-8H-[1,4]diazepino[5′,6′:4,5]thieno[3,2-f]quinolin-8-one (120 mg, 0.24 mmol) in 1,4-dioxane (3.0 mL) was added 6 M HCl (aq., (3.0 mL). The resulting solution was stirred at 40° C. overnight. LCMS showed the reaction went to completion. The reaction mixture was concentrated under reduced pressure to afford the crude product, which was used directly in the next step without further purification. MS (ESI) [M+H+H₂O]⁺ 466.3.

3-(4-(4-(2-(3-(1-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)azetidin-1-yl)ethyl)piperazin-1-yl)phenyl)piperidine-2,6-dione. To a stirring solution of (R)-2-(3-(1-(10-methyl-8-oxo-9,10,11,12-tetrahydro-8H-[1,4]diazepino[5′,6′:4,5]thieno[3,2-f]quinolin-3-yl)-1H-1,2,3-triazol-4-yl)azetidin-1-yl)acetaldehyde (80 mg, 0.18 mmol) in DCM (2.5 mL) was added 3-(4-piperazin-1-ylphenyl)piperidine-2,6-dione (49 mg, 0.18 mmol). The resulting mixture was stirred at rt overnight. Then, NaBH(OAc)₃ (152 mg, 0.72 mmol) was added, and the resulting solution was stirred at rt for 1 h. LCMS showed the reaction went to completion. The reaction mixture was concentrated under reduced pressure and purified by preparative HPLC (0% to 100% MeCN in water w/ 0.1% TFA) to the product, 3-(4-(4-(2-(3-(1-((R)-10-methyl-8-oxo-9,10,11,12-tetrahydro-8H-[1,4]diazepino[5′,6′:4,5]thieno[3,2-f]quinolin-3-yl)-1H-1,2,3-triazol-4-yl)azetidin-1-yl)ethyl)piperazin-1-yl)phenyl)piperidine-2,6-dione (1.7 mg, 1% yield), as a yellow solid. MS (ESI) [M+H]⁺ 705.2 ¹H NMR (400 MHz, DMSO-d₆) δ ppm 10.77 (s, 1H), 9.46 (d, J=9.2 Hz, 1H), 8.97 (s, 1H), 8.35 (d, J=9.2 Hz, 1H), 8.26 (d, J=9.2 Hz, 1H), 8.15 (d, J=4.4 Hz, 1H), 7.99 (d, J=9.2 Hz, 1H), 7.19 (t, J=6.0 Hz, 1H), 7.08-7.01 (m, 2H), 6.93-6.85 (m, 2H), 3.83-3.62 (m, 6H), 3.51-3.48 (m, 4H), 3.121 (s, 2H), 2.67-2.60 (m, 4H), 2.58-2.54 (m, 4H), 2.49-2.40 (m, 1H), 2.36 (t, J=7.0 Hz, 2H), 2.19-1.95 (m, 2H), 1.20 (d, J=6.8 Hz, 3H).

### Example 54: Synthesis of 3-(4-(1-(2-(3-(1-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)azetidin-1-yl)ethyl)piperidin-4-yl)phenyl)piperidine-2,6-dione

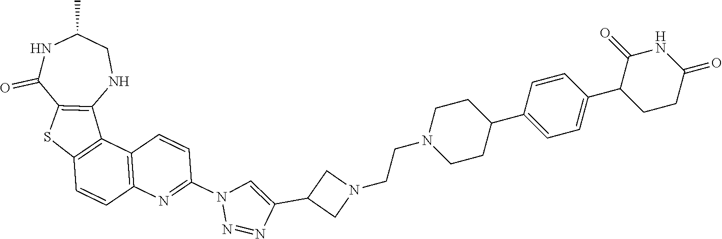 [SMILES: C[C@@H]1CNc2c(sc3ccc4nc(-n5cc(C6CN(CCN7CCC(c8ccc(C9CCC(=O)NC9=O)cc8)CC7)C6)nn5)ccc4c23)C(=O)N1]

3-(4-(1-(2-(3-(1-((R)-10-Methyl-8-oxo-9,10,11,12-tetrahydro-8H-[1,4]diazepino[5′,6′:4,5]thieno[3,2-f]quinolin-3-yl)-1H-1,2,3-triazol-4-yl)azetidin-1-yl)ethyl)piperidin-4-yl)phenyl)piperidine-2,6-dione. To a solution of (R)-2-(3-(1-(10-methyl-8-oxo-9,10,11,12-tetrahydro-8H-[1,4]diazepino[5′,6′:4,5]thieno[3,2-f]quinolin-3-yl)-1H-1,2,3-triazol-4-yl)azetidin-1-yl)acetaldehyde (80 mg, 0.18 mmol) in DCM (2.5 mL) was added 3-[4-(4-piperidyl)phenyl]piperidine-2,6-dione (49 mg, 0.18 mmol). The resulting mixture was stirred at room temperature overnight. Then, NaBH(OAc)₃ (152 mg, 0.72 mmol, 4.0 equiv.) was added, and the resulting solution was stirred at rt for 1 h. LCMS showed the reaction went to completion. The reaction mixture was concentrated under reduced pressure and purified by preparative HPLC (0% to 100% MeCN in water w/ 0.1% TFA) to afford the product, 3-(4-(1-(2-(3-(1-((R)-10-methyl-8-oxo-9,10,11,12-tetrahydro-8H-[1,4]diazepino[5′,6′:4,5]thieno[3,2-f]quinolin-3-yl)-1H-1,2,3-triazol-4-yl)azetidin-1-yl)ethyl)piperidin-4-yl)phenyl)piperidine-2,6-dione (3.7 mg, 0.002 mmol, 3% yield) as a yellow solid. MS (ESI) [M+H]⁺ 704.2 ¹H NMR (400 MHz, DMSO-d₆) δ ppm 10.79 (s, 1H), 9.47 (d, J=9.2 Hz, 1H), 8.96 (s, 1H), 8.35 (d, J=9.2 Hz, 1H), 8.26 (d, J=8.8 Hz, 1H), 8.14 (d, J=4.0 Hz, 1H), 7.99 (d, J=9.2 Hz, 1H), 7.23-7.05 (m, 5H), 3.86-3.76 (m, 2H), 3.72-3.58 (m, 3H), 3.49 (t, J=6.0 Hz, 3H), 3.27 (d, J=6.8 Hz, 2H), 3.00 (d, J=10.8 Hz, 2H), 2.71-2.57 (m, 3H), 2.35-2.29 (m, 2H), 2.23-2.09 (m, 1H), 2.08-1.97 (m, 3H), 1.77-1.56 (m, 4H), 1.20 (d, J=6.8 Hz, 4H).

### Example 55: Synthesis of 3-(4-(4-(2-(4-(((R)-10-Methyl-8-oxo-8,9,10,11-tetrahydro-[1,4]oxazepino[7′,6′:4,5]thieno[3,2-f]quinolin-3-yl)ethynyl)piperidin-1-yl)-2-oxoacetyl)piperazin-1-yl)phenyl)piperidine-2,6-dione

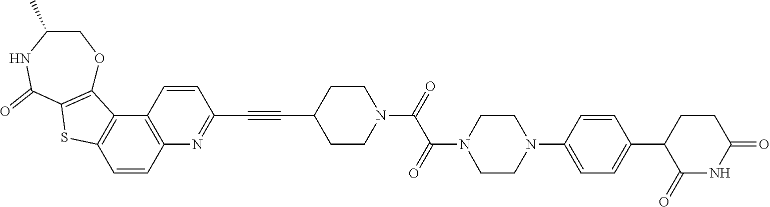 [SMILES: C[C@@H]1COc2c(sc3ccc4nc(C#CC5CCN(C(=O)C(=O)N6CCN(c7ccc(C8CCC(=O)NC8=O)cc7)CC6)CC5)ccc4c23)C(=O)N1]

tert-Butyl (R)-3-((1-(tert-butoxycarbonyl)piperidin-4-yl)ethynyl)-10-methyl-8-oxo-10,11-dihydro-[1,4]oxazepino[7′,6′:4,5]thieno[3,2-f]quinoline-9(8H)-carboxylate. To a solution of tert-butyl (R)-3-chloro-10-methyl-8-oxo-10,11-dihydro-[1,4]oxazepino[7′,6′:4,5]thieno[3,2-f]quinoline-9(8H)-carboxylate (200 mg, 0.48 mmol), tert-butyl 4-ethynylpiperidine-1-carboxylate (150 mg, 0.72 mmol), CuI (36 mg, 0.19 mmol) and TEA (308 mg, 2.39 mmol) in THE (4.0 mL) was added Pd(PPh₃)₂Cl₂ (67 mg, 0.10 mmol). The resulting mixture was stirred at 70° C. for 4 h under a nitrogen atmosphere, and LCMS showed the reaction went to completion. The reaction mixture was diluted with EtOAc. The solid was removed by filtration, and the filtrate was concentrated under reduced pressure and purified by silica gel column chromatography (2:1 petroleum ether/ethyl acetate) to afford the product, tert-butyl (R)-3-((1-(tert-butoxycarbonyl)piperidin-4-yl)ethynyl)-10-methyl-8-oxo-10,11-dihydro-[1,4]oxazepino[7′,6′:4,5]thieno[3,2-f]quinoline-9(8H)-carboxylate (250 mg, 88% yield), as a brown solid. MS (ESI) [M+H]⁺ 592.

(R)-10-Methyl-3-(piperidin-4-ylethynyl)-10,11-dihydro-[1,4]oxazepino[7′,6′:4,5]thieno[3,2-f]quinolin-8(9H)-one. To a solution of tert-butyl (R)-3-((1-(tert-butoxycarbonyl)piperidin-4-yl)ethynyl)-10-methyl-8-oxo-10,11-dihydro-[1,4]oxazepino[7′,6′:4,5]thieno[3,2-f]quinoline-9(8H)-carboxylate (250 mg, 0.42 mmol) in DCM (4.0 mL) was added dropwise with TFA (1.0 mL). The resulting solution was stirred for 1 h at rt, and LCMS showed the reaction went to completion. The reaction mixture was concentrated under reduced pressure to give afford crude product, (R)-10-methyl-3-(piperidin-4-ylethynyl)-10,11-dihydro-[1,4]oxazepino[7′,6′:4,5]thieno[3,2-f]quinolin-8(9H)-one (150 mg, 0.38 mmol, 90% yield) as a yellow solid. The material was used in the next step without further purification. MS (ESI) [M+H]⁺ 392.1.

Ethyl (R)-2-(4-((10-methyl-8-oxo-8,9,10,11-tetrahydro-[1,4]oxazepino[7′,6′:4,5]thieno[3,2-f]quinolin-3-yl)ethynyl)piperidin-1-yl)-2-oxoacetate. To a solution of (R)-10-methyl-3-(piperidin-4-ylethynyl)-10,11-dihydro-[1,4]oxazepino[7′,6′:4,5]thieno[3,2-(]quinolin-8(9H)-one (100 mg, 0.26 mmol) and DIPEA (99 mg, 0.77 mmol) in THF (3.0 mL) was added ethyl 2-chloro-2-oxo-acetate (52 mg, 0.38 mmol). The resulting solution was stirred for 1 h at rt. LCMS showed the reaction went to completion. The resulting mixture was concentrated under reduced pressure and was purified by reverse phase column chromatography (0% to 100% MeCN in water w/ 0.1% TFA) to afford the product, ethyl (R)-2-(4-((10-methyl-8-oxo-8,9,10,11-tetrahydro-[1,4]oxazepino[7′,6′:4,5]thieno[3,2-f]quinolin-3-yl)ethynyl)piperidin-1-yl)-2-oxoacetate (60 mg, 0.12 mmol, 46%) as a yellow solid. MS (ESI) [M+H]⁺ 478.3.

(R)-2-(4-((10-methyl-8-oxo-8,9,10,11-tetrahydro-[1,4]oxazepino[7′,6′:4,5]thieno[3,2-f]quinolin-3-yl)ethynyl)piperidin-1-yl)-2-oxoacetic acid. To a solution of ethyl (R)-2-(4-((10-methyl-8-oxo-8,9,10,11-tetrahydro-[1,4]oxazepino[7′,6′:4,5]thieno[3,2-f]quinolin-3-yl)ethynyl)piperidin-1-yl)-2-oxoacetate (55 mg, 0.11 mmol) in THF (1.0 mL) and water (1.0 mL) was added LiOH·H₂O (5 mg, 0.22 mmol). The resulting solution was stirred at rt for 1 h. LCMS showed the reaction went to completion. The reaction mixture was diluted with DMSO and then purified by reverse phase column chromatography (0% to 100% MeCN in water w/ 0.1% TFA) to afford the product, (R)-2-(4-((10-methyl-8-oxo-8,9,10,11-tetrahydro-[1,4]oxazepino[7′,6′:4,5]thieno[3,2-f]quinolin-3-yl)ethynyl)piperidin-1-yl)-2-oxoacetic acid (50 mg, 99% yield) as a yellow solid. MS (ESI) [M+H]⁺ 464.3.

3-(4-(4-(2-(4-(((R)-10-Methyl-8-oxo-8,9,10,11-tetrahydro-[1,4]oxazepino[7′,6′:4,5]thieno[3,2-f]quinolin-3-yl)ethynyl)piperidin-1-yl)-2-oxoacetyl)piperazin-1-yl)phenyl)piperidine-2,6-dione. To a solution of (R)-2-(4-((10-methyl-8-oxo-8,9,10,11-tetrahydro-[1,4]oxazepino[7′,6′:4,5]thieno[3,2-f]quinolin-3-yl)ethynyl)piperidin-1-yl)-2-oxoacetic acid (40 mg, 0.09 mmol) and 3-(4-piperazin-1-ylphenyl)piperidine-2,6-dione (35 mg, 0.13 mmol) in DMF (2.0 mL) was added TCFH (49 mg, 0.13 mmol) and NMI (22 mg, 0.27 mmol). The resulting solution was stirred for 2 h at rt, and LCMS showed the reaction went to completion. The reaction mixture was diluted with DMSO, and the precipitate was removed by filtration. The filtrate was concentrated, and purified by preparative HPLC (0% to 100% MeCN in water w/ 0.1% TFA) to afford the product, 3-(4-(4-(2-(4-(((R)-10-methyl-8-oxo-8,9,10,11-tetrahydro-[1,4]oxazepino[7′,6′:4,5]thieno[3,2-f]quinolin-3-yl)ethynyl)piperidin-1-yl)-2-oxoacetyl)piperazin-1-yl)phenyl)piperidine-2,6-dione (15.3 mg, 22% yield) as a yellow solid. MS (ESI) [M+H]⁺ 719.3 H NMR (400 MHz, DMSO-d₆) δ ppm 10.80 (s, 1H), 9.21 (d, J=8.8 Hz, 1H), 8.50 (d, J=4.0 Hz, 1H), 8.23 (d, J=9.2 Hz, 1H), 7.96 (d, J=9.2 Hz, 1H), 7.73 (d, J=8.8 Hz, 1H), 7.09 (d, J=8.8 Hz, 2H), 6.94 (d, J=9.2 Hz, 2H), 4.60 (d, J=4.4 Hz, 2H), 3.98-3.42 (m, 9H), 3.41-3.02 (m, 7H), 2.72-2.40 (m, 2H), 2.24-1.92 (m, 3H), 1.80-1.62 (m, 2H), 1.27 (d, J=6.8 Hz, 3H).

### Example 56: Biological Assays

### Example 56.1: MK2 Degradation Assay in MK2 Overexpressed HiBiT-Tagged Cell Line

A MK2-LDD degrader to be tested was dispensed into a white 384-well tissue-culture treated plate using an acoustic liquid handler. Dilutions based on a 25 μL assay volume were prepared in duplicate 10 point 3-fold serial dilutions starting with a 10 M dose. Negative control wells containing only 0.2% DMSO were also included to calculate 100% signal. All wells were backfilled to a final DMSO concentration of 0.2% to ensure DMSO uniformity across wells. Cells stably expressing HiBiT-tagged protein of interest were then washed, trypsinized, and counted. These cells were then resuspended in fresh medium to give the proper concentration. This was done so that when cells are plated at a 25 μL seeding volume the assay would be conducted within the linear range from optimization of cell based HiBiT assay. Following the above procedure, 25 μL of cells expressing HiBiT-tagged protein of interest were dispensed per well. Cells were then seeded into the 384-well plate that was pre-spotted with compounds. The resulting cells were incubated overnight at 37° C./5% CO₂. The 384-well plates were then removed from the incubator and left at room temperature for 30 min. The Nano-Glo HiBiT Lytic Detection Reagent was prepared according to manufacturer's instructions, 25 μL of Nano-Glo HiBiT Lytic Detection Reagent were added per well of a 384-well plate. The resulting plates were incubated for 30 min at room temperature. The luminescence signal was read using a plate reader. All luminescence values were normalized to the DMSO control wells. The average value of the DMSO control wells was set to equal 100% of the relative HiBiT-tagged target protein levels. The luminescence values were plotted using a graphing software. The MK2-LDD degrader concentration was plotted on the x-axis and the corresponding relative protein of interest levels on the y-axis. Graphing software was to determine the EC₅₀ value (the half-maximum effective concentration) of a MK2-LDD for the degradation of the HiBiT-tagged substrate. The software used a four-parameter logistic model (sigmoidal dose-response model) (FIT=(A+{(B−A)/1+[(C/x)^{D}]})) where C was the inflection point (EC₅₀), D was the correlation coefficient, and A and B were the low and high limits of the fit, respectively) to calculate the EC₅₀. The Yₘᵢₙ value was calculated by determining the lowest percentage of target protein remaining following MK2-LDD treatment.

### Example 56.2. Mitogen-Activated Protein Kinase-Activated Protein Kinase 2 AssayQuant Technologies® Assay for Compound Potency Assessment (MK2 OMNIA Biochemical Assay)

The protocol below describes a continuous-read kinase assay optimized to measure potency of compounds against p38a activated, mitogen-activated protein kinase-activated protein kinase 2 (MAPKAP-K2 or MK2) enzyme.

[Reagent] used: [MK-2]=0.05 nM, [ATP]=100 μM and [AQT0425]=10 μM

A 1.25× stock solution of MK2 (PV3317, from Invitrogen) and a 5× stock solution of ATP and Sox conjugated peptide substrate, AQT0425 (CSKS-AQT0425B, from AssayQuant Technologies), were prepared in 1× kinase reaction buffer consisting of 50 mM HEPES, pH 7.5, 0.01% Brij-35, 0.55 mM EGTA, 10 mM MgCl2 and 1 mM DTT.

10 μL of the ATP and substrate solution mix was added to a Coming (3574) 384-well, white, non-binding surface microtiter plate containing 0.5 μL of serially diluted test compounds prepared in DMSO. The reactions were started with the addition of 40 μL of the enzyme solution and monitored every 71 seconds for 240 min at kex 360/λem 485 in a Synergy H4 plate reader from BioTek at room temperature.

The initial linear portions of the net progress curves were fit according to a linear equation to yield the slope and percentage of inhibition (% inhibition) at each compound concentration. The net progress curves obtained during the entirety of reactions were also fit according to an ascending single-exponential equation (Eq. 1) to yield V_{obs} values at each compound concentration. Plots of % Inhibition versus degrader concentrations were fit according to a dose-response equation (Eq. 2) to generate IC₅₀ and Hill slope values while plots of V_{obs} versus degrader concentration were fit according to Equation 3 (Eq. 3) to generate apparent K_{inact} values using the GraphPad PRISM software.

$$F = V \text{?} \frac{( 1 - \text{?} \text{?} )}{\text{?}} \qquad ( Eq . 1 )$$

$$\text{?} \text{indicates text missing or illegible when filed}$$

where F is the fluorescence intensity from the plate reader, V₀ is a constant reflecting the relationship between the instrument readout and product concentration, t is time, e is Euler's number, and V_{obs} is the observed inactivation rate constant.

$$\% Inhibition = \frac{100}{1 + ( \frac{{IC}_{50}}{[ J ]} ) \text{?}} \qquad ( Eq . 2 )$$

$$\text{?} \text{indicates text missing or illegible when filed}$$

where % Inhibition is percentage of inhibition, IC₅₀ is half maximal inhibitory concentration, [I] is the degraders concentration, and n is the Hill slope.

$${k}_{obs} = \frac{{k}_{intact}}{{K}_{I}} \frac{[ f ]}{2} \qquad ( Eq . 3 )$$

where k_{obs} is the observed inactivation rate constant, K_{inact} is the apparent inactivation rate constant, K₁ is the apparent inhibition constant, and [I] is the degrader concentration.

Using these assays, the EC₅₀ and IC₅₀ values of the following compounds were determined. See Table A. Compounds having an EC₅₀ of <10 nM in the HiBiT assay are denoted A; compounds having an EC₅₀ of 10-100 nM in the HiBiT assay are denoted B; compounds having an EC₅₀ of 101-500 nM in the HiBiT assay are denoted C; compounds having an EC₅₀ of >500 nM in the HiBiT assay are denoted D; compounds having an IC₅₀ of <150 nM in the OMNIA assay are denoted E; compounds having an IC₅₀ of 151-500 nM in the OMNIA assay are denoted F; compounds having an IC₅₀ of 501-1000 nM in the OMNIA assay are denoted G.

TABLE A

| Example | MK2 Omnia | HiBiT 24 h MK2 |
|---|---|---|
| No. | IC50 (nM) | EC₅₀ (μM) |
| 1 | E | A |
| 2 | E | A |
| 3 | E | A |
| 4 | E | A |
| 5 | E | A |
| 6 | E | A |
| 7 | E | A |
| 8 | E | A |
| 9 | E | B |
| 10 | F | B |
| 11 | E | B |
| 12 | E | A |
| 13 | E | B |
| 14 | E | A |
| 15 | E | B |
| 16 | E | C |
| 17 | E | B |
| 18 | E | B |
| 19 | E | B |
| 20 | E | B |
| 21 | F | C |
| 22 | F | A |
| 23 | E | A |
| 24 | E | B |
| 25 | F | C |
| 26 | E | B |
| 27 | E | B |
| 28 | E | C |
| 29 | E | B |
| 30 | E | B |
| 31 | E | B |
| 32 | F | B |
| 33 | E | C |
| 34 | F | B |
| 35 | E | B |
| 36 | F | B |
| 37 | E | B |
| 38 | F | B |
| 39 | F | C |
| 40 | F | D |
| 41 | E | B |
| 42 | E | C |
| 43 | E | B |
| 44 | E | A |
| 45 | E | A |
| 46 | E | B |
| 47 | E | B |
| 48 | E | B |
| 49 | E | A |
| 50 | E | A |
| 51 | E | A |
| 52 | E | A |
| 53 | E | D |
| 54 | E | B |
| 55 | G | D |

### EMBODIMENTS

Embodiment 1. A compound of Formula I

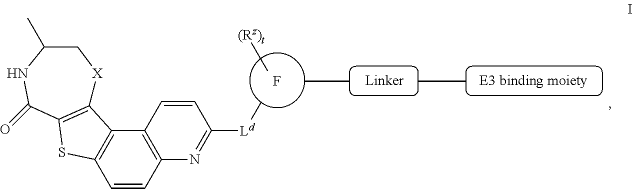

or a pharmaceutically acceptable salt thereof, wherein:
the Linker is a bivalent group;
the E3 binding moiety is a moiety that binds to an E3 ubiquitin ligase protein;
Ring F is phenylene, a 5- to 6-membered heteroarylene ring having 1-4 heteroatoms independently selected from nitrogen, oxygen, and sulfur, or a 3- to 7-membered saturated or partially unsaturated heterocyclylene having 1-3 heteroatoms independently selected from nitrogen, oxygen, and sulfur;
L^{d} is selected from a covalent bond, —O—, —S—, —N(R)—, and C₁₋₆ aliphatic;
X is selected from —O—, —S—, and —N(R)—;
R^{z} is selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur;
each R is independently hydrogen or optionally substituted C₁₋₆ aliphatic; and
t is 0, 1, 2, or 3.

Embodiment 2. The compound according to Embodiment 1, wherein Ring F is phenylene.

Embodiment 3. The compound according to Embodiment 1 or Embodiment 2, wherein L^{d} is a covalent bond.

Embodiment 4. The compound according to any one of Embodiments 1-3, wherein X is —O—.

Embodiment 5. The compound according to any one of Embodiments 1-3, wherein X is —N(R)—.

Embodiment 6. The compound according to Embodiment 5, wherein R is hydrogen.

Embodiment 7. The compound according to any one of Embodiments 1-6, wherein R^{z} is selected from halogen, —OR, —SR, —CN, —NO₂, —SO₂NR, —SO₂R, —SOR, —C(O)R, —CO₂R, and —C(O)N(R)₂.

Embodiment 8. The compound according to any one of Embodiments 1-6, wherein R^{z} is selected from halogen, —OR, —SR, —CN, and —NO₂.

Embodiment 9. The compound according to any one of Embodiments 1-8, wherein R^{z} is halogen.

Embodiment 10. The compound according to any one of Embodiments 1-6, wherein t is 0.

Embodiment 11. The compound according to any one of Embodiments 1-9, wherein t is 0.

Embodiment 12. The compound according to Embodiment 1, wherein the compound is a compound of any of Formulae I-a, I-a-i, I-a-ii, I-a-iii, I-a-iv, I-a-v, I-b, I-b-i, I-b-ii, I-b-iii, I-b-iv, I-b-v, I-c, I-c-i, I-c-ii, I-c-iii, I-c-iv, I-c-v, I-d, I-d-i, and I-d-ii:

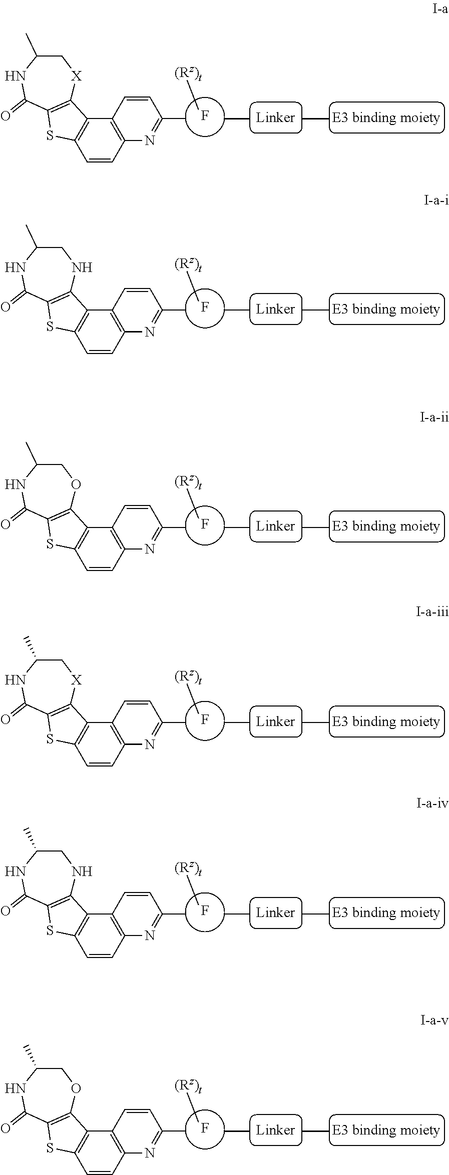

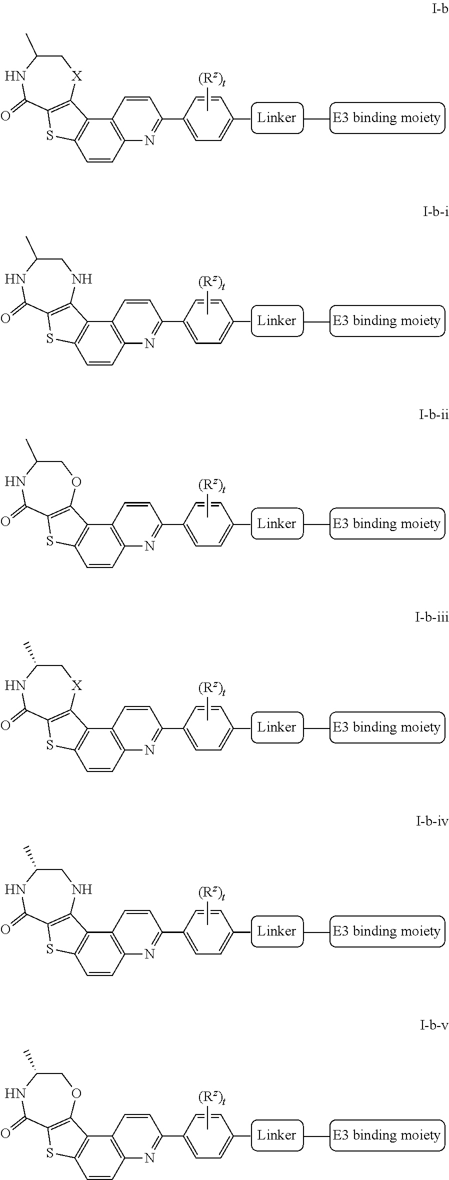

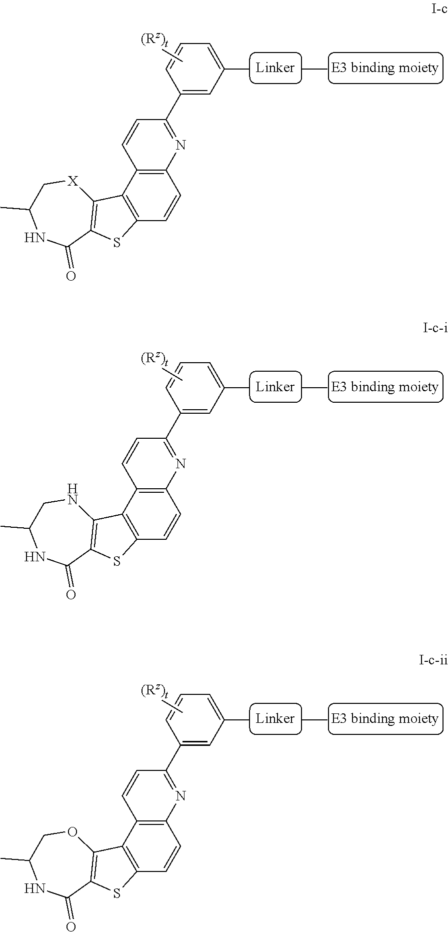

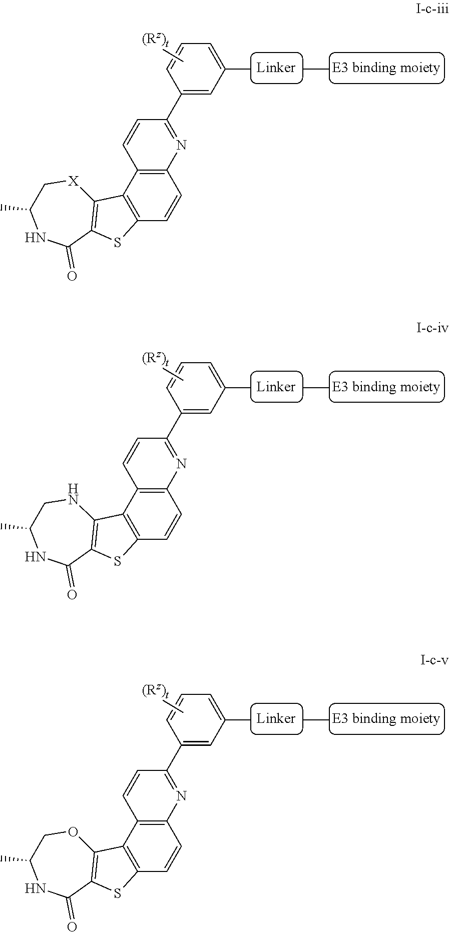

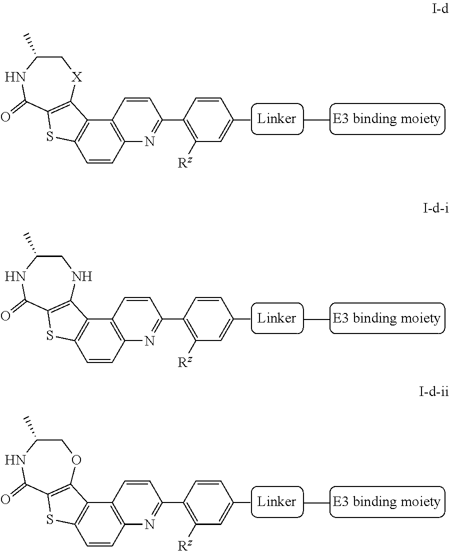

or a pharmaceutically acceptable salt thereof.

Embodiment 13. The compound according to any one of Embodiments 1-12, wherein:
the Linker is an optionally substituted bivalent C₂₋₂₀ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}, wherein
R^{L} is independently selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur.

Embodiment 14. The compound according to Embodiment 13, wherein the Linker is an optionally substituted bivalent C₂₋₂₀ straight or branched aliphatic chain, wherein two, three, or four methylene units of the aliphatic chain are optionally and independently replaced by a group selected from —N(R)—, —O—, —C(O)—, —N(R)C(O)—, —C(O)N(R)—, —OC(O)—, —C(O)O—, and a bivalent 3- to 6-membered monocyclic saturated or partially unsaturated ring having 0-2 heteroatoms independently selected from nitrogen, oxygen, and sulfur, wherein the monocyclic ring is substituted by 0-4 instances of R^{L}.

Embodiment 15. The compound according to Embodiment 13, wherein the Linker is an optionally substituted bivalent C₂₋₂₀ straight or branched aliphatic chain, wherein two, three, or four methylene units of the aliphatic chain are optionally and independently replaced by a group selected from —C(O)N(R)—, —N(R)—, —C(O)—,

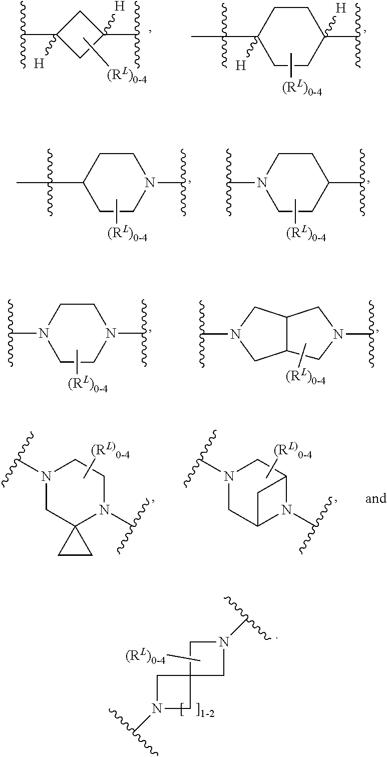

Embodiment 16. The compound according to Embodiment 13, wherein the Linker is an optionally substituted bivalent C₂₋₂₀ straight or branched aliphatic chain, wherein two, three, or four methylene units of the aliphatic chain are optionally and independently replaced by a group selected from —C(O)N(R)—, —N(R)—, —C(O)—,

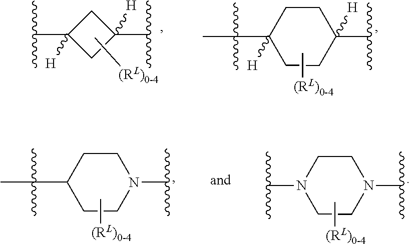

Embodiment 17. The compound according to Embodiment 13, wherein the Linker is selected from the group consisting of:

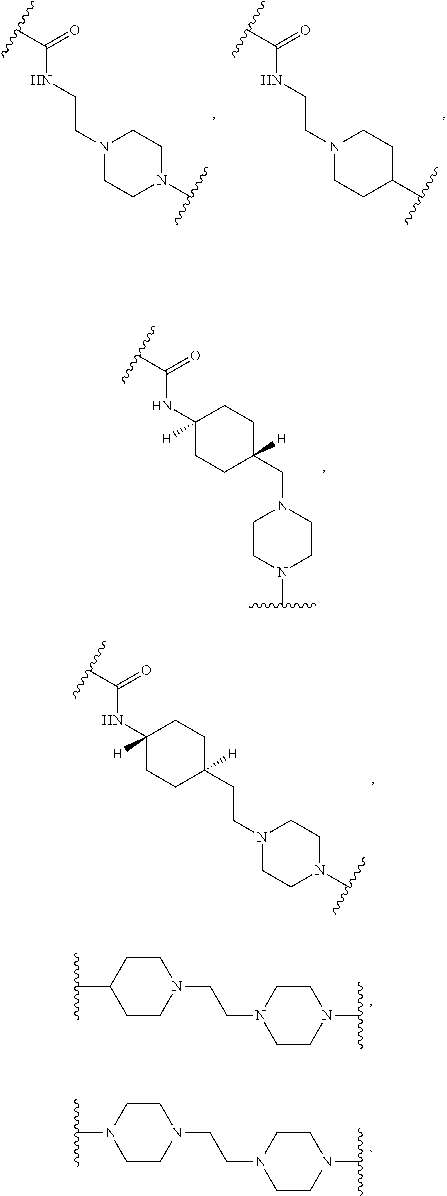 [SMILES: *C(=O)NCCN1CCN(*)CC1 ; *C(=O)NCCN1CCC(*)CC1 ; *C(=O)N[C@H]1CC[C@H](CN2CCN(*)CC2)CC1 ; *C(=O)N[C@H]1CC[C@H](CCN2CCN(*)CC2)CC1 ; *C1CCN(CCN2CCN(*)CC2)CC1 ; *N1CCN(CCN2CCN(*)CC2)CC1]

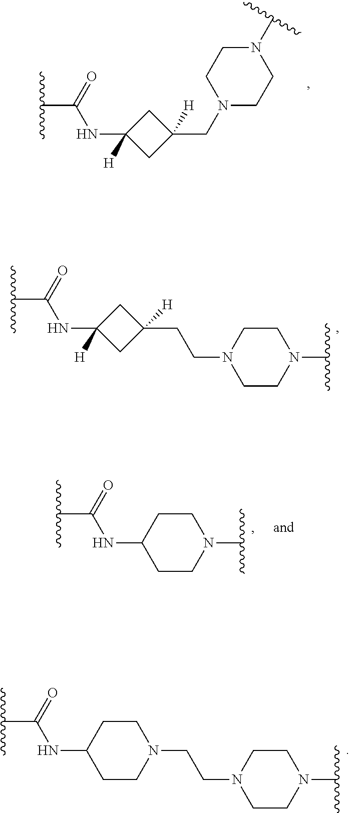 [SMILES: *C(=O)N[C@H]1C[C@H](CN2CCN(*)CC2)C1 ; *C(=O)N[C@H]1C[C@H](CCN2CCN(*)CC2)C1 ; *C(=O)NC1CCN(*)CC1 ; *C(=O)NC1CCN(CCN2CCN(*)CC2)CC1]

Embodiment 18. The compound according to Embodiment 17, wherein the Linker is selected from the group consisting of:

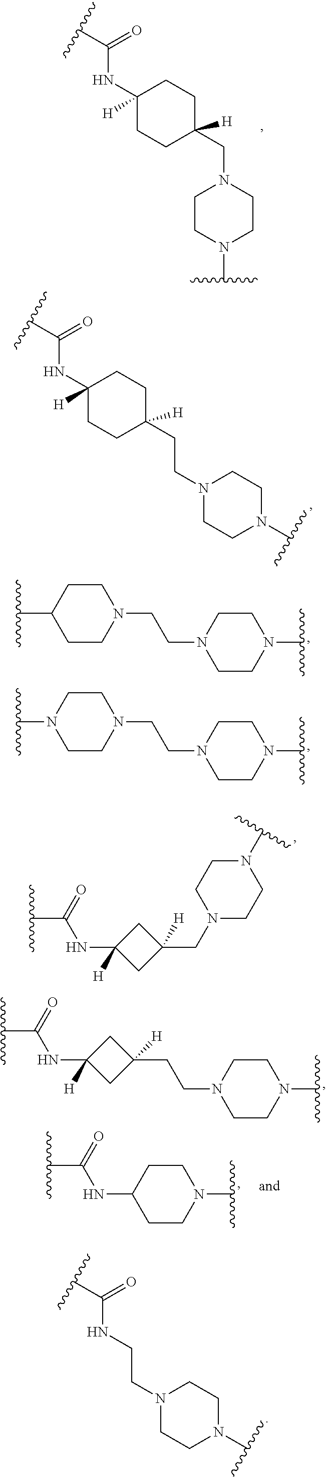

Embodiment 19. The compound according to any one of Embodiments 1-18, wherein the E3 binding moiety is a cereblon protein binding moiety.

Embodiment 20. The compound according to Embodiment 19, wherein the cereblon protein binding moiety is selected from the group consisting of:

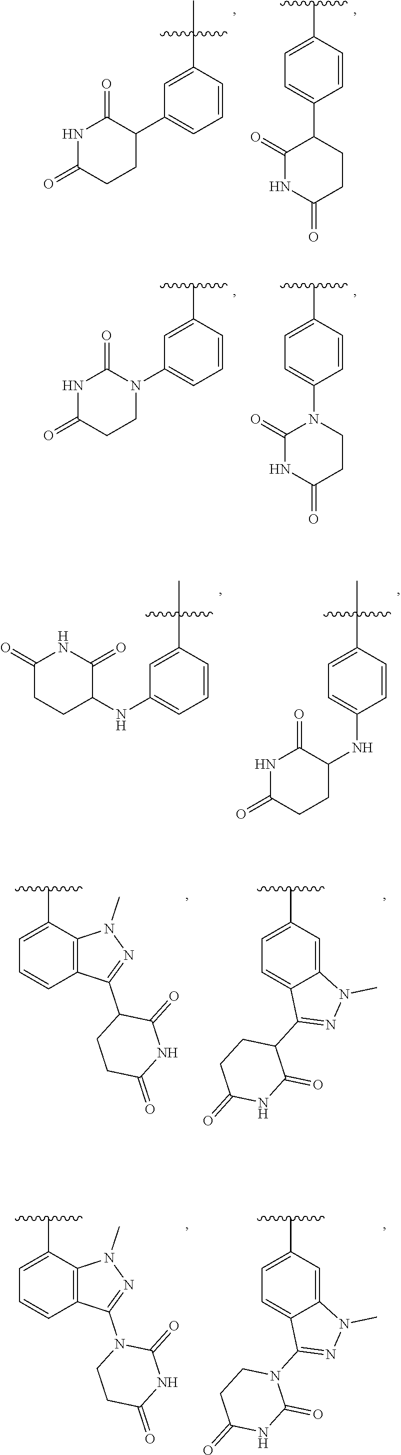

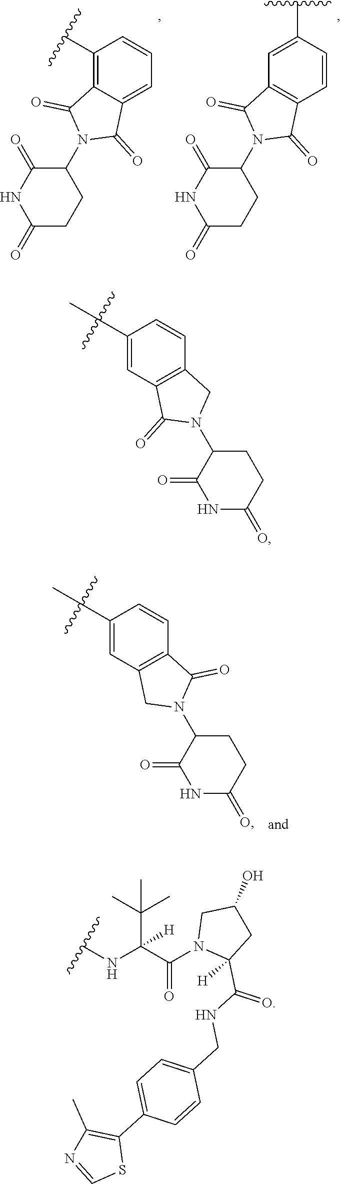 [SMILES: *c1cccc2c1C(=O)N(C1CCC(=O)NC1=O)C2=O ; *c1ccc2c(c1)C(=O)N(C1CCC(=O)NC1=O)C2=O ; *c1ccc2c(c1)C(=O)N(C1CCC(=O)NC1=O)C2 ; *c1ccc2c(c1)CN(C1CCC(=O)NC1=O)C2=O ; *N[C@H](C(=O)N1C[C@H](O)C[C@H]1C(=O)NCc1ccc(-c2scnc2C)cc1)C(C)(C)C]

Embodiment 21. The compound according to Embodiment 20, wherein the cereblon protein binding moiety is selected from:

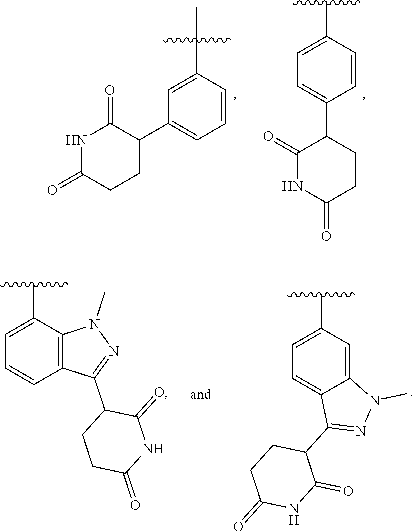

Embodiment 22. The compound according to claim 1, wherein the compound of formula I is selected from:

| | |
|---|---|
| 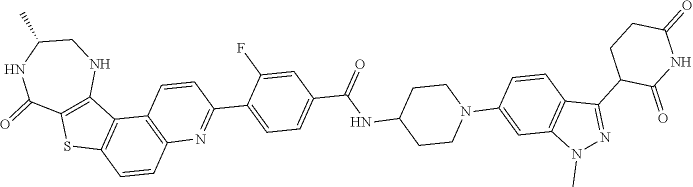 [SMILES: C[C@@H]1CNc2c(sc3ccc4nc(-c5ccc(C(=O)NC6CCN(c7ccc8c(C9CCC(=O)NC9=O)nn(C)c8c7)CC6)cc5F)ccc4c23)C(=O)N1] | Ex 1 |
| 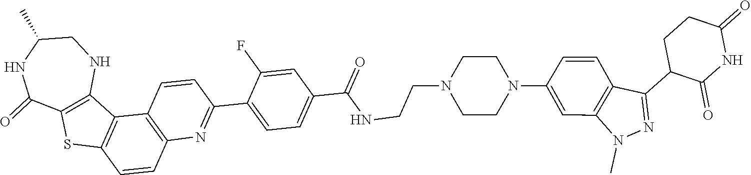 [SMILES: C[C@@H]1CNc2c(sc3ccc4nc(-c5ccc(C(=O)NCCN6CCN(c7ccc8c(C9CCC(=O)NC9=O)nn(C)c8c7)CC6)cc5F)ccc4c23)C(=O)N1] | Ex 2 |
| 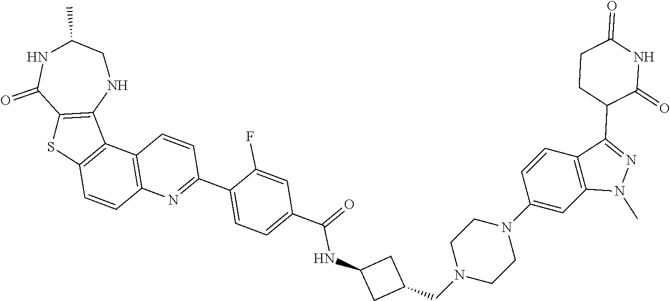 [SMILES: C[C@@H]1CNc2c(sc3ccc4nc(-c5ccc(C(=O)N[C@H]6C[C@H](CN7CCN(c8ccc9c(C%10CCC(=O)NC%10=O)nn(C)c9c8)CC7)C6)cc5F)ccc4c23)C(=O)N1] | Ex 3 |
| 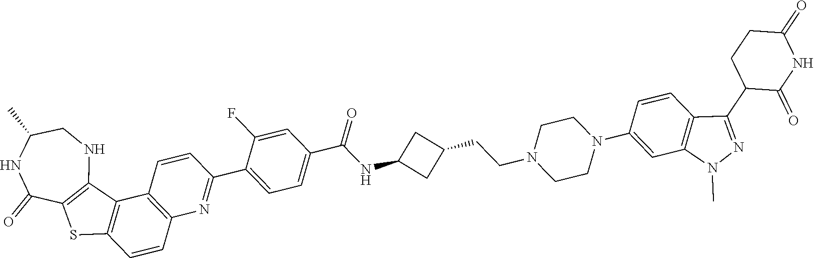 [SMILES: C[C@@H]1CNc2c(sc3ccc4nc(-c5ccc(C(=O)N[C@H]6C[C@H](CCN7CCN(c8ccc9c(C%10CCC(=O)NC%10=O)nn(C)c9c8)CC7)C6)cc5F)ccc4c23)C(=O)N1] | Ex 4 |
| 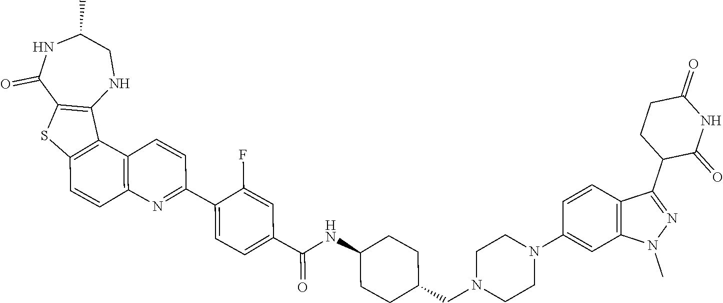 [SMILES: C[C@@H]1CNc2c(sc3ccc4nc(-c5ccc(C(=O)N[C@H]6CC[C@H](CN7CCN(c8ccc9c(C%10CCC(=O)NC%10=O)nn(C)c9c8)CC7)CC6)cc5F)ccc4c23)C(=O)N1] | Ex 5 |
| 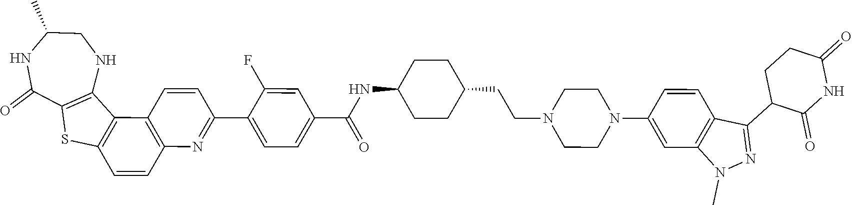 [SMILES: C[C@@H]1CNc2c(sc3ccc4nc(-c5ccc(C(=O)N[C@H]6CC[C@H](CCN7CCN(c8ccc9c(C%10CCC(=O)NC%10=O)nn(C)c9c8)CC7)CC6)cc5F)ccc4c23)C(=O)N1] | Ex 6 |
| 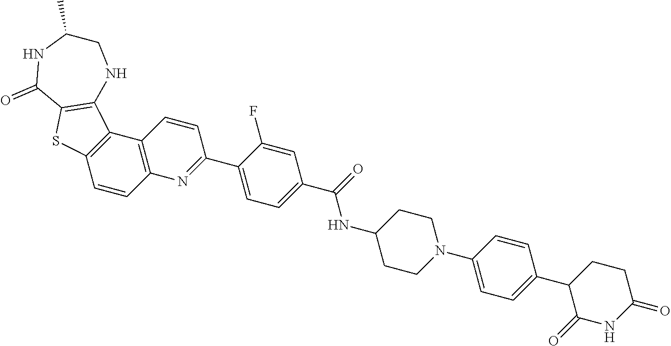 [SMILES: C[C@@H]1CNc2c(sc3ccc4nc(-c5ccc(C(=O)NC6CCN(c7ccc(C8CCC(=O)NC8=O)cc7)CC6)cc5F)ccc4c23)C(=O)N1] | Ex 7 |
| 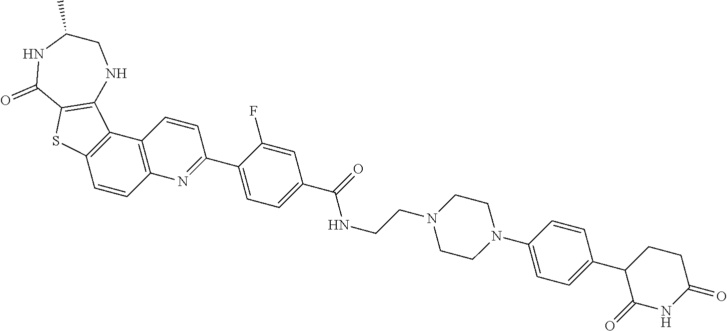 [SMILES: C[C@@H]1CNc2c(sc3ccc4nc(-c5ccc(C(=O)NCCN6CCN(c7ccc(C8CCC(=O)NC8=O)cc7)CC6)cc5F)ccc4c23)C(=O)N1] | Ex 8 |
| 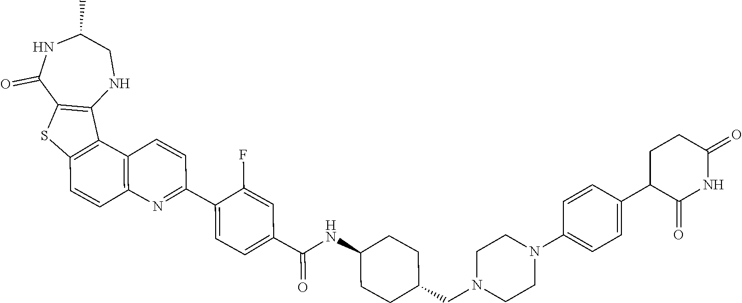 [SMILES: C[C@@H]1CNc2c(sc3ccc4nc(-c5ccc(C(=O)N[C@H]6CC[C@H](CN7CCN(c8ccc(C9CCC(=O)NC9=O)cc8)CC7)CC6)cc5F)ccc4c23)C(=O)N1] | Ex 9 |
| 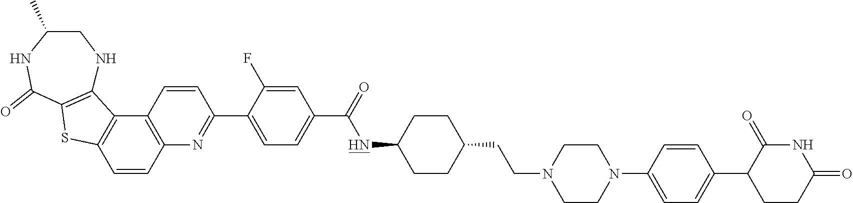 [SMILES: C[C@@H]1CNc2c(sc3ccc4nc(-c5ccc(C(=O)N[C@H]6CC[C@H](CCN7CCN(c8ccc(C9CCC(=O)NC9=O)cc8)CC7)CC6)cc5F)ccc4c23)C(=O)N1] | Ex 10 |
| 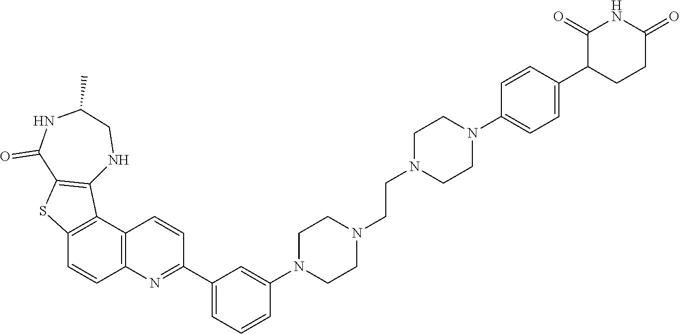 [SMILES: C[C@@H]1CNc2c(sc3ccc4nc(-c5cccc(N6CCN(CCN7CCN(c8ccc(C9CCC(=O)NC9=O)cc8)CC7)CC6)c5)ccc4c23)C(=O)N1] | Ex 11 |
| 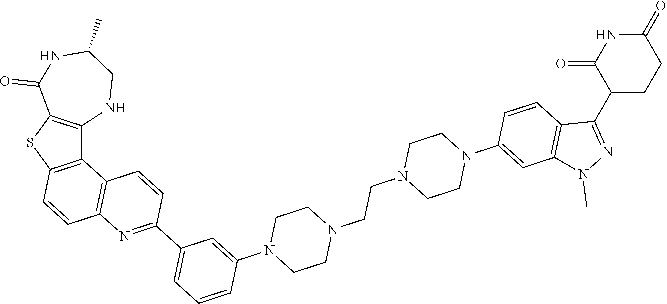 [SMILES: C[C@@H]1CNc2c(sc3ccc4nc(-c5cccc(N6CCN(CCN7CCN(c8ccc9c(C%10CCC(=O)NC%10=O)nn(C)c9c8)CC7)CC6)c5)ccc4c23)C(=O)N1] | Ex 12 |
| 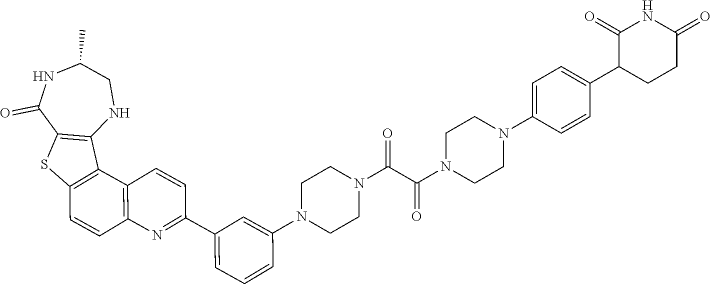 [SMILES: C[C@@H]1CNc2c(sc3ccc4nc(-c5cccc(N6CCN(C(=O)C(=O)N7CCN(c8ccc(C9CCC(=O)NC9=O)cc8)CC7)CC6)c5)ccc4c23)C(=O)N1] | Ex 13 |
| 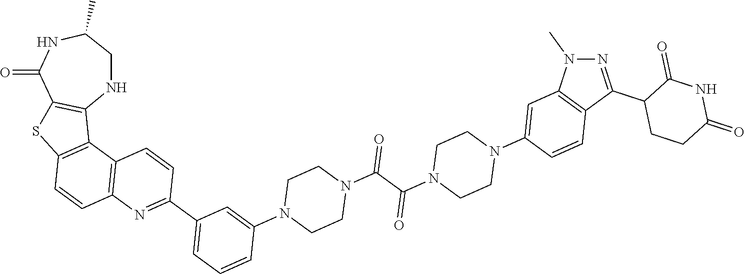 [SMILES: C[C@@H]1CNc2c(sc3ccc4nc(-c5cccc(N6CCN(C(=O)C(=O)N7CCN(c8ccc9c(C%10CCC(=O)NC%10=O)nn(C)c9c8)CC7)CC6)c5)ccc4c23)C(=O)N1] | Ex 14 |
| 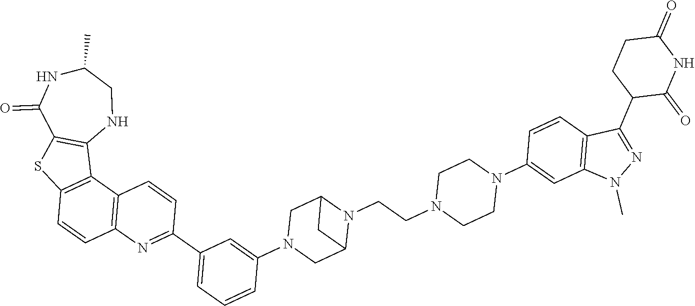 [SMILES: C[C@@H]1CNc2c(sc3ccc4nc(-c5cccc(N6CC7CC(C6)N7CCN6CCN(c7ccc8c(C9CCC(=O)NC9=O)nn(C)c8c7)CC6)c5)ccc4c23)C(=O)N1] | Ex 15 |
| 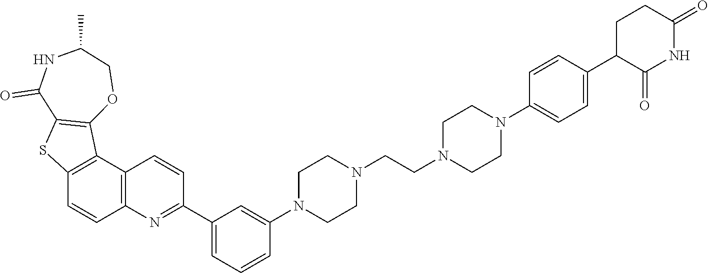 [SMILES: C[C@@H]1COc2c(sc3ccc4nc(-c5cccc(N6CCN(CCN7CCN(c8ccc(C9CCC(=O)NC9=O)cc8)CC7)CC6)c5)ccc4c23)C(=O)N1] | Ex 16 |
| 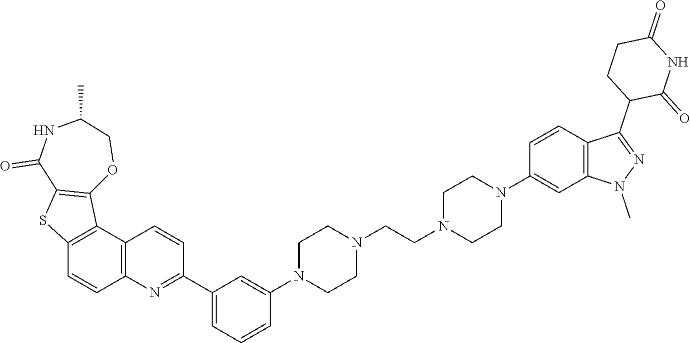 [SMILES: C[C@@H]1COc2c(sc3ccc4nc(-c5cccc(N6CCN(CCN7CCN(c8ccc9c(C%10CCC(=O)NC%10=O)nn(C)c9c8)CC7)CC6)c5)ccc4c23)C(=O)N1] | Ex 17 |
| 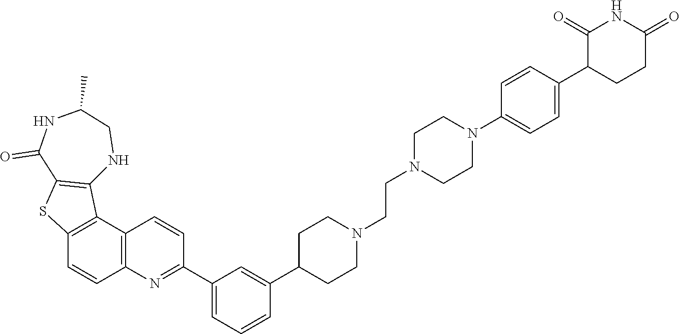 [SMILES: C[C@@H]1CNc2c(sc3ccc4nc(-c5cccc(C6CCN(CCN7CCN(c8ccc(C9CCC(=O)NC9=O)cc8)CC7)CC6)c5)ccc4c23)C(=O)N1] | Ex 18 |
| 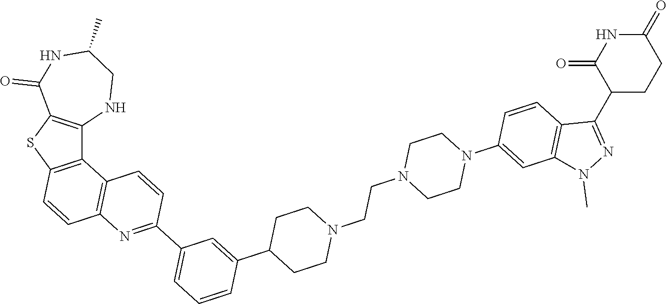 [SMILES: C[C@@H]1CNc2c(sc3ccc4nc(-c5cccc(C6CCN(CCN7CCN(c8ccc9c(C%10CCC(=O)NC%10=O)nn(C)c9c8)CC7)CC6)c5)ccc4c23)C(=O)N1] | Ex 19 |
| 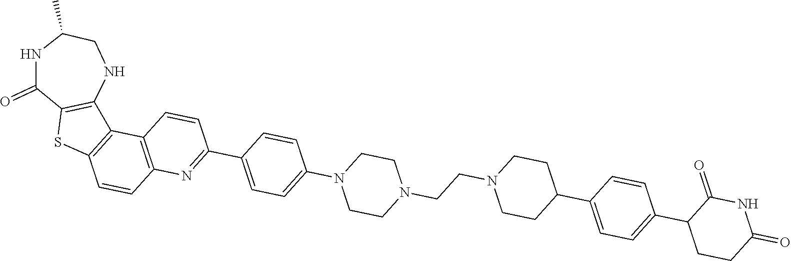 [SMILES: C[C@@H]1CNc2c(sc3ccc4nc(-c5ccc(N6CCN(CCN7CCC(c8ccc(C9CCC(=O)NC9=O)cc8)CC7)CC6)cc5)ccc4c23)C(=O)N1] | Ex 20 |
| 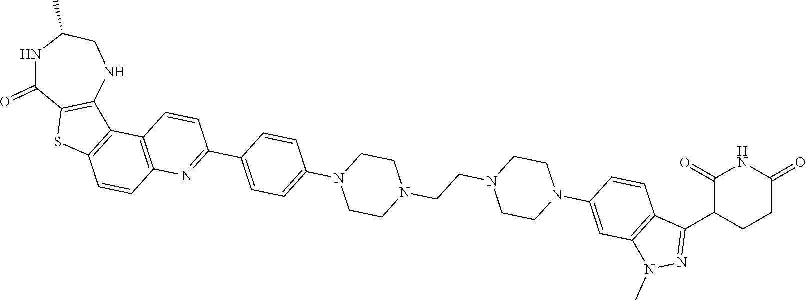 [SMILES: C[C@@H]1CNc2c(sc3ccc4nc(-c5ccc(N6CCN(CCN7CCN(c8ccc9c(C%10CCC(=O)NC%10=O)nn(C)c9c8)CC7)CC6)cc5)ccc4c23)C(=O)N1] | Ex 21 |
| 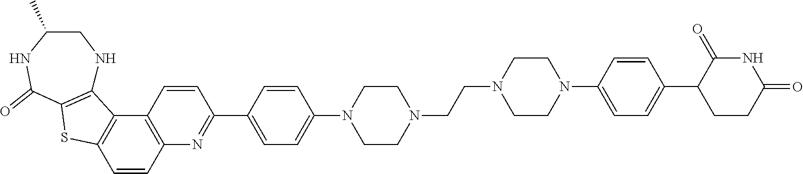 [SMILES: C[C@@H]1CNc2c(sc3ccc4nc(-c5ccc(N6CCN(CCN7CCN(c8ccc(C9CCC(=O)NC9=O)cc8)CC7)CC6)cc5)ccc4c23)C(=O)N1] | Ex 22 |
| 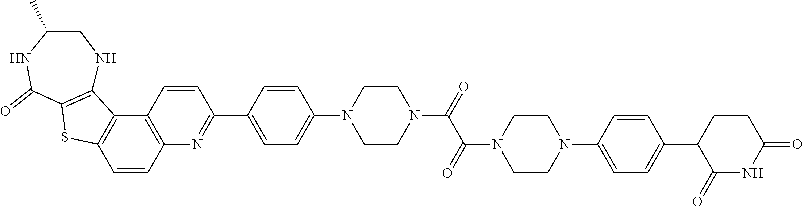 [SMILES: C[C@@H]1CNc2c(sc3ccc4nc(-c5ccc(N6CCN(C(=O)C(=O)N7CCN(c8ccc(C9CCC(=O)NC9=O)cc8)CC7)CC6)cc5)ccc4c23)C(=O)N1] | Ex 23 |
| 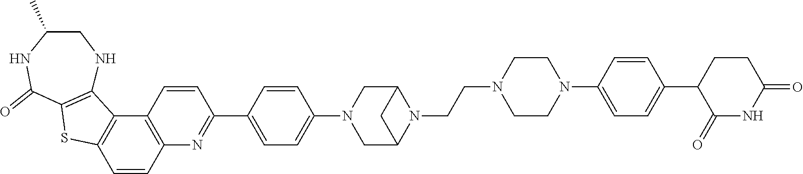 [SMILES: C[C@@H]1CNc2c(sc3ccc4nc(-c5ccc(N6CC7CC(C6)N7CCN6CCN(c7ccc(C8CCC(=O)NC8=O)cc7)CC6)cc5)ccc4c23)C(=O)N1] | Ex 24 |
| 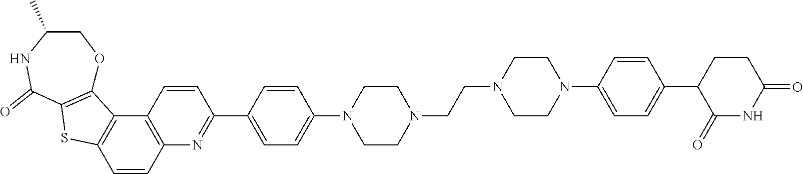 [SMILES: C[C@@H]1COc2c(sc3ccc4nc(-c5ccc(N6CCN(CCN7CCN(c8ccc(C9CCC(=O)NC9=O)cc8)CC7)CC6)cc5)ccc4c23)C(=O)N1] | Ex 25 |
| 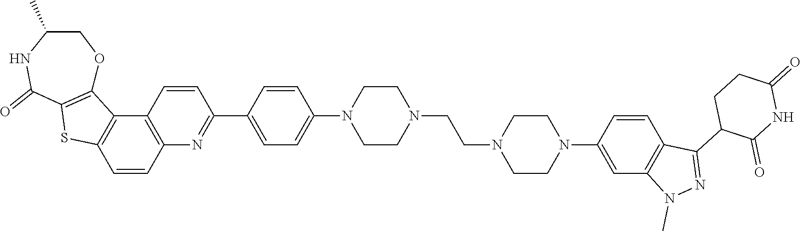 [SMILES: C[C@@H]1COc2c(sc3ccc4nc(-c5ccc(N6CCN(CCN7CCN(c8ccc9c(C%10CCC(=O)NC%10=O)nn(C)c9c8)CC7)CC6)cc5)ccc4c23)C(=O)N1] | Ex 26 |
| 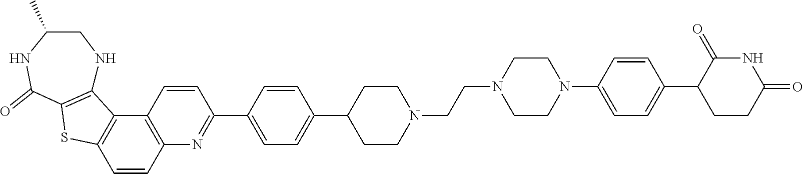 [SMILES: C[C@@H]1CNc2c(sc3ccc4nc(-c5ccc(C6CCN(CCN7CCN(c8ccc(C9CCC(=O)NC9=O)cc8)CC7)CC6)cc5)ccc4c23)C(=O)N1] | Ex 27 |
| 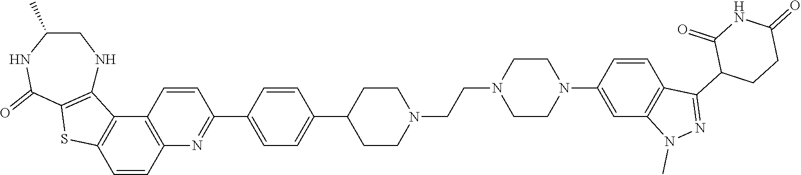 [SMILES: C[C@@H]1CNc2c(sc3ccc4nc(-c5ccc(C6CCN(CCN7CCN(c8ccc9c(C%10CCC(=O)NC%10=O)nn(C)c9c8)CC7)CC6)cc5)ccc4c23)C(=O)N1] | Ex 28 |
| 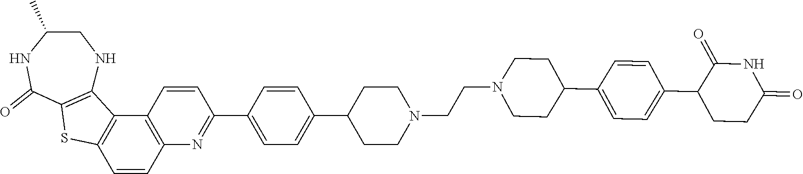 [SMILES: C[C@@H]1CNc2c(sc3ccc4nc(-c5ccc(C6CCN(CCN7CCC(c8ccc(C9CCC(=O)NC9=O)cc8)CC7)CC6)cc5)ccc4c23)C(=O)N1] | Ex 29 |
| 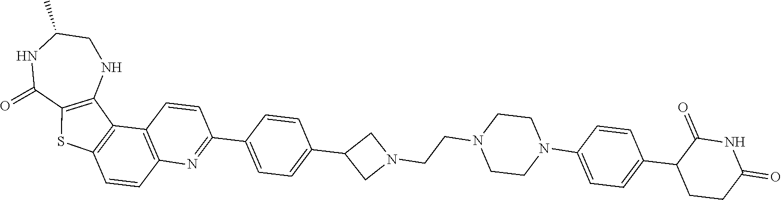 [SMILES: C[C@@H]1CNc2c(sc3ccc4nc(-c5ccc(C6CN(CCN7CCN(c8ccc(C9CCC(=O)NC9=O)cc8)CC7)C6)cc5)ccc4c23)C(=O)N1] | Ex 30 |
| 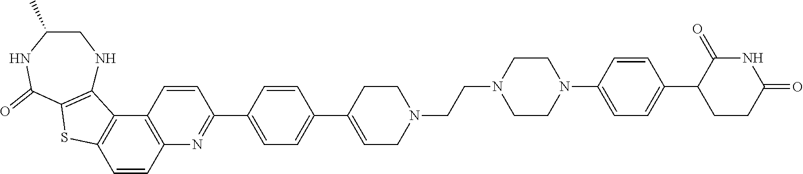 [SMILES: C[C@@H]1CNc2c(sc3ccc4nc(-c5ccc(C6=CCN(CCN7CCN(c8ccc(C9CCC(=O)NC9=O)cc8)CC7)CC6)cc5)ccc4c23)C(=O)N1] | Ex 31 |
| 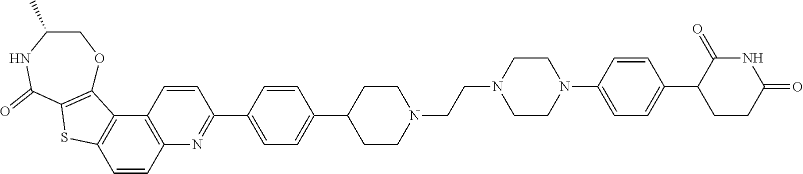 [SMILES: C[C@@H]1COc2c(sc3ccc4nc(-c5ccc(C6CCN(CCN7CCN(c8ccc(C9CCC(=O)NC9=O)cc8)CC7)CC6)cc5)ccc4c23)C(=O)N1] | Ex 32 |
| 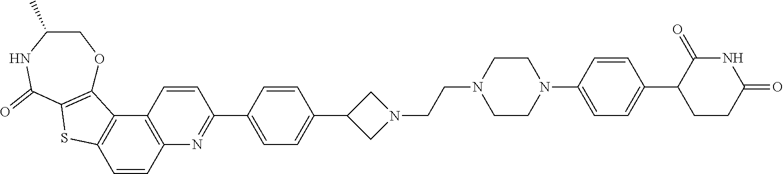 [SMILES: C[C@@H]1COc2c(sc3ccc4nc(-c5ccc(C6CN(CCN7CCN(c8ccc(C9CCC(=O)NC9=O)cc8)CC7)C6)cc5)ccc4c23)C(=O)N1] | Ex 33 |
| 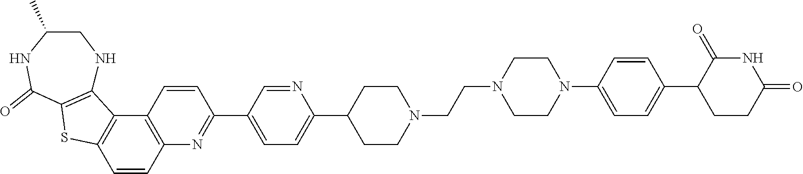 [SMILES: C[C@@H]1CNc2c(sc3ccc4nc(-c5ccc(C6CCN(CCN7CCN(c8ccc(C9CCC(=O)NC9=O)cc8)CC7)CC6)nc5)ccc4c23)C(=O)N1] | Ex 34 |
| 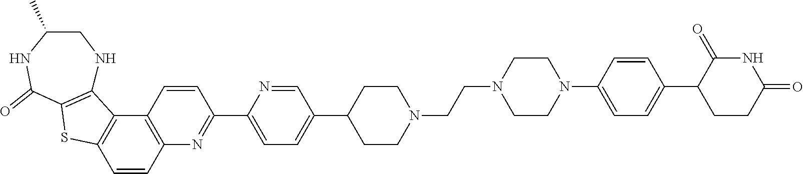 [SMILES: C[C@@H]1CNc2c(sc3ccc4nc(-c5ccc(C6CCN(CCN7CCN(c8ccc(C9CCC(=O)NC9=O)cc8)CC7)CC6)cn5)ccc4c23)C(=O)N1] | Ex 35 |
| 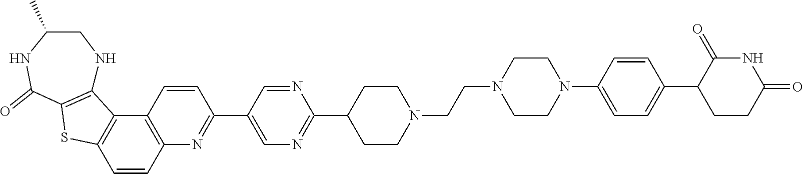 [SMILES: C[C@@H]1CNc2c(sc3ccc4nc(-c5cnc(C6CCN(CCN7CCN(c8ccc(C9CCC(=O)NC9=O)cc8)CC7)CC6)nc5)ccc4c23)C(=O)N1] | Ex 36 |
| 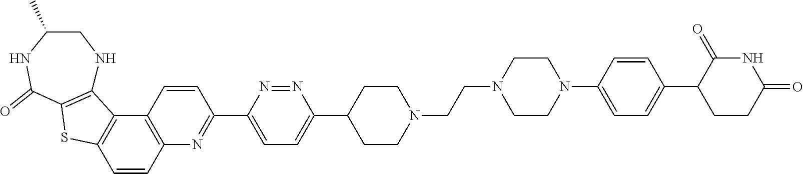 [SMILES: C[C@@H]1CNc2c(sc3ccc4nc(-c5ccc(C6CCN(CCN7CCN(c8ccc(C9CCC(=O)NC9=O)cc8)CC7)CC6)nn5)ccc4c23)C(=O)N1] | Ex 37 |
| 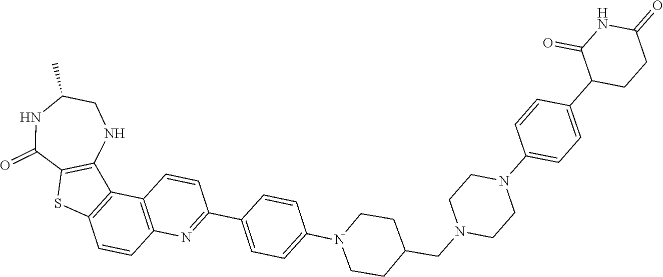 [SMILES: C[C@@H]1CNc2c(sc3ccc4nc(-c5ccc(N6CCC(CN7CCN(c8ccc(C9CCC(=O)NC9=O)cc8)CC7)CC6)cc5)ccc4c23)C(=O)N1] | Ex 38 |
| 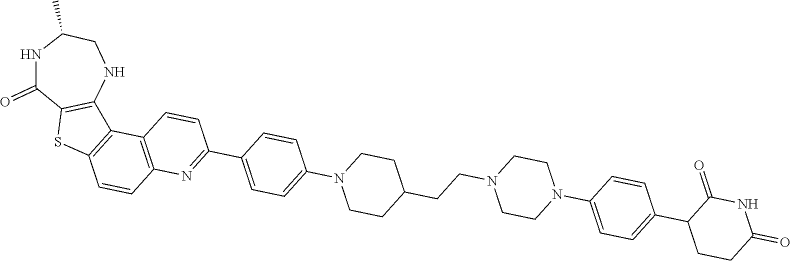 [SMILES: C[C@@H]1CNc2c(sc3ccc4nc(-c5ccc(N6CCC(CCN7CCN(c8ccc(C9CCC(=O)NC9=O)cc8)CC7)CC6)cc5)ccc4c23)C(=O)N1] | Ex 39 |
| 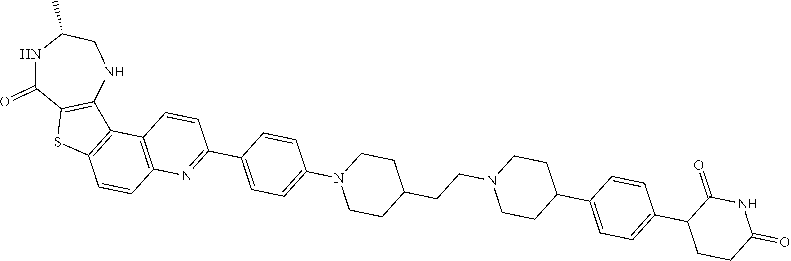 [SMILES: C[C@@H]1CNc2c(sc3ccc4nc(-c5ccc(N6CCC(CCN7CCC(c8ccc(C9CCC(=O)NC9=O)cc8)CC7)CC6)cc5)ccc4c23)C(=O)N1] | Ex 40 |
| 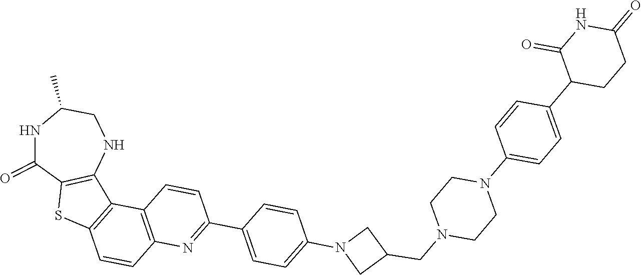 [SMILES: C[C@@H]1CNc2c(sc3ccc4nc(-c5ccc(N6CC(CN7CCN(c8ccc(C9CCC(=O)NC9=O)cc8)CC7)C6)cc5)ccc4c23)C(=O)N1] | Ex 41 |
| 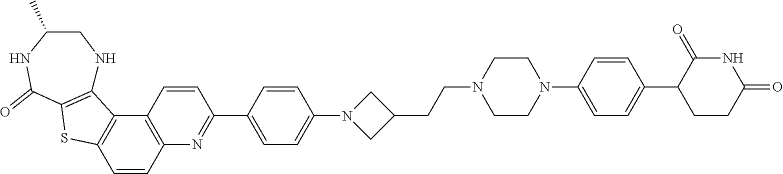 [SMILES: C[C@@H]1CNc2c(sc3ccc4nc(-c5ccc(N6CC(CCN7CCN(c8ccc(C9CCC(=O)NC9=O)cc8)CC7)C6)cc5)ccc4c23)C(=O)N1] | Ex 42 |
| 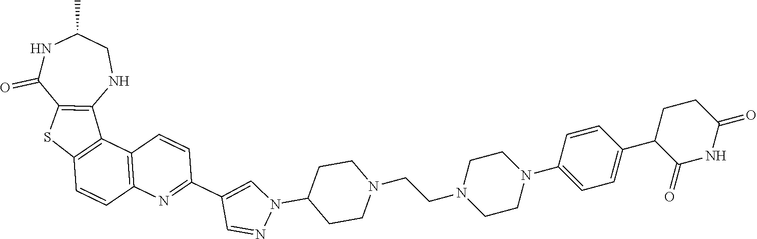 [SMILES: C[C@@H]1CNc2c(sc3ccc4nc(-c5cnn(C6CCN(CCN7CCN(c8ccc(C9CCC(=O)NC9=O)cc8)CC7)CC6)c5)ccc4c23)C(=O)N1] | Ex 43 |
| 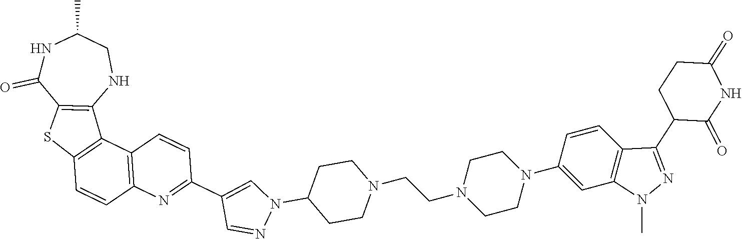 [SMILES: C[C@@H]1CNc2c(sc3ccc4nc(-c5cnn(C6CCN(CCN7CCN(c8ccc9c(C%10CCC(=O)NC%10=O)nn(C)c9c8)CC7)CC6)c5)ccc4c23)C(=O)N1] | Ex 44 |
| 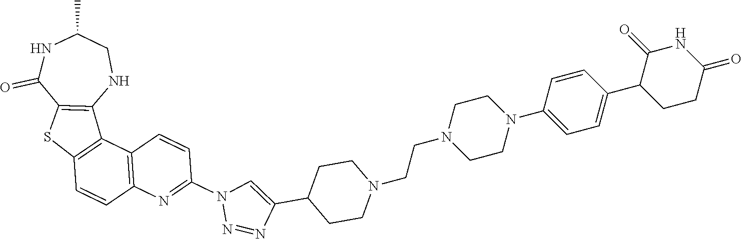 [SMILES: C[C@@H]1CNc2c(sc3ccc4nc(-n5cc(C6CCN(CCN7CCN(c8ccc(C9CCC(=O)NC9=O)cc8)CC7)CC6)nn5)ccc4c23)C(=O)N1] | Ex 45 |
| 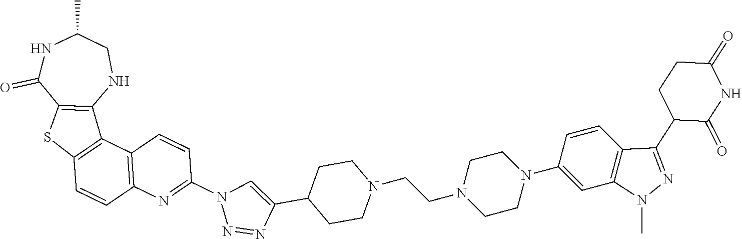 [SMILES: C[C@@H]1CNc2c(sc3ccc4nc(-n5cc(C6CCN(CCN7CCN(c8ccc9c(C%10CCC(=O)NC%10=O)nn(C)c9c8)CC7)CC6)nn5)ccc4c23)C(=O)N1] | Ex 46 |
| 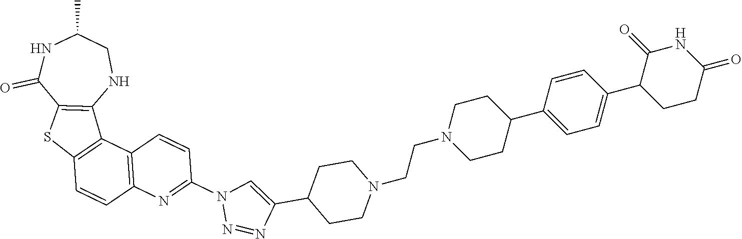 [SMILES: C[C@@H]1CNc2c(sc3ccc4nc(-n5cc(C6CCN(CCN7CCC(c8ccc(C9CCC(=O)NC9=O)cc8)CC7)CC6)nn5)ccc4c23)C(=O)N1] | Ex 47 |
| 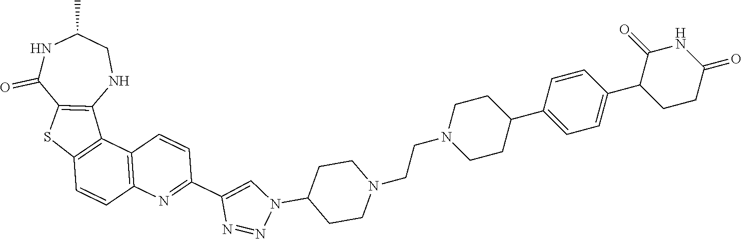 [SMILES: C[C@@H]1CNc2c(sc3ccc4nc(-c5cn(C6CCN(CCN7CCC(c8ccc(C9CCC(=O)NC9=O)cc8)CC7)CC6)nn5)ccc4c23)C(=O)N1] | Ex 48 |
| 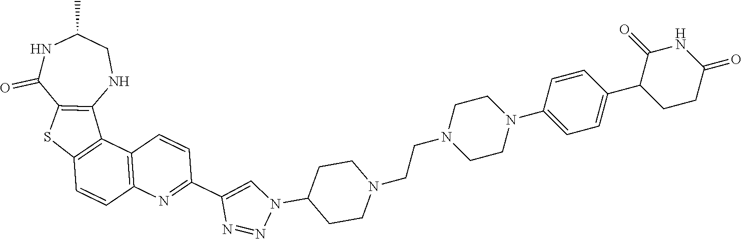 [SMILES: C[C@@H]1CNc2c(sc3ccc4nc(-c5cn(C6CCN(CCN7CCN(c8ccc(C9CCC(=O)NC9=O)cc8)CC7)CC6)nn5)ccc4c23)C(=O)N1] | Ex 49 |
| 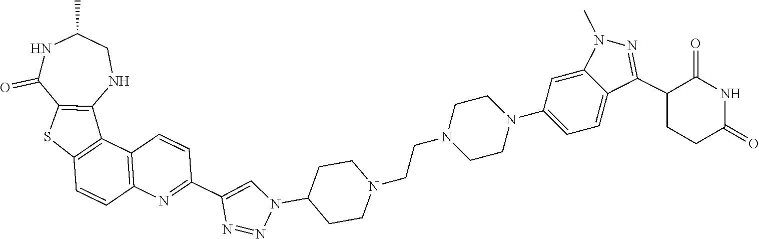 [SMILES: C[C@@H]1CNc2c(sc3ccc4nc(-c5cn(C6CCN(CCN7CCN(c8ccc9c(C%10CCC(=O)NC%10=O)nn(C)c9c8)CC7)CC6)nn5)ccc4c23)C(=O)N1] | Ex 50 |
| 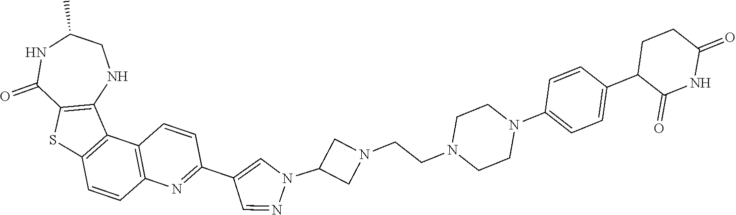 [SMILES: C[C@@H]1CNc2c(sc3ccc4nc(-c5cnn(C6CN(CCN7CCN(c8ccc(C9CCC(=O)NC9=O)cc8)CC7)C6)c5)ccc4c23)C(=O)N1] | Ex 51 |
| 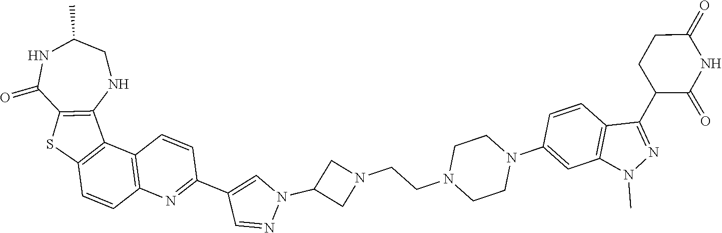 [SMILES: C[C@@H]1CNc2c(sc3ccc4nc(-c5cnn(C6CN(CCN7CCN(c8ccc9c(C%10CCC(=O)NC%10=O)nn(C)c9c8)CC7)C6)c5)ccc4c23)C(=O)N1] | Ex 52 |
| 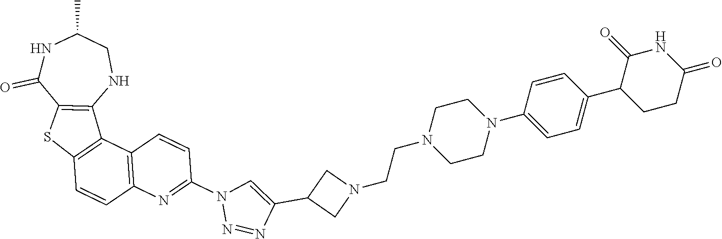 [SMILES: C[C@@H]1CNc2c(sc3ccc4nc(-n5cc(C6CN(CCN7CCN(c8ccc(C9CCC(=O)NC9=O)cc8)CC7)C6)nn5)ccc4c23)C(=O)N1] | Ex 53 |
| 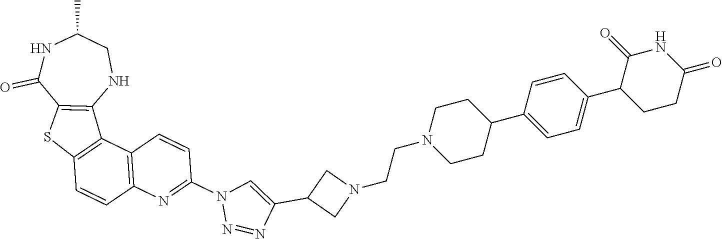 [SMILES: C[C@@H]1CNc2c(sc3ccc4nc(-n5cc(C6CN(CCN7CCC(c8ccc(C9CCC(=O)NC9=O)cc8)CC7)C6)nn5)ccc4c23)C(=O)N1] | Ex 54 |
| 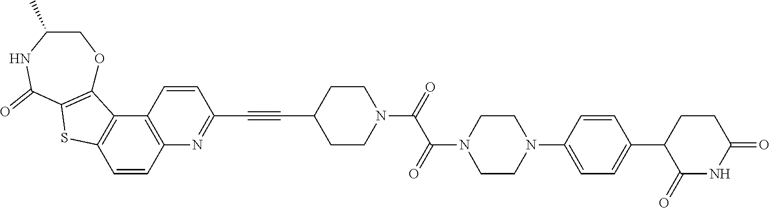 [SMILES: C[C@@H]1COc2c(sc3ccc4nc(C#CC5CCN(C(=O)C(=O)N6CCN(c7ccc(C8CCC(=O)NC8=O)cc7)CC6)CC5)ccc4c23)C(=O)N1] | Ex 55 |

or a pharmaceutically acceptable salt thereof.

Embodiment 23. A pharmaceutical composition comprising a compound according to any one of Embodiments 1-22 and a pharmaceutically acceptable excipient, carrier, or diluent.

Embodiment 24. A method of inhibiting the activity of MK2, or a mutant thereof, the method comprising contacting a biological sample with a compound according to any one of Embodiments 1-22.

Embodiment 25. A method of treating a disease, disorder, or condition mediated by MK2, or a mutant thereof, the method comprising administering to a patient in need thereof a compound according to any one of Embodiments 1-22, or a pharmaceutical composition according to Embodiment 23.

### EQUIVALENTS

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula I: 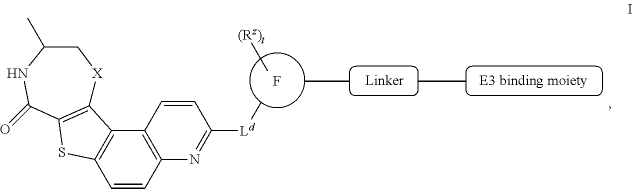
or a pharmaceutically acceptable salt thereof, wherein:
the Linker is an optionally substituted bivalent C₂₋₂₀ straight or branched aliphatic chain, wherein one, two, three, four, or five methylene units of the aliphatic chain are optionally and independently replaced by a group selected from —N(R)—, —O—, —C(O)—, —N(R)C(O)—, —C(O)N(R)—, —OC(O)—, —C(O)O—, a bivalent 3- to 6-membered monocyclic saturated or partially unsaturated ring having 0-2 heteroatoms independently selected from nitrogen, oxygen, and sulfur, a bivalent 6- to 8-membered saturated or partially unsaturated bridged bicyclic, fused bicyclic or spirofused heterocyclic ring having 1-3 heteroatoms independently selected from nitrogen, oxygen, and sulfur, and phenylene, wherein each monocyclic ring, bridged bicyclic ring, fused bicyclic ring, spirofused ring, or phenylene is substituted by 0-4 instances of R^{L}, wherein
R^{L} is independently selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur;
the E3 binding moiety is a moiety that binds to an E3 ubiquitin ligase protein;
Ring F is phenylene, a 5- to 6-membered heteroarylene ring having 1-4 heteroatoms independently selected from nitrogen, oxygen, and sulfur, or a 3- to 7-membered saturated or partially unsaturated heterocyclylene having 1-3 heteroatoms independently selected from nitrogen, oxygen, and sulfur;
L^{d} is selected from a covalent bond, —O—, —S—, —N(R)—, and C₁₋₆ aliphatic;
X is selected from —O—, —S—, and —N(R)—;
R^{z} is selected from halogen, —OR, —SR, —CN, —NO₂, —SO₂NR, —SO₂R, —SOR, —C(O)R, —CO₂R, —C(O)N(R)₂, —NRC(O)R, —NRC(O)OR, —NRC(O)N(R)₂, —NRSO₂R, —N(R)₂, or an optionally substituted group selected from the group consisting of C₁₋₆ aliphatic, phenyl, a 3- to 8-membered saturated or partially unsaturated carbocyclic ring, a 4- to 7-membered heterocyclic ring having 1-2 heteroatoms independently selected from nitrogen, oxygen, or sulfur, and a 5- to 6-membered monocyclic heteroaryl ring having 1-4 heteroatoms independently selected from nitrogen, oxygen, or sulfur;
each R is independently hydrogen or optionally substituted C₁₋₆ aliphatic; and
t is 0, 1, 2, or 3.

2. The compound according to claim 1, wherein Ring F is phenylene.

3. The compound according to claim 1, wherein L^{d} is a covalent bond.

4. The compound according to claim 1, wherein X is —O—.

5. The compound according to claim 1, wherein X is —N(R)—.

6. The compound according to claim 5, wherein R is hydrogen.

7. The compound according to claim 1, wherein R^{z} is selected from halogen, —OR, —SR, —CN, —NO₂, —SO₂NR, —SO₂R, —SOR, —C(O)R, —CO₂R, and —C(O)N(R)₂.

8. The compound according to claim 1, wherein R^{z} is selected from halogen, —OR, —SR, —CN, and —NO₂.

9. The compound according to claim 1, wherein R^{z} is halogen.

10. The compound according to claim 1, wherein t is 0.

11. The compound according to claim 1, wherein t is 1.

12. The compound according to claim 1, wherein the compound is a compound of any of Formulae I-a, I-a-i, I-a-ii, I-a-iii, I-a-iv, I-a-v, I-b, I-b-i, I-b-ii, I-b-iii, I-b-iv, I-b-v, I-c, I-c-i, I-c-ii, I-c-iii, I-c-iv, I-c-v, I-d, I-d-i, and I-d-ii: 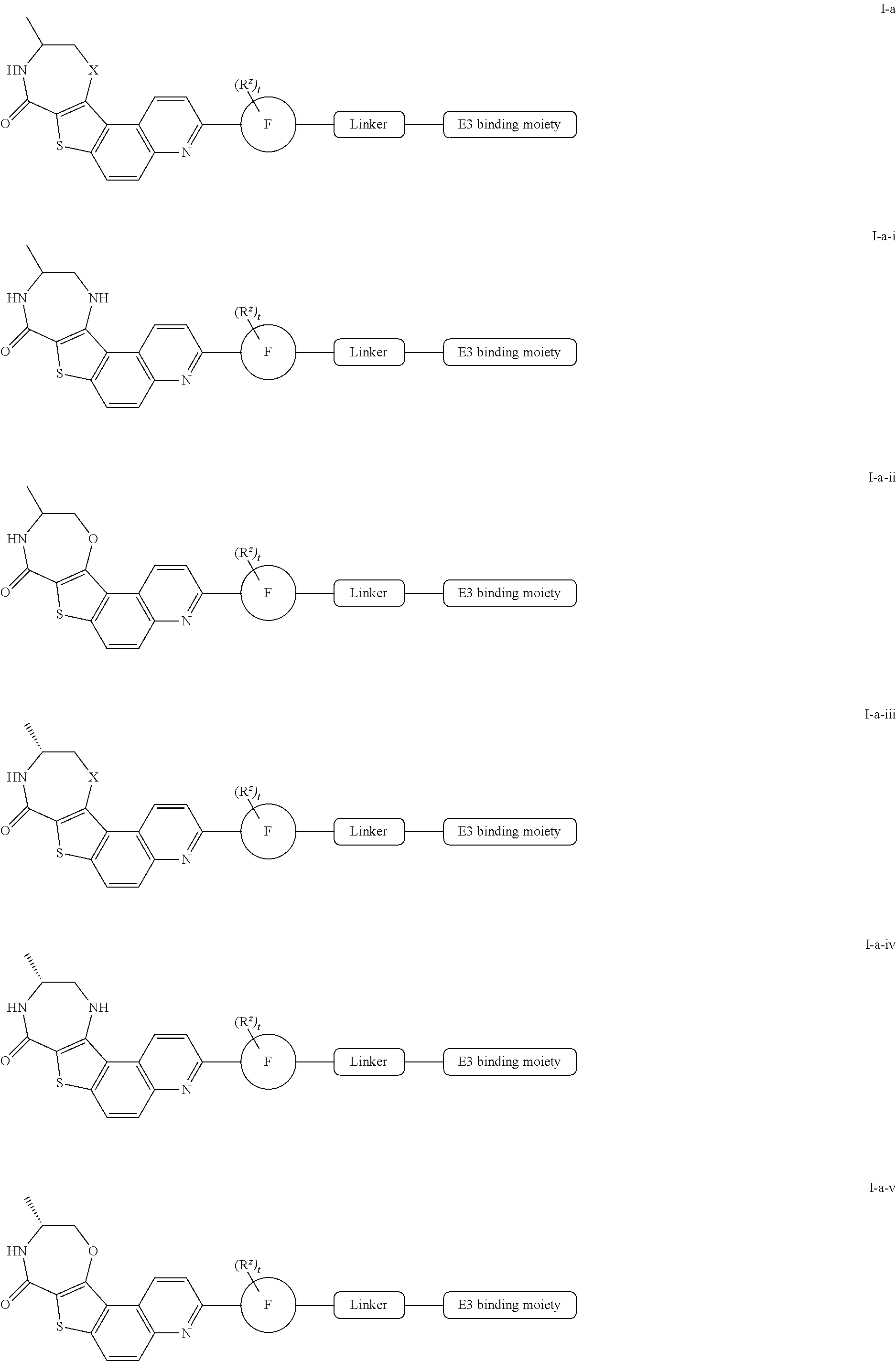 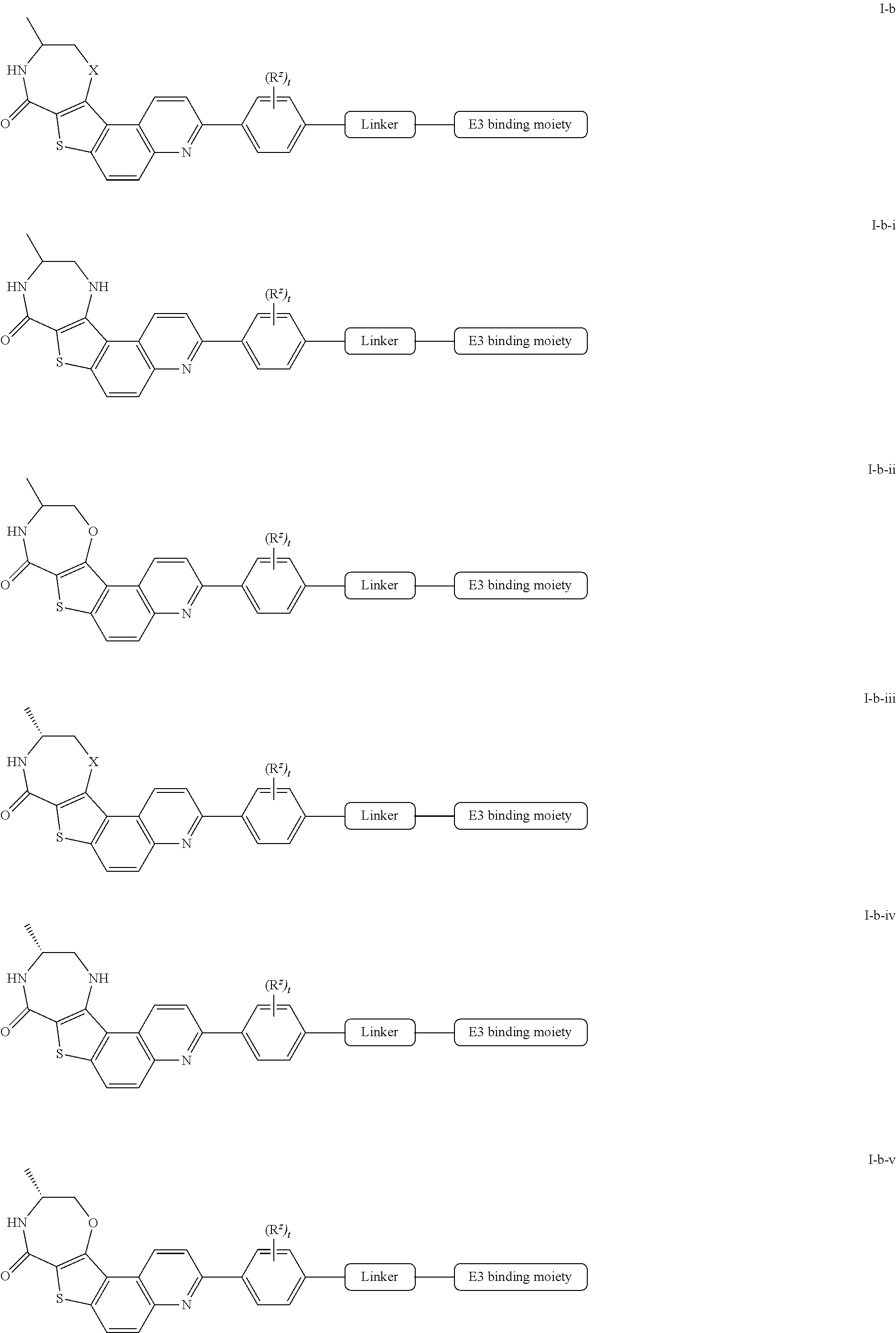 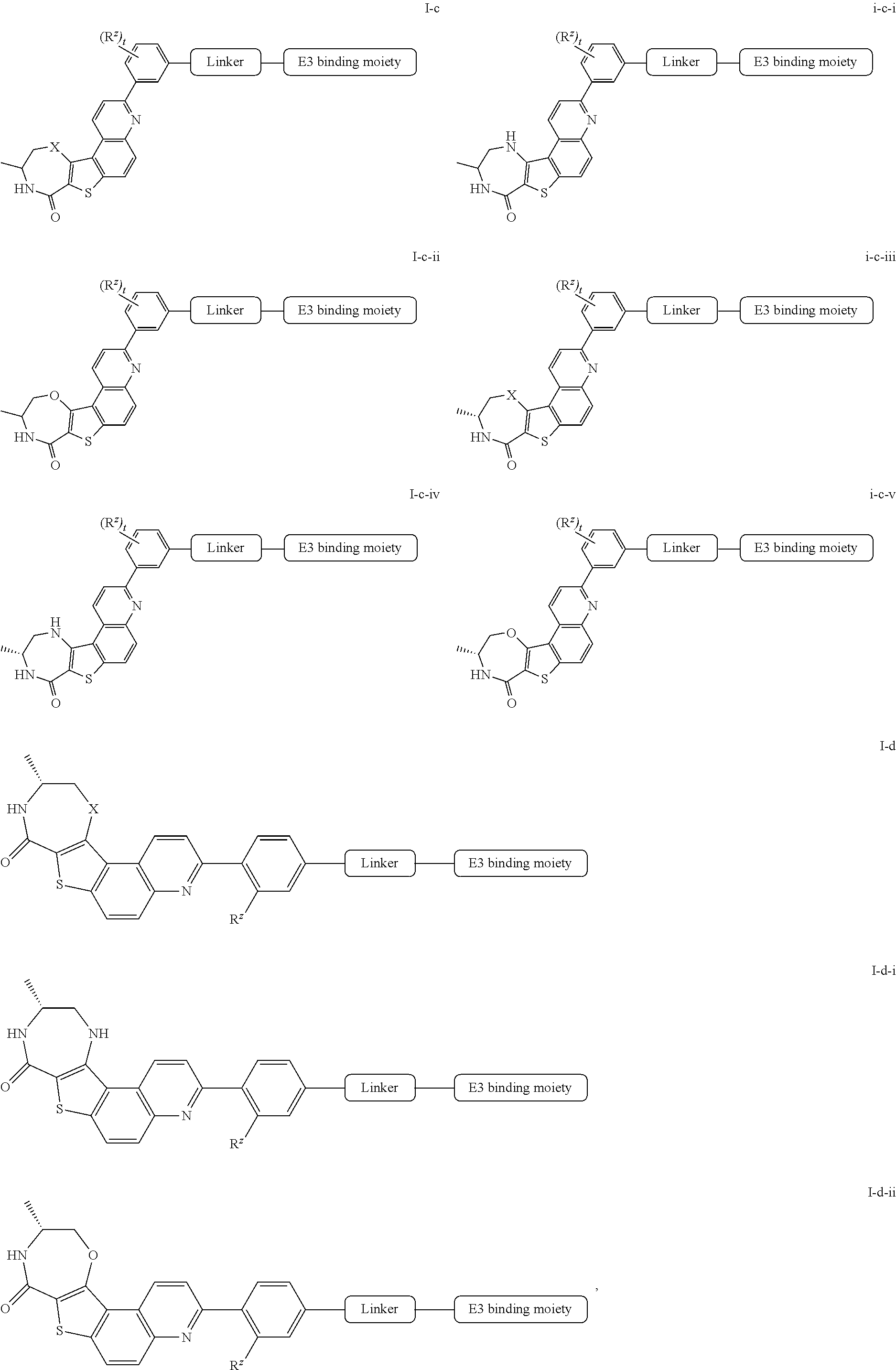
or a pharmaceutically acceptable salt thereof.

13. The compound according to claim 1, wherein the Linker is selected from the group consisting of:

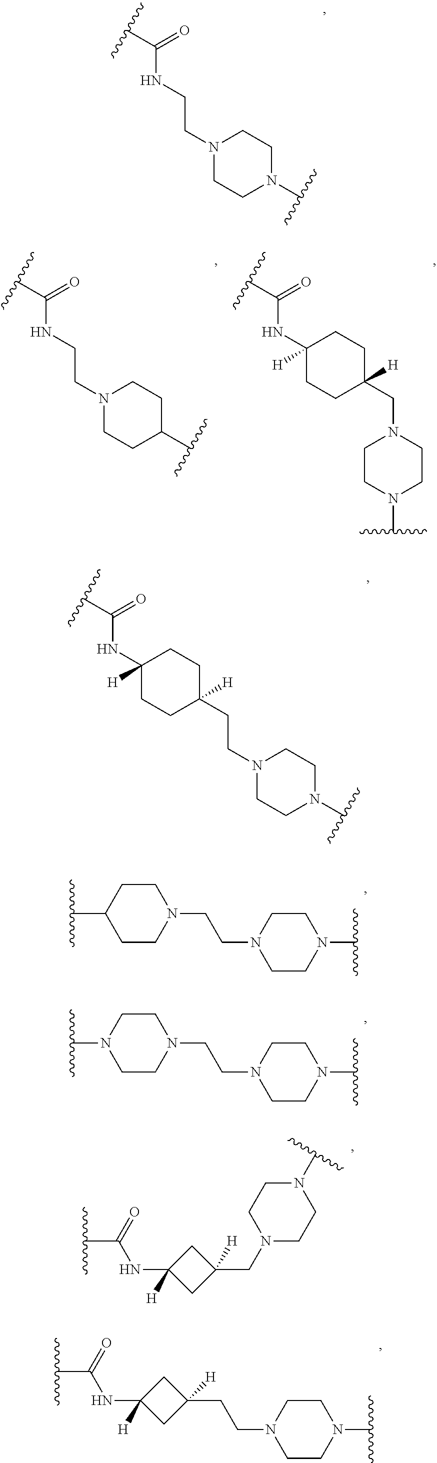

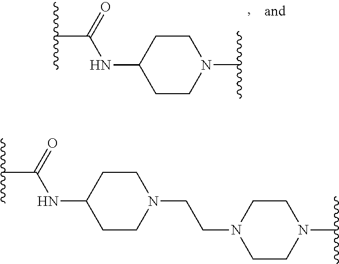 [SMILES: *C(=O)NC1CCN(*)CC1 ; *C(=O)NC1CCN(CCN2CCN(*)CC2)CC1]

14. The compound according to claim 1, wherein the Linker is selected from the group consisting of:

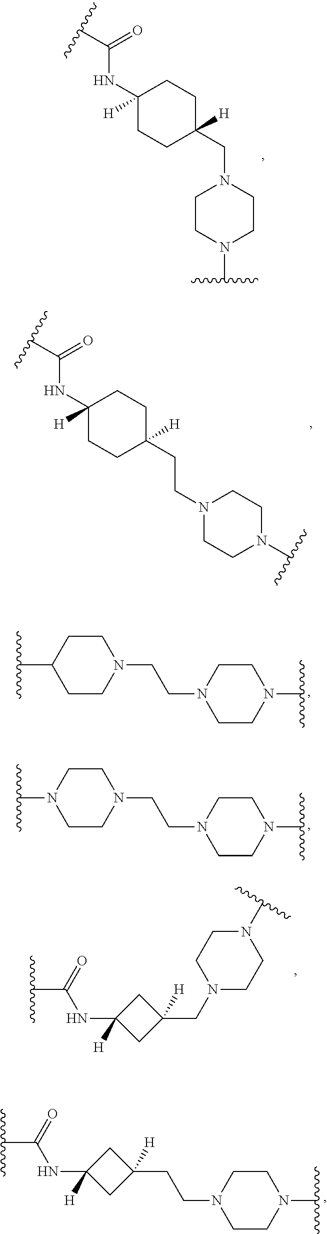 [SMILES: *C(=O)N[C@H]1CC[C@H](CN2CCN(*)CC2)CC1 ; *C(=O)N[C@H]1CC[C@H](CCN2CCN(*)CC2)CC1 ; *C1CCN(CCN2CCN(*)CC2)CC1 ; *N1CCN(CCN2CCN(*)CC2)CC1 ; *C(=O)N[C@H]1C[C@H](CN2CCN(*)CC2)C1 ; *C(=O)N[C@H]1C[C@H](CCN2CCN(*)CC2)C1]

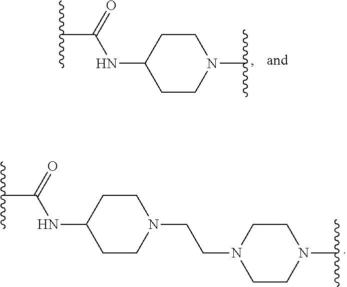 [SMILES: *C(=O)NC1CCN(*)CC1 ; *C(=O)NC1CCN(CCN2CCN(*)CC2)CC1]

15. The compound according to claim 1, wherein the E3 binding moiety is a cereblon protein binding moiety.

16. The compound according to claim 15, wherein the cereblon protein binding moiety is selected from the group consisting of:

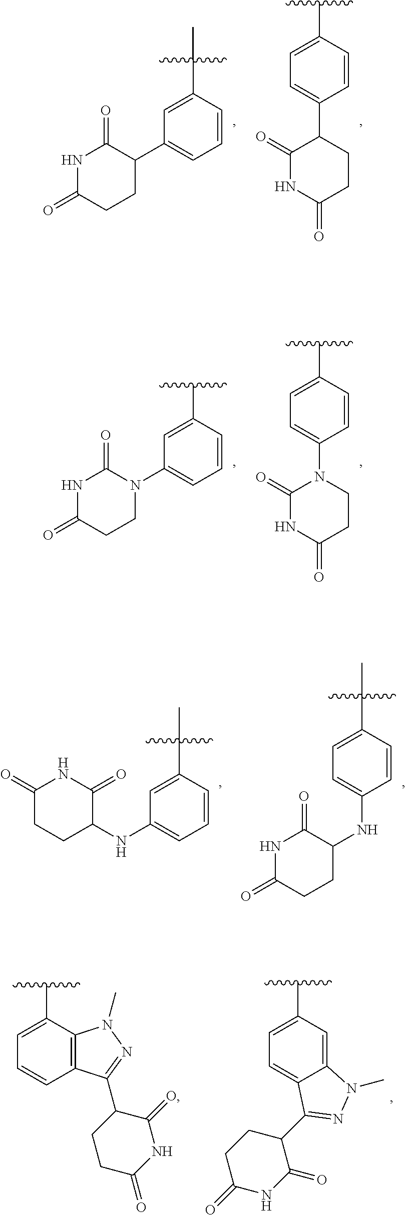

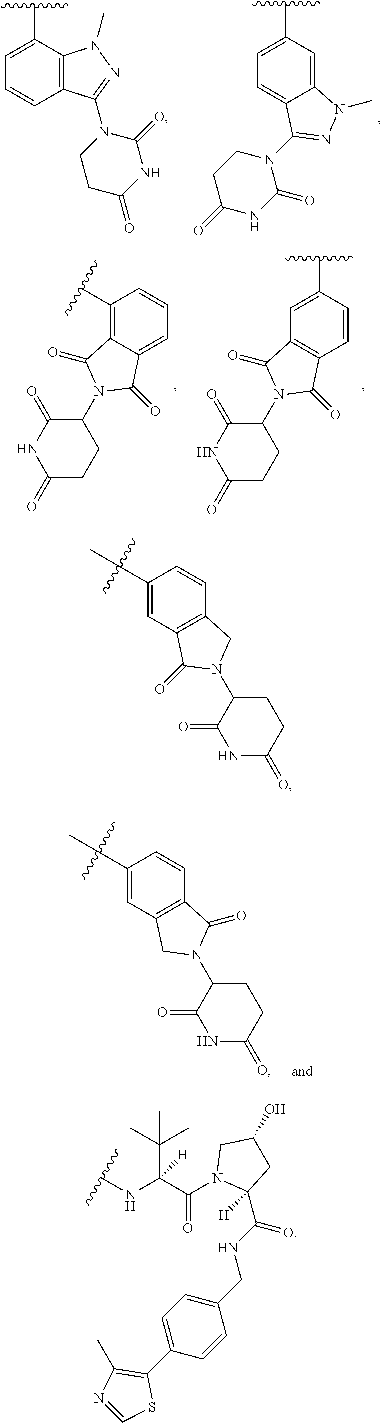

17. The compound according to claim 1, wherein the compound of formula I is selected from:

| | |
|---|---|
| 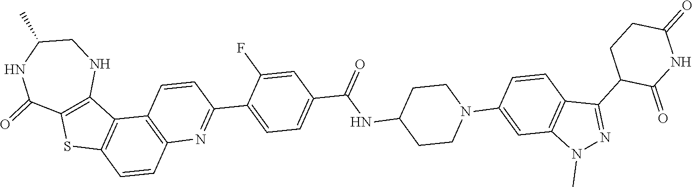 [SMILES: C[C@@H]1CNc2c(sc3ccc4nc(-c5ccc(C(=O)NC6CCN(c7ccc8c(C9CCC(=O)NC9=O)nn(C)c8c7)CC6)cc5F)ccc4c23)C(=O)N1] | Ex 1 |
| 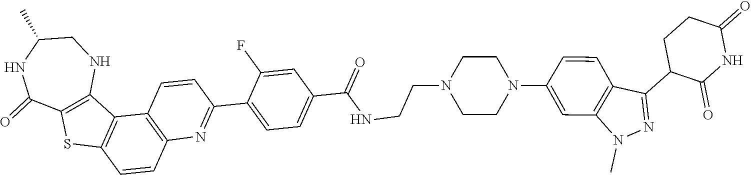 [SMILES: C[C@@H]1CNc2c(sc3ccc4nc(-c5ccc(C(=O)NCCN6CCN(c7ccc8c(C9CCC(=O)NC9=O)nn(C)c8c7)CC6)cc5F)ccc4c23)C(=O)N1] | Ex 2 |
| 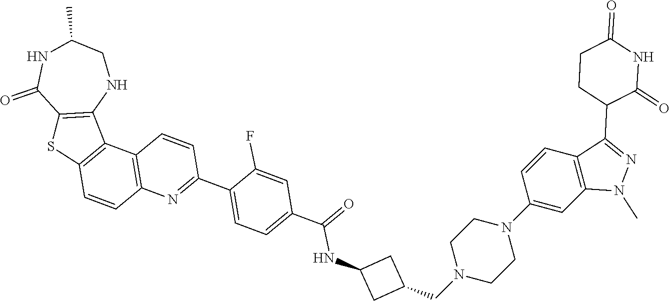 [SMILES: C[C@@H]1CNc2c(sc3ccc4nc(-c5ccc(C(=O)N[C@H]6C[C@H](CN7CCN(c8ccc9c(C%10CCC(=O)NC%10=O)nn(C)c9c8)CC7)C6)cc5F)ccc4c23)C(=O)N1] | Ex 3 |
| 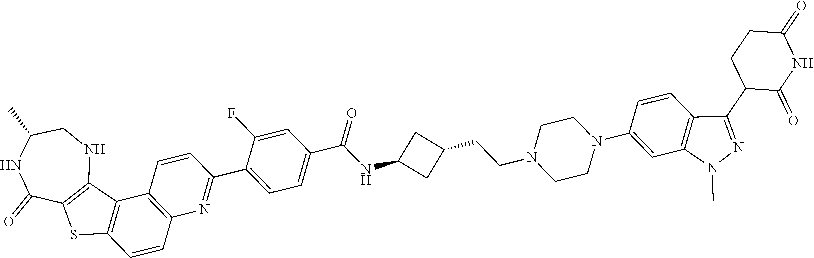 [SMILES: C[C@@H]1CNc2c(sc3ccc4nc(-c5ccc(C(=O)N[C@H]6C[C@H](CCN7CCN(c8ccc9c(C%10CCC(=O)NC%10=O)nn(C)c9c8)CC7)C6)cc5F)ccc4c23)C(=O)N1] | Ex 4 |
| 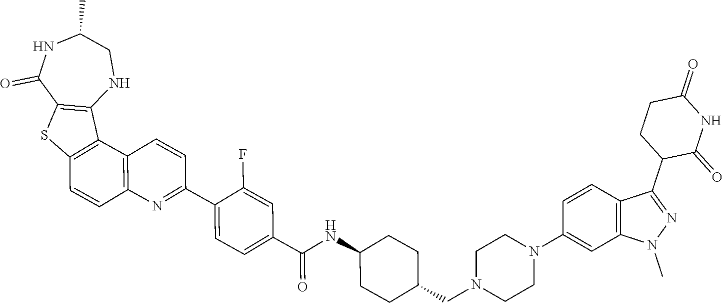 [SMILES: C[C@@H]1CNc2c(sc3ccc4nc(-c5ccc(C(=O)N[C@H]6CC[C@H](CN7CCN(c8ccc9c(C%10CCC(=O)NC%10=O)nn(C)c9c8)CC7)CC6)cc5F)ccc4c23)C(=O)N1] | Ex 5 |
| 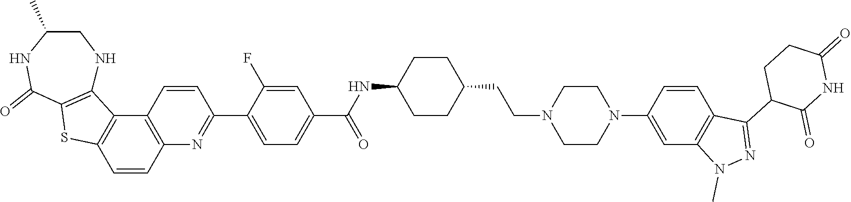 [SMILES: C[C@@H]1CNc2c(sc3ccc4nc(-c5ccc(C(=O)N[C@H]6CC[C@H](CCN7CCN(c8ccc9c(C%10CCC(=O)NC%10=O)nn(C)c9c8)CC7)CC6)cc5F)ccc4c23)C(=O)N1] | Ex 6 |
| 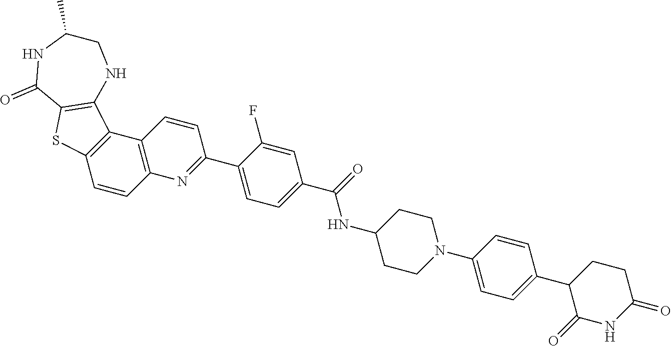 [SMILES: C[C@@H]1CNc2c(sc3ccc4nc(-c5ccc(C(=O)NC6CCN(c7ccc(C8CCC(=O)NC8=O)cc7)CC6)cc5F)ccc4c23)C(=O)N1] | Ex 7 |
| 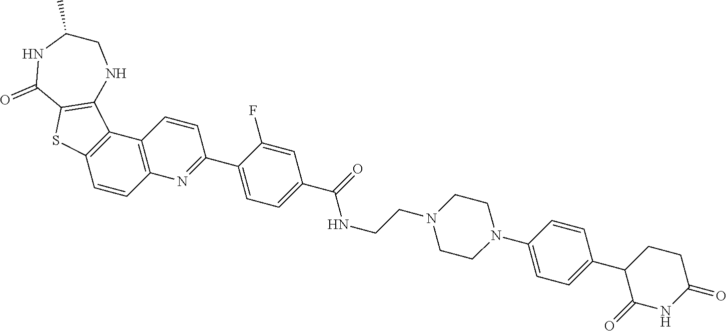 [SMILES: C[C@@H]1CNc2c(sc3ccc4nc(-c5ccc(C(=O)NCCN6CCN(c7ccc(C8CCC(=O)NC8=O)cc7)CC6)cc5F)ccc4c23)C(=O)N1] | Ex 8 |
| 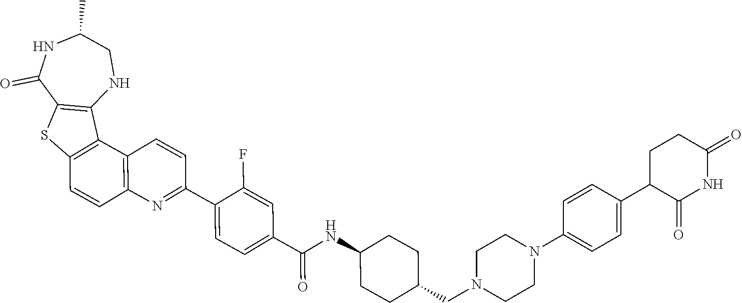 [SMILES: C[C@@H]1CNc2c(sc3ccc4nc(-c5ccc(C(=O)N[C@H]6CC[C@H](CN7CCN(c8ccc(C9CCC(=O)NC9=O)cc8)CC7)CC6)cc5F)ccc4c23)C(=O)N1] | Ex 9 |
| 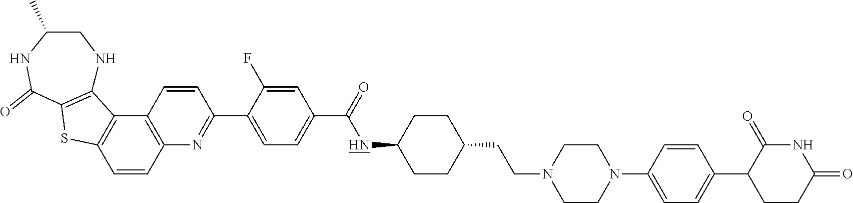 [SMILES: C[C@@H]1CNc2c(sc3ccc4nc(-c5ccc(C(=O)N[C@H]6CC[C@H](CCN7CCN(c8ccc(C9CCC(=O)NC9=O)cc8)CC7)CC6)cc5F)ccc4c23)C(=O)N1] | Ex 10 |
| 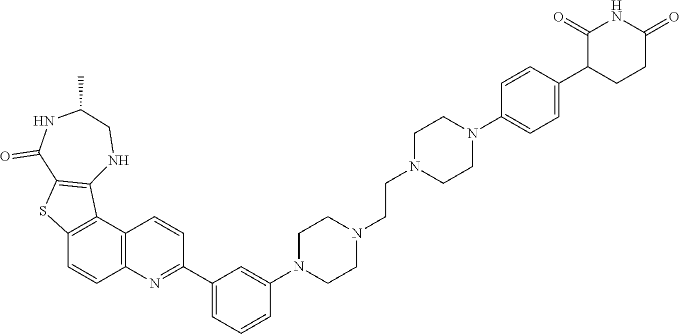 [SMILES: C[C@@H]1CNc2c(sc3ccc4nc(-c5cccc(N6CCN(CCN7CCN(c8ccc(C9CCC(=O)NC9=O)cc8)CC7)CC6)c5)ccc4c23)C(=O)N1] | Ex 11 |
| 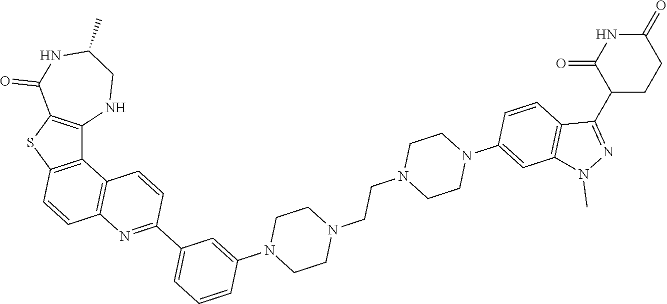 [SMILES: C[C@@H]1CNc2c(sc3ccc4nc(-c5cccc(N6CCN(CCN7CCN(c8ccc9c(C%10CCC(=O)NC%10=O)nn(C)c9c8)CC7)CC6)c5)ccc4c23)C(=O)N1] | Ex 12 |
| 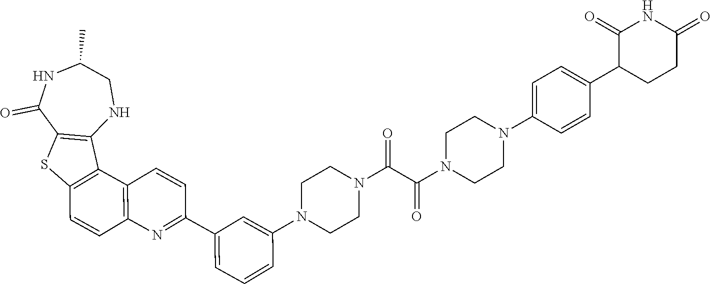 [SMILES: C[C@@H]1CNc2c(sc3ccc4nc(-c5cccc(N6CCN(C(=O)C(=O)N7CCN(c8ccc(C9CCC(=O)NC9=O)cc8)CC7)CC6)c5)ccc4c23)C(=O)N1] | Ex 13 |
| 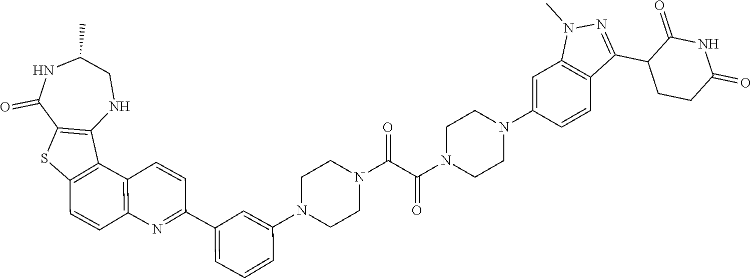 [SMILES: C[C@@H]1CNc2c(sc3ccc4nc(-c5cccc(N6CCN(C(=O)C(=O)N7CCN(c8ccc9c(C%10CCC(=O)NC%10=O)nn(C)c9c8)CC7)CC6)c5)ccc4c23)C(=O)N1] | Ex 14 |
| 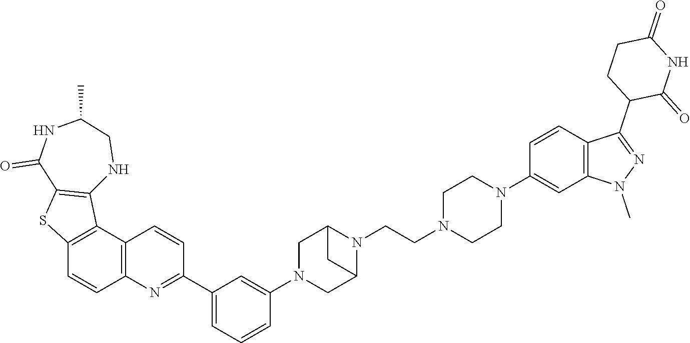 [SMILES: C[C@@H]1CNc2c(sc3ccc4nc(-c5cccc(N6CC7CC(C6)N7CCN6CCN(c7ccc8c(C9CCC(=O)NC9=O)nn(C)c8c7)CC6)c5)ccc4c23)C(=O)N1] | Ex 15 |
| 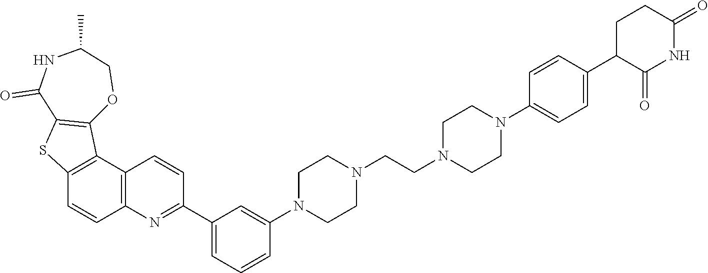 [SMILES: C[C@@H]1COc2c(sc3ccc4nc(-c5cccc(N6CCN(CCN7CCN(c8ccc(C9CCC(=O)NC9=O)cc8)CC7)CC6)c5)ccc4c23)C(=O)N1] | Ex 16 |
| 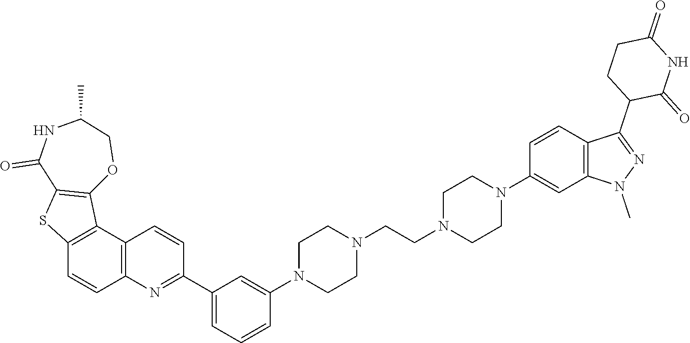 [SMILES: C[C@@H]1COc2c(sc3ccc4nc(-c5cccc(N6CCN(CCN7CCN(c8ccc9c(C%10CCC(=O)NC%10=O)nn(C)c9c8)CC7)CC6)c5)ccc4c23)C(=O)N1] | Ex 17 |
| 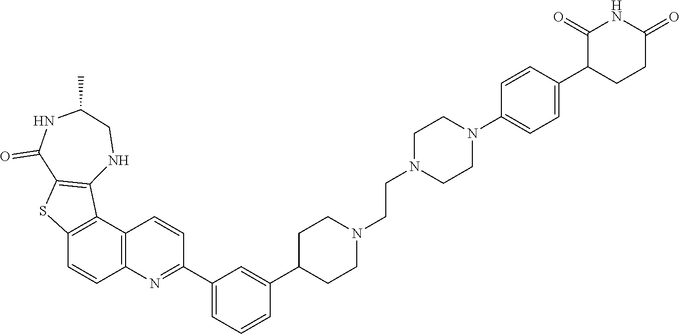 [SMILES: C[C@@H]1CNc2c(sc3ccc4nc(-c5cccc(C6CCN(CCN7CCN(c8ccc(C9CCC(=O)NC9=O)cc8)CC7)CC6)c5)ccc4c23)C(=O)N1] | Ex 18 |
| 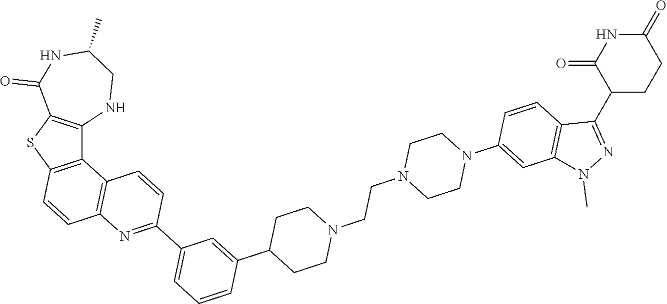 [SMILES: C[C@@H]1CNc2c(sc3ccc4nc(-c5cccc(C6CCN(CCN7CCN(c8ccc9c(C%10CCC(=O)NC%10=O)nn(C)c9c8)CC7)CC6)c5)ccc4c23)C(=O)N1] | Ex 19 |
| 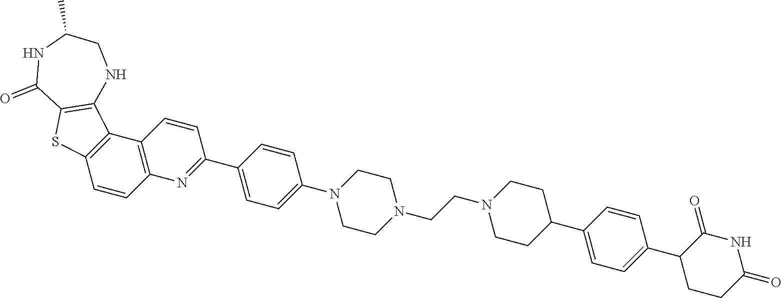 [SMILES: C[C@@H]1CNc2c(sc3ccc4nc(-c5ccc(N6CCN(CCN7CCC(c8ccc(C9CCC(=O)NC9=O)cc8)CC7)CC6)cc5)ccc4c23)C(=O)N1] | Ex 20 |
| 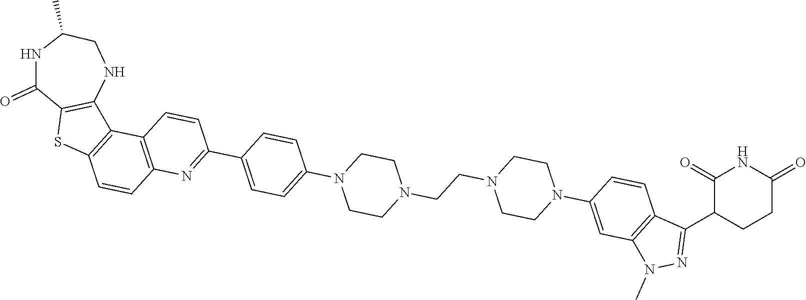 [SMILES: C[C@@H]1CNc2c(sc3ccc4nc(-c5ccc(N6CCN(CCN7CCN(c8ccc9c(C%10CCC(=O)NC%10=O)nn(C)c9c8)CC7)CC6)cc5)ccc4c23)C(=O)N1] | Ex 21 |
| 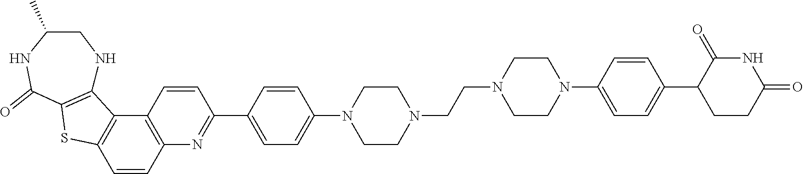 [SMILES: C[C@@H]1CNc2c(sc3ccc4nc(-c5ccc(N6CCN(CCN7CCN(c8ccc(C9CCC(=O)NC9=O)cc8)CC7)CC6)cc5)ccc4c23)C(=O)N1] | Ex 22 |
| 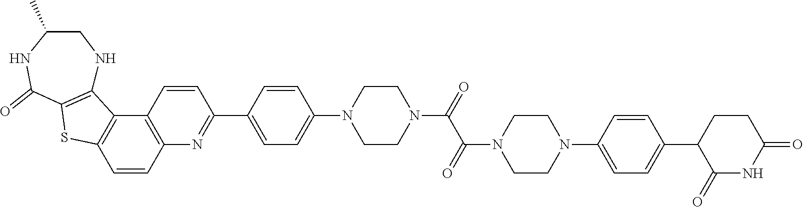 [SMILES: C[C@@H]1CNc2c(sc3ccc4nc(-c5ccc(N6CCN(C(=O)C(=O)N7CCN(c8ccc(C9CCC(=O)NC9=O)cc8)CC7)CC6)cc5)ccc4c23)C(=O)N1] | Ex 23 |
| 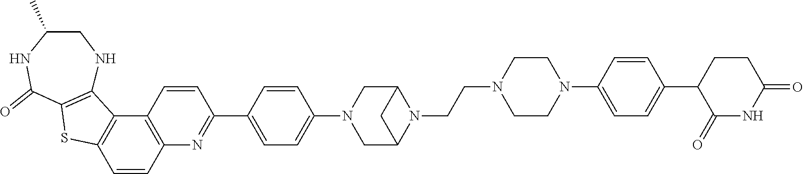 [SMILES: C[C@@H]1CNc2c(sc3ccc4nc(-c5ccc(N6CC7CC(C6)N7CCN6CCN(c7ccc(C8CCC(=O)NC8=O)cc7)CC6)cc5)ccc4c23)C(=O)N1] | Ex 24 |
| 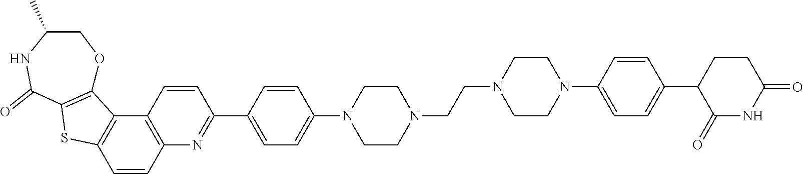 [SMILES: C[C@@H]1COc2c(sc3ccc4nc(-c5ccc(N6CCN(CCN7CCN(c8ccc(C9CCC(=O)NC9=O)cc8)CC7)CC6)cc5)ccc4c23)C(=O)N1] | Ex 25 |
| 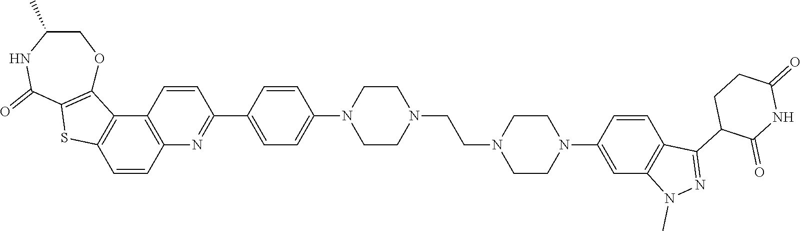 [SMILES: C[C@@H]1COc2c(sc3ccc4nc(-c5ccc(N6CCN(CCN7CCN(c8ccc9c(C%10CCC(=O)NC%10=O)nn(C)c9c8)CC7)CC6)cc5)ccc4c23)C(=O)N1] | Ex 26 |
| 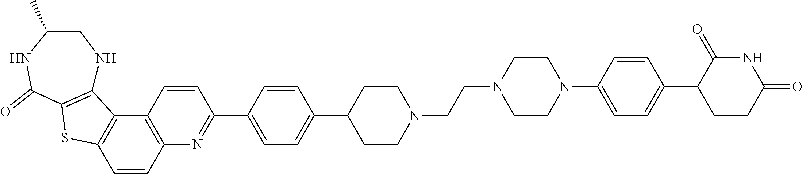 [SMILES: C[C@@H]1CNc2c(sc3ccc4nc(-c5ccc(C6CCN(CCN7CCN(c8ccc(C9CCC(=O)NC9=O)cc8)CC7)CC6)cc5)ccc4c23)C(=O)N1] | Ex 27 |
| 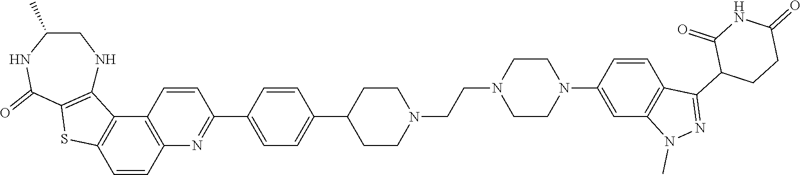 [SMILES: C[C@@H]1CNc2c(sc3ccc4nc(-c5ccc(C6CCN(CCN7CCN(c8ccc9c(C%10CCC(=O)NC%10=O)nn(C)c9c8)CC7)CC6)cc5)ccc4c23)C(=O)N1] | Ex 28 |
| 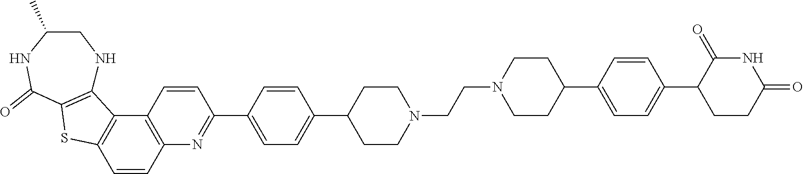 [SMILES: C[C@@H]1CNc2c(sc3ccc4nc(-c5ccc(C6CCN(CCN7CCC(c8ccc(C9CCC(=O)NC9=O)cc8)CC7)CC6)cc5)ccc4c23)C(=O)N1] | Ex 29 |
| 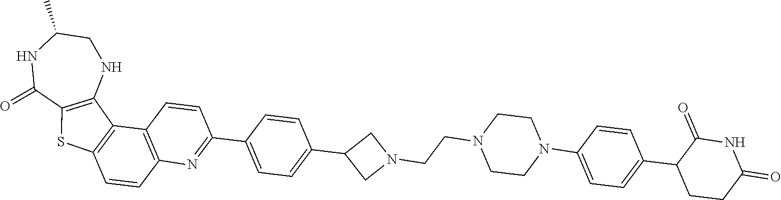 [SMILES: C[C@@H]1CNc2c(sc3ccc4nc(-c5ccc(C6CN(CCN7CCN(c8ccc(C9CCC(=O)NC9=O)cc8)CC7)C6)cc5)ccc4c23)C(=O)N1] | Ex 30 |
| 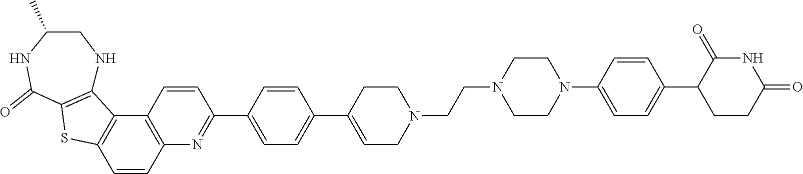 [SMILES: C[C@@H]1CNc2c(sc3ccc4nc(-c5ccc(C6=CCN(CCN7CCN(c8ccc(C9CCC(=O)NC9=O)cc8)CC7)CC6)cc5)ccc4c23)C(=O)N1] | Ex 31 |
| 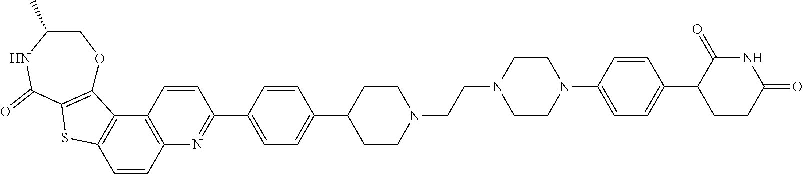 [SMILES: C[C@@H]1COc2c(sc3ccc4nc(-c5ccc(C6CCN(CCN7CCN(c8ccc(C9CCC(=O)NC9=O)cc8)CC7)CC6)cc5)ccc4c23)C(=O)N1] | Ex 32 |
| 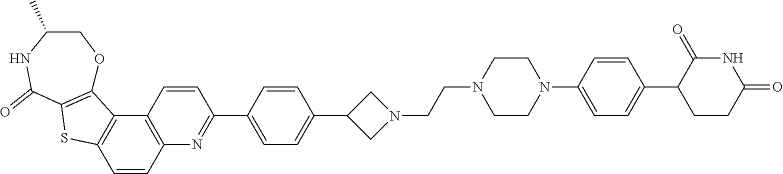 [SMILES: C[C@@H]1COc2c(sc3ccc4nc(-c5ccc(C6CN(CCN7CCN(c8ccc(C9CCC(=O)NC9=O)cc8)CC7)C6)cc5)ccc4c23)C(=O)N1] | Ex 33 |
| 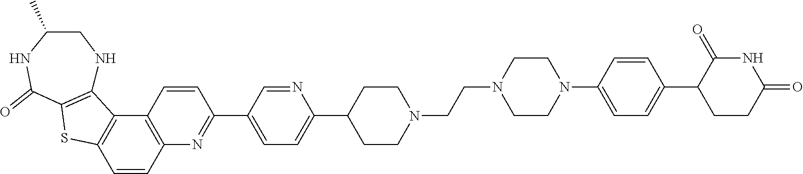 [SMILES: C[C@@H]1CNc2c(sc3ccc4nc(-c5ccc(C6CCN(CCN7CCN(c8ccc(C9CCC(=O)NC9=O)cc8)CC7)CC6)nc5)ccc4c23)C(=O)N1] | Ex 34 |
| 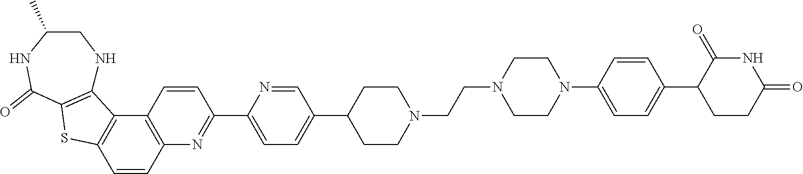 [SMILES: C[C@@H]1CNc2c(sc3ccc4nc(-c5ccc(C6CCN(CCN7CCN(c8ccc(C9CCC(=O)NC9=O)cc8)CC7)CC6)cn5)ccc4c23)C(=O)N1] | Ex 35 |
| 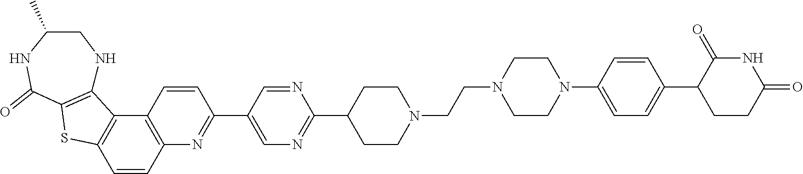 [SMILES: C[C@@H]1CNc2c(sc3ccc4nc(-c5cnc(C6CCN(CCN7CCN(c8ccc(C9CCC(=O)NC9=O)cc8)CC7)CC6)nc5)ccc4c23)C(=O)N1] | Ex 36 |
| 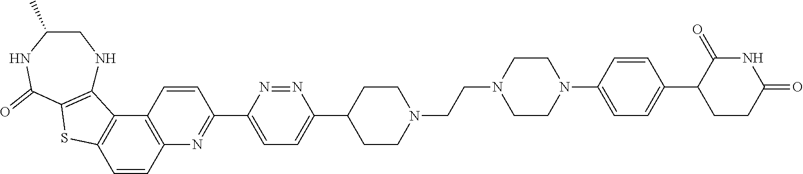 [SMILES: C[C@@H]1CNc2c(sc3ccc4nc(-c5ccc(C6CCN(CCN7CCN(c8ccc(C9CCC(=O)NC9=O)cc8)CC7)CC6)nn5)ccc4c23)C(=O)N1] | Ex 37 |
| 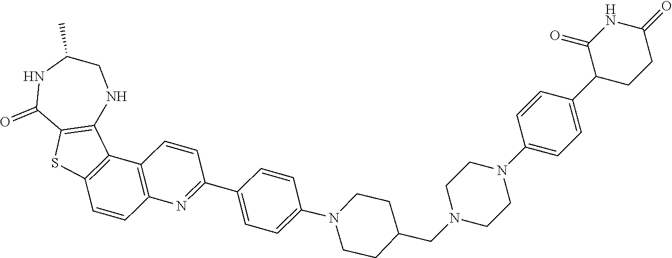 [SMILES: C[C@@H]1CNc2c(sc3ccc4nc(-c5ccc(N6CCC(CN7CCN(c8ccc(C9CCC(=O)NC9=O)cc8)CC7)CC6)cc5)ccc4c23)C(=O)N1] | Ex 38 |
| 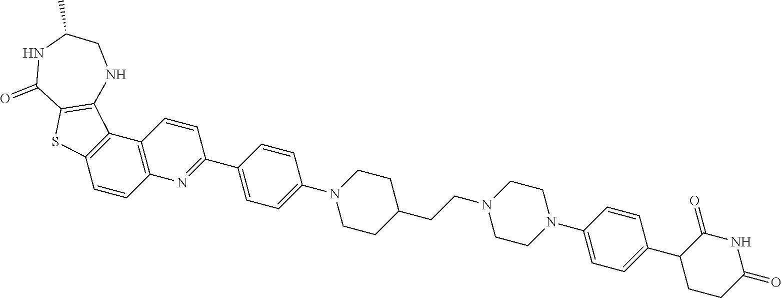 [SMILES: C[C@@H]1CNc2c(sc3ccc4nc(-c5ccc(N6CCC(CCN7CCN(c8ccc(C9CCC(=O)NC9=O)cc8)CC7)CC6)cc5)ccc4c23)C(=O)N1] | Ex 39 |
| 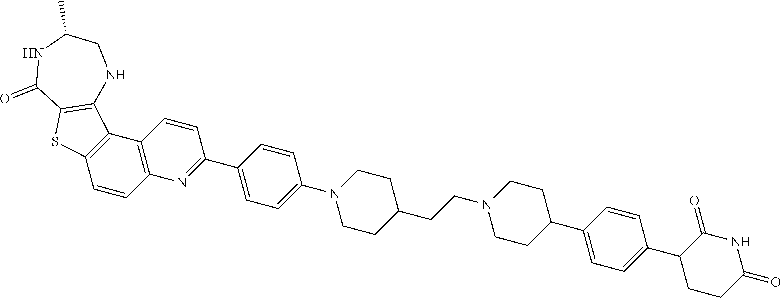 [SMILES: C[C@@H]1CNc2c(sc3ccc4nc(-c5ccc(N6CCC(CCN7CCC(c8ccc(C9CCC(=O)NC9=O)cc8)CC7)CC6)cc5)ccc4c23)C(=O)N1] | Ex 40 |
| 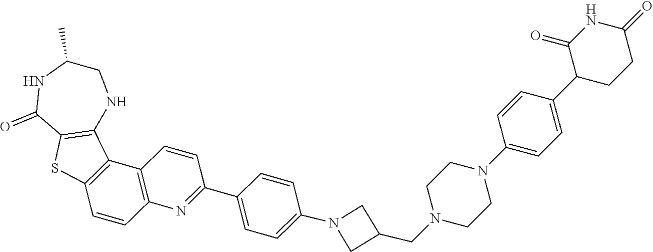 [SMILES: C[C@@H]1CNc2c(sc3ccc4nc(-c5ccc(N6CC(CN7CCN(c8ccc(C9CCC(=O)NC9=O)cc8)CC7)C6)cc5)ccc4c23)C(=O)N1] | Ex 41 |
| 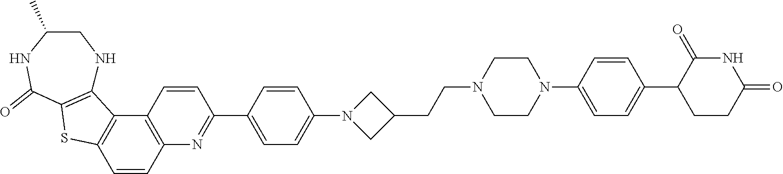 [SMILES: C[C@@H]1CNc2c(sc3ccc4nc(-c5ccc(N6CC(CCN7CCN(c8ccc(C9CCC(=O)NC9=O)cc8)CC7)C6)cc5)ccc4c23)C(=O)N1] | Ex 42 |
| 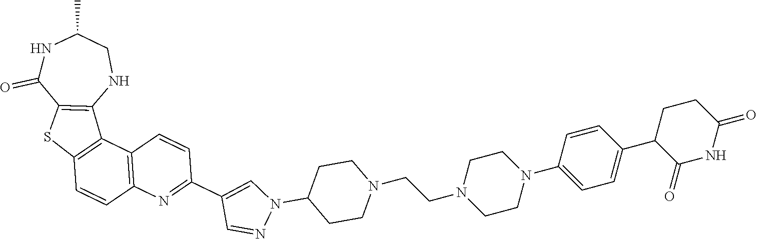 [SMILES: C[C@@H]1CNc2c(sc3ccc4nc(-c5cnn(C6CCN(CCN7CCN(c8ccc(C9CCC(=O)NC9=O)cc8)CC7)CC6)c5)ccc4c23)C(=O)N1] | Ex 43 |
| 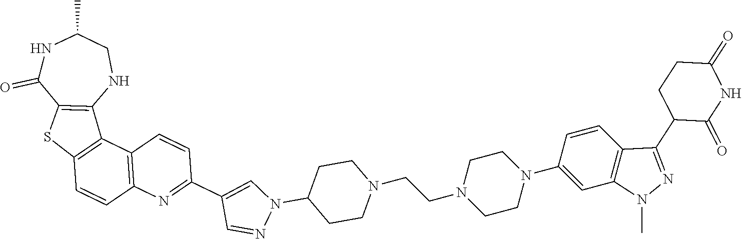 [SMILES: C[C@@H]1CNc2c(sc3ccc4nc(-c5cnn(C6CCN(CCN7CCN(c8ccc9c(C%10CCC(=O)NC%10=O)nn(C)c9c8)CC7)CC6)c5)ccc4c23)C(=O)N1] | Ex 44 |
| 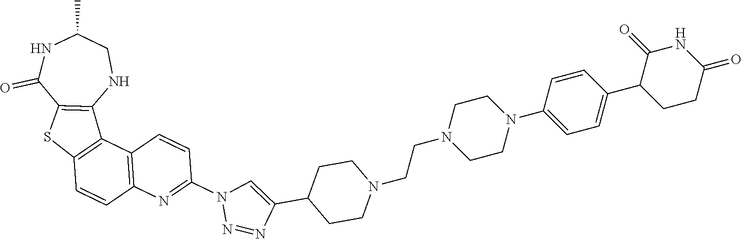 [SMILES: C[C@@H]1CNc2c(sc3ccc4nc(-n5cc(C6CCN(CCN7CCN(c8ccc(C9CCC(=O)NC9=O)cc8)CC7)CC6)nn5)ccc4c23)C(=O)N1] | Ex 45 |
| 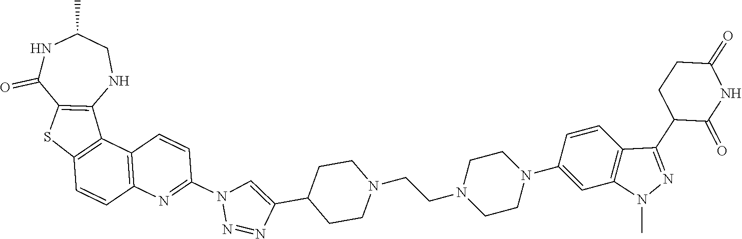 [SMILES: C[C@@H]1CNc2c(sc3ccc4nc(-n5cc(C6CCN(CCN7CCN(c8ccc9c(C%10CCC(=O)NC%10=O)nn(C)c9c8)CC7)CC6)nn5)ccc4c23)C(=O)N1] | Ex 46 |
| 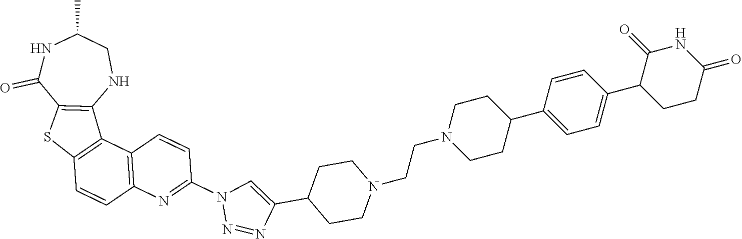 [SMILES: C[C@@H]1CNc2c(sc3ccc4nc(-n5cc(C6CCN(CCN7CCC(c8ccc(C9CCC(=O)NC9=O)cc8)CC7)CC6)nn5)ccc4c23)C(=O)N1] | Ex 47 |
| 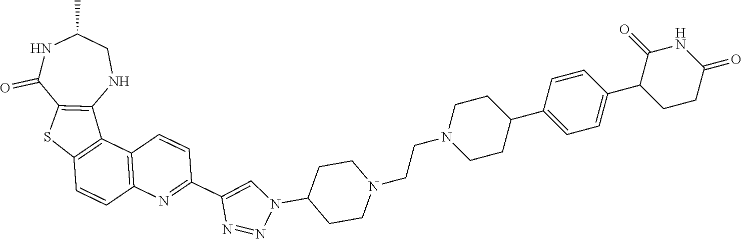 [SMILES: C[C@@H]1CNc2c(sc3ccc4nc(-c5cn(C6CCN(CCN7CCC(c8ccc(C9CCC(=O)NC9=O)cc8)CC7)CC6)nn5)ccc4c23)C(=O)N1] | Ex 48 |
| 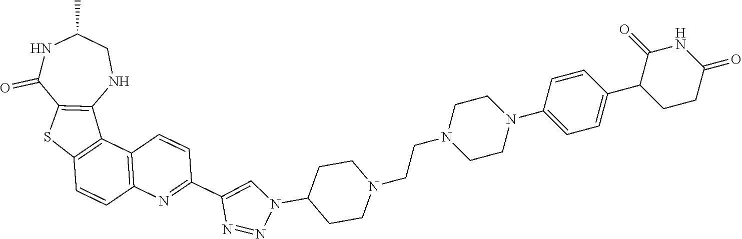 [SMILES: C[C@@H]1CNc2c(sc3ccc4nc(-c5cn(C6CCN(CCN7CCN(c8ccc(C9CCC(=O)NC9=O)cc8)CC7)CC6)nn5)ccc4c23)C(=O)N1] | Ex 49 |
| 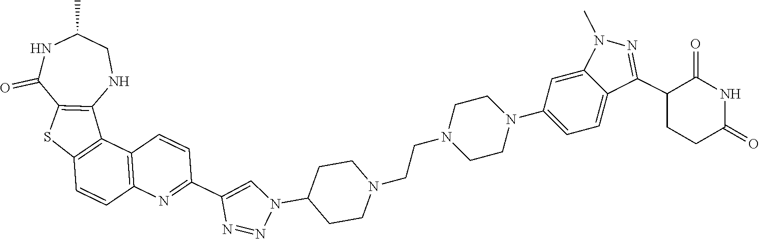 [SMILES: C[C@@H]1CNc2c(sc3ccc4nc(-c5cn(C6CCN(CCN7CCN(c8ccc9c(C%10CCC(=O)NC%10=O)nn(C)c9c8)CC7)CC6)nn5)ccc4c23)C(=O)N1] | Ex 50 |
| 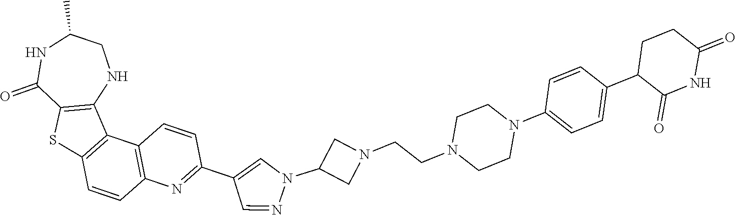 [SMILES: C[C@@H]1CNc2c(sc3ccc4nc(-c5cnn(C6CN(CCN7CCN(c8ccc(C9CCC(=O)NC9=O)cc8)CC7)C6)c5)ccc4c23)C(=O)N1] | Ex 51 |
| 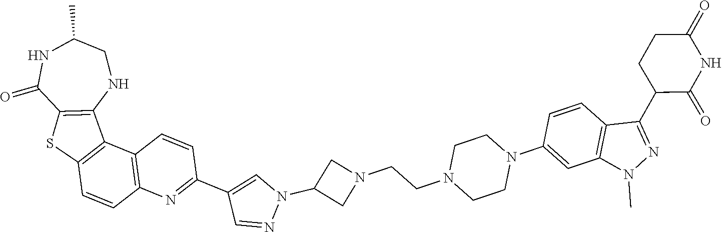 [SMILES: C[C@@H]1CNc2c(sc3ccc4nc(-c5cnn(C6CN(CCN7CCN(c8ccc9c(C%10CCC(=O)NC%10=O)nn(C)c9c8)CC7)C6)c5)ccc4c23)C(=O)N1] | Ex 52 |
| 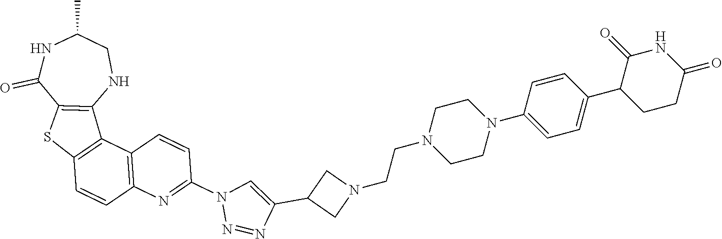 [SMILES: C[C@@H]1CNc2c(sc3ccc4nc(-n5cc(C6CN(CCN7CCN(c8ccc(C9CCC(=O)NC9=O)cc8)CC7)C6)nn5)ccc4c23)C(=O)N1] | Ex 53 |
| 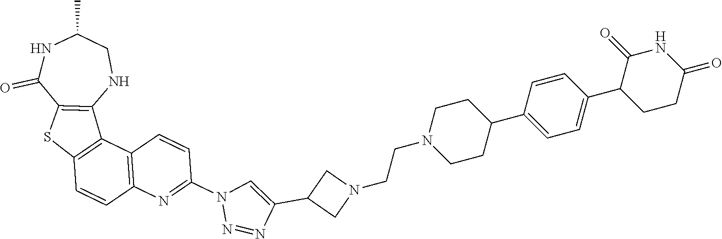 [SMILES: C[C@@H]1CNc2c(sc3ccc4nc(-n5cc(C6CN(CCN7CCC(c8ccc(C9CCC(=O)NC9=O)cc8)CC7)C6)nn5)ccc4c23)C(=O)N1] | Ex 54 |
| 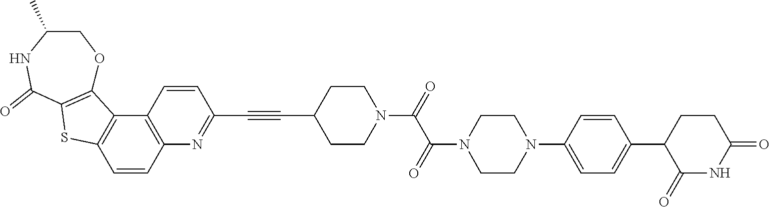 [SMILES: C[C@@H]1COc2c(sc3ccc4nc(C#CC5CCN(C(=O)C(=O)N6CCN(c7ccc(C8CCC(=O)NC8=O)cc7)CC6)CC5)ccc4c23)C(=O)N1] | Ex 55 |

or a pharmaceutically acceptable salt thereof.

18. A pharmaceutical composition comprising a compound according to claim 1, and a pharmaceutically acceptable excipient, carrier, or diluent.

19. A method of inhibiting activity of MK2, or a mutant thereof, the method comprising contacting a biological sample with a compound according to claim 1, or the pharmaceutical composition according to claim 18.

20. A method of treating a disease, disorder, or condition mediated by MK2, or a mutant thereof, the method comprising administering to a patient in need thereof a compound according to claim 1, or a pharmaceutical composition according to claim 18.


SEQUENCE LISTING (1 sequence)

SEQ ID NO: 1 - protein, 400 aa, Homo sapiens
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKEEMTSALATMRVDYEQIKIKKIEDASNPLLLKRRKKARALEAAALAH